(19) United States
(12) Patent Application Publication
(10) Pub. No.: US 2016/0150782 A1
(43) Pub. Date: Jun. 2, 2016

(54) USE OF SELECTED PYRIDONE CARBOXAMIDES OR SALTS THEREOF AS ACTIVE SUBSTANCES AGAINST ABIOTIC PLANT STRESS

(71) Applicant: BAYER CROPSCIENCE AKTIENGESELLSCHAFT, Monheim am Rhein (DE)

(72) Inventors: Lars ARVE, Hofheim (DE); Frank ZIEMER, Kriftel (DE); Dirk SCHMUTZLER, Hattersheim (DE); Jan DITTGEN, Frankfurt (DE); Ines HEINEMANN, Hofheim (DE); Martin JEFFREY HILLS, Idstein (DE)

(21) Appl. No.: 14/903,034

(22) PCT Filed: Jul. 7, 2014
(86) PCT No.: PCT/EP2014/064406

§ 371 Date: Jan. 5, 2016

(30) Foreign Application Priority Data
Jul. 9, 2013 (EP) 13175770.0

(51) Int. Cl. A01N 43/40; C07D 413/12; A01N 43/84; C07D 401/12; C07D 417/06; C07D 417/12; A01N 43/78; C07D 413/06; C07D 401/06; C07D 213/82; C07D 405/12

(52) U.S. Cl. CPC A01N 43/40; C07D 213/82; C07D 413/12; A01N 43/84; C07D 401/12; C07D 405/12; C07D 417/12; A01N 43/78; C07D 413/06; C07D 401/06; C07D 417/06

(57) ABSTRACT

Use of selected pyridone carboxamides or salts thereof as active substances against abiotic plant stress

The use of substituted pyridonecarboxamides of the general formula (I) or salts thereof

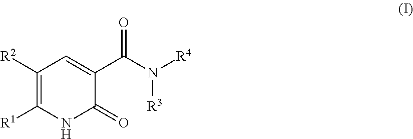

where the radicals in the general formula (I) correspond to the definitions given in the description,
for increasing stress tolerance in plants to abiotic stress, for enhancing plant growth and/or for increasing plant yield, and to specific processes for preparing the aforementioned compounds.

The invention relates to substituted pyridonecarboxamides and analogs thereof, to processes for preparation thereof and to the use thereof for increasing stress tolerance in plants to abiotic stress, especially for enhancing plant growth and/or for increasing plant yield.

It is known that particular pyridonecarboxamides can display crop plant-protective action with respect to agrochemicals which, when applied alone, cause damage to the crop plants (cf. EP 2008003016). Additionally known from the literature are active ingredients from the chemical class of the pyridones having pesticidal properties.

Different biological effects are described; for example, WO 2001/014339 mentions the fungicidal action of particular substituted pyridonecarboxamides, and WO 2005/042492 and WO 2005/042493 describe, inter alia, the fungicidal action of heterocyclylcarboxamides.

EP-A-544151 describes the action of hydroxy-substituted pyridonecarboxamides as herbicides.

EP 1 987 717 describes selected pyridone derivatives and the use thereof as safeners, i.e. for reduction of phytotoxic effects of agrochemicals, especially of herbicides, on crop plants.

WO2001/14339 describes selected heterocyclic aromatic amides and the fungicidal action thereof.

WO2013/037955 describes the use of compounds from the group of the acylsulfonamides, especially N-[4-(cyclopropylcarbamoyl)phenylsulfonyl]-2-methoxybenzamide (cyprosulfamide), for increasing yield in crop plants, either applied alone or in combined application with active ingredients of different active ingredient classes. Pyridonecarboxamides are mentioned in generic form as possible mixing partners.

Additionally known are representatives having pharmacological properties. For instance, WO 2001/055115 describes nicotinanilides as inductors of apoptosis, US 2004/0116479 describes dialkylnicotinamides as inhibitors of angiogenesis and JP 2007186434 describes pyridine analogs as vascular endothelial nitrogen oxide promoters.

In addition, EP-A-522392 describes 6-trifluoromethyl-substituted pyridonecarboxamides as precursors for synthesis of herbicidally active sulfonylureas. Chemistry of Heterocyclic Compounds, vol. 40, no. 9, 2004, 1155-1161 describes N-benzyl-5,6-di methyl-2-oxo-dihydropyridine-3-carboxamide as reaction product.

Use of compounds of this kind for increasing tolerance to abiotic stress not triggered by pesticides, preferably not by herbicides, is not yet known.

It is known that plants react to natural stress conditions, i.e. those not triggered by pesticides, for example cold, heat, drought, injury, attack by pathogens (viruses, bacteria, fungi, insects) etc., but also to herbicides, with specific or unspecific defense mechanisms [Pflanzenbiochemie (Plant Biochemistry), p. 393-462, Spektrum Akademischer Verlag, Heidelberg, Berlin, Oxford, Hans W. Heldt, 1996.; Biochemistry and Molecular Biology of Plants, p. 1102-1203, American Society of Plant Physiologists, Rockville, Md., eds. Buchanan, Gruissem, Jones, 2000].

Numerous proteins in plants, and the genes that code for them, which are involved in defense reactions to abiotic stress (for example cold, heat, drought, salt, flooding) are known. Some of these form part of signal transduction chains (e.g. transcription factors, kinases, phosphatases) or cause a physiological response of the plant cell (e.g. ion transport, detoxification of reactive oxygen species). The signaling chain genes of the abiotic stress reaction include transcription factors of the DREB and CBF classes (Jaglo-Ottosen et al., 1998, Science 280: 104-106). Phosphatases of the ATPK and MP2C type are involved in the reaction to salt stress. In addition, in the event of salt stress, the biosynthesis of osmolytes such as proline or sucrose is frequently activated. This involves, for example, sucrose synthase and proline transporters (Hasegawa et al., 2000, Annu Rev Plant Physiol Plant Mol Biol 51: 463-499). The stress defense of the plants to cold and drought uses some of the same molecular mechanisms. There is a known accumulation of what are called late embryogenesis abundant proteins (LEA proteins), which include the dehydrins as an important class (Ingram and Bartels, 1996, Annu Rev Plant Physiol Plant Mol Biol 47: 277-403, Close, 1997, Physiol Plant 100: 291-296). These are chaperones which stabilize vesicles, proteins and membrane structures in stressed plants (Bray, 1993, Plant Physiol 103: 1035-1040). In addition, there is frequently induction of aldehyde dehydrogenases, which detoxify the reactive oxygen species (ROS) which form in the event of oxidative stress (Kirch et al., 2005, Plant Mol Biol 57: 315-332).

Heat shock factors (HSF) and heat shock proteins (HSP) are activated in the event of heat stress and play a similar role here as chaperones to that of dehydrins in the event of cold and drought stress (Yu et al., 2005, Mol Cells 19: 328-333).

A number of plant-endogenous signaling substances involved in stress tolerance or pathogen defense are already known. Mention should be made here, for example, of salicylic acid, benzoic acid, jasmonic acid or ethylene [Biochemistry and Molecular Biology of Plants, p. 850-929, American Society of Plant Physiologists, Rockville, Md., eds. Buchanan, Gruissem, Jones, 2000]. Some of these substances or the stable synthetic derivatives and derived structures thereof are also effective on external application to plants or in seed dressing, and activate defense reactions which cause elevated stress tolerance or pathogen tolerance of the plant [Sembdner, and Parthier, 1993, Ann. Rev. Plant Physiol. Plant Mol. Biol. 44: 569-589]. It is also known that chemical substances can increase the tolerance of plants to abiotic stress. Such substances are applied either by seed dressing, by leaf spraying or by soil treatment. For instance, an increase in abiotic stress tolerance of crop plants by treatment with elicitors of systemic acquired resistance (SAR) or abscisic acid derivatives is described (Schading and Wei, WO-200028055, Abrams and Gusta, U.S. Pat. No. 5,201,931, Churchill et al., 1998, Plant Growth Regul 25: 35-45) or azibenzolar-S-methyl. Similar effects are also observed on application of fungicides, especially from the group of the strobilurins or of the succinate dehydrogenase inhibitors, and are frequently also accompanied by an increase in yield (Draber et al., DE-3534948, Bartlett et al., 2002, Pest Manag Sci 60: 309). It is likewise known that the herbicide glyphosate in low dosage stimulates the growth of some plant species (Cedergreen, Env. Pollution 2008, 156, 1099).

In addition, effects of growth regulators on the stress tolerance of crop plants have been described (Morrison and Andrews, 1992, J Plant Growth Regul 11: 113-117, RD-259027). In the event of osmotic stress, a protective effect has been observed as a result of application of osmolytes, for example glycine betaine or the biochemical precursors thereof, e.g. choline derivatives (Chen et al., 2000, Plant Cell Environ 23: 609-618, Bergmann et al., DE-4103253). The effect of antioxidants, for example naphthols and xanthines, to increase abiotic stress tolerance in plants has also already been described (Bergmann et al., DD-277832, Bergmann et al., DD-277835). However, the molecular causes of the antistress action of these substances are largely unknown.

It is additionally known that the tolerance of plants to abiotic stress can be increased by a modification of the activity of endogenous poly-ADP-ribose polymerases (PARP) or poly-(ADP-ribose) glycohydrolases (PARG) (de Block et al., The Plant Journal, 2005, 41, 95; Levine et al., FEBS Lett. 1998, 440, 1; WO0004173; WO04090140).

It is thus known that plants possess several endogenous reaction mechanisms which can result in an effective defense against a wide variety of harmful organisms and/or natural abiotic stress.

Since, however, the environmental and economic demands on modern crop treatment compositions are increasing constantly, for example with respect to toxicity, selectivity, application rate, formation of residues and favorable manufacture, there is a constant need to develop novel crop treatment compositions which have advantages over those known, at least in some areas.

It was therefore an object of the present invention to provide further compounds which increase tolerance to natural abiotic stress in plants, i.e. not triggered by pesticides, preferably not by herbicides.

The present invention accordingly provides for the use of substituted pyridonecarboxamides of the general formula (I), or salts thereof,

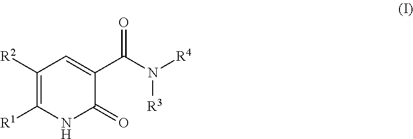

for increasing tolerance to abiotic stress in plants, where
R¹ is (C₁-C₆)-alkyl, (C₂-C₆)-alkenyl, (C₂-C₆)-alkynyl, (C₁-C₆)-haloalkyl, (C₂-C₆)-haloalkenyl, (C₂-C₆)-haloalkynyl, (C₃-C₆)-cycloalkyl, (C₃-C₆)-cycloalkyl-(C₁-C₆)-alkyl, (C₁-C₆)-alkoxy, (C₂-C₆)-alkenyloxy, (C₂-C₆)-alkynyloxy, (C₁-C₆)-haloalkoxy, (C₂-C₆)-haloalkenyloxy, (C₂-C₆)-haloalkynyloxy, cyano, halogen or optionally mono- or polysubstituted phenyl,
R² is (C₁-C₆)-alkyl, (C₂-C₆)-alkenyl, (C₂-C₆)-alkynyl, (C₁-C₆)-haloalkyl, (C₂-C₆)-haloalkenyl, (C₂-C₆)-haloalkynyl, (C₃-C₆)-cycloalkyl, (C₃-C₆)-cycloalkyl-(C₁-C₆)-alkyl, (C₁-C₆)-alkoxy, (C₂-C₆)-alkenyloxy, (C₂-C₆)-alkynyloxy, (C₁-C₆)-haloalkoxy, (C₂-C₆)-haloalkenyloxy, (C₂-C₆)-haloalkynyloxy, cyano, halogen or optionally mono- or polysubstituted phenyl,
R³ and R⁴ are each independently hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
   or
   are (C₃-C₆)-cycloalkyl, (C₄-C₆)cycloalkenyl, (C₃-C₆)-cycloalkyl fused on one side of the ring to a 4- to 6-membered saturated or unsaturated carbocyclic ring, or (C₄-C₆)-cycloalkenyl fused on one side of the ring to a 4- to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
   or
   are phenyl which is unsubstituted or substituted, heteroaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, hydroxycarbonyl, [(C₁-C₄)-alkoxy]-carbonyl and (C₁-C₄)-alkylthio,
   or
R³ and R⁴ together with the directly bonded nitrogen atom are the —N═CR⁵—NR⁶R⁷ group where
R⁵ is hydrogen or (C₁-C₆)-alkyl and
R⁶ and R⁷ are each independently hydrogen or (C₁-C₆)-alkyl, or R⁶, R⁷ together with the directly bonded nitrogen atom form a five- to seven-membered, preferably saturated, heterocyclic ring, for example piperidinyl, pyrrolidinyl or morpholinyl.

The compounds of the formula (I) and their salts, in some cases, are also referred to hereinafter as “compounds (I)” according to the invention or used in accordance with the invention.

The compounds of the formula (I) also include tautomers which can be formed by a hydrogen shift and which are not covered formally in structural terms by the formula (I). Nevertheless, these tautomers are considered to be encompassed by the definition of the inventive compounds of the formula (I). More particularly, the definition of the compounds of the formula (I) encompasses the tautomeric structures of the formula (Ia) (2-hydroxypyridine-3-carboxamides), or salts thereof,

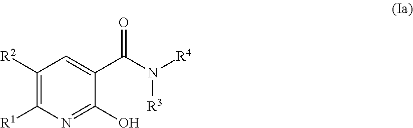

in which
R¹, R², R³ and R⁴ are each as defined in formula (I).

Preference is given to the inventive use of compounds of the general formula (I), or salts thereof, in which
R¹ is (C₁-C₆)-alkyl, (C₁-C₆)-haloalkyl, (C₃-C₆)-cycloalkyl, (C₁-C₆)-alkoxy or (C₁-C₆)-haloalkoxy,
R² is (C₁-C₆)-alkyl, (C₁-C₆)-haloalkyl, (C₃-C₆)-cycloalkyl, (C₁-C₆)-alkoxy, (C₁-C₆)-haloalkoxy or halogen,
R³ and R⁴ are each independently hydrogen, (C₁-C₁₆)-alkyl, (C₂-C₁₆)-alkenyl or (C₂-C₁₆)-alkynyl, where each of the 3 latter radicals unsubstituted or by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
   or
   are (C₃-C₆)-cycloalkyl, (C₄-C₆)cycloalkenyl, (C₃-C₆)-cycloalkyl fused on one side of the ring to a 4- to 6-membered saturated or unsaturated carbocyclic ring, or (C₄-C₆)-cycloalkenyl fused on one side of the ring to a 4- to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
   or
   are phenyl which is unsubstituted or substituted, heteroaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, hydroxycarbonyl, [(C₁-C₄)-alkoxy]-carbonyl and (C₁-C₄)-alkylthio or R³ and R⁴ together with the directly bonded nitrogen atom are the —N═CR⁵—NR⁶R⁷ group and where
R⁵ is hydrogen or (C₁-C₆)-alkyl and
R⁶ and R⁷ are each independently hydrogen or (C₁-C₆)-alkyl, or
R⁶ and R⁷ together with the directly bonded nitrogen atom form a five- to seven-membered, preferably saturated, heterocyclic ring, for example piperidinyl, pyrrolidinyl or morpholinyl.

Particular preference is given to the inventive use of compounds of the general formula (I), or salts thereof, in which
R¹ is (C₁-C₆)-alkyl or (C₁-C₆)-haloalkyl,
R² is (C₁-C₆)-alkyl or halogen,
R³ and R⁴ are each independently hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
   or
   are (C₃-C₆)-cycloalkyl, (C₄-C₆)cycloalkenyl, (C₃-C₆)-cycloalkyl fused on one side of the ring to a 4- to 6-membered saturated or unsaturated carbocyclic ring, or (C₄-C₆)-cycloalkenyl fused on one side of the ring to a 4- to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
   or
   are phenyl which is unsubstituted or substituted, heteroaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, hydroxycarbonyl, [(C₁-C₄)-alkoxy]-carbonyl and (C₁-C₄)-alkylthio.
   or
R³ and R⁴ together with the directly bonded nitrogen atom are the —N═CR⁵—NR⁶R⁷ group and where
R⁵ is hydrogen or (C₁-C₆)-alkyl and R⁶ and R⁷ are each independently hydrogen or (C₁-C₆)-alkyl, or R⁶, R⁷ together with the directly bonded nitrogen atom form a five- to seven-membered, preferably saturated, heterocyclic ring, for example piperidinyl, pyrrolidinyl or morpholinyl.

Very particular preference is given to the inventive use of compounds of the general formula (I), or salts thereof, in which
R¹ is (C₁-C₆)-alkyl or (C₁-C₆)-haloalkyl,
R² is (C₁-C₆)-alkyl or halogen,
R³ and R⁴ are each independently hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted are,
   or
   are phenyl which is unsubstituted or substituted, heteroaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, [(C₁-C₄)-alkoxy]-carbonyl and (C₁-C₄)-alkylthio.

The aforementioned substituted pyridonecarboxamides of the general formula (I) are essentially likewise as yet unknown in the prior art.

Thus, a further part of the invention is that of substituted pyridonecarboxamides of the formula (I), or salts thereof,

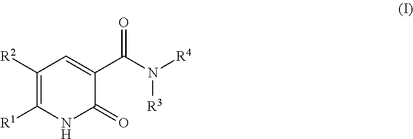

in which
R¹ is (C₁-C₆)-alkyl or (C₁-C₆)-haloalkyl,
R² is (C₁-C₆)-alkyl or halogen,
R³ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted,
   or
   is (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
   or
   is phenyl which is unsubstituted or substituted, heteroaryl which is unsubstituted or substituted and heterocyclyl which is unsubstituted or substituted
   and
R⁴ is (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted,
   or
   is (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
   or
   is phenyl which is unsubstituted or substituted, heteroaryl which is unsubstituted or substituted and heterocyclyl which is unsubstituted or substituted
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which unsubstituted or mean by one or more radicals from the group of halogen, cyano, nitro, (C₁-C₄)-alkyl, (C₁-C₄)-haloalkyl, (C₁-C₄)-alkoxy, (C₁-C₄)-haloalkoxy, hydroxycarbonyl, [(C₁-C₄)-alkoxy]-carbonyl and (C₁-C₄)-alkylthio,
   or
R³ and R⁴ together with the directly bonded nitrogen atom are the —N═CR⁵—NR⁶R⁷ group,
R⁵ is hydrogen or (C₁-C₆)-alkyl and
R⁶ and R⁷ are each independently hydrogen or (C₁-C₆)-alkyl, or R⁶ and R⁷ together with the directly bonded nitrogen atom form a five- to seven-membered, preferably saturated, heterocyclic ring, for example piperidinyl, pyrrolidinyl or morpholinyl are,
   excluding compounds of the general formula (I) in which
   (a) R¹ and R² are methyl, R³ is hydrogen and R⁴ is benzyl, or
   (b) when R¹ is (C₁-C₆)-haloalkyl and R² is simultaneously halogen.

Very particular preference is given to substituted pyridonecarboxamides of the general formula (I), or salts thereof, in which
R¹ is (C₁-C₆)-alkyl or (C₁-C₆)-haloalkyl,
R² is methyl,
R³ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted,
   or
   is phenyl which is unsubstituted or substituted, hetaryl which is unsubstituted or substituted and heterocyclyl which is unsubstituted or substituted,
   and
R⁴ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted,
   or
   is phenyl which is unsubstituted or substituted, hetaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, [(C₁-C₄)-alkoxy]-carbonyl and (C₁-C₄)-alkylthio,
   excluding compounds of the general formula (I) in which R¹ and R² are methyl, R³ is hydrogen and R⁴ is benzyl.

Very particular preference is likewise given to substituted pyridonecarboxamides of the general formula (I), or salts thereof, in which
R¹ is (C₁-C₆)-alkyl or (C₁-C₆)-haloalkyl,
R² is ethyl,
R³ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   and
R⁴ is (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, [(C₁-C₄)-alkoxy]-carbonyl and (C₁-C₄)-alkylthio.

Very particular preference is likewise given to substituted pyridonecarboxamides of the general formula (I), or salts thereof, in which
R¹ is (C₁-C₆)-alkyl or (C₁-C₆)-haloalkyl,
R² is n-propyl,
R³ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   and
R⁴ is (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, [(C₁-C₄)-alkoxy]-carbonyl and (C₁-C₄)-alkylthio are.

Very particular preference is likewise given to substituted pyridonecarboxamides of the general formula (I), or salts thereof, in which
R¹ is (C₁-C₆)-alkyl,
R² is chlorine,
R³ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   and
R⁴ is (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms,
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, [(C₁-C₄)-alkoxy]-carbonyl and (C₁-C₄)-alkylthio.

Very particular preference is likewise given to substituted pyridonecarboxamides of the general formula (I), or salts thereof, in which
R¹ is (C₁-C₆)-alkyl,
R² is bromine,
R³ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   and
R⁴ is (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, [(C₁-C₄)-alkoxy]-carbonyl and (C₁-C₄)-alkylthio.

Very particular preference is likewise given to substituted pyridonecarboxamides of the general formula (I), or salts thereof, in which
R¹ is (C₁-C₆)-alkyl,
R² is iodine,
R³ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   and
R⁴ is (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted, is
   or
   is phenyl which is unsubstituted or substituted, hetaryl which is unsubstituted or substituted and heterocyclyl which is unsubstituted or substituted,
   or
R³ is hydrogen or (C₁-C₆)-alkyl
   and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
   or
R³ and R⁴ together with the directly bonded nitrogen atom are an amino acid residue, specifically those which occur naturally in their racemic and respective D and L forms
   or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, [(C₁-C₄)-alkoxy]-carbonyl and (C₁-C₄)-alkylthio.

Thus, the invention further provides a spray solution for treatment of plants, comprising an amount, effective for increasing the resistance of plants to abiotic stress factors, of one or more of the aforementioned substituted pyridonecarboxamides, excluding the compound of the general formula (I) in which (a) simultaneously R¹ and R² are methyl and R³ is hydrogen and R⁴ is benzyl and (b) compounds of the general formula (I) in which simultaneously R¹ is (C₁-C₆)-haloalkyl and R² is halogen.

With regard to the inventive compounds, the terms used above and further below will be elucidated. These are familiar to the person skilled in the art and especially have the definitions elucidated hereinafter:

The term “halogen” means, for example, fluorine, chlorine, bromine or iodine.

If the term is used for a radical, “halogen” means, for example, a fluorine, chlorine, bromine or iodine atom.

According to the invention, alkyl means a straight-chain or branched open-chain, saturated hydrocarbyl radical which has optionally been mono- or polysubstituted. Preferred substituents are halogen atoms, alkoxy groups, haloalkoxy groups, cyano groups, alkylthio groups, haloalkylthio groups or nitro groups, particular preference being given to fluorine, chlorine, bromine or iodine.

Fluoroalkyl means a straight-chain or branched open-chain, saturated and fluorine-substituted hydrocarbyl radical where at least one fluorine atom is at one of the possible positions.

Perfluoroalkyl means a straight-chain or branched open-chain, saturated and fully fluorine-substituted hydrocarbyl radical, for example CF₃, CF₂CF₃, CF₂CF₂CF₃.

Partly fluorinated alkyl means a straight-chain or branched, saturated hydrocarbon which is mono- or polysubstituted by fluorine, where the fluorine atoms in question may be present as substituents on one or more different carbon atoms of the straight-chain or branched hydrocarbon chain, for example CHFCH₃, CH₂CH₂F, CH₂CH₂CF₃, CHF₂, CH₂F, CHFCF₂CF₃.

Partly fluorinated haloalkyl means a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbon chain. Partly fluorinated haloalkyl also includes full substitution of the straight or branched chain by halogen including at least one fluorine atom.

Haloalkyl, -alkenyl and -alkynyl mean, respectively, alkyl, alkenyl and alkynyl partly or fully substituted by identical or different halogen atoms, e.g. monohaloalkyl, for example CH₂CH₂Cl, CH₂CH₂Br, CHClCH₃, CH₂Cl, CH₂F; perhaloalkyl, for example CCl₃, CClF₂, CFCl₂, CF₂CClF₂, CF₂CClFCF₃; polyhaloalkyl, for example CH₂CHFCl, CF₂CClFH, CF₂CBrFH, CH₂CF₃; the term “perhaloalkyl” also includes the term “perfluoroalkyl”, and the term “polyhaloalkyl” also includes the terms “partly fluorinated alkyl” and “partly fluorinated haloalkyl”.

Haloalkoxy is, for example, OCF₃, OCHF₂, OCH₂F, OCF₂CF₃, OCH₂CF₃ and OCH₂CH₂Cl; the situation is equivalent for haloalkenyl and other halogen-substituted radicals.

The expression “(C₁-C₄)-alkyl” is a brief notation for alkyl having one to four carbon atoms according to the range stated for carbon atoms, i.e. comprises the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also include straight-chain or branched alkyl radicals having a greater number of carbon atoms, i.e. in the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbyl radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

Alkenyl especially also includes straight-chain or branched open-chain hydrocarbyl radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl is, for example, vinyl which may optionally be substituted by further alkyl radicals, for example prop-1-en-1-yl, but-1-en-1-yl, allyl, 1-methylprop-2-en-1-yl, 2-methylprop-2-en-1-yl, but-2-en-1-yl, 1-methylbut-3-en-1-yl and 1-methylbut-2-en-1-yl, 2-methylprop-1-en-1-yl, 1-methylprop-1-en-1-yl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl, pentenyl, 2-methylpentenyl or hexenyl.

Alkynyl especially also includes straight-chain or branched open-chain hydrocarbyl radicals having more than one triple bond, or else having one or more triple bonds and one or more double bonds, for example 1,3-butatrienyl or 3-penten-1-yn-1-yl. (C₂-C₆)-Alkynyl is, for example, ethynyl, propargyl, 1-methylprop-2-yn-1-yl, 2-butynyl, 2-pentynyl or 2-hexynyl, preferably propargyl, but-2-yn-1-yl, but-3-yn-1-yl or 1-methylbut-3-yn-1-yl.

The term “cycloalkyl” means a carbocyclic saturated ring system having preferably 3-8 ring carbon atoms, for example cyclopropyl, cyclobutyl, cyclopentyl or cyclohexyl.

Cycloalkenyl is a carbocyclic, nonaromatic, partially unsaturated ring system having preferably 4-8 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl.

The term “aryl” means a mono-, bi- or polycyclic aromatic system having preferably 6 to 14 and especially 6 to 10 ring carbon atoms, for example phenyl, naphthyl, anthryl, phenanthrenyl and the like, preferably phenyl optionally mono- or polysubstituted by a radical from the group of halogen, nitro, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, (C₃-C₆)-cycloalkyl (C₁-C₆)-alkyl, [(C₁-C₄)-alkoxy]-carbonyl, [(C₁-C₄)-alkoxy]-carbonyl-(C₁-C₄)-alkyl, hydroxycarbonyl and hydroxycarbonyl-(C₁-C₄)-alkyl.

The term “optionally substituted aryl” also includes polycyclic systems, such as tetrahydronaphthyl, indenyl, indanyl, fluorenyl, biphenylyl, where the bonding site is on the aromatic system.

In systematic terms, “aryl” is generally also encompassed by the term “optionally substituted phenyl”.

A heterocyclic radical or ring (heterocyclyl) may be saturated, unsaturated or heteroaromatic and unsubstituted or substituted, for example, by a radical from the group consisting of halogen, nitro, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulfoxy, (C₁-C₄)-alkylsulfone, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl, (C₃-C₆)-cycloalkyl (C₁-C₆)-alkyl, [(C₁-C₄)-alkoxy]-carbonyl, [(C₁-C₄)-alkoxy]-carbonyl-(C₁-C₄)-alkyl, hydroxycarbonyl and hydroxycarbonyl-(C₁-C₄)-alkyl; it preferably contains one or more heteroatoms in the ring, preferably from the group of N, O and S; it is an aliphatic heterocyclyl radical having 3 to 7 ring atoms or a heteroaromatic radical having 5 or 6 ring atoms and contains 1, 2 or 3 heteroatoms. The heterocyclic radical may, for example, be a heteroaromatic radical or ring (heteroaryl), for example a mono-, bi- or polycyclic aromatic system in which at least 1 ring contains one or more heteroatoms, for example pyridyl, pyrimidinyl, pyridazinyl, pyrazinyl, triazinyl, thienyl, thiazolyl, oxazolyl, furyl, pyrrolyl, pyrazolyl and imidazolyl, or it is a partially or fully hydrogenated radical, such as oxiranyl, oxetanyl, pyrrolidyl, piperidyl, piperazinyl, dioxolanyl, morpholinyl, tetrahydrofuryl. Suitable substituents for a substituted heterocyclic radical are the substituents specified later on below, and additionally also oxo. The oxo group may also occur on the ring heteroatoms which are able to exist in different oxidation states, as in the case of N and S, for example.

Alkoxy means an alkyl radical bonded via an oxygen atom, alkenyloxy means an alkenyl radical bonded via an oxygen atom, alkynyloxy means an alkynyl radical bonded via an oxygen atom, cycloalkyloxy means a cycloalkyl radical bonded via an oxygen atom, and cycloalkenyloxy means a cycloalkenyl radical bonded via an oxygen atom.

According to the invention, “alkylthio”—alone or as part of a chemical group—is straight-chain or branched S-alkyl, preferably having 1 to 8 or having 1 to 6 carbon atoms, for example methylthio, ethylthio, n-propylthio, isopropylthio, n-butylthio, isobutylthio, sec-butylthio and tert-butylthio. Alkenylthio means an alkenyl radical bonded via a sulfur atom, alkynylthio is an alkynyl radical bonded via a sulfur atom, cycloalkylthio is a cycloalkyl radical bonded via a sulfur atom, and cycloalkenylthio is a cycloalkenyl radical bonded via a sulfur atom.

According to the invention, “alkylsulfinyl”—alone or as part of a chemical group—is straight-chain or branched alkylsulfinyl, preferably having 1 to 8 or having 1 to 6 carbon atoms, for example methylsulfinyl, ethylsulfinyl, n-propylsulfinyl, isopropylsulfinyl, n-butylsulfinyl, isobutylsulfinyl, sec-butylsulfinyl and tert-butylsulfinyl.

According to the invention, “alkylsulfonyl”—alone or as part of a chemical group—is straight-chain or branched alkylsulfonyl, preferably having 1 to 8 or having 1 to 6 carbon atoms, for example methylsulfonyl, ethylsulfonyl, n-propylsulfonyl, isopropylsulfonyl, n-butylsulfonyl, isobutylsulfonyl, sec-butylsulfonyl and tert-butylsulfonyl.

According to the invention, “cycloalkylsulfonyl”—alone or as part of a chemical group—is optionally substituted cycloalkylsulfonyl, preferably having 3 to 6 carbon atoms, for example cyclopropylsulfonyl, cyclobutylsulfonyl, cyclopentylsulfonyl or cyclohexylsulfonyl.

According to the invention, “arylsulfonyl” is optionally substituted phenylsulfonyl or optionally substituted polycyclic arylsulfonyl, for example substituted by halogen, alkyl, haloalkyl, haloalkoxy or alkoxy groups.

The term “sulfilimine” represents a group with a nitrogen-sulfur double bond, in which nitrogen and sulfur have further substitution, the nitrogen atom preferably by a further-substituted carbonyl group and the sulfur preferably by two identical or mixed alkyl, aryl and cycloalkyl substituents, for example in the form of an N-(di-n-butylsulfanylidene), N-(diisopropylsulfanylidene), N-(di-n-propylsulfanylidene), N-(di-n-pentylsulfanylidene), N-(diisobutylsulfanylidene), N-(cyclobutylisopropylsulfanylidene), N-(n-propylisopropylsulfanylidene), N-(cyclopropylisopropylsulfanylidene) or N-(isobutylisopropylsulfanylidene) unit.

Depending on the nature of the substituents and the manner in which they are attached, the compounds of the general formula (I) may be present as stereoisomers. The possible stereoisomers defined by the specific three-dimensional form thereof, such as enantiomers, diastereomers, Z and E isomers, are all encompassed by the general formula (I). If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

The radical definitions stated above, in general terms or listed within areas of preference, apply both to the end products of the formula (I) and correspondingly to the starting materials or the intermediates required for the preparation in each case. These radical definitions can be exchanged for one another as desired, i.e. including combinations between the given preferred ranges.

The term “crop plants” as used here refers to cultivated plants which are used as plants for obtaining foods, animal feeds or for industrial purposes.

The compounds of the general formula (I) can be prepared by reacting, for example,
(a) a carboxylic acid of the general formula (II)

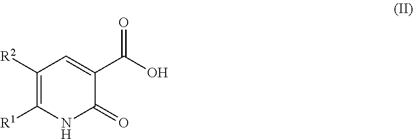

in which R¹ and R² in the compound of the formula (I) to be prepared are each as defined according to the above radical definition,
with an amine of the formula (III) or salt thereof

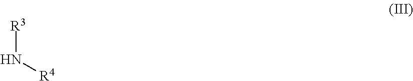

in which R³ and R⁴ in the compound of the formula (I) to be prepared are each as defined according to the above radical definition,
optionally in the presence of a carboxylic acid-activating reagent, for example N,N-carbonyldiimidazole (CDI), or a dehydrating agent, for example dicyclohexylcarbodiimide (DCC), to give the compound of the formula (I) or
(b) a carboxylic ester of the general formula (IV)

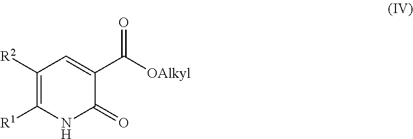

in which R¹ and R² in the compound of the formula (I) to be prepared are each as defined according to the above radical definition and “alkyl” is an alkyl radical, for example methyl or ethyl, with an amine of the formula (III) or salt thereof,

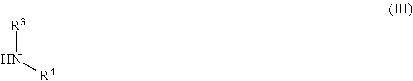

in which R³ and R⁴ in the compound of the formula (I) to be prepared are each as defined according to the above radical definition, to give the compound of the formula (I) or
(c) a carbonyl halide or carboxylic anhydride of the general formula (V),

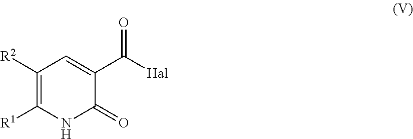

in which R¹ and R² in the compound of the formula (I) to be prepared are each as defined according to the above radical definition and Hal is a halogen atom, for example chlorine, or an acyloxy radical, with an amine of the formula (III) or salt thereof,

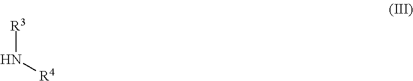

in which R³ and R⁴ are each as defined in the compound of the formula (I) to be prepared, to give the compound of the formula (I),
(d) if R³ and R⁴ in the compound of the formula (I) to be prepared according to the above radical definition are each hydrogen,
   a compound of the formula (VI)

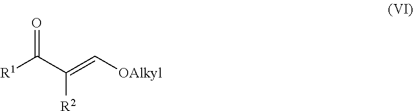

in which R¹ and R² in the compound of the formula (I) to be prepared are each as defined according to the above radical definition and “alkyl” is an alkyl radical, for example methyl or ethyl,
with malonamide to give the compound of the formula (I).

The amide formations according to variant (a) can be conducted, for example, in an inert organic solvent within a temperature range between 0° C. and 150° C., preferably 0° C. and 50° C. Suitable organic solvents are, for example, polar protic or aprotic solvents such as ethers, for example diethyl ether, tetrahydrofuran and dioxane, or nitriles such as acetonitrile, or amides such as dimethylformamide.

The amide formations according to variant (b) can be conducted, for example, in an inert organic solvent within a temperature range between 0° C. and 150° C., preferably 50° C. and 100° C. Suitable organic solvents are, for example, polar protic or aprotic solvents such as ethers, for example tetrahydrofuran and dioxane, or nitriles such as acetonitrile, or amides such as dimethylformamide. Preference is given, however, to amide formation according to variant (b) at elevated temperatures by reaction of the co-reactants in substance.

The amide formations according to variant (c) can be conducted, for example, in the presence of an acid binder in an inert organic solvent within a temperature range between 0° C. and 150° C., preferably 0° C. and 50° C. Suitable organic solvents are, for example, polar protic or aprotic solvents such as ethers, for example diethyl ether, tetrahydrofuran and dioxane, or nitriles such as acetonitrile, or amides such as dimethylformamide. Acid binders are, for example, alkali metal or alkaline earth metal carbonates, for example sodium carbonate, potassium carbonate or calcium carbonate, alkali metal or alkaline earth metal hydroxides, such as sodium hydroxide, potassium hydroxide or calcium hydroxide, or alkali metal hydrides or amides, such as sodium hydride or potassium hydride or sodium amide or potassium amide, or else organic bases such as triethylamine, pyridine, dimethylaminopyridine, DBU (1,8-diazabicyclo[5.4.0]-undec-7-ene), DBN (1,5-diazabicyclo[4.3.0]non-5-ene) and 1,4-diazabicyclo[2.2.2]octane.

The amide formations according to variant (d) can be conducted analogously to the methods as described in EP 522392 and Helv. Chim. Acta 71 (1988) 596-601 and GB 2305174. The malonamide can generally be converted to a reactive salt in an organic anhydrous polar protic or aprotic solvent, for example in an alcohol, with a strong base such as an alkali metal, alkali metal hydride or alkali metal alkoxide, and then reacted with the compound of the formula (VI). The reaction with the compound (VI) can generally be conducted within a temperature range between 0° C. and the boiling point of the solvent (according to the solvent, for instance, up to 150° C.).

The compounds of the general formulae (II), (III), (IV) and (V) are either commercially available or can be prepared by or analogously to methods known to the person skilled in the art (e.g. Helv. Chim. Acta 71 (1988) 596; EP 502740; EP 522392).

For example, the compounds of the formula (IVa)

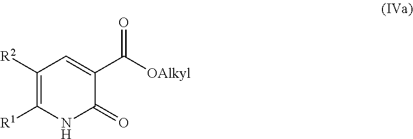

in which R¹ and R² in the compound of the formula (I) to be prepared are each as defined according to the above radical definition
are obtainable by reacting alkoxyvinyl ethers of the formula (VI) in which R¹ and R² in the compound of the formula (I) to be prepared are each as defined according to the above radical definition with alkyl malonamates of the formula (VII).

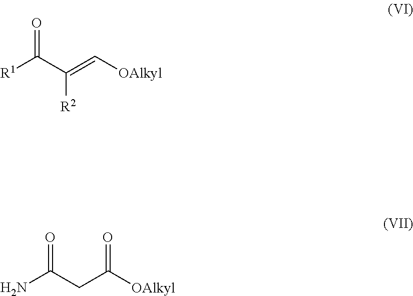

The reactants of the formula (VI) in which R¹ and R² in the compound of the formula (I) to be prepared are each as defined according to the above radical definition are either commercially available or can be prepared by known methods (for example Synthesis 2000, 738-742; J. Fluor. Chem., 107, 2001, 285-300; Organometallics 15, 1996, 5374-5379).

The compounds of the formula (IV) in which R² is a halogen atom can be prepared by customary halogenations from the compounds of the formula (IVa).

Halogenating agents employed for pyridines are, for example, chlorine (J. Org. Chem. 23, 1958, 1614), bromine (Synth. Commun. 19, 1989, 553-560; US P 2532055), iodine (Tetrahedron Lett. 45, 2004, 6633-6636), sodium hypochlorite (J. Org. Chem. 49, 1984, 4784-4786; J. Med. Chem. 36, 1993, 2676-2688, US P 4960896), sodium hypobromite (J. Med. Chem. 32, 1989, 2178-2199), thionyl chloride (Organic Letters, 6, 2004, 3-5), N-chlorosuccinimide (J. Med. Chem. 46, 2003, 702-715), N-bromosuccinimide (Chem. Pharm. Bull. 48, 2000, 1847-1853), N-iodosuccinimide (J. Med. Chem. 36, 1993, 2676-2788).

In addition, the compounds of the general formula (IV) in which R¹ and R² in the compound of the formula (I) to be prepared are defined according to the above radical definition can be prepared from the compounds of the general formula (IVa) in which R¹ and R² in the compound of the formula (I) to be prepared are defined according to the above radical definition by successive nitration (e.g. J. Med. Chem. 36, 1993, 2676-2688; J. Heterocycl. Chem. 33, 1996, 287-294), reduction (e.g. J. Med. Chem. 33, 1990, 1859-1865), diazotization and subsequent conversion of the diazonium salts by means of Sandmeyer or Schiemannn reaction.

The ¹H NMR spectroscopy data reported for the chemical examples described in the section which follows (400 MHz in the case of ¹H NMR, solvent: CDCl₃ or DMSO-d₆ (also referred to hereinafter equally as DMSO), internal standard: tetramethylsilane δ=0.00 ppm) were obtained with a Bruker instrument. The signals referenced have the following meanings: br.=broad; s=singlet, d=doublet, t=triplet, m=multiplet, q=quartet, sext=sextet, sept=septet.

### PREPARATION EXAMPLES

### Example 32, According to Table A Below

### 5,6-Dimethyl-2-oxo-N-(pentan-2-yl)-1,2-dihydropyridine3-carboxamide

### 32.1) (E/Z)-Sodium 2-methyl-3-oxobut-1-en-1-olate

Under an argon atmosphere, sodium (31.9 g, 1.39 mol) was dissolved at room temperature in methanol (197.8 g, 250 mL, 6.17 mol), 800 mL of diethyl ether were added and the mixture was cooled down to 0° C. in an ice bath. To this was added dropwise a mixture of 2-butanone (100.0 g, 1.39 mol) and ethyl formate (102.7 g, 1.39 mol). After the addition had ended, the reaction mixture was stirred at room temperature for a further 16 h. The precipitated solid was filtered off with suction, washed with diethyl ether, dried under reduced pressure at 55° C. for 2 h. 81.8 g (48% of theory) of a colorless solid were obtained.

¹H-NMR (400 MHz, DMSO-d₆): δ=9.07 (s, 1H), 1.89 (s, 3H), 1.39 (s, 3H).

### 32.2) 5,6-Dimethyl-2-oxo-1,2-dihydropyridine-3-carbonitrile

To a solution of (E/Z)-sodium 2-methyl-3-oxobut-1-en-1-olate (77.6 g, 0.64 mol) in 480 mL of water was added cyanoacetamide (49.0 g, 0.70 mol). Added to the mixture was a piperidine acetate solution (prepared from acetic acid (9.16 g, 8.75 mL, 0.15 mol), 21.2 mL of water and pyridine (13.0 g, 15.1 mL, 0.15 mol)), and the mixture was heated under reflux for 4 h and stirred at room temperature for a further 16 h. Acetic acid (75.5 g, 72 mL, 1.26 mol) was added to the reaction mixture, and a pale yellowish solid precipitated out. The latter was filtered off with suction, washed with water and dried under reduced pressure at 55° C. for 2 h. 66.4 g (70% of theory) of a pale yellowish solid were obtained.

¹H-NMR (400 MHz, DMSO-d₆): δ=7.87 (s, 1H), 2.23 (s, 3H), 1.97 (s, 3H).

### 32.3) 5,6-Dimethyl-2-oxo-1,2-dihydropyridine-3-carboxylic acid

A mixture of 5,6-dimethyl-2-oxo-1,2-dihydropyridine-3-carbonitrile (43.0 g, 0.29 mol) and 6 N sodium hydroxide solution (200 mL, 1.20 mol) was heated in an autoclave to 145° C. for 4 h. After the reaction had ended, the reaction mixture was adjusted to pH 1-2 with conc. hydrochloric acid while cooling with an ice bath, and a solid precipitated out. The latter was filtered off with suction, washed with water and dried under reduced pressure at 55° C. for 2 h. 34.8 g (68% of theory) of a colorless solid were obtained.

¹H-NMR (400 MHz, DMSO-d₆): δ=14.9 (s, 1H), 13.2 (br.s, 1H), 8.18 (s, 1H), 2.34 (s, 3H), 2.1 (s, 3H).

### 32.4) 5,6-Dimethyl-2-oxo-N-(pentan-2-yl)-1,2-dihydropyridine-3-carboxamide

A mixture of 5,6-dimethyl-2-oxo-1,2-dihydropyridine-3-carboxylic acid (300 mg, 1.80 mmol) and 1,1-carbonyldiimidazole (CDI, 162.2 mg, 1.56 mmol) was initially charged in 3.00 mL of THF and stirred at 75° C. for 1 h. After cooling to room temperature, 2-pentylamine (312.9 mg, 5.00 mL, 3.59 mmol) was added and the reaction mixture was stirred at room temperature for 16 h. After addition of dil. hydrochloric acid (0.5 N), the mixture was extracted with dichloromethane and the organic phases were dried. After removal of the solvents in vacuo and column chromatography purification, 178.3 mg (40% of theory) of a colorless solid were obtained.

¹H-NMR (400 MHz, CDCl₃): δ=12.7 (br.s, 1H), 9.36 (br.s, 1H), 8.42 (s, 1H), 4.16, (sept., 1H), 2.35 (s, 3H), 2.15 (s, 3H), 1.60-1.30 (m, 4H), 1.21 (d, 3H), 0.97 (t, 3H).

### Example 665, According to Table A Below

### N-Cyclopropyl-5-ethyl-2-oxo-6-(pentafluoroethyl)-1,2-dihydropyridine-3-carboxamide

### 665.1) (E/Z)-4-(Ethoxymethylene)-1,1,1,2,2-pentafluorohexan-3-one

(E/Z)-1-Butene ethyl ether (21.0 g, 0.21 mol) was initially charged together with pyridine (16.6 g, 17 mL, 0.21 mol) in 600 mL of acetonitrile and the mixture was cooled to 10° C. Pentafluoroacetic anhydride (65.0 g, 41.4 mL, 0.21 mol) was slowly added dropwise to the mixture and, after the addition had ended, stirring was continued at room temperature for another 16 h. Water was added and the reaction mixture was extracted with dichloromethane. The organic phases were washed with dil. hydrochloric acid and water and dried, and the solvents were distilled off. This gave 50.5 g (98% of theory) as a colorless oil.

¹H-NMR (400 MHz, CDCl₃): δ=7.60 (s, 1H), 4.21 (q, 2H), 2.32 (q, 2H), 1.41 (t, 3H), 0.95 (t, 3H).

### 665.2) 5-Ethyl-2-oxo-6-(pentafluoroethyl)-1,2-dihydropyridine-3-carboxylic acid methyl ester

Under a nitrogen atmosphere, sodium (5.60 g, 0.24 mol) was dissolved at room temperature in 600 mL of methanol. After addition of (E/Z)-4-(ethoxymethylene)-1,1,1,2,2-pentafluorohexan-3-one (50.0 g, 0.20 mol) and methyl malonamate (23.8 g, 30 mL, 0.20 mol), the reaction mixture was heated under reflux for 4 h. After the reaction had ended, the methanol was distilled off, water was added to the residue and the latter was extracted with dichloromethane. The aqueous phase was adjusted to pH 2 with dil. hydrochloric acid, and a solid precipitated out. After removal of the solid by suction filtration and drying in vacuo, 33.2 g (55% of theory) were obtained.

¹H-NMR (400 MHz, CDCl₃): δ=11.9 (br.s, 1H), 8.21 (s, 1H), 3.86 (s, 3H), 2.74-2.71 (m, 2H), 1.17 (t, 3H).

### 665.3) N-Cyclopropyl-5-ethyl-2-oxo-6-(pentafluoroethyl)-1,2-dihydropyridine-3-carboxamide

A mixture of 5-ethyl-2-oxo-6-(pentafluoroethyl)-1,2-dihydropyridine-3-carboxylic acid methyl ester (500 mg, 1.67 mmol) and cyclopropylamine (114 mg, 1.00 mL, 2.00 mmol) in 5.00 mL of acetonitrile was stirred at room temperature for 16 h. After the reaction had ended, the mixture was adjusted to pH 2 with dil. hydrochloric acid and the precipitated solid was filtered off with suction. Drying in vacuo gave 346 mg (64% of theory) as a colorless solid.

¹H-NMR (400 MHz, DMSO-d₆): δ=13.0 (br.s, 1H), 8.73 (br.s, 1H), 8.25 (s, 1H), 2.90-2.82 (m, 1H), 2.75-2.62 (m, 2H), 1.17 (t, 3H), 0.80-0.70 (m, 2H), 0.61-0.55 (m, 2H).

### Example 219, According to Table A Below

### N,5-Dimethyl-2-oxo-N-phenyl-6-(trifluoromethyl)-1,2-dihydropyridine-3-carboxamide

### 2193.1) (E/Z)-4-Ethoxy-1,1-trifluoro-3-methylbut-3-en-2-one

(E/Z)-1-Ethyl propyl ether (25.0 g, 32.2 mL, 0.28 mol) was initially charged together with pyridine (22.5 g, 23 mL, 0.28 mol) in 500 mL of chloroform and the mixture was cooled to 5° C. Pentafluoroacetic anhydride (60.0 g, 40.3 mL, 0.28 mol) was slowly added dropwise to the mixture and, after the addition had ended, stirring was continued at room temperature for another 16 h. Water was added and the reaction mixture was extracted with dichloromethane. The organic phases were washed with dil. hydrochloric acid and water and dried, and the solvents were distilled off. This gave 43.8 g (84% of theory) as a colorless oil.

¹H-NMR (400 MHz, CDCl₃): δ=7.55 (s, 1H), 4.20 (q, 2H), 1.70 (s, 3H), 1.40 (t, 3H).

### 2193.2) 5-Methyl-2-oxo-6-(trifluoromethyl)-1,2-dihydropyridine-3-carboxylic acid methyl ester

Under an argon atmosphere, sodium (8.69 g, 0.38 mol) was dissolved at room temperature in 1 L of methanol. After addition of (E/Z)-4-ethoxy-1,1,1-trifluoro-3-methylbut-3-en-2-one (66.1 g, 0.31 mol) and methyl malonamate (36.9 g, 46.6 mL, 0.31 mol), the reaction mixture was heated under reflux for 4 h. After the reaction had ended, the methanol was distilled off, water was added to the residue and the latter was extracted with dichloromethane. The aqueous phase was adjusted to pH 2 with dil. hydrochloric acid, and a solid precipitated out. After removal of the solid by suction filtration and drying in vacuo, 30.7 g (42% of theory) were obtained.

¹H-NMR (400 MHz, DMSO-d₆): δ=12.0 (br.s, 1H), 8.20 (s, 1H), 3.85 (s, 3H), 2.34 (s, 3H).

### 2193.3) 5-Methyl-2-oxo-6-(trifluoromethyl)-1,2-dihydropyridine-3-carboxylic acid

5-Methyl-2-oxo-6-(trifluoromethyl)-1,2-dihydropyridine-3-carboxylic acid methyl ester (8.00 g, 34.0 mmol) was dissolved in 350 mL of methanol, and 70 mL of an aqueous solution of lithium hydroxide monohydrate (2.14 g, 51.0 mmol) were added. The reaction mixture was stirred at room temperature for a further 16 h. After the reaction had ended, the reaction mixture was adjusted to pH 1-2 with conc. hydrochloric acid while cooling with an ice bath, and a solid precipitated out. The latter was filtered off with suction, washed with water and dried at 55° C. for 2 h. 6.30 g (84% of theory) of a colorless solid were obtained.

¹H-NMR (400 MHz, DMSO-d₆): δ=8.22 (s, 1H), 2.35 (s, 3H).

### 2193.4) N,5-Dimethyl-2-oxo-N-phenyl-6-(trifluoromethyl)-1,2-dihydropyridine-3-carboxamide

5-Methyl-2-oxo-6-(trifluoromethyl)-1,2-dihydropyridine-3-carboxylic acid (150 mg, 0.69 mmol) was initially charged in 5 mL of THF, and 1,1-carbonyldiimidazole (CDI, 220.0 mg, 1.36 mmol) was added. The reaction mixture was stirred at room temperature for 1 h. After addition of N-methylaniline (145.0 mg, 1.36 mmol), the mixture was stirred at room temperature for 16 h. After addition of water, the mixture was extracted with dichloromethane and the organic phases were dried. After removal of the solvents in vacuo and column chromatography purification, 85.0 mg (40% of theory) of a colorless oil were obtained.

¹H-NMR (400 MHz, DMSO-d₆): δ=11.6 (br.s, 1H), 7.67 (br.s, 1H), 7.30-7.15 (br.m, 5H), 2.33 (br.s, 3H), 2.20 (br.s, 3H).

### Example 1744, According to Table A Below

### 1-({5′-chloro-6′-[chloro(difluoro)methyl]-2′-oxo-1′,2′-dihydropyridin-3′-yl}carbonyl)-piperidine-2-carboxylic acid ethyl ester

### 1744.1) (E/Z)-4-Butoxy-1-chloro-1,1-difluorobut-3-en-2-one

(E/Z)-1-Butene ethyl ether (41.2 g, 53 mL, 0.40 mol) was initially charged together with pyridine (31.9 g, 33 mL, 0.40 mol) in 990 mL of chloroform and the mixture was cooled to 5° C. Chlorodifluoroacetic anhydride (100.0 g, 71.4 mL, 0.40 mol) was slowly added dropwise to the mixture and, after the addition had ended, stirring was continued at room temperature for another 16 h. Water was added and the reaction mixture was extracted with dichloromethane. The organic phases were washed with dil. hydrochloric acid and water and dried, and the solvents were distilled off. This gave 75.7 mg (88% of theory) as a colorless oil.

¹H-NMR (400 MHz, CDCl₃): δ=7.90 (d, 1H), 5.87 (d, 1H), 4.02 (t, 2H), 1.73 (pent., 2H), 1.43 (sext., 2H), 0.94 (t, 3H).

### 1744.2) 6-[Chloro(difluoro)methyl]-2-oxo-1,2-dihydropyridine-3-carboxylic acid methyl ester

Under an argon atmosphere, sodium (7.02 g, 0.31 mol) was dissolved at room temperature in 750 mL of methanol. After addition of (E/Z)-4-butoxy-1-chloro-1,1-difluorobut-3-en-2-one (54.1 g, 0.25 mol) and methyl malonamate (29.8 g, 41.4 mL, 0.25 mol), the reaction mixture was heated under reflux for 4 h. After the reaction had ended, the methanol was distilled off, water was added to the residue and the latter was extracted with dichloromethane. The aqueous phase was adjusted to pH 2 with dil. hydrochloric acid, and a solid precipitated out. After removal of the solid by suction filtration and drying in vacuo, 42.4 g (70% of theory) were obtained.

¹H-NMR (400 MHz, CDCl₃): δ=12.5 (br.s, 1H), 8.31 (d, 1H), 7.38 (d, 2H), 3.85 (s, 3H).

### 1744.3) 5-Chloro-6-[chloro(difluoro)methyl]-2-oxo-1,2-dihydropyridine-3-carboxylic acid methyl ester

6-[Chloro(difluoro)methyl]-2-oxo-1,2-dihydropyridine-3-carboxylic acid methyl ester (15.0 g, 63.1 mmol) was initially charged in 750 mL of N,N-dimethylformamide, and N-chlorosuccinimide (12.6 g, 94.7 mmol) was added in portions. The reaction mixture was stirred at room temperature for 16 h. After addition of water, the precipitated solid was filtered off and dried. 10.6 g (62% of theory) were obtained.

¹H-NMR (400 MHz, DMSO-d₆): δ=12.7 (br.s, 1H), 8.35 (s, 1H), 3.85 (s, 3H).

### 1744.4) 5-Chloro-6-[chloro(difluoro)methyl]-2-oxo-1,2-dihydropyridine-3-carboxylic acid

5-Chloro-6-[chloro(difluoro)methyl]-2-oxo-1,2-dihydropyridine-3-carboxylic acid methyl ester (5.00 g, 18.4 mmol) was dissolved in 50 mL of methanol, and 50 mL of an aqueous solution of lithium hydroxide monohydrate (1.16 g, 27.6 mmol) were added. The reaction mixture was stirred at room temperature for a further 16 h. After the reaction had ended, the reaction mixture was adjusted to pH 1-2 with conc. hydrochloric acid while cooling with an ice bath, and a solid precipitated out. The latter was filtered off with suction, washed with water and dried at 55° C. for 2 h. 4.03 g (81% of theory) of a colorless solid were obtained.

¹H-NMR (400 MHz, DMSO-d₆): δ=8.30 (s, 1H).

### 1744.5) 1-({5′-chloro-6′-[chloro(difluoro)methyl]-2′-oxo-1′,2′-dihydropyridin-3′-yl}carbonyl)-piperidine-2-carboxylic acid ethyl ester

5-Chloro-6-[chloro(difluoro)methyl]-2-oxo-1,2-dihydropyridine-3-carboxylic acid (200 mg, 0.78 mmol) was initially charged in 5 mL of THF, and 1,1-carbonyldiimidazole (CDI, 251.0 mg, 1.55 mmol) was added. The reaction mixture was stirred at room temperature for 1 h. After addition of ethyl piperidine-2-carboxylate (244.0 mg, 1.55 mmol), the mixture was stirred at room temperature for 16 h. After addition of water, the mixture was extracted with ethyl acetate and the organic phases were dried. After removal of the solvents in vacuo and column chromatography purification, 59.0 mg (19% of theory) of a colorless solid were obtained.

¹H-NMR (400 MHz, DMSO-d₆): δ=12.6 (s, 1H), 7.93 (s, 1H), 5.26-5.20 (m, 1H), 4.22-4.13 (m, 2H), 3.20-3.10 (m, 1H), 2.48-2.43 (m, 1H), 2.21-2.12 (m, 1H), 1.74-1.60 (m, 4H), 146-1.35 (m, 1H), 1.23 (t, 3H).

Table A below lists, by way of example, a series of further compounds of the general formula (I) which can be obtained in an analogous manner to the above examples and the methods mentioned further up.

In the table:

| | |
|---|---|
| Bu = butyl | Et = ethyl |
| Me = methyl | Ph = phenyl |
| Pr = propyl | s = secondary |
| i = iso | c = cyclo |
| t = tertiary | |

This applies correspondingly to composite terms such as

iPr = isopropyl
iBu = isobutyl
sBu = sec-butyl
tBu = tert-butyl
cPr = cyclopropyl
cPentyl = cyclopentyl
cHexyl = cyclohexyl

If Table A lists an alkyl radical without further specification, it is the straight-chain alkyl radical, i.e., for example, Bu=n-Bu=n-butyl.

The numerical indices in the formulae expressed in table A are not subscripted but arranged at the same line height and in the same font size as the atom symbols.

For example, the formula CF3 in the table corresponds to the formula CF₃ according to the customary notation with subscripted index, or the formula CH2CH(CH2CH3)2 corresponds to the formula CH₂CH(CH₂CH₃)₂ with subscripted indices.

TABLE A
Compounds of the formula (I)
(I)

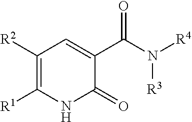

| Ex. | R¹ | R² | R³ | R⁴ |
|---|---|---|---|---|
| 1 | Me | Me | H | Me |
| 2 | Me | Me | H | Et |
| 3 | Me | Me | H | Pr |
| 4 | Me | Me | H | iPr |
| 5 | Me | Me | H | cPr |
| 6 | Me | Me | H | Bu |
| 7 | Me | Me | H | cBu |
| 8 | Me | Me | H | tBu |
| 9 | Me | Me | H | cPentyl |
| 10 | Me | Me | H | cHexyl |
| 11 | Me | Me | H | CH2(CH2)3CH3 |
| 12 | Me | Me | H | CH2(CH2)4CH3 |
| 13 | Me | Me | H | CH2—cPr |
| 14 | Me | Me | H | CH2—CN |
| 15 | Me | Me | H | CH2—C(CH3)3 |
| 16 | Me | Me | H | CH2CF2CF3 |
| 17 | Me | Me | H | CH2CF3 |
| 18 | Me | Me | H | CH2(CF2)2CF3 |
| 19 | Me | Me | H | CH2CH(CH3)CH2CH3 |
| 20 | Me | Me | H | CH2C(CH3)2CH2F |
| 21 | Me | Me | H | CH2CH(CH3)2 |
| 22 | Me | Me | H | CH2CH(CH2CH3)2 |
| 23 | Me | Me | H | CH2CH2CH(CH3)2 |
| 24 | Me | Me | H | CH2CH2C(CH3)3 |
| 25 | Me | Me | H | CH2CH═CH2 |
| 26 | Me | Me | H | CH2CH═CHCH3 |
| 27 | Me | Me | H | CH2—C(CH3)═CH2 |
| 28 | Me | Me | H | CH2—C≡CH |
| 29 | Me | Me | H | CH(CH3)CH2CH3 |
| 30 | Me | Me | H | CH(CH3)cPr |
| 31 | Me | Me | H | CH(CH3)CF3 |
| 32 | Me | Me | H | CH(CH3)(CH2)2CH3 |
| 33 | Me | Me | H | CH(CH3)(CH2)4CH3 |
| 34 | Me | Me | H | CH(CH3)(CH2)5CH3 |
| 35 | Me | Me | H | CH(CH2CH3)(CH2)3CH3 |
| 36 | Me | Me | H | CH(CH3)CH2CH(CH3)2 |
| 37 | Me | Me | H | CH(CH3)C(CH3)3 |
| 38 | Me | Me | H | CH(CH3)CH(CH3)2 |
| 39 | Me | Me | H | CH(CH3)CH2CH2CH(CH3)2 |
| 40 | Me | Me | H | CH(CH2CH3)2 |
| 41 | Me | Me | H | C(CH3)2CH2CH3 |
| 42 | Me | Me | H | C(CH3)2CH2C(CH3)3 |
| 43 | Me | Me | H | CH2—CH(OMe)2 |
| 44 | Me | Me | H | CH2—CH(OEt)2 |
| 45 | Me | Me | H | CH2CH2—OH |
| 46 | Me | Me | H | CH2CH2—OMe |
| 47 | Me | Me | H | CH2CH2—OEt |
| 48 | Me | Me | H | CH2CH2—SMe |
| 49 | Me | Me | H | CH2CH2—CN |
| 50 | Me | Me | H | CH2CH2—NMe2 |
| 51 | Me | Me | H | CH2CH2-Morpholin-4-yl |
| 52 | Me | Me | H | CH(CH3)CH2—OMe |
| 53 | Me | Me | H | CH(CH3)CH2—NMe2 |
| 54 | Me | Me | H | CH2CH2CH2—OMe |
| 55 | Me | Me | H | CH2CH2CH2—SMe |
| 56 | Me | Me | H | CH2CH2CH2—OEt |
| 57 | Me | Me | H | CH2CH2CH2—OiPr |
| 58 | Me | Me | H | CH2CH2CH2—OBu |
| 59 | Me | Me | H | CH2—COOMe |
| 60 | Me | Me | H | CH2—COOH |
| 61 | Me | Me | H | CH(CH3)COOMe |
| 62 | Me | Me | H | CH(CH3)COOEt |
| 63 | Me | Me | H | CH2CH2—COOMe |
| 64 | Me | Me | H | CH(COOMe)2 |
| 65 | Me | Me | H | CH(COOEt)CH2—CH(CH3)2 |
| 66 | Me | Me | H | CH(COOMe)CH(CH3)2 |
| 67 | Me | Me | H | COCH2OMe |
| 68 | Me | Me | H | CH(cPr)2 |
| 69 | Me | Me | H | Ph |
| 70 | Me | Me | H | CH2PH |
| 71 | Me | Me | H | CH2-Pyridin-3-yl |
| 72 | Me | Me | H | CH2—6-Cl-Pyridin-3-yl |
| 73 | Me | Me | H | CH(CH3)Ph |
| 74 | Me | Me | H | CH2CH2—Ph |
| 75 | Me | Me | H | CH2—2-CF3—Ph |
| 76 | Me | Me | H | CH2CH2CHPh2 |
| 77 | Me | Me | H | O—CH2CH3 |
| 78 | Me | Me | H | O—CH3 |
| 79 | Me | Me | H | O—CH2CH═CH2 |
| 80 | Me | Me | H | O—tBu |
| 81 | Me | Me | H | O—Pr |
| 82 | Me | Me | H | O—CH2cPr |
| 83 | Me | Me | H | O—CH2CH(CH3)2 |
| 84 | Me | Me | H | O—CH2CF3 |
| 85 | Me | Me | H | O—CH(CH3)cPr |
| 86 | Me | Me | H | O—CH2CH2Cl |
| 87 | Me | Me | H | O—CH2C≡CH |
| 88 | Me | Me | H | O—CH2C≡CCH3 |
| 89 | Me | Me | H | O—CH(CH3)C≡CH |
| 90 | Me | Me | H | Indan-1-yl |
| 91 | Me | Me | H | Tetrahydrofuran-2-ylmethyl |
| 92 | Me | Me | Morpholin-4-yl | |
| 93 | Me | Me | 2-(Methoxycarbonyl)piperidin-1-yl | |
| 94 | Me | Me | 2-(Ethoxycarbonyl)piperidin-1-yl | |
| 95 | Me | Me | Thiazolidin-3-yl | |
| 96 | Me | Me | Pyrrolidin-1-yl | |
| 97 | Me | Me | 2-Methylpyrrolidin-1-yl | |
| 98 | Me | Me | ═CH—N(CH3)2 | |
| 99 | Me | Me | ═C(CH3)N(CH3)2 | |
| 100 | Me | Me | ═CH—N(C2H5)2 | |
| 101 | Me | Me | ═C(CH3)N(C2H5)2 | |
| 102 | Me | Me | ═CH-Piperidine | |
| 103 | Me | Me | ═CH-Morpholine | |
| 104 | Me | Me | ═CH-Pyrrolidine | |
| 105 | Me | Me | Me | Me |
| 106 | Me | Me | Me | Et |
| 107 | Me | Me | Et | Me |
| 108 | Me | Me | Et | Et |
| 109 | Me | Me | Me | Ph |
| 110 | Me | Me | Me | CH2CO2Me |
| 111 | CF3 | Me | H | Me |
| 112 | CF3 | Me | H | Et |
| 113 | CF3 | Me | H | Pr |
| 114 | CF3 | Me | H | iPr |
| 115 | CF3 | Me | H | cPr |
| 116 | CF3 | Me | H | Bu |
| 117 | CF3 | Me | H | cBu |
| 118 | CF3 | Me | H | tBu |
| 119 | CF3 | Me | H | cPentyl |
| 120 | CF3 | Me | H | cHexyl |
| 121 | CF3 | Me | H | CH2(CH2)3CH3 |
| 122 | CF3 | Me | H | CH2(CH2)4CH3 |
| 123 | CF3 | Me | H | CH2—cPr |
| 124 | CF3 | Me | H | CH2—CN |
| 125 | CF3 | Me | H | CH2—C(CH3)3 |
| 126 | CF3 | Me | H | CH2CF2CF3 |
| 127 | CF3 | Me | H | CH2CF3 |
| 128 | CF3 | Me | H | CH2(CF2)2CF3 |
| 129 | CF3 | Me | H | CH2CH(CH3)CH2CH3 |
| 130 | CF3 | Me | H | CH2C(CH3)2CH2F |
| 131 | CF3 | Me | H | CH2CH(CH3)2 |
| 132 | CF3 | Me | H | CH2CH(CH2CH3)2 |
| 133 | CF3 | Me | H | CH2CH2CH(CH3)2 |
| 134 | CF3 | Me | H | CH2CH2C(CH3)3 |
| 135 | CF3 | Me | H | CH2CH═CH2 |
| 136 | CF3 | Me | H | CH2CH═CHCH3 |
| 137 | CF3 | Me | H | CH2—C(CH3)═CH2 |
| 138 | CF3 | Me | H | CH2—C≡CH |
| 139 | CF3 | Me | H | CH(CH3)CH2CH3 |
| 140 | CF3 | Me | H | CH(CH3)cPr |
| 141 | CF3 | Me | H | CH(CH3)CF3 |
| 142 | CF3 | Me | H | CH(CH3)(CH2)2CH3 |
| 143 | CF3 | Me | H | CH(CH3)(CH2)4CH3 |
| 144 | CF3 | Me | H | CH(CH3)(CH2)5CH3 |
| 145 | CF3 | Me | H | CH(CH2CH3)(CH2)3CH3 |
| 146 | CF3 | Me | H | CH(CH3)CH2CH(CH3)2 |
| 147 | CF3 | Me | H | CH(CH3)C(CH3)3 |
| 148 | CF3 | Me | H | CH(CH3)CH(CH3)2 |
| 149 | CF3 | Me | H | CH(CH3)CH2CH2CH(CH3)2 |
| 150 | CF3 | Me | H | CH(CH2CH3)2 |
| 151 | CF3 | Me | H | C(CH3)2CH2CH3 |
| 152 | CF3 | Me | H | C(CH3)2CH2C(CH3)3 |
| 153 | CF3 | Me | H | CH2—CH(OMe)2 |
| 154 | CF3 | Me | H | CH2—CH(OEt)2 |
| 155 | CF3 | Me | H | CH2CH2—OH |
| 156 | CF3 | Me | H | CH2CH2—OMe |
| 157 | CF3 | Me | H | CH2CH2—OEt |
| 158 | CF3 | Me | H | CH2CH2—SMe |
| 159 | CF3 | Me | H | CH2CH2—CN |
| 160 | CF3 | Me | H | CH2CH2—NMe2 |
| 161 | CF3 | Me | H | CH2CH2-Morpholin-4-yl |
| 162 | CF3 | Me | H | CH(CH3)CH2—OMe |
| 163 | CF3 | Me | H | CH(CH3)CH2—NMe2 |
| 164 | CF3 | Me | H | CH2CH2CH2—OMe |
| 165 | CF3 | Me | H | CH2CH2CH2—SMe |
| 166 | CF3 | Me | H | CH2CH2CH2—OEt |
| 167 | CF3 | Me | H | CH2CH2CH2—OiPr |
| 168 | CF3 | Me | H | CH2CH2CH2—OBu |
| 169 | CF3 | Me | H | CH2—COOMe |
| 170 | CF3 | Me | H | CH2—COOH |
| 171 | CF3 | Me | H | CH(CH3)COOMe |
| 172 | CF3 | Me | H | CH(CH3)COOEt |
| 173 | CF3 | Me | H | CH2CH2—COOMe |
| 174 | CF3 | Me | H | CH(COOMe)2 |
| 175 | CF3 | Me | H | CH(COOEt)CH2—CH(CH3)2 |
| 176 | CF3 | Me | H | CH(COOMe)CH(CH3)2 |
| 177 | CF3 | Me | H | COCH2OMe |
| 178 | CF3 | Me | H | CH(cPr)2 |
| 179 | CF3 | Me | H | Ph |
| 180 | CF3 | Me | H | CH2PH |
| 181 | CF3 | Me | H | CH2-Pyridin-3-yl |
| 182 | CF3 | Me | H | CH2—6-Cl-Pyridin-3-yl |
| 183 | CF3 | Me | H | CH(CH3)Ph |
| 184 | CF3 | Me | H | CH2CH2—Ph |
| 185 | CF3 | Me | H | CH2—2-CF3—Ph |
| 186 | CF3 | Me | H | CH2CH2CHPh2 |
| 187 | CF3 | Me | H | O—CH2CH3 |
| 188 | CF3 | Me | H | O—CH3 |
| 189 | CF3 | Me | H | O—CH2CH═CH2 |
| 190 | CF3 | Me | H | O—tBu |
| 191 | CF3 | Me | H | O—Pr |
| 192 | CF3 | Me | H | O—CH2cPr |
| 193 | CF3 | Me | H | O—CH2CH(CH3)2 |
| 194 | CF3 | Me | H | O—CH2CF3 |
| 195 | CF3 | Me | H | O—CH(CH3)cPr |
| 196 | CF3 | Me | H | O—CH2CH2Cl |
| 197 | CF3 | Me | H | O—CH2C≡CH |
| 198 | CF3 | Me | H | O—CH2C≡CCH3 |
| 199 | CF3 | Me | H | O—CH(CH3)C≡CH |
| 200 | CF3 | Me | H | Indan-1-yl |
| 201 | CF3 | Me | H | Tetrahydrofuran-2-ylmethyl |
| 202 | CF3 | Me | Morpholin-4-yl | |
| 203 | CF3 | Me | 2-(Methoxycarbonyl)piperidin-1-yl | |
| 204 | CF3 | Me | 2-(Ethoxycarbonyl)piperidin-1-yl | |
| 205 | CF3 | Me | Thiazolidin-3-yl | |
| 206 | CF3 | Me | Pyrrolidin-1-yl | |
| 207 | CF3 | Me | 2-Methylpyrrolidin-1-yl | |
| 208 | CF3 | Me | ═CH—N(CH3)2 | |
| 209 | CF3 | Me | ═C(CH3)N(CH3)2 | |
| 210 | CF3 | Me | ═CH—N(C2H5)2 | |
| 211 | CF3 | Me | ═C(CH3)N(C2H5)2 | |
| 212 | CF3 | Me | ═CH-Piperidine | |
| 213 | CF3 | Me | ═CH-Morpholine | |
| 214 | CF3 | Me | ═CH-Pyrrolidine | |
| 215 | CF3 | Me | Me | Me |
| 216 | CF3 | Me | Me | Et |
| 217 | CF3 | Me | Et | Me |
| 218 | CF3 | Me | Et | Et |
| 219 | CF3 | Me | Me | Ph |
| 220 | CF3 | Me | Me | CH2CO2Me |
| 221 | CF2CF3 | Me | H | Me |
| 222 | CF2CF3 | Me | H | Et |
| 223 | CF2CF3 | Me | H | Pr |
| 224 | CF2CF3 | Me | H | iPr |
| 225 | CF2CF3 | Me | H | cPr |
| 226 | CF2CF3 | Me | H | Bu |
| 227 | CF2CF3 | Me | H | cBu |
| 228 | CF2CF3 | Me | H | tBu |
| 229 | CF2CF3 | Me | H | cPentyl |
| 230 | CF2CF3 | Me | H | cHexyl |
| 231 | CF2CF3 | Me | H | CH2(CH2)3CH3 |
| 232 | CF2CF3 | Me | H | CH2(CH2)4CH3 |
| 233 | CF2CF3 | Me | H | CH2—cPr |
| 234 | CF2CF3 | Me | H | CH2—CN |
| 235 | CF2CF3 | Me | H | CH2—C(CH3)3 |
| 236 | CF2CF3 | Me | H | CH2CF2CF3 |
| 237 | CF2CF3 | Me | H | CH2CF3 |
| 238 | CF2CF3 | Me | H | CH2(CF2)2CF3 |
| 239 | CF2CF3 | Me | H | CH2CH(CH3)CH2CH3 |
| 240 | CF2CF3 | Me | H | CH2C(CH3)2CH2F |
| 241 | CF2CF3 | Me | H | CH2CH(CH3)2 |
| 242 | CF2CF3 | Me | H | CH2CH(CH2CH3)2 |
| 243 | CF2CF3 | Me | H | CH2CH2CH(CH3)2 |
| 244 | CF2CF3 | Me | H | CH2CH2C(CH3)3 |
| 245 | CF2CF3 | Me | H | CH2CH═CH2 |
| 246 | CF2CF3 | Me | H | CH2CH═CHCH3 |
| 247 | CF2CF3 | Me | H | CH2—C(CH3)═CH2 |
| 248 | CF2CF3 | Me | H | CH2—C≡CH |
| 249 | CF2CF3 | Me | H | CH(CH3)CH2CH3 |
| 250 | CF2CF3 | Me | H | CH(CH3)cPr |
| 251 | CF2CF3 | Me | H | CH(CH3)CF3 |
| 252 | CF2CF3 | Me | H | CH(CH3)(CH2)2CH3 |
| 253 | CF2CF3 | Me | H | CH(CH3)(CH2)4CH3 |
| 254 | CF2CF3 | Me | H | CH(CH3)(CH2)5CH3 |
| 255 | CF2CF3 | Me | H | CH(CH2CH3)(CH2)3CH3 |
| 256 | CF2CF3 | Me | H | CH(CH3)CH2CH(CH3)2 |
| 257 | CF2CF3 | Me | H | CH(CH3)C(CH3)3 |
| 258 | CF2CF3 | Me | H | CH(CH3)CH(CH3)2 |
| 259 | CF2CF3 | Me | H | CH(CH3)CH2CH2CH(CH3)2 |
| 260 | CF2CF3 | Me | H | CH(CH2CH3)2 |
| 261 | CF2CF3 | Me | H | C(CH3)2CH2CH3 |
| 262 | CF2CF3 | Me | H | C(CH3)2CH2C(CH3)3 |
| 263 | CF2CF3 | Me | H | CH2—CH(OMe)2 |
| 264 | CF2CF3 | Me | H | CH2—CH(OEt)2 |
| 265 | CF2CF3 | Me | H | CH2CH2—OH |
| 266 | CF2CF3 | Me | H | CH2CH2—OMe |
| 267 | CF2CF3 | Me | H | CH2CH2—OEt |
| 268 | CF2CF3 | Me | H | CH2CH2—SMe |
| 269 | CF2CF3 | Me | H | CH2CH2—CN |
| 270 | CF2CF3 | Me | H | CH2CH2—NMe2 |
| 271 | CF2CF3 | Me | H | CH2CH2-Morpholin-4-yl |
| 272 | CF2CF3 | Me | H | CH(CH3)CH2—OMe |
| 273 | CF2CF3 | Me | H | CH(CH3)CH2—NMe2 |
| 274 | CF2CF3 | Me | H | CH2CH2CH2—OMe |
| 275 | CF2CF3 | Me | H | CH2CH2CH2—SMe |
| 276 | CF2CF3 | Me | H | CH2CH2CH2—OEt |
| 277 | CF2CF3 | Me | H | CH2CH2CH2—OiPr |
| 278 | CF2CF3 | Me | H | CH2CH2CH2—OBu |
| 279 | CF2CF3 | Me | H | CH2—COOMe |
| 280 | CF2CF3 | Me | H | CH2—COOH |
| 281 | CF2CF3 | Me | H | CH(CH3)COOMe |
| 282 | CF2CF3 | Me | H | CH(CH3)COOEt |
| 283 | CF2CF3 | Me | H | CH2CH2—COOMe |
| 284 | CF2CF3 | Me | H | CH(COOMe)2 |
| 285 | CF2CF3 | Me | H | CH(COOEt)CH2—CH(CH3)2 |
| 286 | CF2CF3 | Me | H | CH(COOMe)CH(CH3)2 |
| 287 | CF2CF3 | Me | H | COCH2OMe |
| 288 | CF2CF3 | Me | H | CH(cPr)2 |
| 289 | CF2CF3 | Me | H | Ph |
| 290 | CF2CF3 | Me | H | CH2PH |
| 291 | CF2CF3 | Me | H | CH2-Pyridin-3-yl |
| 292 | CF2CF3 | Me | H | CH2—6-Cl-Pyridin-3-yl |
| 293 | CF2CF3 | Me | H | CH(CH3)Ph |
| 294 | CF2CF3 | Me | H | CH2CH2—Ph |
| 295 | CF2CF3 | Me | H | CH2—2-CF3—Ph |
| 296 | CF2CF3 | Me | H | CH2CH2CHPh2 |
| 297 | CF2CF3 | Me | H | O—CH2CH3 |
| 298 | CF2CF3 | Me | H | O—CH3 |
| 299 | CF2CF3 | Me | H | O—CH2CH═CH2 |
| 300 | CF2CF3 | Me | H | O—tBu |
| 301 | CF2CF3 | Me | H | O—Pr |
| 302 | CF2CF3 | Me | H | O—CH2cPr |
| 303 | CF2CF3 | Me | H | O—CH2CH(CH3)2 |
| 304 | CF2CF3 | Me | H | O—CH2CF3 |
| 305 | CF2CF3 | Me | H | O—CH(CH3)cPr |
| 306 | CF2CF3 | Me | H | O—CH2CH2Cl |
| 307 | CF2CF3 | Me | H | O—CH2C≡CH |
| 308 | CF2CF3 | Me | H | O—CH2C≡CCH3 |
| 309 | CF2CF3 | Me | H | O—CH(CH3)C≡CH |
| 310 | CF2CF3 | Me | H | Indan-1-yl |
| 311 | CF2CF3 | Me | H | Tetrahydrofuran-2-ylmethyl |
| 312 | CF2CF3 | Me | Morpholin-4-yl | |
| 313 | CF2CF3 | Me | 2-(Methoxycarbonyl)piperidin-1-yl | |
| 314 | CF2CF3 | Me | 2-(Ethoxycarbonyl)piperidin-1-yl | |
| 315 | CF2CF3 | Me | Thiazolidin-3-yl | |
| 316 | CF2CF3 | Me | Pyrrolidin-1-yl | |
| 317 | CF2CF3 | Me | 2-Methylpyrrolidin-1-yl | |
| 318 | CF2CF3 | Me | ═CH—N(CH3)2 | |
| 319 | CF2CF3 | Me | ═C(CH3)N(CH3)2 | |
| 320 | CF2CF3 | Me | ═CH—N(C2H5)2 | |
| 321 | CF2CF3 | Me | ═C(CH3)N(C2H5)2 | |
| 322 | CF2CF3 | Me | ═CH-Piperidine | |
| 323 | CF2CF3 | Me | ═CH-Morpholine | |
| 324 | CF2CF3 | Me | ═CH-Pyrrolidine | |
| 325 | CF2CF3 | Me | Me | Me |
| 326 | CF2CF3 | Me | Me | Et |
| 327 | CF2CF3 | Me | Et | Me |
| 328 | CF2CF3 | Me | Et | Et |
| 329 | CF2CF3 | Me | Me | Ph |
| 330 | CF2CF3 | Me | Me | CH2CO2Me |
| 331 | CF2CF2Cl | Me | H | Me |
| 332 | CF2CF2Cl | Me | H | Et |
| 333 | CF2CF2Cl | Me | H | Pr |
| 334 | CF2CF2Cl | Me | H | iPr |
| 335 | CF2CF2Cl | Me | H | cPr |
| 336 | CF2CF2Cl | Me | H | Bu |
| 337 | CF2CF2Cl | Me | H | cBu |
| 338 | CF2CF2Cl | Me | H | tBu |
| 339 | CF2CF2Cl | Me | H | cPentyl |
| 340 | CF2CF2Cl | Me | H | cHexyl |
| 341 | CF2CF2Cl | Me | H | CH2(CH2)3CH3 |
| 342 | CF2CF2Cl | Me | H | CH2(CH2)4CH3 |
| 343 | CF2CF2Cl | Me | H | CH2—cPr |
| 344 | CF2CF2Cl | Me | H | CH2—CN |
| 345 | CF2CF2Cl | Me | H | CH2—C(CH3)3 |
| 346 | CF2CF2Cl | Me | H | CH2CF2CF3 |
| 347 | CF2CF2Cl | Me | H | CH2CF3 |
| 348 | CF2CF2Cl | Me | H | CH2(CF2)2CF3 |
| 349 | CF2CF2Cl | Me | H | CH2CH(CH3)CH2CH3 |
| 350 | CF2CF2Cl | Me | H | CH2C(CH3)2CH2F |
| 351 | CF2CF2Cl | Me | H | CH2CH(CH3)2 |
| 352 | CF2CF2Cl | Me | H | CH2CH(CH2CH3)2 |
| 353 | CF2CF2Cl | Me | H | CH2CH2CH(CH3)2 |
| 354 | CF2CF2Cl | Me | H | CH2CH2C(CH3)3 |
| 354 | CF2CF2Cl | Me | H | CH2CH═CH2 |
| 355 | CF2CF2Cl | Me | H | CH2CH═CHCH3 |
| 356 | CF2CF2Cl | Me | H | CH2—C(CH3)═CH2 |
| 357 | CF2CF2Cl | Me | H | CH2—C≡CH |
| 358 | CF2CF2Cl | Me | H | CH(CH3)CH2CH3 |
| 359 | CF2CF2Cl | Me | H | CH(CH3)cPr |
| 360 | CF2CF2Cl | Me | H | CH(CH3)CF3 |
| 361 | CF2CF2Cl | Me | H | CH(CH3)(CH2)2CH3 |
| 362 | CF2CF2Cl | Me | H | CH(CH3)(CH2)4CH3 |
| 363 | CF2CF2Cl | Me | H | CH(CH3)(CH2)5CH3 |
| 364 | CF2CF2Cl | Me | H | CH(CH2CH3)(CH2)3CH3 |
| 365 | CF2CF2Cl | Me | H | CH(CH3)CH2CH(CH3)2 |
| 366 | CF2CF2Cl | Me | H | CH(CH3)C(CH3)3 |
| 367 | CF2CF2Cl | Me | H | CH(CH3)CH(CH3)2 |
| 368 | CF2CF2Cl | Me | H | CH(CH3)CH2CH2CH(CH3)2 |
| 369 | CF2CF2Cl | Me | H | CH(CH2CH3)2 |
| 370 | CF2CF2Cl | Me | H | C(CH3)2CH2CH3 |
| 371 | CF2CF2Cl | Me | H | C(CH3)2CH2C(CH3)3 |
| 372 | CF2CF2Cl | Me | H | CH2—CH(OMe)2 |
| 373 | CF2CF2Cl | Me | H | CH2—CH(OEt)2 |
| 374 | CF2CF2Cl | Me | H | CH2CH2—OH |
| 375 | CF2CF2Cl | Me | H | CH2CH2—OMe |
| 376 | CF2CF2Cl | Me | H | CH2CH2—OEt |
| 377 | CF2CF2Cl | Me | H | CH2CH2—SMe |
| 378 | CF2CF2Cl | Me | H | CH2CH2—CN |
| 379 | CF2CF2Cl | Me | H | CH2CH2—NMe2 |
| 380 | CF2CF2Cl | Me | H | CH2CH2-Morpholin-4-yl |
| 381 | CF2CF2Cl | Me | H | CH(CH3)CH2—OMe |
| 382 | CF2CF2Cl | Me | H | CH(CH3)CH2—NMe2 |
| 383 | CF2CF2Cl | Me | H | CH2CH2CH2—OMe |
| 384 | CF2CF2Cl | Me | H | CH2CH2CH2—SMe |
| 385 | CF2CF2Cl | Me | H | CH2CH2CH2—OEt |
| 386 | CF2CF2Cl | Me | H | CH2CH2CH2—OiPr |
| 387 | CF2CF2Cl | Me | H | CH2CH2CH2—OBu |
| 388 | CF2CF2Cl | Me | H | CH2—COOMe |
| 389 | CF2CF2Cl | Me | H | CH2—COOH |
| 390 | CF2CF2Cl | Me | H | CH(CH3)COOMe |
| 391 | CF2CF2Cl | Me | H | CH(CH3)COOEt |
| 392 | CF2CF2Cl | Me | H | CH2CH2—COOMe |
| 393 | CF2CF2Cl | Me | H | CH(COOMe)2 |
| 394 | CF2CF2Cl | Me | H | CH(COOEt)CH2—CH(CH3)2 |
| 395 | CF2CF2Cl | Me | H | CH(COOMe)CH(CH3)2 |
| 396 | CF2CF2Cl | Me | H | COCH2OMe |
| 397 | CF2CF2Cl | Me | H | CH(cPr)2 |
| 398 | CF2CF2Cl | Me | H | Ph |
| 399 | CF2CF2Cl | Me | H | CH2PH |
| 400 | CF2CF2Cl | Me | H | CH2-Pyridin-3-yl |
| 401 | CF2CF2Cl | Me | H | CH2—6-Cl-Pyridin-3-yl |
| 402 | CF2CF2Cl | Me | H | CH(CH3)Ph |
| 403 | CF2CF2Cl | Me | H | CH2CH2—Ph |
| 404 | CF2CF2Cl | Me | H | CH2—2-CF3—Ph |
| 405 | CF2CF2Cl | Me | H | CH2CH2CHPh2 |
| 406 | CF2CF2Cl | Me | H | O—CH2CH3 |
| 407 | CF2CF2Cl | Me | H | O—CH3 |
| 408 | CF2CF2Cl | Me | H | O—CH2CH═CH2 |
| 409 | CF2CF2Cl | Me | H | O—tBu |
| 410 | CF2CF2Cl | Me | H | O—Pr |
| 411 | CF2CF2Cl | Me | H | O—CH2cPr |
| 412 | CF2CF2Cl | Me | H | O—CH2CH(CH3)2 |
| 413 | CF2CF2Cl | Me | H | O—CH2CF3 |
| 414 | CF2CF2Cl | Me | H | O—CH(CH3)cPr |
| 415 | CF2CF2Cl | Me | H | O—CH2CH2Cl |
| 416 | CF2CF2Cl | Me | H | O—CH2C≡CH |
| 417 | CF2CF2Cl | Me | H | O—CH2C≡CCH3 |
| 418 | CF2CF2Cl | Me | H | O—CH(CH3)C≡CH |
| 419 | CF2CF2Cl | Me | H | Indan-1-yl |
| 420 | CF2CF2Cl | Me | H | Tetrahydrofuran-2-ylmethyl |
| 421 | CF2CF2Cl | Me | Morpholin-4-yl | |
| 422 | CF2CF2Cl | Me | 2-(Methoxycarbonyppiperidin-1-yl | |
| 423 | CF2CF2Cl | Me | 2-(Ethoxycarbonyl)piperidin-1-yl | |
| 424 | CF2CF2Cl | Me | Thiazolidin-3-yl | |
| 425 | CF2CF2Cl | Me | Pyrrolidin-1-yl | |
| 426 | CF2CF2Cl | Me | 2-Methylpyrrolidin-1-yl | |
| 427 | CF2CF2Cl | Me | ═CH—N(CH3)2 | |
| 428 | CF2CF2Cl | Me | ═C(CH3)N(CH3)2 | |
| 429 | CF2CF2Cl | Me | ═CH—N(C2H5)2 | |
| 430 | CF2CF2Cl | Me | ═C(CH3)N(C2H5)2 | |
| 431 | CF2CF2Cl | Me | ═CH-Piperidine | |
| 432 | CF2CF2Cl | Me | ═CH-Morpholine | |
| 433 | CF2CF2Cl | Me | ═CH-Pyrrolidine | |
| 434 | CF2CF2Cl | Me | Me | Me |
| 435 | CF2CF2Cl | Me | Me | Et |
| 436 | CF2CF2Cl | Me | Et | Me |
| 437 | CF2CF2Cl | Me | Et | Et |
| 438 | CF2CF2Cl | Me | Me | Ph |
| 439 | CF2CF2Cl | Me | Me | CH2CO2Me |
| 440 | CF2CF2Cl | Me | H | Me |
| 441 | CF2Cl | Me | H | Me |
| 442 | CF2Cl | Me | H | Et |
| 443 | CF2Cl | Me | H | Pr |
| 444 | CF2Cl | Me | H | iPr |
| 445 | CF2Cl | Me | H | cPr |
| 446 | CF2Cl | Me | H | Bu |
| 447 | CF2Cl | Me | H | cBu |
| 448 | CF2Cl | Me | H | tBu |
| 449 | CF2Cl | Me | H | cPentyl |
| 450 | CF2Cl | Me | H | cHexyl |
| 451 | CF2Cl | Me | H | CH2(CH2)3CH3 |
| 452 | CF2Cl | Me | H | CH2(CH2)4CH3 |
| 453 | CF2Cl | Me | H | CH2—cPr |
| 454 | CF2Cl | Me | H | CH2—CN |
| 455 | CF2Cl | Me | H | CH2—C(CH3)3 |
| 456 | CF2Cl | Me | H | CH2CF2CF3 |
| 457 | CF2Cl | Me | H | CH2CF3 |
| 458 | CF2Cl | Me | H | CH2(CF2)2CF3 |
| 459 | CF2Cl | Me | H | CH2CH(CH3)CH2CH3 |
| 460 | CF2Cl | Me | H | CH2C(CH3)2CH2F |
| 461 | CF2Cl | Me | H | CH2CH(CH3)2 |
| 462 | CF2Cl | Me | H | CH2CH(CH2CH3)2 |
| 463 | CF2Cl | Me | H | CH2CH2CH(CH3)2 |
| 464 | CF2Cl | Me | H | CH2CH2C(CH3)3 |
| 465 | CF2Cl | Me | H | CH2CH═CH2 |
| 466 | CF2Cl | Me | H | CH2CH═CHCH3 |
| 467 | CF2Cl | Me | H | CH2—C(CH3)═CH2 |
| 468 | CF2Cl | Me | H | CH2—C≡CH |
| 469 | CF2Cl | Me | H | CH(CH3)CH2CH3 |
| 470 | CF2Cl | Me | H | CH(CH3)cPr |
| 471 | CF2Cl | Me | H | CH(CH3)CF3 |
| 472 | CF2Cl | Me | H | CH(CH3)(CH2)2CH3 |
| 473 | CF2Cl | Me | H | CH(CH3)(CH2)4CH3 |
| 474 | CF2Cl | Me | H | CH(CH3)(CH2)5CH3 |
| 475 | CF2Cl | Me | H | CH(CH2CH3)(CH2)3CH3 |
| 476 | CF2Cl | Me | H | CH(CH3)CH2CH(CH3)2 |
| 477 | CF2Cl | Me | H | CH(CH3)C(CH3)3 |
| 478 | CF2Cl | Me | H | CH(CH3)CH(CH3)2 |
| 479 | CF2Cl | Me | H | CH(CH3)CH2CH2CH(CH3)2 |
| 480 | CF2Cl | Me | H | CH(CH2CH3)2 |
| 481 | CF2Cl | Me | H | C(CH3)2CH2CH3 |
| 482 | CF2Cl | Me | H | C(CH3)2CH2C(CH3)3 |
| 483 | CF2Cl | Me | H | CH2—CH(OMe)2 |
| 484 | CF2Cl | Me | H | CH2—CH(OEt)2 |
| 485 | CF2Cl | Me | H | CH2CH2—OH |
| 486 | CF2Cl | Me | H | CH2CH2—OMe |
| 487 | CF2Cl | Me | H | CH2CH2—OEt |
| 488 | CF2Cl | Me | H | CH2CH2—SMe |
| 489 | CF2Cl | Me | H | CH2CH2—CN |
| 490 | CF2Cl | Me | H | CH2CH2—NMe2 |
| 491 | CF2Cl | Me | H | CH2CH2-Morpholin-4-yl |
| 492 | CF2Cl | Me | H | CH(CH3)CH2—OMe |
| 493 | CF2Cl | Me | H | CH(CH3)CH2—NMe2 |
| 494 | CF2Cl | Me | H | CH2CH2CH2—OMe |
| 495 | CF2Cl | Me | H | CH2CH2CH2—SMe |
| 496 | CF2Cl | Me | H | CH2CH2CH2—OEt |
| 497 | CF2Cl | Me | H | CH2CH2CH2—OiPr |
| 498 | CF2Cl | Me | H | CH2CH2CH2—OBu |
| 499 | CF2Cl | Me | H | CH2—COOMe |
| 500 | CF2Cl | Me | H | CH2—COOH |
| 501 | CF2Cl | Me | H | CH(CH3)COOMe |
| 502 | CF2Cl | Me | H | CH(CH3)COOEt |
| 503 | CF2Cl | Me | H | CH2CH2—COOMe |
| 504 | CF2Cl | Me | H | CH(COOMe)2 |
| 505 | CF2Cl | Me | H | CH(COOEt)CH2—CH(CH3)2 |
| 506 | CF2Cl | Me | H | CH(COOMe)CH(CH3)2 |
| 507 | CF2Cl | Me | H | COCH2OMe |
| 508 | CF2Cl | Me | H | CH(cPr)2 |
| 509 | CF2Cl | Me | H | Ph |
| 510 | CF2Cl | Me | H | CH2PH |
| 511 | CF2Cl | Me | H | CH2-Pyridin-3-yl |
| 512 | CF2Cl | Me | H | CH2—6-Cl-Pyridin-3-yl |
| 513 | CF2Cl | Me | H | CH(CH3)Ph |
| 514 | CF2Cl | Me | H | CH2CH2—Ph |
| 515 | CF2Cl | Me | H | CH2—2-CF3—Ph |
| 516 | CF2Cl | Me | H | CH2CH2CHPh2 |
| 517 | CF2Cl | Me | H | O—CH2CH3 |
| 518 | CF2Cl | Me | H | O—CH3 |
| 519 | CF2Cl | Me | H | O—CH2CH═CH2 |
| 520 | CF2Cl | Me | H | O—tBu |
| 521 | CF2Cl | Me | H | O—Pr |
| 522 | CF2Cl | Me | H | O—CH2cPr |
| 523 | CF2Cl | Me | H | O—CH2CH(CH3)2 |
| 524 | CF2Cl | Me | H | O—CH2CF3 |
| 525 | CF2Cl | Me | H | O—CH(CH3)cPr |
| 526 | CF2Cl | Me | H | O—CH2CH2Cl |
| 527 | CF2Cl | Me | H | O—CH2C≡CH |
| 528 | CF2Cl | Me | H | O—CH2C≡CCH3 |
| 529 | CF2Cl | Me | H | O—CH(CH3)C≡CH |
| 530 | CF2Cl | Me | H | Indan-1-yl |
| 531 | CF2Cl | Me | H | Tetrahydrofuran-2-ylmethyl |
| 532 | CF2Cl | Me | Morpholin-4-yl | |
| 533 | CF2Cl | Me | 2-(Methoxycarbonyl)piperidin-1-yl | |
| 534 | CF2Cl | Me | 2-(Ethoxycarbonyl)piperidin-1-yl | |
| 535 | CF2Cl | Me | Thiazolidin-3-yl | |
| 536 | CF2Cl | Me | Pyrrolidin-1-yl | |
| 537 | CF2Cl | Me | 2-Methylpyrrolidin-1-yl | |
| 538 | CF2Cl | Me | ═CH—N(CH3)2 | |
| 539 | CF2Cl | Me | ═C(CH3)N(CH3)2 | |
| 540 | CF2Cl | Me | ═CH—N(C2H5)2 | |
| 541 | CF2Cl | Me | ═C(CH3)N(C2H5)2 | |
| 542 | CF2Cl | Me | ═CH-Piperidine | |
| 543 | CF2Cl | Me | ═CH-Morpholine | |
| 544 | CF2Cl | Me | ═CH-Pyrrolidine | |
| 545 | CF2Cl | Me | Me | Me |
| 546 | CF2Cl | Me | Me | Et |
| 547 | CF2Cl | Me | Et | Me |
| 548 | CF2Cl | Me | Et | Et |
| 549 | CF2Cl | Me | Me | Ph |
| 550 | CF2Cl | Me | Me | CH2CO2Me |
| 551 | CF3 | Et | H | Me |
| 552 | CF3 | Et | H | Et |
| 553 | CF3 | Et | H | Pr |
| 554 | CF3 | Et | H | iPr |
| 555 | CF3 | Et | H | cPr |
| 556 | CF3 | Et | H | Bu |
| 557 | CF3 | Et | H | cBu |
| 558 | CF3 | Et | H | tBu |
| 559 | CF3 | Et | H | cPentyl |
| 560 | CF3 | Et | H | cHexyl |
| 561 | CF3 | Et | H | CH2(CH2)3CH3 |
| 562 | CF3 | Et | H | CH2(CH2)4CH3 |
| 563 | CF3 | Et | H | CH2—cPr |
| 564 | CF3 | Et | H | CH2CN |
| 565 | CF3 | Et | H | CH2—C(CH3)3 |
| 567 | CF3 | Et | H | CH2CF2CF3 |
| 568 | CF3 | Et | H | CH2CF3 |
| 569 | CF3 | Et | H | CH2(CF2)2CF3 |
| 570 | CF3 | Et | H | CH2CH(CH3)CH2CH3 |
| 571 | CF3 | Et | H | CH2C(CH3)2CH2F |
| 572 | CF3 | Et | H | CH2CH(CH3)2 |
| 573 | CF3 | Et | H | CH2CH(CH2CH3)2 |
| 574 | CF3 | Et | H | CH2CH2CH(CH3)2 |
| 575 | CF3 | Et | H | CH2CH2C(CH3)3 |
| 576 | CF3 | Et | H | CH2CH═CH2 |
| 577 | CF3 | Et | H | CH2CH═CHCH3 |
| 578 | CF3 | Et | H | CH2—C(CH3)═CH2 |
| 579 | CF3 | Et | H | CH2—C≡CH |
| 580 | CF3 | Et | H | CH(CH3)CH2CH3 |
| 581 | CF3 | Et | H | CH(CH3)cPr |
| 582 | CF3 | Et | H | CH(CH3)CF3 |
| 583 | CF3 | Et | H | CH(CH3)(CH2)2CH3 |
| 584 | CF3 | Et | H | CH(CH3)(CH2)4CH3 |
| 585 | CF3 | Et | H | CH(CH3)(CH2)5CH3 |
| 586 | CF3 | Et | H | CH(CH2CH3)(CH2)3CH3 |
| 587 | CF3 | Et | H | CH(CH3)CH2CH(CH3)2 |
| 588 | CF3 | Et | H | CH(CH3)C(CH3)3 |
| 589 | CF3 | Et | H | CH(CH3)CH(CH3)2 |
| 590 | CF3 | Et | H | CH(CH3)CH2CH2CH(CH3)2 |
| 591 | CF3 | Et | H | CH(CH2CH3)2 |
| 592 | CF3 | Et | H | C(CH3)2CH2CH3 |
| 593 | CF3 | Et | H | C(CH3)2CH2C(CH3)3 |
| 594 | CF3 | Et | H | CH2—CH(OMe)2 |
| 595 | CF3 | Et | H | CH2—CH(OEt)2 |
| 596 | CF3 | Et | H | CH2CH2—OH |
| 597 | CF3 | Et | H | CH2CH2—OMe |
| 598 | CF3 | Et | H | CH2CH2—OEt |
| 599 | CF3 | Et | H | CH2CH2—SMe |
| 600 | CF3 | Et | H | CH2CH2—CN |
| 601 | CF3 | Et | H | CH2CH2—NMe2 |
| 602 | CF3 | Et | H | CH2CH2-Morpholin-4-yl |
| 603 | CF3 | Et | H | CH(CH3)CH2—OMe |
| 604 | CF3 | Et | H | CH(CH3)CH2—NMe2 |
| 605 | CF3 | Et | H | CH2CH2CH2—OMe |
| 606 | CF3 | Et | H | CH2CH2CH2—SMe |
| 607 | CF3 | Et | H | CH2CH2CH2—OEt |
| 608 | CF3 | Et | H | CH2CH2CH2—OiPr |
| 609 | CF3 | Et | H | CH2CH2CH2—OBu |
| 610 | CF3 | Et | H | CH2—COOMe |
| 611 | CF3 | Et | H | CH2—COOH |
| 612 | CF3 | Et | H | CH(CH3)COOMe |
| 613 | CF3 | Et | H | CH(CH3)COOEt |
| 614 | CF3 | Et | H | CH2CH2—COOMe |
| 615 | CF3 | Et | H | CH(COOMe)2 |
| 616 | CF3 | Et | H | CH(COOEt)CH2—CH(CH3)2 |
| 617 | CF3 | Et | H | CH(COOMe)CH(CH3)2 |
| 618 | CF3 | Et | H | COCH2OMe |
| 619 | CF3 | Et | H | CH(cPr)2 |
| 620 | CF3 | Et | H | Ph |
| 621 | CF3 | Et | H | CH2PH |
| 622 | CF3 | Et | H | CH2-Pyridin-3-yl |
| 623 | CF3 | Et | H | CH2—6-Cl-Pyridin-3-yl |
| 624 | CF3 | Et | H | CH(CH3)Ph |
| 625 | CF3 | Et | H | CH2CH2—Ph |
| 626 | CF3 | Et | H | CH2—2-CF3—Ph |
| 627 | CF3 | Et | H | CH2CH2CHPh2 |
| 628 | CF3 | Et | H | O—CH2CH3 |
| 629 | CF3 | Et | H | O—CH3 |
| 630 | CF3 | Et | H | O—CH2CH═CH2 |
| 631 | CF3 | Et | H | O—tBu |
| 632 | CF3 | Et | H | O—Pr |
| 633 | CF3 | Et | H | O—CH2cPr |
| 634 | CF3 | Et | H | O—CH2CH(CH3)2 |
| 635 | CF3 | Et | H | O—CH2CF3 |
| 636 | CF3 | Et | H | O—CH(CH3)cPr |
| 637 | CF3 | Et | H | O—CH2CH2Cl |
| 638 | CF3 | Et | H | O—CH2C≡CH |
| 639 | CF3 | Et | H | O—CH2C≡CCH3 |
| 640 | CF3 | Et | H | O—CH(CH3)C≡CH |
| 641 | CF3 | Et | H | Indan-1-yl |
| 642 | CF3 | Et | H | Tetrahydrofuran-2-ylmethyl |
| 643 | CF3 | Et | Morpholin-4-yl | |
| 644 | CF3 | Et | 2-(Methoxycarbonyl)piperidin-1-yl | |
| 645 | CF3 | Et | 2-(Ethoxycarbonyl)piperidin-1-yl | |
| 646 | CF3 | Et | Thiazolidin-3-yl | |
| 647 | CF3 | Et | Pyrrolidin-1-yl | |
| 648 | CF3 | Et | 2-Methylpyrrolidin-1-yl | |
| 649 | CF3 | Et | ═CH—N(CH3)2 | |
| 650 | CF3 | Et | ═C(CH3)N(CH3)2 | |
| 651 | CF3 | Et | ═CH—N(C2H5)2 | |
| 652 | CF3 | Et | ═C(CH3)N(C2H5)2 | |
| 653 | CF3 | Et | ═CH-Piperidine | |
| 654 | CF3 | Et | ═CH-Morpholine | |
| 655 | CF3 | Et | ═CH-Pyrrolidine | |
| 656 | CF3 | Et | Me | Me |
| 657 | CF3 | Et | Me | Et |
| 658 | CF3 | Et | Et | Me |
| 659 | CF3 | Et | Et | Et |
| 660 | CF3 | Et | Me | Ph |
| 661 | CF2CF3 | Et | H | Me |
| 662 | CF2CF3 | Et | H | Et |
| 663 | CF2CF3 | Et | H | Pr |
| 664 | CF2CF3 | Et | H | iPr |
| 665 | CF2CF3 | Et | H | cPr |
| 666 | CF2CF3 | Et | H | Bu |
| 667 | CF2CF3 | Et | H | cBu |
| 668 | CF2CF3 | Et | H | tBu |
| 669 | CF2CF3 | Et | H | cPentyl |
| 670 | CF2CF3 | Et | H | cHexyl |
| 671 | CF2CF3 | Et | H | CH2(CH2)3CH3 |
| 672 | CF2CF3 | Et | H | CH2(CH2)4CH3 |
| 673 | CF2CF3 | Et | H | CH2—cPr |
| 674 | CF2CF3 | Et | H | CH2—CN |
| 675 | CF2CF3 | Et | H | CH2—C(CH3)3 |
| 676 | CF2CF3 | Et | H | CH2CF2CF3 |
| 677 | CF2CF3 | Et | H | CH2CF3 |
| 678 | CF2CF3 | Et | H | CH2(CF2)2CF3 |
| 679 | CF2CF3 | Et | H | CH2CH(CH3)CH2CH3 |
| 680 | CF2CF3 | Et | H | CH2C(CH3)2CH2F |
| 681 | CF2CF3 | Et | H | CH2CH(CH3)2 |
| 682 | CF2CF3 | Et | H | CH2CH(CH2CH3)2 |
| 683 | CF2CF3 | Et | H | CH2CH2CH(CH3)2 |
| 684 | CF2CF3 | Et | H | CH2CH2C(CH3)3 |
| 685 | CF2CF3 | Et | H | CH2CH═CH2 |
| 686 | CF2CF3 | Et | H | CH2CH═CHCH3 |
| 687 | CF2CF3 | Et | H | CH2—C(CH3)═CH2 |
| 688 | CF2CF3 | Et | H | CH2—C≡CH |
| 689 | CF2CF3 | Et | H | CH(CH3)CH2CH3 |
| 690 | CF2CF3 | Et | H | CH(CH3)cPr |
| 691 | CF2CF3 | Et | H | CH(CH3)CF3 |
| 692 | CF2CF3 | Et | H | CH(CH3)(CH2)2CH3 |
| 693 | CF2CF3 | Et | H | CH(CH3)(CH2)4CH3 |
| 694 | CF2CF3 | Et | H | CH(CH3)(CH2)5CH3 |
| 695 | CF2CF3 | Et | H | CH(CH2CH3)(CH2)3CH3 |
| 696 | CF2CF3 | Et | H | CH(CH3)CH2CH(CH3)2 |
| 697 | CF2CF3 | Et | H | CH(CH3)C(CH3)3 |
| 698 | CF2CF3 | Et | H | CH(CH3)CH(CH3)2 |
| 699 | CF2CF3 | Et | H | CH(CH3)CH2CH2CH(CH3)2 |
| 700 | CF2CF3 | Et | H | CH(CH2CH3)2 |
| 701 | CF2CF3 | Et | H | C(CH3)2CH2CH3 |
| 702 | CF2CF3 | Et | H | C(CH3)2CH2C(CH3)3 |
| 703 | CF2CF3 | Et | H | CH2—CH(OMe)2 |
| 704 | CF2CF3 | Et | H | CH2—CH(OEt)2 |
| 705 | CF2CF3 | Et | H | CH2CH2—OH |
| 706 | CF2CF3 | Et | H | CH2CH2—OMe |
| 707 | CF2CF3 | Et | H | CH2CH2—OEt |
| 708 | CF2CF3 | Et | H | CH2CH2—SMe |
| 709 | CF2CF3 | Et | H | CH2CH2—CN |
| 710 | CF2CF3 | Et | H | CH2CH2—NMe2 |
| 711 | CF2CF3 | Et | H | CH2CH2-Morpholin-4-yl |
| 712 | CF2CF3 | Et | H | CH(CH3)CH2—OMe |
| 713 | CF2CF3 | Et | H | CH(CH3)CH2—NMe2 |
| 714 | CF2CF3 | Et | H | CH2CH2CH2—OMe |
| 715 | CF2CF3 | Et | H | CH2CH2CH2—SMe |
| 716 | CF2CF3 | Et | H | CH2CH2CH2—OEt |
| 717 | CF2CF3 | Et | H | CH2CH2CH2—OiPr |
| 718 | CF2CF3 | Et | H | CH2CH2CH2—OBu |
| 719 | CF2CF3 | Et | H | CH2—COOMe |
| 720 | CF2CF3 | Et | H | CH2—COOH |
| 721 | CF2CF3 | Et | H | CH(CH3)COOMe |
| 722 | CF2CF3 | Et | H | CH(CH3)COOEt |
| 723 | CF2CF3 | Et | H | CH2CH2—COOMe |
| 724 | CF2CF3 | Et | H | CH(COOMe)2 |
| 725 | CF2CF3 | Et | H | CH(COOEt)CH2—CH(CH3)2 |
| 726 | CF2CF3 | Et | H | CH(COOMe)CH(CH3)2 |
| 727 | CF2CF3 | Et | H | COCH2OMe |
| 728 | CF2CF3 | Et | H | CH(cPr)2 |
| 729 | CF2CF3 | Et | H | Ph |
| 730 | CF2CF3 | Et | H | CH2PH |
| 731 | CF2CF3 | Et | H | CH2-Pyridin-3-yl |
| 732 | CF2CF3 | Et | H | CH2—6-Cl-Pyridin-3-yl |
| 733 | CF2CF3 | Et | H | CH(CH3)Ph |
| 734 | CF2CF3 | Et | H | CH2CH2—Ph |
| 735 | CF2CF3 | Et | H | CH2—2-CF3—Ph |
| 736 | CF2CF3 | Et | H | CH2CH2CHPh2 |
| 737 | CF2CF3 | Et | H | O—CH2CH3 |
| 738 | CF2CF3 | Et | H | O—CH3 |
| 739 | CF2CF3 | Et | H | O—CH2CH═CH2 |
| 740 | CF2CF3 | Et | H | O—tBu |
| 741 | CF2CF3 | Et | H | O—Pr |
| 742 | CF2CF3 | Et | H | O—CH2cPr |
| 743 | CF2CF3 | Et | H | O—CH2CH(CH3)2 |
| 744 | CF2CF3 | Et | H | O—CH2CF3 |
| 745 | CF2CF3 | Et | H | O—CH(CH3)cPr |
| 746 | CF2CF3 | Et | H | O—CH2CH2Cl |
| 747 | CF2CF3 | Et | H | O—CH2C≡CH |
| 748 | CF2CF3 | Et | H | O—CH2C≡CCH3 |
| 749 | CF2CF3 | Et | H | O—CH(CH3)C≡CH |
| 750 | CF2CF3 | Et | H | Indan-1-yl |
| 751 | CF2CF3 | Et | H | Tetrahydrofuran-2-ylmethyl |
| 752 | CF2CF3 | Et | Morpholin-4-yl | |
| 753 | CF2CF3 | Et | 2-(Methoxycarbonyl)piperidin-1-yl | |
| 754 | CF2CF3 | Et | 2-(Ethoxycarbonyl)piperidin-1-yl | |
| 755 | CF2CF3 | Et | Thiazolidin-3-yl | |
| 756 | CF2CF3 | Et | Pyrrolidin-1-yl | |
| 757 | CF2CF3 | Et | 2-Methylpyrrolidin-1-yl | |
| 758 | CF2CF3 | Et | ═CH—N(CH3)2 | |
| 759 | CF2CF3 | Et | ═C(CH3)N(CH3)2 | |
| 760 | CF2CF3 | Et | ═CH—N(C2H5)2 | |
| 761 | CF2CF3 | Et | ═C(CH3)N(C2H5)2 | |
| 762 | CF2CF3 | Et | ═CH-Piperidine | |
| 763 | CF2CF3 | Et | ═CH-Morpholine | |
| 764 | CF2CF3 | Et | ═CH-Pyrrolidine | |
| 765 | CF2CF3 | Et | Me | Me |
| 766 | CF2CF3 | Et | Me | Et |
| 767 | CF2CF3 | Et | Et | Me |
| 768 | CF2CF3 | Et | Et | Et |
| 769 | CF2CF3 | Et | Me | Ph |
| 770 | CF2CF3 | Et | Me | CH2CO2Me |
| 771 | CF2Cl | Et | H | Me |
| 772 | CF2Cl | Et | H | Et |
| 773 | CF2Cl | Et | H | Pr |
| 774 | CF2Cl | Et | H | iPr |
| 775 | CF2Cl | Et | H | cPr |
| 776 | CF2Cl | Et | H | Bu |
| 777 | CF2Cl | Et | H | cBu |
| 778 | CF2Cl | Et | H | tBu |
| 779 | CF2Cl | Et | H | cPentyl |
| 780 | CF2Cl | Et | H | cHexyl |
| 781 | CF2Cl | Et | H | CH2(CH2)3CH3 |
| 782 | CF2Cl | Et | H | CH2(CH2)4CH3 |
| 783 | CF2Cl | Et | H | CH2—cPr |
| 784 | CF2Cl | Et | H | CH2—CN |
| 785 | CF2Cl | Et | H | CH2—C(CH3)3 |
| 786 | CF2Cl | Et | H | CH2CF2CF3 |
| 787 | CF2Cl | Et | H | CH2CF3 |
| 788 | CF2Cl | Et | H | CH2(CF2)2CF3 |
| 789 | CF2Cl | Et | H | CH2CH(CH3)CH2CH3 |
| 790 | CF2Cl | Et | H | CH2C(CH3)2CH2F |
| 791 | CF2Cl | Et | H | CH2CH(CH3)2 |
| 792 | CF2Cl | Et | H | CH2CH(CH2CH3)2 |
| 793 | CF2Cl | Et | H | CH2CH2CH(CH3)2 |
| 794 | CF2Cl | Et | H | CH2CH2C(CH3)3 |
| 795 | CF2Cl | Et | H | CH2CH═CH2 |
| 796 | CF2Cl | Et | H | CH2CH═CHCH3 |
| 797 | CF2Cl | Et | H | CH2—C(CH3)═CH2 |
| 798 | CF2Cl | Et | H | CH2—C≡CH |
| 799 | CF2Cl | Et | H | CH(CH3)CH2CH3 |
| 800 | CF2Cl | Et | H | CH(CH3)cPr |
| 801 | CF2Cl | Et | H | CH(CH3)CF3 |
| 802 | CF2Cl | Et | H | CH(CH3)(CH2)2CH3 |
| 803 | CF2Cl | Et | H | CH(CH3)(CH2)4CH3 |
| 804 | CF2Cl | Et | H | CH(CH3)(CH2)5CH3 |
| 805 | CF2Cl | Et | H | CH(CH2CH3)(CH2)3CH3 |
| 806 | CF2Cl | Et | H | CH(CH3)CH2CH(CH3)2 |
| 807 | CF2Cl | Et | H | CH(CH3)C(CH3)3 |
| 808 | CF2Cl | Et | H | CH(CH3)CH(CH3)2 |
| 809 | CF2Cl | Et | H | CH(CH3)CH2CH2CH(CH3)2 |
| 810 | CF2Cl | Et | H | CH(CH2CH3)2 |
| 811 | CF2Cl | Et | H | C(CH3)2CH2CH3 |
| 812 | CF2Cl | Et | H | C(CH3)2CH2C(CH3)3 |
| 813 | CF2Cl | Et | H | CH2—CH(OMe)2 |
| 814 | CF2Cl | Et | H | CH2—CH(OEt)2 |
| 815 | CF2Cl | Et | H | CH2CH2—OH |
| 816 | CF2Cl | Et | H | CH2CH2—OMe |
| 817 | CF2Cl | Et | H | CH2CH2—OEt |
| 818 | CF2Cl | Et | H | CH2CH2—SMe |
| 819 | CF2Cl | Et | H | CH2CH2—CN |
| 820 | CF2Cl | Et | H | CH2CH2—NMe2 |
| 821 | CF2Cl | Et | H | CH2CH2-Morpholin-4-yl |
| 822 | CF2Cl | Et | H | CH(CH3)CH2—OMe |
| 823 | CF2Cl | Et | H | CH(CH3)CH2—NMe2 |
| 824 | CF2Cl | Et | H | CH2CH2CH2—OMe |
| 825 | CF2Cl | Et | H | CH2CH2CH2—SMe |
| 826 | CF2Cl | Et | H | CH2CH2CH2—OEt |
| 827 | CF2Cl | Et | H | CH2CH2CH2—OiPr |
| 828 | CF2Cl | Et | H | CH2CH2CH2—OBu |
| 829 | CF2Cl | Et | H | CH2—COOMe |
| 830 | CF2Cl | Et | H | CH2—COOH |
| 831 | CF2Cl | Et | H | CH(CH3)COOMe |
| 832 | CF2Cl | Et | H | CH(CH3)COOEt |
| 833 | CF2Cl | Et | H | CH2CH2—COOMe |
| 834 | CF2Cl | Et | H | CH(COOMe)2 |
| 835 | CF2Cl | Et | H | CH(COOEt)CH2—CH(CH3)2 |
| 836 | CF2Cl | Et | H | CH(COOMe)CH(CH3)2 |
| 837 | CF2Cl | Et | H | COCH2OMe |
| 838 | CF2Cl | Et | H | CH(cPr)2 |
| 839 | CF2Cl | Et | H | Ph |
| 840 | CF2Cl | Et | H | CH2PH |
| 841 | CF2Cl | Et | H | CH2-Pyridin-3-yl |
| 842 | CF2Cl | Et | H | CH2—6-Cl-Pyridin-3-yl |
| 843 | CF2Cl | Et | H | CH(CH3)Ph |
| 844 | CF2Cl | Et | H | CH2CH2—Ph |
| 845 | CF2Cl | Et | H | CH2—2-CF3—Ph |
| 846 | CF2Cl | Et | H | CH2CH2CHPh2 |
| 847 | CF2Cl | Et | H | O—CH2CH3 |
| 848 | CF2Cl | Et | H | O—CH3 |
| 849 | CF2Cl | Et | H | O—CH2CH═CH2 |
| 850 | CF2Cl | Et | H | O—tBu |
| 851 | CF2Cl | Et | H | O—Pr |
| 852 | CF2Cl | Et | H | O—CH2cPr |
| 853 | CF2Cl | Et | H | O—CH2CH(CH3)2 |
| 854 | CF2Cl | Et | H | O—CH2CF3 |
| 855 | CF2Cl | Et | H | O—CH(CH3)cPr |
| 856 | CF2Cl | Et | H | O—CH2CH2Cl |
| 857 | CF2Cl | Et | H | O—CH2C≡CH |
| 858 | CF2Cl | Et | H | O—CH2C≡CCH3 |
| 859 | CF2Cl | Et | H | O—CH(CH3)C≡CH |
| 860 | CF2Cl | Et | H | Indan-1-yl |
| 861 | CF2Cl | Et | H | Tetrahydrofuran-2-ylmethyl |
| 862 | CF2Cl | Et | Morpholin-4-yl | |
| 863 | CF2Cl | Et | 2-(Methoxycarbonyl)piperidin-1-yl | |
| 864 | CF2Cl | Et | 2-(Ethoxycarbonyl)piperidin-1-yl | |
| 865 | CF2Cl | Et | Thiazolidin-3-yl | |
| 866 | CF2Cl | Et | Pyrrolidin-1-yl | |
| 867 | CF2Cl | Et | 2-Methylpyrrolidin-1-yl | |
| 868 | CF2Cl | Et | ═CH—N(CH3)2 | |
| 869 | CF2Cl | Et | ═C(CH3)N(CH3)2 | |
| 870 | CF2Cl | Et | ═CH—N(C2H5)2 | |
| 871 | CF2Cl | Et | ═C(CH3)N(C2H5)2 | |
| 872 | CF2Cl | Et | ═CH-Piperidine | |
| 873 | CF2Cl | Et | ═CH-Morpholine | |
| 874 | CF2Cl | Et | ═CH-Pyrrolidine | |
| 875 | CF2Cl | Et | Me | Me |
| 876 | CF2Cl | Et | Me | Et |
| 877 | CF2Cl | Et | Et | Me |
| 878 | CF2Cl | Et | Et | Et |
| 879 | CF2Cl | Et | Me | Ph |
| 880 | CF2Cl | Et | Me | CH2CO2Me |
| 881 | CF2CF2Cl | Et | H | Me |
| 882 | CF2CF2Cl | Et | H | Et |
| 883 | CF2CF2Cl | Et | H | Pr |
| 884 | CF2CF2Cl | Et | H | iPr |
| 885 | CF2CF2Cl | Et | H | cPr |
| 886 | CF2CF2Cl | Et | H | Bu |
| 887 | CF2CF2Cl | Et | H | cBu |
| 888 | CF2CF2Cl | Et | H | tBu |
| 889 | CF2CF2Cl | Et | H | cPentyl |
| 890 | CF2CF2Cl | Et | H | cHexyl |
| 891 | CF2CF2Cl | Et | H | CH2(CH2)3CH3 |
| 892 | CF2CF2Cl | Et | H | CH2(CH2)4CH3 |
| 893 | CF2CF2Cl | Et | H | CH2—cPr |
| 894 | CF2CF2Cl | Et | H | CH2—CN |
| 895 | CF2CF2Cl | Et | H | CH2—C(CH3)3 |
| 896 | CF2CF2Cl | Et | H | CH2CF2CF3 |
| 897 | CF2CF2Cl | Et | H | CH2CF3 |
| 898 | CF2CF2Cl | Et | H | CH2(CF2)2CF3 |
| 899 | CF2CF2Cl | Et | H | CH2CH(CH3)CH2CH3 |
| 900 | CF2CF2Cl | Et | H | CH2C(CH3)2CH2F |
| 901 | CF2CF2Cl | Et | H | CH2CH(CH3)2 |
| 902 | CF2CF2Cl | Et | H | CH2CH(CH2CH3)2 |
| 903 | CF2CF2Cl | Et | H | CH2CH2CH(CH3)2 |
| 904 | CF2CF2Cl | Et | H | CH2CH2C(CH3)3 |
| 905 | CF2CF2Cl | Et | H | CH2CH═CH2 |
| 906 | CF2CF2Cl | Et | H | CH2CH═CHCH3 |
| 907 | CF2CF2Cl | Et | H | CH2—C(CH3)═CH2 |
| 908 | CF2CF2Cl | Et | H | CH2—C≡CH |
| 909 | CF2CF2Cl | Et | H | CH(CH3)CH2CH3 |
| 910 | CF2CF2Cl | Et | H | CH(CH3)cPr |
| 911 | CF2CF2Cl | Et | H | CH(CH3)CF3 |
| 912 | CF2CF2Cl | Et | H | CH(CH3)(CH2)2CH3 |
| 913 | CF2CF2Cl | Et | H | CH(CH3)(CH2)4CH3 |
| 914 | CF2CF2Cl | Et | H | CH(CH3)(CH2)5CH3 |
| 915 | CF2CF2Cl | Et | H | CH(CH2CH3)(CH2)3CH3 |
| 916 | CF2CF2Cl | Et | H | CH(CH3)CH2CH(CH3)2 |
| 917 | CF2CF2Cl | Et | H | CH(CH3)C(CH3)3 |
| 918 | CF2CF2Cl | Et | H | CH(CH3)CH(CH3)2 |
| 919 | CF2CF2Cl | Et | H | CH(CH3)CH2CH2CH(CH3)2 |
| 920 | CF2CF2Cl | Et | H | CH(CH2CH3)2 |
| 921 | CF2CF2Cl | Et | H | C(CH3)2CH2CH3 |
| 922 | CF2CF2Cl | Et | H | C(CH3)2CH2C(CH3)3 |
| 923 | CF2CF2Cl | Et | H | CH2—CH(OMe)2 |
| 924 | CF2CF2Cl | Et | H | CH2—CH(OEt)2 |
| 925 | CF2CF2Cl | Et | H | CH2CH2—OH |
| 926 | CF2CF2Cl | Et | H | CH2CH2—OMe |
| 927 | CF2CF2Cl | Et | H | CH2CH2—OEt |
| 928 | CF2CF2Cl | Et | H | CH2CH2—SMe |
| 929 | CF2CF2Cl | Et | H | CH2CH2—CN |
| 930 | CF2CF2Cl | Et | H | CH2CH2—NMe2 |
| 931 | CF2CF2Cl | Et | H | CH2CH2-Morpholin-4-yl |
| 932 | CF2CF2Cl | Et | H | CH(CH3)CH2—OMe |
| 933 | CF2CF2Cl | Et | H | CH(CH3)CH2—NMe2 |
| 934 | CF2CF2Cl | Et | H | CH2CH2CH2—OMe |
| 935 | CF2CF2Cl | Et | H | CH2CH2CH2—SMe |
| 936 | CF2CF2Cl | Et | H | CH2CH2CH2—OEt |
| 937 | CF2CF2Cl | Et | H | CH2CH2CH2—OiPr |
| 938 | CF2CF2Cl | Et | H | CH2CH2CH2—OBu |
| 939 | CF2CF2Cl | Et | H | CH2—COOMe |
| 940 | CF2CF2Cl | Et | H | CH2—COOH |
| 941 | CF2CF2Cl | Et | H | CH(CH3)COOMe |
| 942 | CF2CF2Cl | Et | H | CH(CH3)COOEt |
| 943 | CF2CF2Cl | Et | H | CH2CH2—COOMe |
| 944 | CF2CF2Cl | Et | H | CH(COOMe)2 |
| 945 | CF2CF2Cl | Et | H | CH(COOEt)CH2—CH(CH3)2 |
| 946 | CF2CF2Cl | Et | H | CH(COOMe)CH(CH3)2 |
| 947 | CF2CF2Cl | Et | H | COCH2OMe |
| 948 | CF2CF2Cl | Et | H | CH(cPr)2 |
| 949 | CF2CF2Cl | Et | H | Ph |
| 950 | CF2CF2Cl | Et | H | CH2PH |
| 951 | CF2CF2Cl | Et | H | CH2-Pyridin-3-yl |
| 952 | CF2CF2Cl | Et | H | CH2—6-Cl-Pyridin-3-yl |
| 953 | CF2CF2Cl | Et | H | CH(CH3)Ph |
| 954 | CF2CF2Cl | Et | H | CH2CH2—Ph |
| 955 | CF2CF2Cl | Et | H | CH2—2-CF3—Ph |
| 956 | CF2CF2Cl | Et | H | CH2CH2CHPh2 |
| 957 | CF2CF2Cl | Et | H | O—CH2CH3 |
| 958 | CF2CF2Cl | Et | H | O—CH3 |
| 959 | CF2CF2Cl | Et | H | O—CH2CH═CH2 |
| 960 | CF2CF2Cl | Et | H | O—tBu |
| 961 | CF2CF2Cl | Et | H | O—Pr |
| 962 | CF2CF2Cl | Et | H | O—CH2cPr |
| 963 | CF2CF2Cl | Et | H | O—CH2CH(CH3)2 |
| 964 | CF2CF2Cl | Et | H | O—CH2CF3 |
| 965 | CF2CF2Cl | Et | H | O—CH(CH3)cPr |
| 966 | CF2CF2Cl | Et | H | O—CH2CH2Cl |
| 967 | CF2CF2Cl | Et | H | O—CH2C≡CH |
| 968 | CF2CF2Cl | Et | H | O—CH2C≡CCH3 |
| 969 | CF2CF2Cl | Et | H | O—CH(CH3)C≡CH |
| 970 | CF2CF2Cl | Et | H | Indan-1-yl |
| 971 | CF2CF2Cl | Et | H | Tetrahydrofuran-2-ylmethyl |
| 972 | CF2CF2Cl | Et | Morpholin-4-yl | |
| 973 | CF2CF2Cl | Et | 2-(Methoxycarbonyl)piperidin-1-yl | |
| 974 | CF2CF2Cl | Et | 2-(Ethoxycarbonyl)piperidin-1-yl | |
| 975 | CF2CF2Cl | Et | Thiazolidin-3-yl | |
| 976 | CF2CF2Cl | Et | Pyrrolidin-1-yl | |
| 977 | CF2CF2Cl | Et | 2-Methylpyrrolidin-1-yl | |
| 978 | CF2CF2Cl | Et | ═CH—N(CH3)2 | |
| 979 | CF2CF2Cl | Et | ═C(CH3)N(CH3)2 | |
| 980 | CF2CF2Cl | Et | ═CH—N(C2H5)2 | |
| 981 | CF2CF2Cl | Et | ═C(CH3)N(C2H5)2 | |
| 982 | CF2CF2Cl | Et | ═CH-Piperidine | |
| 983 | CF2CF2Cl | Et | ═CH-Morpholine | |
| 984 | CF2CF2Cl | Et | ═CH-Pyrrolidine | |
| 985 | CF2CF2Cl | Et | Me | Me |
| 986 | CF2CF2Cl | Et | Me | Et |
| 987 | CF2CF2Cl | Et | Et | Me |
| 988 | CF2CF2Cl | Et | Et | Et |
| 989 | CF2CF2Cl | Et | Me | Ph |
| 990 | CF2CF2Cl | Et | Me | CH2CO2Me |
| 991 | CF3 | Pr | H | Me |
| 992 | CF3 | Pr | H | Et |
| 993 | CF3 | Pr | H | Pr |
| 994 | CF3 | Pr | H | iPr |
| 995 | CF3 | Pr | H | cPr |
| 996 | CF3 | Pr | H | Bu |
| 997 | CF3 | Pr | H | cBu |
| 998 | CF3 | Pr | H | tBu |
| 999 | CF3 | Pr | H | cPentyl |
| 1000 | CF3 | Pr | H | cHexyl |
| 1001 | CF3 | Pr | H | CH2(CH2)3CH3 |
| 1002 | CF3 | Pr | H | CH2(CH2)4CH3 |
| 1003 | CF3 | Pr | H | CH2—cPr |
| 1004 | CF3 | Pr | H | CH2—CN |
| 1005 | CF3 | Pr | H | CH2—C(CH3)3 |
| 1006 | CF3 | Pr | H | CH2CF2CF3 |
| 1007 | CF3 | Pr | H | CH2CF3 |
| 1008 | CF3 | Pr | H | CH2(CF2)2CF3 |
| 1009 | CF3 | Pr | H | CH2CH(CH3)CH2CH3 |
| 1010 | CF3 | Pr | H | CH2C(CH3)2CH2F |
| 1011 | CF3 | Pr | H | CH2CH(CH3)2 |
| 1012 | CF3 | Pr | H | CH2CH(CH2CH3)2 |
| 1013 | CF3 | Pr | H | CH2CH2CH(CH3)2 |
| 1014 | CF3 | Pr | H | CH2CH2C(CH3)3 |
| 1015 | CF3 | Pr | H | CH2CH═CH2 |
| 1016 | CF3 | Pr | H | CH2CH═CHCH3 |
| 1017 | CF3 | Pr | H | CH2—C(CH3)═CH2 |
| 1018 | CF3 | Pr | H | CH2—C≡CH |
| 1019 | CF3 | Pr | H | CH(CH3)CH2CH3 |
| 1020 | CF3 | Pr | H | CH(CH3)cPr |
| 1021 | CF3 | Pr | H | CH(CH3)CF3 |
| 1022 | CF3 | Pr | H | CH(CH3)(CH2)2CH3 |
| 1023 | CF3 | Pr | H | CH(CH3)(CH2)4CH3 |
| 1024 | CF3 | Pr | H | CH(CH3)(CH2)5CH3 |
| 1025 | CF3 | Pr | H | CH(CH2CH3)(CH2)3CH3 |
| 1026 | CF3 | Pr | H | CH(CH3)CH2CH(CH3)2 |
| 1027 | CF3 | Pr | H | CH(CH3)C(CH3)3 |
| 1028 | CF3 | Pr | H | CH(CH3)CH(CH3)2 |
| 1029 | CF3 | Pr | H | CH(CH3)CH2CH2CH(CH3)2 |
| 1030 | CF3 | Pr | H | CH(CH2CH3)2 |
| 1031 | CF3 | Pr | H | C(CH3)2CH2CH3 |
| 1032 | CF3 | Pr | H | C(CH3)2CH2C(CH3)3 |
| 1033 | CF3 | Pr | H | CH2—CH(OMe)2 |
| 1034 | CF3 | Pr | H | CH2—CH(OEt)2 |
| 1035 | CF3 | Pr | H | CH2CH2—OH |
| 1036 | CF3 | Pr | H | CH2CH2—OMe |
| 1037 | CF3 | Pr | H | CH2CH2—OEt |
| 1038 | CF3 | Pr | H | CH2CH2—SMe |
| 1039 | CF3 | Pr | H | CH2CH2—CN |
| 1040 | CF3 | Pr | H | CH2CH2—NMe2 |
| 1041 | CF3 | Pr | H | CH2CH2-Morpholin-4-yl |
| 1042 | CF3 | Pr | H | CH(CH3)CH2—OMe |
| 1043 | CF3 | Pr | H | CH(CH3)CH2—NMe2 |
| 1044 | CF3 | Pr | H | CH2CH2CH2—OMe |
| 1045 | CF3 | Pr | H | CH2CH2CH2—SMe |
| 1046 | CF3 | Pr | H | CH2CH2CH2—OEt |
| 1047 | CF3 | Pr | H | CH2CH2CH2—OiPr |
| 1048 | CF3 | Pr | H | CH2CH2CH2—OBu |
| 1049 | CF3 | Pr | H | CH2—COOMe |
| 1050 | CF3 | Pr | H | CH2—COOH |
| 1051 | CF3 | Pr | H | CH(CH3)COOMe |
| 1052 | CF3 | Pr | H | CH(CH3)COOEt |
| 1053 | CF3 | Pr | H | CH2CH2—COOMe |
| 1054 | CF3 | Pr | H | CH(COOMe)2 |
| 1055 | CF3 | Pr | H | CH(COOEt)CH2—CH(CH3)2 |
| 1056 | CF3 | Pr | H | CH(COOMe)CH(CH3)2 |
| 1057 | CF3 | Pr | H | COCH2OMe |
| 1058 | CF3 | Pr | H | CH(cPr)2 |
| 1059 | CF3 | Pr | H | Ph |
| 1060 | CF3 | Pr | H | CH2PH |
| 1061 | CF3 | Pr | H | CH2-Pyridin-3-yl |
| 1062 | CF3 | Pr | H | CH2—6-Cl-Pyridin-3-yl |
| 1063 | CF3 | Pr | H | CH(CH3)Ph |
| 1064 | CF3 | Pr | H | CH2CH2—Ph |
| 1065 | CF3 | Pr | H | CH2—2-CF3—Ph |
| 1066 | CF3 | Pr | H | CH2CH2CHPh2 |
| 1067 | CF3 | Pr | H | O—CH2CH3 |
| 1068 | CF3 | Pr | H | O—CH3 |
| 1069 | CF3 | Pr | H | O—CH2CH═CH2 |
| 1070 | CF3 | Pr | H | O—tBu |
| 1071 | CF3 | Pr | H | O—Pr |
| 1072 | CF3 | Pr | H | O—CH2cPr |
| 1073 | CF3 | Pr | H | O—CH2CH(CH3)2 |
| 1074 | CF3 | Pr | H | O—CH2CF3 |
| 1075 | CF3 | Pr | H | O—CH(CH3)cPr |
| 1076 | CF3 | Pr | H | O—CH2CH2Cl |
| 1077 | CF3 | Pr | H | O—CH2C≡CH |
| 1078 | CF3 | Pr | H | O—CH2C≡CCH3 |
| 1079 | CF3 | Pr | H | O—CH(CH3)C≡CH |
| 1080 | CF3 | Pr | H | Indan-1-yl |
| 1081 | CF3 | Pr | H | Tetrahydrofuran-2-ylmethyl |
| 1082 | CF3 | Pr | Morpholin-4-yl | |
| 1083 | CF3 | Pr | 2-(Methoxycarbonyl)piperidin-1-yl | |
| 1084 | CF3 | Pr | 2-(Ethoxycarbonyl)piperidin-1-yl | |
| 1085 | CF3 | Pr | Thiazolidin-3-yl | |
| 1086 | CF3 | Pr | Pyrrolidin-1-yl | |
| 1087 | CF3 | Pr | 2-Methylpyrrolidin-1-yl | |
| 1088 | CF3 | Pr | ═CH—N(CH3)2 | |
| 1089 | CF3 | Pr | ═C(CH3)N(CH3)2 | |
| 1090 | CF3 | Pr | ═CH—N(C2H5)2 | |
| 1091 | CF3 | Pr | ═C(CH3)N(C2H5)2 | |
| 1092 | CF3 | Pr | ═CH-Piperidine | |
| 1093 | CF3 | Pr | ═CH-Morpholine | |
| 1094 | CF3 | Pr | ═CH-Pyrrolidine | |
| 1095 | CF3 | Pr | Me | Me |
| 1096 | CF3 | Pr | Me | Et |
| 1097 | CF3 | Pr | Et | Me |
| 1098 | CF3 | Pr | Et | Et |
| 1099 | CF3 | Pr | Me | Ph |
| 1100 | CF3 | Pr | Me | CH2CO2Me |
| 1101 | CF2CF3 | Pr | H | Me |
| 1102 | CF2CF3 | Pr | H | Et |
| 1103 | CF2CF3 | Pr | H | Pr |
| 1104 | CF2CF3 | Pr | H | iPr |
| 1105 | CF2CF3 | Pr | H | cPr |
| 1106 | CF2CF3 | Pr | H | Bu |
| 1107 | CF2CF3 | Pr | H | cBu |
| 1108 | CF2CF3 | Pr | H | tBu |
| 1109 | CF2CF3 | Pr | H | cPentyl |
| 1110 | CF2CF3 | Pr | H | cHexyl |
| 1111 | CF2CF3 | Pr | H | CH2(CH2)3CH3 |
| 1112 | CF2CF3 | Pr | H | CH2(CH2)4CH3 |
| 1113 | CF2CF3 | Pr | H | CH2—cPr |
| 1114 | CF2CF3 | Pr | H | CH2—CN |
| 1115 | CF2CF3 | Pr | H | CH2—C(CH3)3 |
| 1116 | CF2CF3 | Pr | H | CH2CF2CF3 |
| 1117 | CF2CF3 | Pr | H | CH2CF3 |
| 1118 | CF2CF3 | Pr | H | CH2(CF2)2CF3 |
| 1119 | CF2CF3 | Pr | H | CH2CH(CH3)CH2CH3 |
| 1120 | CF2CF3 | Pr | H | CH2C(CH3)2CH2F |
| 1121 | CF2CF3 | Pr | H | CH2CH(CH3)2 |
| 1122 | CF2CF3 | Pr | H | CH2CH(CH2CH3)2 |
| 1123 | CF2CF3 | Pr | H | CH2CH2CH(CH3)2 |
| 1124 | CF2CF3 | Pr | H | CH2CH2C(CH3)3 |
| 1125 | CF2CF3 | Pr | H | CH2CH═CH2 |
| 1126 | CF2CF3 | Pr | H | CH2CH═CHCH3 |
| 1127 | CF2CF3 | Pr | H | CH2—C(CH3)═CH2 |
| 1128 | CF2CF3 | Pr | H | CH2—C≡CH |
| 1129 | CF2CF3 | Pr | H | CH(CH3)CH2CH3 |
| 1130 | CF2CF3 | Pr | H | CH(CH3)cPr |
| 1131 | CF2CF3 | Pr | H | CH(CH3)CF3 |
| 1132 | CF2CF3 | Pr | H | CH(CH3)(CH2)2CH3 |
| 1133 | CF2CF3 | Pr | H | CH(CH3)(CH2)4CH3 |
| 1134 | CF2CF3 | Pr | H | CH(CH3)(CH2)5CH3 |
| 1135 | CF2CF3 | Pr | H | CH(CH2CH3)(CH2)3CH3 |
| 1136 | CF2CF3 | Pr | H | CH(CH3)CH2CH(CH3)2 |
| 1137 | CF2CF3 | Pr | H | CH(CH3)C(CH3)3 |
| 1138 | CF2CF3 | Pr | H | CH(CH3)CH(CH3)2 |
| 1139 | CF2CF3 | Pr | H | CH(CH3)CH2CH2CH(CH3)2 |
| 1140 | CF2CF3 | Pr | H | CH(CH2CH3)2 |
| 1141 | CF2CF3 | Pr | H | C(CH3)2CH2CH3 |
| 1142 | CF2CF3 | Pr | H | C(CH3)2CH2C(CH3)3 |
| 1143 | CF2CF3 | Pr | H | CH2—CH(OMe)2 |
| 1144 | CF2CF3 | Pr | H | CH2—CH(OEt)2 |
| 1145 | CF2CF3 | Pr | H | CH2CH2—OH |
| 1146 | CF2CF3 | Pr | H | CH2CH2—OMe |
| 1147 | CF2CF3 | Pr | H | CH2CH2—OEt |
| 1148 | CF2CF3 | Pr | H | CH2CH2—SMe |
| 1149 | CF2CF3 | Pr | H | CH2CH2—CN |
| 1150 | CF2CF3 | Pr | H | CH2CH2—NMe2 |
| 1151 | CF2CF3 | Pr | H | CH2CH2-Morpholin-4-yl |
| 1152 | CF2CF3 | Pr | H | CH(CH3)CH2—OMe |
| 1153 | CF2CF3 | Pr | H | CH(CH3)CH2—NMe2 |
| 1154 | CF2CF3 | Pr | H | CH2CH2CH2—OMe |
| 1155 | CF2CF3 | Pr | H | CH2CH2CH2—SMe |
| 1156 | CF2CF3 | Pr | H | CH2CH2CH2—OEt |
| 1157 | CF2CF3 | Pr | H | CH2CH2CH2—OiPr |
| 1158 | CF2CF3 | Pr | H | CH2CH2CH2—OBu |
| 1159 | CF2CF3 | Pr | H | CH2—COOMe |
| 1160 | CF2CF3 | Pr | H | CH2—COOH |
| 1161 | CF2CF3 | Pr | H | CH(CH3)COOMe |
| 1162 | CF2CF3 | Pr | H | CH(CH3)COOEt |
| 1163 | CF2CF3 | Pr | H | CH2CH2—COOMe |
| 1164 | CF2CF3 | Pr | H | CH(COOMe)2 |
| 1165 | CF2CF3 | Pr | H | CH(COOEt)CH2—CH(CH3)2 |
| 1166 | CF2CF3 | Pr | H | CH(COOMe)CH(CH3)2 |
| 1167 | CF2CF3 | Pr | H | COCH2OMe |
| 1168 | CF2CF3 | Pr | H | CH(cPr)2 |
| 1169 | CF2CF3 | Pr | H | Ph |
| 1170 | CF2CF3 | Pr | H | CH2PH |
| 1171 | CF2CF3 | Pr | H | CH2-Pyridin-3-yl |
| 1172 | CF2CF3 | Pr | H | CH2—6-Cl-Pyridin-3-yl |
| 1173 | CF2CF3 | Pr | H | CH(CH3)Ph |
| 1174 | CF2CF3 | Pr | H | CH2CH2—Ph |
| 1175 | CF2CF3 | Pr | H | CH2—2-CF3—Ph |
| 1176 | CF2CF3 | Pr | H | CH2CH2CHPh2 |
| 1177 | CF2CF3 | Pr | H | O—CH2CH3 |
| 1178 | CF2CF3 | Pr | H | O—CH3 |
| 1179 | CF2CF3 | Pr | H | O—CH2CH═CH2 |
| 1180 | CF2CF3 | Pr | H | O—tBu |
| 1181 | CF2CF3 | Pr | H | O—Pr |
| 1182 | CF2CF3 | Pr | H | O—CH2cPr |
| 1183 | CF2CF3 | Pr | H | O—CH2CH(CH3)2 |
| 1184 | CF2CF3 | Pr | H | O—CH2CF3 |
| 1185 | CF2CF3 | Pr | H | O—CH(CH3)cPr |
| 1186 | CF2CF3 | Pr | H | O—CH2CH2Cl |
| 1187 | CF2CF3 | Pr | H | O—CH2C≡CH |
| 1188 | CF2CF3 | Pr | H | O—CH2C≡CCH3 |
| 1189 | CF2CF3 | Pr | H | O—CH(CH3)C≡CH |
| 1190 | CF2CF3 | Pr | H | Indan-1-yl |
| 1191 | CF2CF3 | Pr | H | Tetrahydrofuran-2-ylmethyl |
| 1192 | CF2CF3 | Pr | Morpholin-4-yl | |
| 1193 | CF2CF3 | Pr | 2-(Methoxycarbonyl)piperidin-1-yl | |
| 1194 | CF2CF3 | Pr | 2-(Ethoxycarbonyl)piperidin-1-yl | |
| 1195 | CF2CF3 | Pr | Thiazolidin-3-yl | |
| 1196 | CF2CF3 | Pr | Pyrrolidin-1-yl | |
| 1197 | CF2CF3 | Pr | 2-Methylpyrrolidin-1-yl | |
| 1198 | CF2CF3 | Pr | ═CH—N(CH3)2 | |
| 1199 | CF2CF3 | Pr | ═C(CH3)N(CH3)2 | |
| 1200 | CF2CF3 | Pr | ═CH—N(C2H5)2 | |
| 1201 | CF2CF3 | Pr | ═C(CH3)N(C2H5)2 | |
| 1202 | CF2CF3 | Pr | ═CH-Piperidine | |
| 1203 | CF2CF3 | Pr | ═CH-Morpholine | |
| 1204 | CF2CF3 | Pr | ═CH-Pyrrolidine | |
| 1205 | CF2CF3 | Pr | Me | Me |
| 1206 | CF2CF3 | Pr | Me | Et |
| 1207 | CF2CF3 | Pr | Et | Me |
| 1208 | CF2CF3 | Pr | Et | Et |
| 1209 | CF2CF3 | Pr | Me | Ph |
| 1210 | CF2CF3 | Pr | Me | CH2CO2Me |
| 1211 | CF3 | Cl | H | Me |
| 1212 | CF3 | Cl | H | Et |
| 1213 | CF3 | Cl | H | Pr |
| 1214 | CF3 | Cl | H | iPr |
| 1215 | CF3 | Cl | H | cPr |
| 1216 | CF3 | Cl | H | Bu |
| 1217 | CF3 | Cl | H | cBu |
| 1218 | CF3 | Cl | H | tBu |
| 1219 | CF3 | Cl | H | cPentyl |
| 1220 | CF3 | Cl | H | cHexyl |
| 1221 | CF3 | Cl | H | CH2(CH2)3CH3 |
| 1222 | CF3 | Cl | H | CH2(CH2)4CH3 |
| 1223 | CF3 | Cl | H | CH2—cPr |
| 1224 | CF3 | Cl | H | CH2—CN |
| 1225 | CF3 | Cl | H | CH2—C(CH3)3 |
| 1226 | CF3 | Cl | H | CH2CF2CF3 |
| 1227 | CF3 | Cl | H | CH2CF3 |
| 1228 | CF3 | Cl | H | CH2(CF2)2CF3 |
| 1229 | CF3 | Cl | H | CH2CH(CH3)CH2CH3 |
| 1230 | CF3 | Cl | H | CH2C(CH3)2CH2F |
| 1231 | CF3 | Cl | H | CH2CH(CH3)2 |
| 1232 | CF3 | Cl | H | CH2CH(CH2CH3)2 |
| 1233 | CF3 | Cl | H | CH2CH2CH(CH3)2 |
| 1234 | CF3 | Cl | H | CH2CH2C(CH3)3 |
| 1235 | CF3 | Cl | H | CH2CH═CH2 |
| 1236 | CF3 | Cl | H | CH2CH═CHCH3 |
| 1237 | CF3 | Cl | H | CH2—C(CH3)═CH2 |
| 1238 | CF3 | Cl | H | CH2—C≡CH |
| 1239 | CF3 | Cl | H | CH(CH3)CH2CH3 |
| 1240 | CF3 | Cl | H | CH(CH3)cPr |
| 1241 | CF3 | Cl | H | CH(CH3)CF3 |
| 1242 | CF3 | Cl | H | CH(CH3)(CH2)2CH3 |
| 1243 | CF3 | Cl | H | CH(CH3)(CH2)4CH3 |
| 1244 | CF3 | Cl | H | CH(CH3)(CH2)5CH3 |
| 1245 | CF3 | Cl | H | CH(CH2CH3)(CH2)3CH3 |
| 1246 | CF3 | Cl | H | CH(CH3)CH2CH(CH3)2 |
| 1247 | CF3 | Cl | H | CH(CH3)C(CH3)3 |
| 1248 | CF3 | Cl | H | CH(CH3)CH(CH3)2 |
| 1249 | CF3 | Cl | H | CH(CH3)CH2CH2CH(CH3)2 |
| 1250 | CF3 | Cl | H | CH(CH2CH3)2 |
| 1251 | CF3 | Cl | H | C(CH3)2CH2CH3 |
| 1252 | CF3 | Cl | H | C(CH3)2CH2C(CH3)3 |
| 1253 | CF3 | Cl | H | CH2—CH(OMe)2 |
| 1254 | CF3 | Cl | H | CH2—CH(OEt)2 |
| 1255 | CF3 | Cl | H | CH2CH2—OH |
| 1256 | CF3 | Cl | H | CH2CH2—OMe |
| 1257 | CF3 | Cl | H | CH2CH2—OEt |
| 1258 | CF3 | Cl | H | CH2CH2—SMe |
| 1259 | CF3 | Cl | H | CH2CH2—CN |
| 1260 | CF3 | Cl | H | CH2CH2—NMe2 |
| 1261 | CF3 | Cl | H | CH2CH2-Morpholin-4-yl |
| 1262 | CF3 | Cl | H | CH(CH3)CH2—OMe |
| 1263 | CF3 | Cl | H | CH(CH3)CH2—NMe2 |
| 1264 | CF3 | Cl | H | CH2CH2CH2—OMe |
| 1265 | CF3 | Cl | H | CH2CH2CH2—SMe |
| 1266 | CF3 | Cl | H | CH2CH2CH2—OEt |
| 1267 | CF3 | Cl | H | CH2CH2CH2—OiPr |
| 1268 | CF3 | Cl | H | CH2CH2CH2—OBu |
| 1269 | CF3 | Cl | H | CH2—COOMe |
| 1270 | CF3 | Cl | H | CH2—COOH |
| 1271 | CF3 | Cl | H | CH(CH3)COOMe |
| 1272 | CF3 | Cl | H | CH(CH3)COOEt |
| 1273 | CF3 | Cl | H | CH2CH2—COOMe |
| 1274 | CF3 | Cl | H | CH(COOMe)2 |
| 1275 | CF3 | Cl | H | CH(COOEt)CH2—CH(CH3)2 |
| 1276 | CF3 | Cl | H | CH(COOMe)CH(CH3)2 |
| 1277 | CF3 | Cl | H | COCH2OMe |
| 1278 | CF3 | Cl | H | CH(cPr)2 |
| 1279 | CF3 | Cl | H | Ph |
| 1280 | CF3 | Cl | H | CH2PH |
| 1281 | CF3 | Cl | H | CH2-Pyridin-3-yl |
| 1282 | CF3 | Cl | H | CH2—6-Cl-Pyridin-3-yl |
| 1283 | CF3 | Cl | H | CH(CH3)Ph |
| 1284 | CF3 | Cl | H | CH2CH2—Ph |
| 1285 | CF3 | Cl | H | CH2—2-CF3—Ph |
| 1286 | CF3 | Cl | H | CH2CH2CHPh2 |
| 1287 | CF3 | Cl | H | OCH2CH3 |
| 1288 | CF3 | Cl | H | O—CH3 |
| 1289 | CF3 | Cl | H | O—CH2CH═CH2 |
| 1290 | CF3 | Cl | H | O—tBu |
| 1291 | CF3 | Cl | H | O—Pr |
| 1292 | CF3 | Cl | H | O—CH2cPr |
| 1293 | CF3 | Cl | H | O—CH2CH(CH3)2 |
| 1294 | CF3 | Cl | H | O—CH2CF3 |
| 1295 | CF3 | Cl | H | O—CH(CH3)cPr |
| 1296 | CF3 | Cl | H | O—CH2CH2Cl |
| 1297 | CF3 | Cl | H | O—CH2C≡CH |
| 1298 | CF3 | Cl | H | O—CH2C≡CCH3 |
| 1299 | CF3 | Cl | H | O—CH(CH3)C≡CH |
| 1300 | CF3 | Cl | H | Indan-1-yl |
| 1301 | CF3 | Cl | H | Tetrahydrofuran-2-ylmethyl |
| 1302 | CF3 | Cl | Morpholin-4-yl | |
| 1303 | CF3 | Cl | 2-(Methoxycarbonyl)piperidin-1-yl | |
| 1304 | CF3 | Cl | 2-(Ethoxycarbonyl)piperidin-1-yl | |
| 1305 | CF3 | Cl | Thiazolidin-3-yl | |
| 1306 | CF3 | Cl | Pyrrolidin-1-yl | |
| 1307 | CF3 | Cl | 2-Methylpyrrolidin-1-yl | |
| 1308 | CF3 | Cl | ═CH—N(CH3)2 | |
| 1309 | CF3 | Cl | ═C(CH3)N(CH3)2 | |
| 1310 | CF3 | Cl | ═CH—N(C2H5)2 | |
| 1311 | CF3 | Cl | ═C(CH3)N(C2H5)2 | |
| 1312 | CF3 | Cl | ═CH-Piperidine | |
| 1313 | CF3 | Cl | ═CH-Morpholine | |
| 1314 | CF3 | Cl | ═CH-Pyrrolidine | |
| 1315 | CF3 | Cl | Me | Me |
| 1316 | CF3 | Cl | Me | Et |
| 1317 | CF3 | Cl | Et | Me |
| 1318 | CF3 | Cl | Et | Et |
| 1319 | CF3 | Cl | Me | Ph |
| 1320 | CF3 | Cl | Me | CH2CO2Me |
| 1321 | CF2CF3 | Cl | H | Me |
| 1322 | CF2CF3 | Cl | H | Et |
| 1323 | CF2CF3 | Cl | H | Pr |
| 1324 | CF2CF3 | Cl | H | iPr |
| 1325 | CF2CF3 | Cl | H | cPr |
| 1326 | CF2CF3 | Cl | H | Bu |
| 1327 | CF2CF3 | Cl | H | cBu |
| 1328 | CF2CF3 | Cl | H | tBu |
| 1329 | CF2CF3 | Cl | H | cPentyl |
| 1330 | CF2CF3 | Cl | H | cHexyl |
| 1331 | CF2CF3 | Cl | H | CH2(CH2)3CH3 |
| 1332 | CF2CF3 | Cl | H | CH2(CH2)4CH3 |
| 1333 | CF2CF3 | Cl | H | CH2—cPr |
| 1334 | CF2CF3 | Cl | H | CH2—CN |
| 1335 | CF2CF3 | Cl | H | CH2—C(CH3)3 |
| 1336 | CF2CF3 | Cl | H | CH2CF2CF3 |
| 1337 | CF2CF3 | Cl | H | CH2CF3 |
| 1338 | CF2CF3 | Cl | H | CH2(CF2)2CF3 |
| 1339 | CF2CF3 | Cl | H | CH2CH(CH3)CH2CH3 |
| 1340 | CF2CF3 | Cl | H | CH2C(CH3)2CH2F |
| 1341 | CF2CF3 | Cl | H | CH2CH(CH3)2 |
| 1342 | CF2CF3 | Cl | H | CH2CH(CH2CH3)2 |
| 1343 | CF2CF3 | Cl | H | CH2CH2CH(CH3)2 |
| 1344 | CF2CF3 | Cl | H | CH2CH2C(CH3)3 |
| 1345 | CF2CF3 | Cl | H | CH2CH═CH2 |
| 1346 | CF2CF3 | Cl | H | CH2CH═CHCH3 |
| 1347 | CF2CF3 | Cl | H | CH2—C(CH3)═CH2 |
| 1348 | CF2CF3 | Cl | H | CH2—C≡CH |
| 1349 | CF2CF3 | Cl | H | CH(CH3)CH2CH3 |
| 1350 | CF2CF3 | Cl | H | CH(CH3)cPr |
| 1351 | CF2CF3 | Cl | H | CH(CH3)CF3 |
| 1352 | CF2CF3 | Cl | H | CH(CH3)(CH2)2CH3 |
| 1353 | CF2CF3 | Cl | H | CH(CH3)(CH2)4CH3 |
| 1354 | CF2CF3 | Cl | H | CH(CH3)(CH2)5CH3 |
| 1355 | CF2CF3 | Cl | H | CH(CH2CH3)(CH2)3CH3 |
| 1356 | CF2CF3 | Cl | H | CH(CH3)CH2CH(CH3)2 |
| 1357 | CF2CF3 | Cl | H | CH(CH3)C(CH3)3 |
| 1358 | CF2CF3 | Cl | H | CH(CH3)CH(CH3)2 |
| 1359 | CF2CF3 | Cl | H | CH(CH3)CH2CH2CH(CH3)2 |
| 1360 | CF2CF3 | Cl | H | CH(CH2CH3)2 |
| 1361 | CF2CF3 | Cl | H | C(CH3)2CH2CH3 |
| 1362 | CF2CF3 | Cl | H | C(CH3)2CH2C(CH3)3 |
| 1363 | CF2CF3 | Cl | H | CH2—CH(OMe)2 |
| 1364 | CF2CF3 | Cl | H | CH2—CH(OEt)2 |
| 1365 | CF2CF3 | Cl | H | CH2CH2—OH |
| 1366 | CF2CF3 | Cl | H | CH2CH2—OMe |
| 1367 | CF2CF3 | Cl | H | CH2CH2—OEt |
| 1368 | CF2CF3 | Cl | H | CH2CH2—SMe |
| 1369 | CF2CF3 | Cl | H | CH2CH2—CN |
| 1370 | CF2CF3 | Cl | H | CH2CH2—NMe2 |
| 1371 | CF2CF3 | Cl | H | CH2CH2-Morpholin-4-yl |
| 1372 | CF2CF3 | Cl | H | CH(CH3)CH2—OMe |
| 1373 | CF2CF3 | Cl | H | CH(CH3)CH2—NMe2 |
| 1374 | CF2CF3 | Cl | H | CH2CH2CH2—OMe |
| 1375 | CF2CF3 | Cl | H | CH2CH2CH2—SMe |
| 1376 | CF2CF3 | Cl | H | CH2CH2CH2—OEt |
| 1377 | CF2CF3 | Cl | H | CH2CH2CH2—OiPr |
| 1378 | CF2CF3 | Cl | H | CH2CH2CH2—OBu |
| 1379 | CF2CF3 | Cl | H | CH2—COOMe |
| 1380 | CF2CF3 | Cl | H | CH2—COOH |
| 1381 | CF2CF3 | Cl | H | CH(CH3)COOMe |
| 1382 | CF2CF3 | Cl | H | CH(CH3)COOEt |
| 1383 | CF2CF3 | Cl | H | CH2CH2—COOMe |
| 1384 | CF2CF3 | Cl | H | CH(COOMe)2 |
| 1385 | CF2CF3 | Cl | H | CH(COOEt)CH2—CH(CH3)2 |
| 1386 | CF2CF3 | Cl | H | CH(COOMe)CH(CH3)2 |
| 1387 | CF2CF3 | Cl | H | COCH2OMe |
| 1388 | CF2CF3 | Cl | H | CH(cPr)2 |
| 1389 | CF2CF3 | Cl | H | Ph |
| 1390 | CF2CF3 | Cl | H | CH2PH |
| 1391 | CF2CF3 | Cl | H | CH2-Pyridin-3-yl |
| 1392 | CF2CF3 | Cl | H | CH2—6-Cl-Pyridin-3-yl |
| 1393 | CF2CF3 | Cl | H | CH(CH3)Ph |
| 1394 | CF2CF3 | Cl | H | CH2CH2—Ph |
| 1395 | CF2CF3 | Cl | H | CH2—2-CF3—Ph |
| 1396 | CF2CF3 | Cl | H | CH2CH2CHPh2 |
| 1397 | CF2CF3 | Cl | H | O—CH2CH3 |
| 1398 | CF2CF3 | Cl | H | O—CH3 |
| 1399 | CF2CF3 | Cl | H | O—CH2CH═CH2 |
| 1400 | CF2CF3 | Cl | H | O—tBu |
| 1401 | CF2CF3 | Cl | H | O—Pr |
| 1402 | CF2CF3 | Cl | H | O—CH2cPr |
| 1403 | CF2CF3 | Cl | H | O—CH2CH(CH3)2 |
| 1404 | CF2CF3 | Cl | H | O—CH2CF3 |
| 1405 | CF2CF3 | Cl | H | O—CH(CH3)cPr |
| 1406 | CF2CF3 | Cl | H | O—CH2CH2Cl |
| 1407 | CF2CF3 | Cl | H | O—CH2C≡CH |
| 1408 | CF2CF3 | Cl | H | O—CH2C≡CCH3 |
| 1409 | CF2CF3 | Cl | H | O—CH(CH3)C≡CH |
| 1410 | CF2CF3 | Cl | H | Indan-1-yl |
| 1411 | CF2CF3 | Cl | H | Tetrahydrofuran-2-ylmethyl |
| 1412 | CF2CF3 | Cl | Morpholin-4-yl | |
| 1413 | CF2CF3 | Cl | 2-(Methoxycarbonyl)piperidin-1-yl | |
| 1414 | CF2CF3 | Cl | 2-(Ethoxycarbonyl)piperidin-1-yl | |
| 1415 | CF2CF3 | Cl | Thiazolidin-3-yl | |
| 1416 | CF2CF3 | Cl | Pyrrolidin-1-yl | |
| 1417 | CF2CF3 | Cl | 2-Methylpyrrolidin-1-yl | |
| 1418 | CF2CF3 | Cl | ═CH—N(CH3)2 | |
| 1419 | CF2CF3 | Cl | ═C(CH3)N(CH3)2 | |
| 1420 | CF2CF3 | Cl | ═CH—N(C2H5)2 | |
| 1421 | CF2CF3 | Cl | ═C(CH3)N(C2H5)2 | |
| 1422 | CF2CF3 | Cl | ═CH-Piperidine | |
| 1423 | CF2CF3 | Cl | ═CH-Morpholine | |
| 1424 | CF2CF3 | Cl | ═CH-Pyrrolidine | |
| 1425 | CF2CF3 | Cl | Me | Me |
| 1426 | CF2CF3 | Cl | Me | Et |
| 1427 | CF2CF3 | Cl | Et | Me |
| 1428 | CF2CF3 | Cl | Et | Et |
| 1429 | CF2CF3 | Cl | Me | Ph |
| 1430 | CF2CF3 | Cl | Me | CH2CO2Me |
| 1431 | CHFCF3 | Cl | H | Me |
| 1432 | CHFCF3 | Cl | H | Et |
| 1433 | CHFCF3 | Cl | H | Pr |
| 1434 | CHFCF3 | Cl | H | iPr |
| 1435 | CHFCF3 | Cl | H | cPr |
| 1436 | CHFCF3 | Cl | H | Bu |
| 1437 | CHFCF3 | Cl | H | cBu |
| 1438 | CHFCF3 | Cl | H | tBu |
| 1439 | CHFCF3 | Cl | H | cPentyl |
| 1440 | CHFCF3 | Cl | H | cHexyl |
| 1441 | CHFCF3 | Cl | H | CH2(CH2)3CH3 |
| 1442 | CHFCF3 | Cl | H | CH2(CH2)4CH3 |
| 1443 | CHFCF3 | Cl | H | CH2—cPr |
| 1444 | CHFCF3 | Cl | H | CH2—CN |
| 1445 | CHFCF3 | Cl | H | CH2—C(CH3)3 |
| 1446 | CHFCF3 | Cl | H | CH2CF2CF3 |
| 1447 | CHFCF3 | Cl | H | CH2CF3 |
| 1448 | CHFCF3 | Cl | H | CH2(CF2)2CF3 |
| 1449 | CHFCF3 | Cl | H | CH2CH(CH3)CH2CH3 |
| 1450 | CHFCF3 | Cl | H | CH2C(CH3)2CH2F |
| 1451 | CHFCF3 | Cl | H | CH2CH(CH3)2 |
| 1452 | CHFCF3 | Cl | H | CH2CH(CH2CH3)2 |
| 1453 | CHFCF3 | Cl | H | CH2CH2CH(CH3)2 |
| 1454 | CHFCF3 | Cl | H | CH2CH2C(CH3)3 |
| 1455 | CHFCF3 | Cl | H | CH2CH═CH2 |
| 1456 | CHFCF3 | Cl | H | CH2CH═CHCH3 |
| 1457 | CHFCF3 | Cl | H | CH2—C(CH3)═CH2 |
| 1458 | CHFCF3 | Cl | H | CH2—C≡CH |
| 1459 | CHFCF3 | Cl | H | CH(CH3)CH2CH3 |
| 1460 | CHFCF3 | Cl | H | CH(CH3)cPr |
| 1461 | CHFCF3 | Cl | H | CH(CH3)CF3 |
| 1462 | CHFCF3 | Cl | H | CH(CH3)(CH2)2CH3 |
| 1463 | CHFCF3 | Cl | H | CH(CH3)(CH2)4CH3 |
| 1464 | CHFCF3 | Cl | H | CH(CH3)(CH2)5CH3 |
| 1465 | CHFCF3 | Cl | H | CH(CH2CH3)(CH2)3CH3 |
| 1466 | CHFCF3 | Cl | H | CH(CH3)CH2CH(CH3)2 |
| 1467 | CHFCF3 | Cl | H | CH(CH3)C(CH3)3 |
| 1468 | CHFCF3 | Cl | H | CH(CH3)CH(CH3)2 |
| 1469 | CHFCF3 | Cl | H | CH(CH3)CH2CH2CH(CH3)2 |
| 1470 | CHFCF3 | Cl | H | CH(CH2CH3)2 |
| 1471 | CHFCF3 | Cl | H | C(CH3)2CH2CH3 |
| 1472 | CHFCF3 | Cl | H | C(CH3)2CH2C(CH3)3 |
| 1473 | CHFCF3 | Cl | H | CH2—CH(OMe)2 |
| 1474 | CHFCF3 | Cl | H | CH2—CH(OEt)2 |
| 1475 | CHFCF3 | Cl | H | CH2CH2—OH |
| 1476 | CHFCF3 | Cl | H | CH2CH2—OMe |
| 1477 | CHFCF3 | Cl | H | CH2CH2—OEt |
| 1478 | CHFCF3 | Cl | H | CH2CH2—SMe |
| 1479 | CHFCF3 | Cl | H | CH2CH2—CN |
| 1480 | CHFCF3 | Cl | H | CH2CH2—NMe2 |
| 1481 | CHFCF3 | Cl | H | CH2CH2-Morpholin-4-yl |
| 1482 | CHFCF3 | Cl | H | CH(CH3)CH2—OMe |
| 1483 | CHFCF3 | Cl | H | CH(CH3)CH2—NMe2 |
| 1484 | CHFCF3 | Cl | H | CH2CH2CH2—OMe |
| 1485 | CHFCF3 | Cl | H | CH2CH2CH2—SMe |
| 1486 | CHFCF3 | Cl | H | CH2CH2CH2—OEt |
| 1487 | CHFCF3 | Cl | H | CH2CH2CH2—OiPr |
| 1488 | CHFCF3 | Cl | H | CH2CH2CH2—OBu |
| 1489 | CHFCF3 | Cl | H | CH2—COOMe |
| 1490 | CHFCF3 | Cl | H | CH2—COOH |
| 1491 | CHFCF3 | Cl | H | CH(CH3)COOMe |
| 1492 | CHFCF3 | Cl | H | CH(CH3)COOEt |
| 1493 | CHFCF3 | Cl | H | CH2CH2—COOMe |
| 1494 | CHFCF3 | Cl | H | CH(COOMe)2 |
| 1495 | CHFCF3 | Cl | H | CH(COOEt)CH2—CH(CH3)2 |
| 1496 | CHFCF3 | Cl | H | CH(COOMe)CH(CH3)2 |
| 1497 | CHFCF3 | Cl | H | COCH2OMe |
| 1498 | CHFCF3 | Cl | H | CH(cPr)2 |
| 1499 | CHFCF3 | Cl | H | Ph |
| 1500 | CHFCF3 | Cl | H | CH2PH |
| 1501 | CHFCF3 | Cl | H | CH2-Pyridin-3-yl |
| 1502 | CHFCF3 | Cl | H | CH2—6-Cl-Pyridin-3-yl |
| 1503 | CHFCF3 | Cl | H | CH(CH3)Ph |
| 1504 | CHFCF3 | Cl | H | CH2CH2—Ph |
| 1505 | CHFCF3 | Cl | H | CH2—2-CF3—Ph |
| 1506 | CHFCF3 | Cl | H | CH2CH2CHPh2 |
| 1507 | CHFCF3 | Cl | H | O—CH2CH3 |
| 1508 | CHFCF3 | Cl | H | O—CH3 |
| 1509 | CHFCF3 | Cl | H | O—CH2CH═CH2 |
| 1510 | CHFCF3 | Cl | H | O—tBu |
| 1511 | CHFCF3 | Cl | H | O—Pr |
| 1512 | CHFCF3 | Cl | H | O—CH2cPr |
| 1513 | CHFCF3 | Cl | H | O—CH2CH(CH3)2 |
| 1514 | CHFCF3 | Cl | H | O—CH2CF3 |
| 1515 | CHFCF3 | Cl | H | O—CH(CH3)cPr |
| 1516 | CHFCF3 | Cl | H | O—CH2CH2Cl |
| 1517 | CHFCF3 | Cl | H | O—CH2C≡CH |
| 1518 | CHFCF3 | Cl | H | O—CH2C≡CCH3 |
| 1519 | CHFCF3 | Cl | H | O—CH(CH3)C≡CH |
| 1520 | CHFCF3 | Cl | H | Indan-1-yl |
| 1521 | CHFCF3 | Cl | H | Tetrahydrofuran-2-ylmethyl |
| 1522 | CHFCF3 | Cl | Morpholin-4-yl | |
| 1523 | CHFCF3 | Cl | 2-(Methoxycarbonyl)piperidin-1-yl | |
| 1524 | CHFCF3 | Cl | 2-(Ethoxycarbonyl)piperidin-1-yl | |
| 1525 | CHFCF3 | Cl | Thiazolidin-3-yl | |
| 1526 | CHFCF3 | Cl | Pyrrolidin-1-yl | |
| 1527 | CHFCF3 | Cl | 2-Methylpyrrolidin-1-yl | |
| 1528 | CHFCF3 | Cl | ═CH—N(CH3)2 | |
| 1529 | CHFCF3 | Cl | ═C(CH3)N(CH3)2 | |
| 1530 | CHFCF3 | Cl | ═CH—N(C2H5)2 | |
| 1531 | CHFCF3 | Cl | ═C(CH3)N(C2H5)2 | |
| 1532 | CHFCF3 | Cl | ═CH-Piperidine | |
| 1533 | CHFCF3 | Cl | ═CH-Morpholine | |
| 1534 | CHFCF3 | Cl | ═CH-Pyrrolidine | |
| 1535 | CHFCF3 | Cl | Me | Me |
| 1536 | CHFCF3 | Cl | Me | Et |
| 1537 | CHFCF3 | Cl | Et | Me |
| 1538 | CHFCF3 | Cl | Et | Et |
| 1539 | CHFCF3 | Cl | Me | Ph |
| 1540 | CHFCF3 | Cl | Me | CH2CO2Me |
| 1541 | CF2CF2Cl | Cl | H | Me |
| 1542 | CF2CF2Cl | Cl | H | Et |
| 1543 | CF2CF2Cl | Cl | H | Pr |
| 1544 | CF2CF2Cl | Cl | H | iPr |
| 1545 | CF2CF2Cl | Cl | H | cPr |
| 1546 | CF2CF2Cl | Cl | H | Bu |
| 1547 | CF2CF2Cl | Cl | H | cBu |
| 1548 | CF2CF2Cl | Cl | H | tBu |
| 1549 | CF2CF2Cl | Cl | H | cPentyl |
| 1550 | CF2CF2Cl | Cl | H | cHexyl |
| 1551 | CF2CF2Cl | Cl | H | CH2(CH2)3CH3 |
| 1552 | CF2CF2Cl | Cl | H | CH2(CH2)4CH3 |
| 1553 | CF2CF2Cl | Cl | H | CH2—cPr |
| 1554 | CF2CF2Cl | Cl | H | CH2—CN |
| 1555 | CF2CF2Cl | Cl | H | CH2—C(CH3)3 |
| 1556 | CF2CF2Cl | Cl | H | CH2CF2CF3 |
| 1557 | CF2CF2Cl | Cl | H | CH2CF3 |
| 1558 | CF2CF2Cl | Cl | H | CH2(CF2)2CF3 |
| 1559 | CF2CF2Cl | Cl | H | CH2CH(CH3)CH2CH3 |
| 1560 | CF2CF2Cl | Cl | H | CH2C(CH3)2CH2F |
| 1561 | CF2CF2Cl | Cl | H | CH2CH(CH3)2 |
| 1562 | CF2CF2Cl | Cl | H | CH2CH(CH2CH3)2 |
| 1563 | CF2CF2Cl | Cl | H | CH2CH2CH(CH3)2 |
| 1564 | CF2CF2Cl | Cl | H | CH2CH2C(CH3)3 |
| 1565 | CF2CF2Cl | Cl | H | CH2CH═CH2 |
| 1566 | CF2CF2Cl | Cl | H | CH2CH═CHCH3 |
| 1567 | CF2CF2Cl | Cl | H | CH2—C(CH3)═CH2 |
| 1568 | CF2CF2Cl | Cl | H | CH2—C≡CH |
| 1569 | CF2CF2Cl | Cl | H | CH(CH3)CH2CH3 |
| 1570 | CF2CF2Cl | Cl | H | CH(CH3)cPr |
| 1571 | CF2CF2Cl | Cl | H | CH(CH3)CF3 |
| 1572 | CF2CF2Cl | Cl | H | CH(CH3)(CH2)2CH3 |
| 1573 | CF2CF2Cl | Cl | H | CH(CH3)(CH2)4CH3 |
| 1574 | CF2CF2Cl | Cl | H | CH(CH3)(CH2)5CH3 |
| 1575 | CF2CF2Cl | Cl | H | CH(CH2CH3)(CH2)3CH3 |
| 1576 | CF2CF2Cl | Cl | H | CH(CH3)CH2CH(CH3)2 |
| 1577 | CF2CF2Cl | Cl | H | CH(CH3)C(CH3)3 |
| 1578 | CF2CF2Cl | Cl | H | CH(CH3)CH(CH3)2 |
| 1579 | CF2CF2Cl | Cl | H | CH(CH3)CH2CH2CH(CH3)2 |
| 1580 | CF2CF2Cl | Cl | H | CH(CH2CH3)2 |
| 1581 | CF2CF2Cl | Cl | H | C(CH3)2CH2CH3 |
| 1582 | CF2CF2Cl | Cl | H | C(CH3)2CH2C(CH3)3 |
| 1583 | CF2CF2Cl | Cl | H | CH2—CH(OMe)2 |
| 1584 | CF2CF2Cl | Cl | H | CH2—CH(OEt)2 |
| 1585 | CF2CF2Cl | Cl | H | CH2CH2—OH |
| 1586 | CF2CF2Cl | Cl | H | CH2CH2—OMe |
| 1587 | CF2CF2Cl | Cl | H | CH2CH2—OEt |
| 1588 | CF2CF2Cl | Cl | H | CH2CH2—SMe |
| 1589 | CF2CF2Cl | Cl | H | CH2CH2—CN |
| 1590 | CF2CF2Cl | Cl | H | CH2CH2—NMe2 |
| 1591 | CF2CF2Cl | Cl | H | CH2CH2-Morpholin-4-yl |
| 1592 | CF2CF2Cl | Cl | H | CH(CH3)CH2—OMe |
| 1593 | CF2CF2Cl | Cl | H | CH(CH3)CH2—NMe2 |
| 1594 | CF2CF2Cl | Cl | H | CH2CH2CH2—OMe |
| 1595 | CF2CF2Cl | Cl | H | CH2CH2CH2—SMe |
| 1596 | CF2CF2Cl | Cl | H | CH2CH2CH2—OEt |
| 1597 | CF2CF2Cl | Cl | H | CH2CH2CH2—OiPr |
| 1598 | CF2CF2Cl | Cl | H | CH2CH2CH2—OBu |
| 1599 | CF2CF2Cl | Cl | H | CH2—COOMe |
| 1600 | CF2CF2Cl | Cl | H | CH2—COOH |
| 1601 | CF2CF2Cl | Cl | H | CH(CH3)COOMe |
| 1602 | CF2CF2Cl | Cl | H | CH(CH3)COOEt |
| 1603 | CF2CF2Cl | Cl | H | CH2CH2—COOMe |
| 1604 | CF2CF2Cl | Cl | H | CH(COOMe)2 |
| 1605 | CF2CF2Cl | Cl | H | CH(COOEt)CH2—CH(CH3)2 |
| 1606 | CF2CF2Cl | Cl | H | CH(COOMe)CH(CH3)2 |
| 1607 | CF2CF2Cl | Cl | H | COCH2OMe |
| 1608 | CF2CF2Cl | Cl | H | CH(cPr)2 |
| 1609 | CF2CF2Cl | Cl | H | Ph |
| 1610 | CF2CF2Cl | Cl | H | CH2PH |
| 1611 | CF2CF2Cl | Cl | H | CH2-Pyridin-3-yl |
| 1612 | CF2CF2Cl | Cl | H | CH2—6-Cl-Pyridin-3-yl |
| 1613 | CF2CF2Cl | Cl | H | CH(CH3)Ph |
| 1614 | CF2CF2Cl | Cl | H | CH2CH2—Ph |
| 1615 | CF2CF2Cl | Cl | H | CH2—2-CF3—Ph |
| 1616 | CF2CF2Cl | Cl | H | CH2CH2CHPh2 |
| 1617 | CF2CF2Cl | Cl | H | O—CH2CH3 |
| 1618 | CF2CF2Cl | Cl | H | O—CH3 |
| 1619 | CF2CF2Cl | Cl | H | O—CH2CH═CH2 |
| 1620 | CF2CF2Cl | Cl | H | O—tBu |
| 1621 | CF2CF2Cl | Cl | H | O—Pr |
| 1622 | CF2CF2Cl | Cl | H | O—CH2cPr |
| 1623 | CF2CF2Cl | Cl | H | O—CH2CH(CH3)2 |
| 1624 | CF2CF2Cl | Cl | H | O—CH2CF3 |
| 1625 | CF2CF2Cl | Cl | H | O—CH(CH3)cPr |
| 1626 | CF2CF2Cl | Cl | H | O—CH2CH2Cl |
| 1627 | CF2CF2Cl | Cl | H | O—CH2C≡CH |
| 1628 | CF2CF2Cl | Cl | H | O—CH2C≡CCH3 |
| 1629 | CF2CF2Cl | Cl | H | O—CH(CH3)C≡CH |
| 1630 | CF2CF2Cl | Cl | H | Indan-1-yl |
| 1631 | CF2CF2Cl | Cl | H | Tetrahydrofuran-2-ylmethyl |
| 1632 | CF2CF2Cl | Cl | Morpholin-4-yl | |
| 1633 | CF2CF2Cl | Cl | 2-(Methoxycarbonyl)piperidin-1-yl | |
| 1634 | CF2CF2Cl | Cl | 2-(Ethoxycarbonyl)piperidin-1-yl | |
| 1635 | CF2CF2Cl | Cl | Thiazolidin-3-yl | |
| 1636 | CF2CF2Cl | Cl | Pyrrolidin-1-yl | |
| 1637 | CF2CF2Cl | Cl | 2-Methylpyrrolidin-1-yl | |
| 1638 | CF2CF2Cl | Cl | ═CH—N(CH3)2 | |
| 1639 | CF2CF2Cl | Cl | ═C(CH3)N(CH3)2 | |
| 1640 | CF2CF2Cl | Cl | ═CH—N(C2H5)2 | |
| 1641 | CF2CF2Cl | Cl | ═C(CH3)N(C2H5)2 | |
| 1642 | CF2CF2Cl | Cl | ═CH-Piperidine | |
| 1643 | CF2CF2Cl | Cl | ═CH-Morpholine | |
| 1644 | CF2CF2Cl | Cl | ═CH-Pyrrolidine | |
| 1645 | CF2CF2Cl | Cl | Me | Me |
| 1646 | CF2CF2Cl | Cl | Me | Et |
| 1647 | CF2CF2Cl | Cl | Et | Me |
| 1648 | CF2CF2Cl | Cl | Et | Et |
| 1649 | CF2CF2Cl | Cl | Me | Ph |
| 1650 | CF2CF2Cl | Cl | Me | CH2CO2Me |
| 1651 | CF2Cl | Cl | H | Me |
| 1652 | CF2Cl | Cl | H | Et |
| 1653 | CF2Cl | Cl | H | Pr |
| 1654 | CF2Cl | Cl | H | iPr |
| 1655 | CF2Cl | Cl | H | cPr |
| 1656 | CF2Cl | Cl | H | Bu |
| 1657 | CF2Cl | Cl | H | cBu |
| 1658 | CF2Cl | Cl | H | tBu |
| 1659 | CF2Cl | Cl | H | cPentyl |
| 1660 | CF2Cl | Cl | H | cHexyl |
| 1661 | CF2Cl | Cl | H | CH2(CH2)3CH3 |
| 1662 | CF2Cl | Cl | H | CH2(CH2)4CH3 |
| 1663 | CF2Cl | Cl | H | CH2—cPr |
| 1664 | CF2Cl | Cl | H | CH2—CN |
| 1665 | CF2Cl | Cl | H | CH2—C(CH3)3 |
| 1666 | CF2Cl | Cl | H | CH2CF2CF3 |
| 1667 | CF2Cl | Cl | H | CH2CF3 |
| 1668 | CF2Cl | Cl | H | CH2(CF2)2CF3 |
| 1669 | CF2Cl | Cl | H | CH2CH(CH3)CH2CH3 |
| 1670 | CF2Cl | Cl | H | CH2C(CH3)2CH2F |
| 1671 | CF2Cl | Cl | H | CH2CH(CH3)2 |
| 1672 | CF2Cl | Cl | H | CH2CH(CH2CH3)2 |
| 1673 | CF2Cl | Cl | H | CH2CH2CH(CH3)2 |
| 1674 | CF2Cl | Cl | H | CH2CH2C(CH3)3 |
| 1675 | CF2Cl | Cl | H | CH2CH═CH2 |
| 1676 | CF2Cl | Cl | H | CH2CH═CHCH3 |
| 1677 | CF2Cl | Cl | H | CH2—C(CH3)═CH2 |
| 1678 | CF2Cl | Cl | H | CH2—C≡CH |
| 1679 | CF2Cl | Cl | H | CH(CH3)CH2CH3 |
| 1680 | CF2Cl | Cl | H | CH(CH3)cPr |
| 1681 | CF2Cl | Cl | H | CH(CH3)CF3 |
| 1682 | CF2Cl | Cl | H | CH(CH3)(CH2)2CH3 |
| 1683 | CF2Cl | Cl | H | CH(CH3)(CH2)4CH3 |
| 1684 | CF2Cl | Cl | H | CH(CH3)(CH2)5CH3 |
| 1685 | CF2Cl | Cl | H | CH(CH2CH3)(CH2)3CH3 |
| 1686 | CF2Cl | Cl | H | CH(CH3)CH2CH(CH3)2 |
| 1687 | CF2Cl | Cl | H | CH(CH3)C(CH3)3 |
| 1688 | CF2Cl | Cl | H | CH(CH3)CH(CH3)2 |
| 1689 | CF2Cl | Cl | H | CH(CH3)CH2CH2CH(CH3)2 |
| 1690 | CF2Cl | Cl | H | CH(CH2CH3)2 |
| 1691 | CF2Cl | Cl | H | C(CH3)2CH2CH3 |
| 1692 | CF2Cl | Cl | H | C(CH3)2CH2C(CH3)3 |
| 1693 | CF2Cl | Cl | H | CH2—CH(OMe)2 |
| 1694 | CF2Cl | Cl | H | CH2—CH(OEt)2 |
| 1695 | CF2Cl | Cl | H | CH2CH2—OH |
| 1696 | CF2Cl | Cl | H | CH2CH2—OMe |
| 1697 | CF2Cl | Cl | H | CH2CH2—OEt |
| 1698 | CF2Cl | Cl | H | CH2CH2—SMe |
| 1699 | CF2Cl | Cl | H | CH2CH2—CN |
| 1700 | CF2Cl | Cl | H | CH2CH2—NMe2 |
| 1701 | CF2Cl | Cl | H | CH2CH2-Morpholin-4-yl |
| 1702 | CF2Cl | Cl | H | CH(CH3)CH2—OMe |
| 1703 | CF2Cl | Cl | H | CH(CH3)CH2—NMe2 |
| 1704 | CF2Cl | Cl | H | CH2CH2CH2—OMe |
| 1705 | CF2Cl | Cl | H | CH2CH2CH2—SMe |
| 1706 | CF2Cl | Cl | H | CH2CH2CH2—OEt |
| 1707 | CF2Cl | Cl | H | CH2CH2CH2—OiPr |
| 1708 | CF2Cl | Cl | H | CH2CH2CH2—OBu |
| 1709 | CF2Cl | Cl | H | CH2—COOMe |
| 1710 | CF2Cl | Cl | H | CH2—COOH |
| 1711 | CF2Cl | Cl | H | CH(CH3)COOMe |
| 1712 | CF2Cl | Cl | H | CH(CH3)COOEt |
| 1713 | CF2Cl | Cl | H | CH2CH2—COOMe |
| 1714 | CF2Cl | Cl | H | CH(COOMe)2 |
| 1715 | CF2Cl | Cl | H | CH(COOEt)CH2—CH(CH3)2 |
| 1716 | CF2Cl | Cl | H | CH(COOMe)CH(CH3)2 |
| 1717 | CF2Cl | Cl | H | COCH2OMe |
| 1718 | CF2Cl | Cl | H | CH(cPr)2 |
| 1719 | CF2Cl | Cl | H | Ph |
| 1720 | CF2Cl | Cl | H | CH2PH |
| 1721 | CF2Cl | Cl | H | CH2-Pyridin-3-yl |
| 1722 | CF2Cl | Cl | H | CH2—6-Cl-Pyridin-3-yl |
| 1723 | CF2Cl | Cl | H | CH(CH3)Ph |
| 1724 | CF2Cl | Cl | H | CH2CH2—Ph |
| 1725 | CF2Cl | Cl | H | CH2—2-CF3—Ph |
| 1726 | CF2Cl | Cl | H | CH2CH2CHPh2 |
| 1727 | CF2Cl | Cl | H | O—CH2CH3 |
| 1728 | CF2Cl | Cl | H | O—CH3 |
| 1729 | CF2Cl | Cl | H | O—CH2CH═CH2 |
| 1730 | CF2Cl | Cl | H | O—tBu |
| 1731 | CF2Cl | Cl | H | O—Pr |
| 1732 | CF2Cl | Cl | H | O—CH2cPr |
| 1733 | CF2Cl | Cl | H | O—CH2CH(CH3)2 |
| 1734 | CF2Cl | Cl | H | O—CH2CF3 |
| 1735 | CF2Cl | Cl | H | O—CH(CH3)cPr |
| 1736 | CF2Cl | Cl | H | O—CH2CH2Cl |
| 1737 | CF2Cl | Cl | H | O—CH2C≡CH |
| 1738 | CF2Cl | Cl | H | O—CH2C≡CCH3 |
| 1739 | CF2Cl | Cl | H | O—CH(CH3)C≡CH |
| 1740 | CF2Cl | Cl | H | Indan-1-yl |
| 1741 | CF2Cl | Cl | H | Tetrahydrofuran-2-ylmethyl |
| 1742 | CF2Cl | Cl | Morpholin-4-yl | |
| 1743 | CF2Cl | Cl | 2-(Methoxycarbonyl)piperidin-1-yl | |
| 1744 | CF2Cl | Cl | 2-(Ethoxycarbonyl)piperidin-1-yl | |
| 1745 | CF2Cl | Cl | Thiazolidin-3-yl | |
| 1746 | CF2Cl | Cl | Pyrrolidin-1-yl | |
| 1747 | CF2Cl | Cl | 2-Methylpyrrolidin-1-yl | |
| 1748 | CF2Cl | Cl | ═CH—N(CH3)2 | |
| 1749 | CF2Cl | Cl | ═C(CH3)N(CH3)2 | |
| 1750 | CF2Cl | Cl | ═CH—N(C2H5)2 | |
| 1751 | CF2Cl | Cl | ═C(CH3)N(C2H5)2 | |
| 1752 | CF2Cl | Cl | ═CH-Piperidine | |
| 1753 | CF2Cl | Cl | ═CH-Morpholine | |
| 1754 | CF2Cl | Cl | ═CH-Pyrrolidine | |
| 1755 | CF2Cl | Cl | Me | Me |
| 1756 | CF2Cl | Cl | Me | Et |
| 1757 | CF2Cl | Cl | Et | Me |
| 1758 | CF2Cl | Cl | Et | Et |
| 1759 | CF2Cl | Cl | Me | Ph |
| 1760 | CF2Cl | Cl | Me | CH2CO2Me |
| 1761 | CF3 | Br | H | Me |
| 1762 | CF3 | Br | H | Et |
| 1763 | CF3 | Br | H | Pr |
| 1764 | CF3 | Br | H | iPr |
| 1765 | CF3 | Br | H | cPr |
| 1766 | CF3 | Br | H | Bu |
| 1767 | CF3 | Br | H | cBu |
| 1768 | CF3 | Br | H | tBu |
| 1769 | CF3 | Br | H | cPentyl |
| 1770 | CF3 | Br | H | cHexyl |
| 1771 | CF3 | Br | H | CH2(CH2)3CH3 |
| 1772 | CF3 | Br | H | CH2(CH2)4CH3 |
| 1773 | CF3 | Br | H | CH2—cPr |
| 1774 | CF3 | Br | H | CH2—CN |
| 1775 | CF3 | Br | H | CH2—C(CH3)3 |
| 1776 | CF3 | Br | H | CH2CF2CF3 |
| 1777 | CF3 | Br | H | CH2CF3 |
| 1778 | CF3 | Br | H | CH2(CF2)2CF3 |
| 1779 | CF3 | Br | H | CH2CH(CH3)CH2CH3 |
| 1780 | CF3 | Br | H | CH2C(CH3)2CH2F |
| 1781 | CF3 | Br | H | CH2CH(CH3)2 |
| 1782 | CF3 | Br | H | CH2CH(CH2CH3)2 |
| 1783 | CF3 | Br | H | CH2CH2CH(CH3)2 |
| 1784 | CF3 | Br | H | CH2CH2C(CH3)3 |
| 1785 | CF3 | Br | H | CH2CH═CH2 |
| 1786 | CF3 | Br | H | CH2CH═CHCH3 |
| 1787 | CF3 | Br | H | CH2—C(CH3)═CH2 |
| 1788 | CF3 | Br | H | CH2—C≡CH |
| 1789 | CF3 | Br | H | CH(CH3)CH2CH3 |
| 1790 | CF3 | Br | H | CH(CH3)cPr |
| 1791 | CF3 | Br | H | CH(CH3)CF3 |
| 1792 | CF3 | Br | H | CH(CH3)(CH2)2CH3 |
| 1793 | CF3 | Br | H | CH(CH3)(CH2)4CH3 |
| 1794 | CF3 | Br | H | CH(CH3)(CH2)5CH3 |
| 1795 | CF3 | Br | H | CH(CH2CH3)(CH2)3CH3 |
| 1796 | CF3 | Br | H | CH(CH3)CH2CH(CH3)2 |
| 1797 | CF3 | Br | H | CH(CH3)C(CH3)3 |
| 1798 | CF3 | Br | H | CH(CH3)CH(CH3)2 |
| 1799 | CF3 | Br | H | CH(CH3)CH2CH2CH(CH3)2 |
| 1800 | CF3 | Br | H | CH(CH2CH3)2 |
| 1801 | CF3 | Br | H | C(CH3)2CH2CH3 |
| 1802 | CF3 | Br | H | C(CH3)2CH2C(CH3)3 |
| 1803 | CF3 | Br | H | CH2—CH(OMe)2 |
| 1804 | CF3 | Br | H | CH2—CH(OEt)2 |
| 1805 | CF3 | Br | H | CH2CH2—OH |
| 1806 | CF3 | Br | H | CH2CH2—OMe |
| 1807 | CF3 | Br | H | CH2CH2—OEt |
| 1808 | CF3 | Br | H | CH2CH2—SMe |
| 1809 | CF3 | Br | H | CH2CH2—CN |
| 1810 | CF3 | Br | H | CH2CH2—NMe2 |
| 1811 | CF3 | Br | H | CH2CH2-Morpholin-4-yl |
| 1812 | CF3 | Br | H | CH(CH3)CH2—OMe |
| 1813 | CF3 | Br | H | CH(CH3)CH2—NMe2 |
| 1814 | CF3 | Br | H | CH2CH2CH2—OMe |
| 1815 | CF3 | Br | H | CH2CH2CH2—SMe |
| 1816 | CF3 | Br | H | CH2CH2CH2—OEt |
| 1817 | CF3 | Br | H | CH2CH2CH2—OiPr |
| 1818 | CF3 | Br | H | CH2CH2CH2—OBu |
| 1819 | CF3 | Br | H | CH2—COOMe |
| 1820 | CF3 | Br | H | CH2—COOH |
| 1821 | CF3 | Br | H | CH(CH3)COOMe |
| 1822 | CF3 | Br | H | CH(CH3)COOEt |
| 1823 | CF3 | Br | H | CH2CH2—COOMe |
| 1824 | CF3 | Br | H | CH(COOMe)2 |
| 1825 | CF3 | Br | H | CH(COOEt)CH2—CH(CH3)2 |
| 1826 | CF3 | Br | H | CH(COOMe)CH(CH3)2 |
| 1827 | CF3 | Br | H | COCH2OMe |
| 1828 | CF3 | Br | H | CH(cPr)2 |
| 1829 | CF3 | Br | H | Ph |
| 1830 | CF3 | Br | H | CH2PH |
| 1831 | CF3 | Br | H | CH2-Pyridin-3-yl |
| 1832 | CF3 | Br | H | CH2—6-Cl-Pyridin-3-yl |
| 1833 | CF3 | Br | H | CH(CH3)Ph |
| 1834 | CF3 | Br | H | CH2CH2—Ph |
| 1835 | CF3 | Br | H | CH2—2-CF3—Ph |
| 1836 | CF3 | Br | H | CH2CH2CHPh2 |
| 1837 | CF3 | Br | H | O—CH2CH3 |
| 1838 | CF3 | Br | H | O—CH3 |
| 1839 | CF3 | Br | H | O—CH2CH═CH2 |
| 1840 | CF3 | Br | H | O—tBu |
| 1841 | CF3 | Br | H | O—Pr |
| 1842 | CF3 | Br | H | O—CH2cPr |
| 1843 | CF3 | Br | H | O—CH2CH(CH3)2 |
| 1844 | CF3 | Br | H | O—CH2CF3 |
| 1845 | CF3 | Br | H | O—CH(CH3)cPr |
| 1846 | CF3 | Br | H | O—CH2CH2Cl |
| 1847 | CF3 | Br | H | O—CH2C≡CH |
| 1848 | CF3 | Br | H | O—CH2C≡CCH3 |
| 1849 | CF3 | Br | H | O—CH(CH3)C≡CH |
| 1850 | CF3 | Br | H | Indan-1-yl |
| 1851 | CF3 | Br | H | Tetrahydrofuran-2-ylmethyl |
| 1852 | CF3 | Br | Morpholin-4-yl | |
| 1853 | CF3 | Br | 2-(Methoxycarbonyl)piperidin-1-yl | |
| 1854 | CF3 | Br | 2-(Ethoxycarbonyl)piperidin-1-yl | |
| 1855 | CF3 | Br | Thiazolidin-3-yl | |
| 1856 | CF3 | Br | Pyrrolidin-1-yl | |
| 1857 | CF3 | Br | 2-Methylpyrrolidin-1-yl | |
| 1858 | CF3 | Br | ═CH—N(CH3)2 | |
| 1859 | CF3 | Br | ═C(CH3)N(CH3)2 | |
| 1860 | CF3 | Br | ═CH—N(C2H5)2 | |
| 1861 | CF3 | Br | ═C(CH3)N(C2H5)2 | |
| 1862 | CF3 | Br | ═CH-Piperidine | |
| 1863 | CF3 | Br | ═CH-Morpholine | |
| 1864 | CF3 | Br | ═CH-Pyrrolidine | |
| 1865 | CF3 | Br | Me | Me |
| 1866 | CF3 | Br | Me | Et |
| 1867 | CF3 | Br | Et | Me |
| 1868 | CF3 | Br | Et | Et |
| 1869 | CF3 | Br | Me | Ph |
| 1870 | CF3 | Br | Me | CH2CO2Me |
| 1871 | CF2CHF2 | Br | H | Me |
| 1872 | CF2CHF2 | Br | H | Et |
| 1873 | CF2CHF2 | Br | H | Pr |
| 1874 | CF2CHF2 | Br | H | iPr |
| 1875 | CF2CHF2 | Br | H | cPr |
| 1876 | CF2CHF2 | Br | H | Bu |
| 1877 | CF2CHF2 | Br | H | cBu |
| 1878 | CF2CHF2 | Br | H | tBu |
| 1879 | CF2CHF2 | Br | H | cPentyl |
| 1880 | CF2CHF2 | Br | H | cHexyl |
| 1881 | CF2CHF2 | Br | H | CH2(CH2)3CH3 |
| 1882 | CF2CHF2 | Br | H | CH2(CH2)4CH3 |
| 1883 | CF2CHF2 | Br | H | CH2—cPr |
| 1884 | CF2CHF2 | Br | H | CH2—CN |
| 1885 | CF2CHF2 | Br | H | CH2—C(CH3)3 |
| 1886 | CF2CHF2 | Br | H | CH2CF2CF3 |
| 1887 | CF2CHF2 | Br | H | CH2CF3 |
| 1888 | CF2CHF2 | Br | H | CH2(CF2)2CF3 |
| 1889 | CF2CHF2 | Br | H | CH2CH(CH3)CH2CH3 |
| 1890 | CF2CHF2 | Br | H | CH2C(CH3)2CH2F |
| 1891 | CF2CHF2 | Br | H | CH2CH(CH3)2 |
| 1892 | CF2CHF2 | Br | H | CH2CH(CH2CH3)2 |
| 1893 | CF2CHF2 | Br | H | CH2CH2CH(CH3)2 |
| 1894 | CF2CHF2 | Br | H | CH2CH2C(CH3)3 |
| 1895 | CF2CHF2 | Br | H | CH2CH═CH2 |
| 1896 | CF2CHF2 | Br | H | CH2CH═CHCH3 |
| 1897 | CF2CHF2 | Br | H | CH2—C(CH3)═CH2 |
| 1898 | CF2CHF2 | Br | H | CH2—C≡CH |
| 1899 | CF2CHF2 | Br | H | CH(CH3)CH2CH3 |
| 1900 | CF2CHF2 | Br | H | CH(CH3)cPr |
| 1901 | CF2CHF2 | Br | H | CH(CH3)CF3 |
| 1902 | CF2CHF2 | Br | H | CH(CH3)(CH2)2CH3 |
| 1903 | CF2CHF2 | Br | H | CH(CH3)(CH2)4CH3 |
| 1904 | CF2CHF2 | Br | H | CH(CH3)(CH2)5CH3 |
| 1905 | CF2CHF2 | Br | H | CH(CH2CH3)(CH2)3CH3 |
| 1906 | CF2CHF2 | Br | H | CH(CH3)CH2CH(CH3)2 |
| 1907 | CF2CHF2 | Br | H | CH(CH3)C(CH3)3 |
| 1908 | CF2CHF2 | Br | H | CH(CH3)CH(CH3)2 |
| 1909 | CF2CHF2 | Br | H | CH(CH3)CH2CH2CH(CH3)2 |
| 1910 | CF2CHF2 | Br | H | CH(CH2CH3)2 |
| 1911 | CF2CHF2 | Br | H | C(CH3)2CH2CH3 |
| 1912 | CF2CHF2 | Br | H | C(CH3)2CH2C(CH3)3 |
| 1913 | CF2CHF2 | Br | H | CH2—CH(OMe)2 |
| 1914 | CF2CHF2 | Br | H | CH2—CH(OEt)2 |
| 1915 | CF2CHF2 | Br | H | CH2CH2—OH |
| 1916 | CF2CHF2 | Br | H | CH2CH2—OMe |
| 1917 | CF2CHF2 | Br | H | CH2CH2—OEt |
| 1918 | CF2CHF2 | Br | H | CH2CH2—SMe |
| 1919 | CF2CHF2 | Br | H | CH2CH2—CN |
| 1920 | CF2CHF2 | Br | H | CH2CH2—NMe2 |
| 1921 | CF2CHF2 | Br | H | CH2CH2-Morpholin-4-yl |
| 1922 | CF2CHF2 | Br | H | CH(CH3)CH2—OMe |
| 1923 | CF2CHF2 | Br | H | CH(CH3)CH2—NMe2 |
| 1924 | CF2CHF2 | Br | H | CH2CH2CH2—OMe |
| 1925 | CF2CHF2 | Br | H | CH2CH2CH2—SMe |
| 1926 | CF2CHF2 | Br | H | CH2CH2CH2—OEt |
| 1927 | CF2CHF2 | Br | H | CH2CH2CH2—OiPr |
| 1928 | CF2CHF2 | Br | H | CH2CH2CH2—OBu |
| 1929 | CF2CHF2 | Br | H | CH2—COOMe |
| 1930 | CF2CHF2 | Br | H | CH2—COOH |
| 1931 | CF2CHF2 | Br | H | CH(CH3)COOMe |
| 1932 | CF2CHF2 | Br | H | CH(CH3)COOEt |
| 1933 | CF2CHF2 | Br | H | CH2CH2—COOMe |
| 1934 | CF2CHF2 | Br | H | CH(COOMe)2 |
| 1935 | CF2CHF2 | Br | H | CH(COOEt)CH2—CH(CH3)2 |
| 1936 | CF2CHF2 | Br | H | CH(COOMe)CH(CH3)2 |
| 1937 | CF2CHF2 | Br | H | COCH2OMe |
| 1938 | CF2CHF2 | Br | H | CH(cPr)2 |
| 1939 | CF2CHF2 | Br | H | Ph |
| 1940 | CF2CHF2 | Br | H | CH2PH |
| 1941 | CF2CHF2 | Br | H | CH2-Pyridin-3-yl |
| 1942 | CF2CHF2 | Br | H | CH2—6-Cl-Pyridin-3-yl |
| 1943 | CF2CHF2 | Br | H | CH(CH3)Ph |
| 1944 | CF2CHF2 | Br | H | CH2CH2—Ph |
| 1945 | CF2CHF2 | Br | H | CH2—2-CF3—Ph |
| 1946 | CF2CHF2 | Br | H | CH2CH2CHPh2 |
| 1947 | CF2CHF2 | Br | H | O—CH2CH3 |
| 1948 | CF2CHF2 | Br | H | O—CH3 |
| 1949 | CF2CHF2 | Br | H | O—CH2CH═CH2 |
| 1950 | CF2CHF2 | Br | H | O—tBu |
| 1951 | CF2CHF2 | Br | H | O—Pr |
| 1952 | CF2CHF2 | Br | H | O—CH2cPr |
| 1953 | CF2CHF2 | Br | H | O—CH2CH(CH3)2 |
| 1954 | CF2CHF2 | Br | H | O—CH2CF3 |
| 1955 | CF2CHF2 | Br | H | O—CH(CH3)cPr |
| 1956 | CF2CHF2 | Br | H | O—CH2CH2Cl |
| 1957 | CF2CHF2 | Br | H | O—CH2C≡CH |
| 1958 | CF2CHF2 | Br | H | O—CH2C≡CCH3 |
| 1959 | CF2CHF2 | Br | H | O—CH(CH3)C≡CH |
| 1960 | CF2CHF2 | Br | H | Indan-1-yl |
| 1961 | CF2CHF2 | Br | H | Tetrahydrofuran-2-ylmethyl |
| 1962 | CF2CHF2 | Br | Morpholin-4-yl | |
| 1963 | CF2CHF2 | Br | 2-(Methoxycarbonyl)piperidin-1-yl | |
| 1964 | CF2CHF2 | Br | 2-(Ethoxycarbonyl)piperidin-1-yl | |
| 1965 | CF2CHF2 | Br | Thiazolidin-3-yl | |
| 1966 | CF2CHF2 | Br | Pyrrolidin-1-yl | |
| 1967 | CF2CHF2 | Br | 2-Methylpyrrolidin-1-yl | |
| 1968 | CF2CHF2 | Br | ═CH—N(CH3)2 | |
| 1969 | CF2CHF2 | Br | ═C(CH3)N(CH3)2 | |
| 1970 | CF2CHF2 | Br | ═CH—N(C2H5)2 | |
| 1971 | CF2CHF2 | Br | ═C(CH3)N(C2H5)2 | |
| 1972 | CF2CHF2 | Br | ═CH-Piperidine | |
| 1973 | CF2CHF2 | Br | ═CH-Morpholine | |
| 1974 | CF2CHF2 | Br | ═CH-Pyrrolidine | |
| 1975 | CF2CHF2 | Br | Me | Me |
| 1976 | CF2CHF2 | Br | Me | Et |
| 1977 | CF2CHF2 | Br | Et | Me |
| 1978 | CF2CHF2 | Br | Et | Et |
| 1979 | CF2CHF2 | Br | Me | Ph |
| 1980 | CF2CHF2 | Br | Me | CH2CO2Me |
| 1981 | CFClCF3 | Br | H | Me |
| 1982 | CFClCF3 | Br | H | Et |
| 1983 | CFClCF3 | Br | H | Pr |
| 1984 | CFClCF3 | Br | H | iPr |
| 1985 | CFClCF3 | Br | H | cPr |
| 1986 | CFClCF3 | Br | H | Bu |
| 1987 | CFClCF3 | Br | H | cBu |
| 1988 | CFClCF3 | Br | H | tBu |
| 1989 | CFClCF3 | Br | H | cPentyl |
| 1990 | CFClCF3 | Br | H | cHexyl |
| 1991 | CFClCF3 | Br | H | CH2(CH2)3CH3 |
| 1992 | CFClCF3 | Br | H | CH2(CH2)4CH3 |
| 1993 | CFClCF3 | Br | H | CH2—cPr |
| 1994 | CFClCF3 | Br | H | CH2—CN |
| 1995 | CFClCF3 | Br | H | CH2—C(CH3)3 |
| 1996 | CFClCF3 | Br | H | CH2CF2CF3 |
| 1997 | CFClCF3 | Br | H | CH2CF3 |
| 1998 | CFClCF3 | Br | H | CH2(CF2)2CF3 |
| 1999 | CFClCF3 | Br | H | CH2CH(CH3)CH2CH3 |
| 2000 | CFClCF3 | Br | H | CH2C(CH3)2CH2F |
| 2001 | CFClCF3 | Br | H | CH2CH(CH3)2 |
| 2002 | CFClCF3 | Br | H | CH2CH(CH2CH3)2 |
| 2003 | CFClCF3 | Br | H | CH2CH2CH(CH3)2 |
| 2004 | CFClCF3 | Br | H | CH2CH2C(CH3)3 |
| 2005 | CFClCF3 | Br | H | CH2CH═CH2 |
| 2006 | CFClCF3 | Br | H | CH2CH═CHCH3 |
| 2007 | CFClCF3 | Br | H | CH2—C(CH3)═CH2 |
| 2008 | CFClCF3 | Br | H | CH2—C≡CH |
| 2009 | CFClCF3 | Br | H | CH(CH3)CH2CH3 |
| 2010 | CFClCF3 | Br | H | CH(CH3)cPr |
| 2011 | CFClCF3 | Br | H | CH(CH3)CF3 |
| 2012 | CFClCF3 | Br | H | CH(CH3)(CH2)2CH3 |
| 2013 | CFClCF3 | Br | H | CH(CH3)(CH2)4CH3 |
| 2014 | CFClCF3 | Br | H | CH(CH3)(CH2)5CH3 |
| 2015 | CFClCF3 | Br | H | CH(CH2CH3)(CH2)3CH3 |
| 2016 | CFClCF3 | Br | H | CH(CH3)CH2CH(CH3)2 |
| 2017 | CFClCF3 | Br | H | CH(CH3)C(CH3)3 |
| 2018 | CFClCF3 | Br | H | CH(CH3)CH(CH3)2 |
| 2019 | CFClCF3 | Br | H | CH(CH3)CH2CH2CH(CH3)2 |
| 2020 | CFClCF3 | Br | H | CH(CH2CH3)2 |
| 2021 | CFClCF3 | Br | H | C(CH3)2CH2CH3 |
| 2022 | CFClCF3 | Br | H | C(CH3)2CH2C(CH3)3 |
| 2023 | CFClCF3 | Br | H | CH2—CH(OMe)2 |
| 2024 | CFClCF3 | Br | H | CH2—CH(OEt)2 |
| 2025 | CFClCF3 | Br | H | CH2CH2—OH |
| 2026 | CFClCF3 | Br | H | CH2CH2—OMe |
| 2027 | CFClCF3 | Br | H | CH2CH2—OEt |
| 2028 | CFClCF3 | Br | H | CH2CH2—SMe |
| 2029 | CFClCF3 | Br | H | CH2CH2—CN |
| 2030 | CFClCF3 | Br | H | CH2CH2—NMe2 |
| 2031 | CFClCF3 | Br | H | CH2CH2-Morpholin-4-yl |
| 2032 | CFClCF3 | Br | H | CH(CH3)CH2—OMe |
| 2033 | CFClCF3 | Br | H | CH(CH3)CH2—NMe2 |
| 2034 | CFClCF3 | Br | H | CH2CH2CH2—OMe |
| 2035 | CFClCF3 | Br | H | CH2CH2CH2—SMe |
| 2036 | CFClCF3 | Br | H | CH2CH2CH2—OEt |
| 2037 | CFClCF3 | Br | H | CH2CH2CH2—OiPr |
| 2038 | CFClCF3 | Br | H | CH2CH2CH2—OBu |
| 2039 | CFClCF3 | Br | H | CH2—COOMe |
| 2040 | CFClCF3 | Br | H | CH2—COOH |
| 2041 | CFClCF3 | Br | H | CH(CH3)COOMe |
| 2042 | CFClCF3 | Br | H | CH(CH3)COOEt |
| 2043 | CFClCF3 | Br | H | CH2CH2—COOMe |
| 2044 | CFClCF3 | Br | H | CH(COOMe)2 |
| 2045 | CFClCF3 | Br | H | CH(COOEt)CH2—CH(CH3)2 |
| 2046 | CFClCF3 | Br | H | CH(COOMe)CH(CH3)2 |
| 2047 | CFClCF3 | Br | H | COCH2OMe |
| 2048 | CFClCF3 | Br | H | CH(cPr)2 |
| 2049 | CFClCF3 | Br | H | Ph |
| 2050 | CFClCF3 | Br | H | CH2PH |
| 2051 | CFClCF3 | Br | H | CH2-Pyridin-3-yl |
| 2052 | CFClCF3 | Br | H | CH2—6-Cl-Pyridin-3-yl |
| 2053 | CFClCF3 | Br | H | CH(CH3)Ph |
| 2054 | CFClCF3 | Br | H | CH2CH2—Ph |
| 2055 | CFClCF3 | Br | H | CH2—2-CF3—Ph |
| 2056 | CFClCF3 | Br | H | CH2CH2CHPh2 |
| 2057 | CFClCF3 | Br | H | O—CH2CH3 |
| 2058 | CFClCF3 | Br | H | O—CH3 |
| 2059 | CFClCF3 | Br | H | O—CH2CH═CH2 |
| 2060 | CFClCF3 | Br | H | O—tBu |
| 2061 | CFClCF3 | Br | H | O—Pr |
| 2062 | CFClCF3 | Br | H | O—CH2cPr |
| 2063 | CFClCF3 | Br | H | O—CH2CH(CH3)2 |
| 2064 | CFClCF3 | Br | H | O—CH2CF3 |
| 2065 | CFClCF3 | Br | H | O—CH(CH3)cPr |
| 2066 | CFClCF3 | Br | H | O—CH2CH2Cl |
| 2067 | CFClCF3 | Br | H | O—CH2C≡CH |
| 2068 | CFClCF3 | Br | H | O—CH2C≡CCH3 |
| 2069 | CFClCF3 | Br | H | O—CH(CH3)C≡CH |
| 2070 | CFClCF3 | Br | H | Indan-1-yl |
| 2071 | CFClCF3 | Br | H | Tetrahydrofuran-2-ylmethyl |
| 2072 | CFClCF3 | Br | Morpholin-4-yl | |
| 2073 | CFClCF3 | Br | 2-(Methoxycarbonyl)piperidin-1-yl | |
| 2074 | CFClCF3 | Br | 2-(Ethoxycarbonyl)piperidin-1-yl | |
| 2075 | CFClCF3 | Br | Thiazolidin-3-yl | |
| 2076 | CFClCF3 | Br | Pyrrolidin-1-yl | |
| 2077 | CFClCF3 | Br | 2-Methylpyrrolidin-1-yl | |
| 2078 | CFClCF3 | Br | ═CH—N(CH3)2 | |
| 2079 | CFClCF3 | Br | ═C(CH3)N(CH3)2 | |
| 2080 | CFClCF3 | Br | ═CH—N(C2H5)2 | |
| 2081 | CFClCF3 | Br | ═C(CH3)N(C2H5)2 | |
| 2082 | CFClCF3 | Br | ═CH-Piperidine | |
| 2083 | CFClCF3 | Br | ═CH-Morpholine | |
| 2084 | CFClCF3 | Br | ═CH-Pyrrolidine | |
| 2085 | CFClCF3 | Br | Me | Me |
| 2086 | CFClCF3 | Br | Me | Et |
| 2087 | CFClCF3 | Br | Et | Me |
| 2088 | CFClCF3 | Br | Et | Et |
| 2089 | CFClCF3 | Br | Me | Ph |
| 2090 | CFClCF3 | Br | Me | CH2CO2Me |
| 2091 | CHFCF3 | Br | H | Me |
| 2092 | CHFCF3 | Br | H | Et |
| 2093 | CHFCF3 | Br | H | Pr |
| 2094 | CHFCF3 | Br | H | iPr |
| 2095 | CHFCF3 | Br | H | cPr |
| 2096 | CHFCF3 | Br | H | Bu |
| 2097 | CHFCF3 | Br | H | cBu |
| 2098 | CHFCF3 | Br | H | tBu |
| 2099 | CHFCF3 | Br | H | cPentyl |
| 2100 | CHFCF3 | Br | H | cHexyl |
| 2101 | CHFCF3 | Br | H | CH2(CH2)3CH3 |
| 2102 | CHFCF3 | Br | H | CH2(CH2)4CH3 |
| 2103 | CHFCF3 | Br | H | CH2—cPr |
| 2104 | CHFCF3 | Br | H | CH2—CN |
| 2105 | CHFCF3 | Br | H | CH2—C(CH3)3 |
| 2106 | CHFCF3 | Br | H | CH2CF2CF3 |
| 2107 | CHFCF3 | Br | H | CH2CF3 |
| 2108 | CHFCF3 | Br | H | CH2(CF2)2CF3 |
| 2109 | CHFCF3 | Br | H | CH2CH(CH3)CH2CH3 |
| 2110 | CHFCF3 | Br | H | CH2C(CH3)2CH2F |
| 2111 | CHFCF3 | Br | H | CH2CH(CH3)2 |
| 2112 | CHFCF3 | Br | H | CH2CH(CH2CH3)2 |
| 2113 | CHFCF3 | Br | H | CH2CH2CH(CH3)2 |
| 2114 | CHFCF3 | Br | H | CH2CH2C(CH3)3 |
| 2115 | CHFCF3 | Br | H | CH2CH═CH2 |
| 2116 | CHFCF3 | Br | H | CH2CH═CHCH3 |
| 2117 | CHFCF3 | Br | H | CH2—C(CH3)═CH2 |
| 2118 | CHFCF3 | Br | H | CH2—C≡CH |
| 2119 | CHFCF3 | Br | H | CH(CH3)CH2CH3 |
| 2120 | CHFCF3 | Br | H | CH(CH3)cPr |
| 2121 | CHFCF3 | Br | H | CH(CH3)CF3 |
| 2122 | CHFCF3 | Br | H | CH(CH3)(CH2)2CH3 |
| 2123 | CHFCF3 | Br | H | CH(CH3)(CH2)4CH3 |
| 2124 | CHFCF3 | Br | H | CH(CH3)(CH2)5CH3 |
| 2125 | CHFCF3 | Br | H | CH(CH2CH3)(CH2)3CH3 |
| 2126 | CHFCF3 | Br | H | CH(CH3)CH2CH(CH3)2 |
| 2127 | CHFCF3 | Br | H | CH(CH3)C(CH3)3 |
| 2128 | CHFCF3 | Br | H | CH(CH3)CH(CH3)2 |
| 2129 | CHFCF3 | Br | H | CH(CH3)CH2CH2CH(CH3)2 |
| 2130 | CHFCF3 | Br | H | CH(CH2CH3)2 |
| 2131 | CHFCF3 | Br | H | C(CH3)2CH2CH3 |
| 2132 | CHFCF3 | Br | H | C(CH3)2CH2C(CH3)3 |
| 2133 | CHFCF3 | Br | H | CH2—CH(OMe)2 |
| 2134 | CHFCF3 | Br | H | CH2—CH(OEt)2 |
| 2135 | CHFCF3 | Br | H | CH2CH2—OH |
| 2136 | CHFCF3 | Br | H | CH2CH2—OMe |
| 2137 | CHFCF3 | Br | H | CH2CH2—OEt |
| 2138 | CHFCF3 | Br | H | CH2CH2—SMe |
| 2139 | CHFCF3 | Br | H | CH2CH2—CN |
| 2140 | CHFCF3 | Br | H | CH2CH2—NMe2 |
| 2141 | CHFCF3 | Br | H | CH2CH2-Morpholin-4-yl |
| 2142 | CHFCF3 | Br | H | CH(CH3)CH2—OMe |
| 2143 | CHFCF3 | Br | H | CH(CH3)CH2—NMe2 |
| 2144 | CHFCF3 | Br | H | CH2CH2CH2—OMe |
| 2145 | CHFCF3 | Br | H | CH2CH2CH2—SMe |
| 2146 | CHFCF3 | Br | H | CH2CH2CH2—OEt |
| 2147 | CHFCF3 | Br | H | CH2CH2CH2—OiPr |
| 2148 | CHFCF3 | Br | H | CH2CH2CH2—OBu |
| 2149 | CHFCF3 | Br | H | CH2—COOMe |
| 2150 | CHFCF3 | Br | H | CH2—COOH |
| 2151 | CHFCF3 | Br | H | CH(CH3)COOMe |
| 2152 | CHFCF3 | Br | H | CH(CH3)COOEt |
| 2153 | CHFCF3 | Br | H | CH2CH2—COOMe |
| 2154 | CHFCF3 | Br | H | CH(COOMe)2 |
| 2155 | CHFCF3 | Br | H | CH(COOEt)CH2—CH(CH3)2 |
| 2156 | CHFCF3 | Br | H | CH(COOMe)CH(CH3)2 |
| 2157 | CHFCF3 | Br | H | COCH2OMe |
| 2158 | CHFCF3 | Br | H | CH(cPr)2 |
| 2159 | CHFCF3 | Br | H | Ph |
| 2160 | CHFCF3 | Br | H | CH2PH |
| 2161 | CHFCF3 | Br | H | CH2-Pyridin-3-yl |
| 2162 | CHFCF3 | Br | H | CH2—6-Cl-Pyridin-3-yl |
| 2163 | CHFCF3 | Br | H | CH(CH3)Ph |
| 2164 | CHFCF3 | Br | H | CH2CH2—Ph |
| 2165 | CHFCF3 | Br | H | CH2—2-CF3—Ph |
| 2166 | CHFCF3 | Br | H | CH2CH2CHPh2 |
| 2167 | CHFCF3 | Br | H | O—CH2CH3 |
| 2168 | CHFCF3 | Br | H | O—CH3 |
| 2169 | CHFCF3 | Br | H | O—CH2CH═CH2 |
| 2170 | CHFCF3 | Br | H | O—tBu |
| 2171 | CHFCF3 | Br | H | O—Pr |
| 2172 | CHFCF3 | Br | H | O—CH2cPr |
| 2173 | CHFCF3 | Br | H | O—CH2CH(CH3)2 |
| 2174 | CHFCF3 | Br | H | O—CH2CF3 |
| 2175 | CHFCF3 | Br | H | O—CH(CH3)cPr |
| 2176 | CHFCF3 | Br | H | O—CH2CH2Cl |
| 2177 | CHFCF3 | Br | H | O—CH2C≡CH |
| 2178 | CHFCF3 | Br | H | O—CH2C≡CCH3 |
| 2179 | CHFCF3 | Br | H | O—CH(CH3)C≡CH |
| 2180 | CHFCF3 | Br | H | Indan-1-yl |
| 2181 | CHFCF3 | Br | H | Tetrahydrofuran-2-ylmethyl |
| 2182 | CHFCF3 | Br | Morpholin-4-yl | |
| 2183 | CHFCF3 | Br | 2-(Methoxycarbonyl)piperidin-1-yl | |
| 2184 | CHFCF3 | Br | 2-(Ethoxycarbonyl)piperidin-1-yl | |
| 2185 | CHFCF3 | Br | Thiazolidin-3-yl | |
| 2186 | CHFCF3 | Br | Pyrrolidin-1-yl | |
| 2187 | CHFCF3 | Br | 2-Methylpyrrolidin-1-yl | |
| 2188 | CHFCF3 | Br | ═CH—N(CH3)2 | |
| 2189 | CHFCF3 | Br | ═C(CH3)N(CH3)2 | |
| 2190 | CHFCF3 | Br | ═CH—N(C2H5)2 | |
| 2191 | CHFCF3 | Br | ═C(CH3)N(C2H5)2 | |
| 2192 | CHFCF3 | Br | ═CH-Piperidine | |
| 2193 | CHFCF3 | Br | ═CH-Morpholine | |
| 2194 | CHFCF3 | Br | ═CH-Pyrrolidine | |
| 2195 | CHFCF3 | Br | Me | Me |
| 2196 | CHFCF3 | Br | Me | Et |
| 2197 | CHFCF3 | Br | Et | Me |
| 2198 | CHFCF3 | Br | Et | Et |
| 2199 | CHFCF3 | Br | Me | Ph |
| 2200 | CHFCF3 | Br | Me | CH2CO2Me |
| 2201 | CHF2 | Br | H | Me |
| 2202 | CHF2 | Br | H | Et |
| 2203 | CHF2 | Br | H | Pr |
| 2204 | CHF2 | Br | H | iPr |
| 2205 | CHF2 | Br | H | cPr |
| 2206 | CF2CF3 | Br | H | Me |
| 2207 | CF2CF3 | Br | H | Et |
| 2208 | CF2CF3 | Br | H | Pr |
| 2209 | CF2CF3 | Br | H | iPr |
| 2210 | CF2CF3 | Br | H | CH(CH3)CH2CH(CH3)2 |
| 2211 | CF2CHF2 | Cl | H | Me |
| 2212 | CF2CHF2 | Cl | H | Et |
| 2213 | CF2CHF2 | Cl | H | Pr |
| 2214 | CF2CHF2 | Cl | H | iPr |
| 2215 | CF2CHF2 | Cl | H | CH2CH2CH2OMe |
| 2216 | CFClCF3 | Cl | H | Me |
| 2217 | CFClCF3 | Cl | H | Et |
| 2218 | CFClCF3 | Cl | H | Pr |
| 2219 | CFClCF3 | Cl | H | iPr |
| 2220 | CF3 | I | H | Me |
| 2221 | CF3 | I | H | Et |
| 2222 | CF3 | I | H | Pr |
| 2223 | CF3 | I | H | CH2C(CH3)3 |
| 2224 | CF3 | I | H | CH2—COOCH3 |
| 2225 | CF3 | I | H | C(CH3)C(CH3)3 |
| 2226 | CHFCF3 | I | H | Me |
| 2227 | CHFCF3 | I | H | Et |
| 2228 | CHFCF3 | I | H | Pr |
| 2229 | CHFCF3 | I | H | iPr |
| 2230 | CHFCF3 | I | H | cPr |
| 2231 | CHFCF3 | I | H | CH(CH3)(CH2)4CH3 |
| 2232 | CHFCF3 | I | H | CH2CH2OMe |
| 2233 | CHFCF3 | I | H | CH2CH2CH2OMe |
| 2234 | CHFCF3 | I | H | CH(CH3)CH2CH3 |
| 2235 | CHFCF3 | I | H | CH(CH3)CH(CH3)2 |
| 2236 | CF2CHF2 | I | H | Me |
| 2237 | CF2CHF2 | I | H | Et |
| 2238 | CF2CHF2 | I | H | CH2—C(CH3)═CH2 |
| 2239 | CF2CHF2 | I | H | CH(CH3)(CH2)4CH3 |
| 2240 | CF2CHF2 | I | H | CH2CH(OMe)2 |
| 2241 | CFClCF3 | Me | H | Me |
| 2242 | CFClCF3 | Me | H | Et |
| 2243 | CFClCF3 | Me | H | Pr |
| 2244 | CFClCF3 | Me | H | iPr |
| 2245 | CFClCF3 | Me | H | cPr |
| 2246 | CFClCF3 | Me | H | CH(CH3)CH2CH(CH3)2 |
| 2247 | C3F7 | Me | H | Me |
| 2248 | C3F7 | Me | H | Et |
| 2249 | C3F7 | Me | H | Pr |
| 2250 | C3F7 | Me | H | iPr |
| 2251 | C3F7 | Me | H | CH(CH3)CF3 |
| 2252 | CF2CF3 | Me | Me | CH2COOH |
| 2253 | CF3 | Pr | H | COcPr |
| 2254 | Me | Me | Me | CH2COOH |
| 2255 | Me | Et | Me | CH2COOH |
| 2256 | Me | Pr | Me | CH2COOH |
| 2257 | CF3 | Me | Me | CH2COOH |
| 2258 | CF2CF3 | Me | Me | CH2COOH |
| 2259 | Me | Cl | Me | CH2COOH |
| 2260 | Me | Br | Me | CH2COOH |
| 2261 | Me | I | Me | CH2COOH |
| 2262 | Me | Cl | Me | Me |
| 2263 | Me | Cl | Me | Et |
| 2264 | Me | Cl | Et | Me |
| 2265 | Me | Cl | Et | Et |
| 2266 | Me | Cl | Me | Ph |
| 2267 | Me | Cl | Me | CH2CO2Me |
| 2268 | Me | Cl | H | Me |
| 2269 | Me | Cl | H | Et |
| 2270 | Me | Cl | H | Pr |
| 2271 | Me | Cl | H | iPr |
| 2272 | Me | Cl | H | cPr |
| 2273 | Me | Cl | H | Bu |
| 2274 | Me | Cl | H | cBu |
| 2275 | Me | Cl | H | tBu |
| 2276 | Me | Cl | H | cPentyl |
| 2277 | Me | Cl | H | cHexyl |
| 2278 | Me | Cl | H | CH2(CH2)3CH3 |
| 2279 | Me | Cl | H | CH2(CH2)4CH3 |
| 2280 | Me | Cl | H | CH2—cPr |
| 2281 | Me | Cl | H | CH2—CN |
| 2282 | Me | Cl | H | CH2—C(CH3)3 |
| 2283 | Me | Cl | H | CH2CF2CF3 |
| 2284 | Me | Cl | H | CH2CF3 |
| 2285 | Me | Cl | H | CH2(CF2)2CF3 |
| 2286 | Me | Cl | H | CH2CH(CH3)CH2CH3 |
| 2287 | Me | Cl | H | CH2C(CH3)2CH2F |
| 2288 | Me | Cl | H | CH2CH(CH3)2 |
| 2289 | Me | Cl | H | CH2CH(CH2CH3)2 |
| 2290 | Me | Cl | H | CH2CH2CH(CH3)2 |
| 2291 | Me | Cl | H | CH2CH2C(CH3)3 |
| 2292 | Me | Cl | H | CH2CH═CH2 |
| 2293 | Me | Cl | H | CH2CH═CHCH3 |
| 2294 | Me | Cl | H | CH2—C(CH3)═CH2 |
| 2295 | Me | Cl | H | CH2—C≡CH |
| 2296 | Me | Cl | H | CH(CH3)CH2CH3 |
| 2297 | Me | Cl | H | CH(CH3)cPr |
| 2298 | Me | Cl | H | CH(CH3)CF3 |
| 2299 | Me | Cl | H | CH(CH3)(CH2)2CH3 |
| 2300 | Me | Cl | H | CH(CH3)(CH2)4CH3 |
| 2301 | Me | Cl | H | CH(CH3)(CH2)5CH3 |
| 2302 | Me | Cl | H | CH(CH2CH3)(CH2)3CH3 |
| 2303 | Me | Cl | H | CH(CH3)CH2CH(CH3)2 |
| 2304 | Me | Cl | H | CH(CH3)C(CH3)3 |
| 2305 | Me | Cl | H | CH(CH3)CH(CH3)2 |
| 2306 | Me | Cl | H | CH(CH3)CH2CH2CH(CH3)2 |
| 2307 | Me | Cl | H | CH(CH2CH3)2 |
| 2308 | Me | Cl | H | C(CH3)2CH2CH3 |
| 2309 | Me | Cl | H | C(CH3)2CH2C(CH3)3 |
| 2310 | Me | Cl | H | CH2—CH(OMe)2 |
| 2311 | Me | Cl | H | CH2—CH(OEt)2 |
| 2312 | Me | Cl | H | CH2CH2—OH |
| 2313 | Me | Cl | H | CH2CH2—OMe |
| 2314 | Me | Cl | H | CH2CH2—OEt |
| 2315 | Me | Cl | H | CH2CH2—SMe |
| 2316 | Me | Cl | H | CH2CH2—CN |
| 2317 | Me | Cl | H | CH2CH2—NMe2 |
| 2318 | Me | Cl | H | CH2CH2-Morpholin-4-yl |
| 2319 | Me | Cl | H | CH(CH3)CH2—OMe |
| 2320 | Me | Cl | H | CH(CH3)CH2—NMe2 |
| 2321 | Me | Cl | H | CH2CH2CH2—OMe |
| 2322 | Me | Cl | H | CH2CH2CH2—SMe |
| 2323 | Me | Cl | H | CH2CH2CH2—OEt |
| 2324 | Me | Cl | H | CH2CH2CH2—OiPr |
| 2325 | Me | Cl | H | CH2CH2CH2—OBu |
| 2326 | Me | Cl | H | CH2—COOMe |
| 2327 | Me | Cl | H | CH2—COOH |
| 2328 | Me | Cl | H | CH(CH3)COOMe |
| 2329 | Me | Cl | H | CH(CH3)COOEt |
| 2330 | Me | Cl | H | CH2CH2—COOMe |
| 2331 | Me | Cl | H | CH(COOMe)2 |
| 2332 | Me | Cl | H | CH(COOEt)CH2—CH(CH3)2 |
| 2333 | Me | Cl | H | CH(COOMe)CH(CH3)2 |
| 2334 | Me | Cl | H | COCH2OMe |
| 2335 | Me | Cl | H | CH(cPr)2 |
| 2336 | Me | Cl | H | Ph |
| 2337 | Me | Cl | H | CH2PH |
| 2338 | Me | Cl | H | CH2-Pyridin-3-yl |
| 2339 | Me | Cl | H | CH2—6-Cl-Pyridin-3-yl |
| 2340 | Me | Cl | H | CH(CH3)Ph |
| 2341 | Me | Cl | H | CH2CH2—Ph |
| 2342 | Me | Cl | H | CH2—2-CF3—Ph |
| 2343 | Me | Cl | H | CH2CH2CHPh2 |
| 2344 | Me | Cl | H | O—CH2CH3 |
| 2345 | Me | Cl | H | O—CH3 |
| 2346 | Me | Cl | H | O—CH2CH═CH2 |
| 2347 | Me | Cl | H | O—tBu |
| 2348 | Me | Cl | H | O—Pr |
| 2349 | Me | Cl | H | O—CH2cPr |
| 2350 | Me | Cl | H | O—CH2CH(CH3)2 |
| 2351 | Me | Cl | H | O—CH2CF3 |
| 2352 | Me | Cl | H | O—CH(CH3)cPr |
| 2353 | Me | Cl | H | O—CH2CH2Cl |
| 2354 | Me | Cl | H | O—CH2C≡CH |
| 2355 | Me | Cl | H | O—CH2C≡CCH3 |
| 2356 | Me | Cl | H | O—CH(CH3)C≡CH |
| 2357 | Me | Cl | H | Indan-1-yl |
| 2358 | Me | Cl | H | Tetrahydrofuran-2-ylmethyl |
| 2359 | Me | Cl | Morpholin-4-yl | |
| 2360 | Me | Cl | 2-(Methoxycarbonyl)piperidin-1-yl | |
| 2361 | Me | Cl | 2-(Ethoxycarbonyl)piperidin-1-yl | |
| 2362 | Me | Cl | Thiazolidin-3-yl | |
| 2363 | Me | Cl | Pyrrolidin-1-yl | |
| 2364 | Me | Cl | 2-Methylpyrrolidin-1-yl | |
| 2365 | Me | Cl | ═CH—N(CH3)2 | |
| 2366 | Me | Cl | ═C(CH3)N(CH3)2 | |
| 2367 | Me | Cl | ═CH—N(C2H5)2 | |
| 2368 | Me | Cl | ═C(CH3)N(C2H5)2 | |
| 2369 | Me | Cl | ═CH-Piperidine | |
| 2370 | Me | Cl | ═CH-Morpholine | |
| 2371 | Me | Cl | ═CH-Pyrrolidine | |
| 2372 | Me | Br | Me | Me |
| 2373 | Me | Br | Me | Et |
| 2374 | Me | Br | Et | Me |
| 2375 | Me | Br | Et | Et |
| 2376 | Me | Br | Me | Ph |
| 2377 | Me | Br | Me | CH2CO2Me |
| 2378 | Me | Br | H | Me |
| 2379 | Me | Br | H | Et |
| 2380 | Me | Br | H | Pr |
| 2381 | Me | Br | H | iPr |
| 2382 | Me | Br | H | cPr |
| 2383 | Me | Br | H | Bu |
| 2384 | Me | Br | H | cBu |
| 2385 | Me | Br | H | tBu |
| 2386 | Me | Br | H | cPentyl |
| 2387 | Me | Br | H | cHexyl |
| 2388 | Me | Br | H | CH2(CH2)3CH3 |
| 2389 | Me | Br | H | CH2(CH2)4CH3 |
| 2390 | Me | Br | H | CH2—cPr |
| 2391 | Me | Br | H | CH2—CN |
| 2392 | Me | Br | H | CH2—C(CH3)3 |
| 2393 | Me | Br | H | CH2CF2CF3 |
| 2394 | Me | Br | H | CH2CF3 |
| 2395 | Me | Br | H | CH2(CF2)2CF3 |
| 2396 | Me | Br | H | CH2CH(CH3)CH2CH3 |
| 2397 | Me | Br | H | CH2C(CH3)2CH2F |
| 2398 | Me | Br | H | CH2CH(CH3)2 |
| 2399 | Me | Br | H | CH2CH(CH2CH3)2 |
| 2400 | Me | Br | H | CH2CH2CH(CH3)2 |
| 2401 | Me | Br | H | CH2CH2C(CH3)3 |
| 2402 | Me | Br | H | CH2CH═CH2 |
| 2403 | Me | Br | H | CH2CH═CHCH3 |
| 2404 | Me | Br | H | CH2—C(CH3)═CH2 |
| 2405 | Me | Br | H | CH2—C≡CH |
| 2406 | Me | Br | H | CH(CH3)CH2CH3 |
| 2407 | Me | Br | H | CH(CH3)cPr |
| 2408 | Me | Br | H | CH(CH3)CF3 |
| 2409 | Me | Br | H | CH(CH3)(CH2)2CH3 |
| 2410 | Me | Br | H | CH(CH3)(CH2)4CH3 |
| 2411 | Me | Br | H | CH(CH3)(CH2)5CH3 |
| 2412 | Me | Br | H | CH(CH2CH3)(CH2)3CH3 |
| 2413 | Me | Br | H | CH(CH3)CH2CH(CH3)2 |
| 2414 | Me | Br | H | CH(CH3)C(CH3)3 |
| 2415 | Me | Br | H | CH(CH3)CH(CH3)2 |
| 2416 | Me | Br | H | CH(CH3)CH2CH2CH(CH3)2 |
| 2417 | Me | Br | H | CH(CH2CH3)2 |
| 2418 | Me | Br | H | C(CH3)2CH2CH3 |
| 2419 | Me | Br | H | C(CH3)2CH2C(CH3)3 |
| 2420 | Me | Br | H | CH2—CH(OMe)2 |
| 2421 | Me | Br | H | CH2—CH(OEt)2 |
| 2422 | Me | Br | H | CH2CH2—OH |
| 2423 | Me | Br | H | CH2CH2—OMe |
| 2424 | Me | Br | H | CH2CH2—OEt |
| 2425 | Me | Br | H | CH2CH2—SMe |
| 2426 | Me | Br | H | CH2CH2—CN |
| 2427 | Me | Br | H | CH2CH2—NMe2 |
| 2428 | Me | Br | H | CH2CH2-Morpholin-4-yl |
| 2429 | Me | Br | H | CH(CH3)CH2—OMe |
| 2430 | Me | Br | H | CH(CH3)CH2—NMe2 |
| 2431 | Me | Br | H | CH2CH2CH2—OMe |
| 2432 | Me | Br | H | CH2CH2CH2—SMe |
| 2433 | Me | Br | H | CH2CH2CH2—OEt |
| 2434 | Me | Br | H | CH2CH2CH2—OiPr |
| 2435 | Me | Br | H | CH2CH2CH2—OBu |
| 2436 | Me | Br | H | CH2—COOMe |
| 2437 | Me | Br | H | CH2—COOH |
| 2438 | Me | Br | H | CH(CH3)COOMe |
| 2439 | Me | Br | H | CH(CH3)COOEt |
| 2440 | Me | Br | H | CH2CH2—COOMe |
| 2441 | Me | Br | H | CH(COOMe)2 |
| 2442 | Me | Br | H | CH(COOEt)CH2—CH(CH3)2 |
| 2443 | Me | Br | H | CH(COOMe)CH(CH3)2 |
| 2444 | Me | Br | H | COCH2OMe |
| 2445 | Me | Br | H | CH(cPr)2 |
| 2446 | Me | Br | H | Ph |
| 2447 | Me | Br | H | CH2PH |
| 2448 | Me | Br | H | CH2-Pyridin-3-yl |
| 2449 | Me | Br | H | CH2—6-Cl-Pyridin-3-yl |
| 2450 | Me | Br | H | CH(CH3)Ph |
| 2451 | Me | Br | H | CH2CH2—Ph |
| 2452 | Me | Br | H | CH2—2-CF3—Ph |
| 2453 | Me | Br | H | CH2CH2CHPh2 |
| 2454 | Me | Br | H | O—CH2CH3 |
| 2455 | Me | Br | H | O—CH3 |
| 2456 | Me | Br | H | O—CH2CH═CH2 |
| 2457 | Me | Br | H | O—tBu |
| 2458 | Me | Br | H | O—Pr |
| 2459 | Me | Br | H | O—CH2cPr |
| 2460 | Me | Br | H | O—CH2CH(CH3)2 |
| 2461 | Me | Br | H | O—CH2CF3 |
| 2462 | Me | Br | H | O—CH(CH3)cPr |
| 2463 | Me | Br | H | O—CH2CH2Cl |
| 2464 | Me | Br | H | O—CH2C≡CH |
| 2465 | Me | Br | H | O—CH2C≡CCH3 |
| 2466 | Me | Br | H | O—CH(CH3)C≡CH |
| 2467 | Me | Br | H | Indan-1-yl |
| 2468 | Me | Br | H | Tetrahydrofuran-2-ylmethyl |
| 2469 | Me | Br | Morpholin-4-yl | |
| 2470 | Me | Br | 2-(Methoxycarbonyl)piperidin-1-yl | |
| 2471 | Me | Br | 2-(Ethoxycarbonyl)piperidin-1-yl | |
| 2472 | Me | Br | Thiazolidin-3-yl | |
| 2473 | Me | Br | Pyrrolidin-1-yl | |
| 2474 | Me | Br | 2-Methylpyrrolidin-1-yl | |
| 2475 | Me | Br | ═CH—N(CH3)2 | |
| 2476 | Me | Br | ═C(CH3)N(CH3)2 | |
| 2477 | Me | Br | ═CH—N(C2H5)2 | |
| 2478 | Me | Br | ═C(CH3)N(C2H5)2 | |
| 2479 | Me | Br | ═CH-Piperidine | |
| 2480 | Me | Br | ═CH-Morpholine | |
| 2481 | Me | Br | ═CH-Pyrrolidine | |
| 2482 | Me | I | Me | Me |
| 2483 | Me | I | Me | Et |
| 2484 | Me | I | Et | Me |
| 2485 | Me | I | Et | Et |
| 2486 | Me | I | Me | Ph |
| 2487 | Me | I | Me | CH2CO2Me |
| 2488 | Me | I | H | Me |
| 2489 | Me | I | H | Et |
| 2490 | Me | I | H | Pr |
| 2491 | Me | I | H | iPr |
| 2492 | Me | I | H | cPr |
| 2493 | Me | I | H | Bu |
| 2494 | Me | I | H | cBu |
| 2495 | Me | I | H | tBu |
| 2496 | Me | I | H | cPentyl |
| 2497 | Me | I | H | cHexyl |
| 2498 | Me | I | H | CH2(CH2)3CH3 |
| 2499 | Me | I | H | CH2(CH2)4CH3 |
| 2500 | Me | I | H | CH2—cPr |
| 2501 | Me | I | H | CH2—CN |
| 2502 | Me | I | H | CH2—C(CH3)3 |
| 2503 | Me | I | H | CH2CF2CF3 |
| 2504 | Me | I | H | CH2CF3 |
| 2505 | Me | I | H | CH2(CF2)2CF3 |
| 2506 | Me | I | H | CH2CH(CH3)CH2CH3 |
| 2507 | Me | I | H | CH2C(CH3)2CH2F |
| 2508 | Me | I | H | CH2CH(CH3)2 |
| 2509 | Me | I | H | CH2CH(CH2CH3)2 |
| 2510 | Me | I | H | CH2CH2CH(CH3)2 |
| 2511 | Me | I | H | CH2CH2C(CH3)3 |
| 2512 | Me | I | H | CH2CH═CH2 |
| 2513 | Me | I | H | CH2CH═CHCH3 |
| 2514 | Me | I | H | CH2—C(CH3)═CH2 |
| 2515 | Me | I | H | CH2—C≡CH |
| 2516 | Me | I | H | CH(CH3)CH2CH3 |
| 2517 | Me | I | H | CH(CH3)cPr |
| 2518 | Me | I | H | CH(CH3)CF3 |
| 2519 | Me | I | H | CH(CH3)(CH2)2CH3 |
| 2520 | Me | I | H | CH(CH3)(CH2)4CH3 |
| 2521 | Me | I | H | CH(CH3)(CH2)5CH3 |
| 2522 | Me | I | H | CH(CH2CH3)(CH2)3CH3 |
| 2523 | Me | I | H | CH(CH3)CH2CH(CH3)2 |
| 2524 | Me | I | H | CH(CH3)C(CH3)3 |
| 2525 | Me | I | H | CH(CH3)CH(CH3)2 |
| 2526 | Me | I | H | CH(CH3)CH2CH2CH(CH3)2 |
| 2527 | Me | I | H | CH(CH2CH3)2 |
| 2528 | Me | I | H | C(CH3)2CH2CH3 |
| 2529 | Me | I | H | C(CH3)2CH2C(CH3)3 |
| 2530 | Me | I | H | CH2—CH(OMe)2 |
| 2531 | Me | I | H | CH2—CH(OEt)2 |
| 2532 | Me | I | H | CH2CH2—OH |
| 2533 | Me | I | H | CH2CH2—OMe |
| 2534 | Me | I | H | CH2CH2—OEt |
| 2535 | Me | I | H | CH2CH2—SMe |
| 2536 | Me | I | H | CH2CH2—CN |
| 2537 | Me | I | H | CH2CH2—NMe2 |
| 2538 | Me | I | H | CH2CH2-Morpholin-4-yl |
| 2539 | Me | I | H | CH(CH3)CH2—OMe |
| 2540 | Me | I | H | CH(CH3)CH2—NMe2 |
| 2541 | Me | I | H | CH2CH2CH2—OMe |
| 2542 | Me | I | H | CH2CH2CH2—SMe |
| 2543 | Me | I | H | CH2CH2CH2—OEt |
| 2544 | Me | I | H | CH2CH2CH2—OiPr |
| 2545 | Me | I | H | CH2CH2CH2—OBu |
| 2546 | Me | I | H | CH2—COOMe |
| 2547 | Me | I | H | CH2—COOH |
| 2548 | Me | I | H | CH(CH3)COOMe |
| 2549 | Me | I | H | CH(CH3)COOEt |
| 2550 | Me | I | H | CH2CH2—COOMe |
| 2551 | Me | I | H | CH(COOMe)2 |
| 2552 | Me | I | H | CH(COOEt)CH2—CH(CH3)2 |
| 2553 | Me | I | H | CH(COOMe)CH(CH3)2 |
| 2554 | Me | I | H | COCH2OMe |
| 2555 | Me | I | H | CH(cPr)2 |
| 2556 | Me | I | H | Ph |
| 2557 | Me | I | H | CH2PH |
| 2558 | Me | I | H | CH2-Pyridin-3-yl |
| 2559 | Me | I | H | CH2—6-Cl-Pyridin-3-yl |
| 2560 | Me | I | H | CH(CH3)Ph |
| 2561 | Me | I | H | CH2CH2—Ph |
| 2562 | Me | I | H | CH2—2-CF3—Ph |
| 2563 | Me | I | H | CH2CH2CHPh2 |
| 2564 | Me | I | H | O—CH2CH3 |
| 2565 | Me | I | H | O—CH3 |
| 2566 | Me | I | H | O—CH2CH═CH2 |
| 2567 | Me | I | H | O—tBu |
| 2568 | Me | I | H | O—Pr |
| 2569 | Me | I | H | O—CH2cPr |
| 2570 | Me | I | H | O—CH2CH(CH3)2 |
| 2571 | Me | I | H | O—CH2CF3 |
| 2572 | Me | I | H | O—CH(CH3)cPr |
| 2573 | Me | I | H | O—CH2CH2Cl |
| 2574 | Me | I | H | O—CH2C≡CH |
| 2575 | Me | I | H | O—CH2C≡CCH3 |
| 2576 | Me | I | H | O—CH(CH3)C≡CH |
| 2577 | Me | I | H | Indan-1-yl |
| 2578 | Me | I | H | Tetrahydrofuran-2-ylmethyl |
| 2579 | Me | I | Morpholin-4-yl | |
| 2580 | Me | I | 2-(Methoxycarbonyl)piperidin-1-yl | |
| 2581 | Me | I | 2-(Ethoxycarbonyl)piperidin-1-yl | |
| 2582 | Me | I | Thiazolidin-3-yl | |
| 2583 | Me | I | Pyrrolidin-1-yl | |
| 2584 | Me | I | 2-Methylpyrrolidin-1-yl | |
| 2585 | Me | I | ═CH—N(CH3)2 | |
| 2586 | Me | I | ═C(CH3)N(CH3)2 | |
| 2587 | Me | I | ═CH—N(C2H5)2 | |
| 2588 | Me | I | ═C(CH3)N(C2H5)2 | |
| 2589 | Me | I | ═CH-Piperidine | |
| 2590 | Me | I | ═CH-Morpholine | |
| 2591 | Me | I | ═CH-Pyrrolidine | |
| 2592 | Me | Et | Me | Me |
| 2593 | Me | Et | Me | Et |
| 2594 | Me | Et | Et | Me |
| 2595 | Me | Et | Et | Et |
| 2596 | Me | Et | Me | Ph |
| 2597 | Me | Et | Me | CH2CO2Me |
| 2598 | Me | Et | H | Me |
| 2599 | Me | Et | H | Et |
| 2600 | Me | Et | H | Pr |
| 2601 | Me | Et | H | iPr |
| 2602 | Me | Et | H | cPr |
| 2603 | Me | Et | H | Bu |
| 2604 | Me | Et | H | cBu |
| 2605 | Me | Et | H | tBu |
| 2606 | Me | Et | H | cPentyl |
| 2607 | Me | Et | H | cHexyl |
| 2608 | Me | Et | H | CH2(CH2)3CH3 |
| 2609 | Me | Et | H | CH2(CH2)4CH3 |
| 2610 | Me | Et | H | CH2—cPr |
| 2611 | Me | Et | H | CH2—CN |
| 2612 | Me | Et | H | CH2—C(CH3)3 |
| 2613 | Me | Et | H | CH2CF2CF3 |
| 2614 | Me | Et | H | CH2CF3 |
| 2615 | Me | Et | H | CH2(CF2)2CF3 |
| 2616 | Me | Et | H | CH2CH(CH3)CH2CH3 |
| 2617 | Me | Et | H | CH2C(CH3)2CH2F |
| 2618 | Me | Et | H | CH2CH(CH3)2 |
| 2619 | Me | Et | H | CH2CH(CH2CH3)2 |
| 2620 | Me | Et | H | CH2CH2CH(CH3)2 |
| 2621 | Me | Et | H | CH2CH2C(CH3)3 |
| 2622 | Me | Et | H | CH2CH═CH2 |
| 2623 | Me | Et | H | CH2CH═CHCH3 |
| 2624 | Me | Et | H | CH2—C(CH3)═CH2 |
| 2625 | Me | Et | H | CH2—C≡CH |
| 2626 | Me | Et | H | CH(CH3)CH2CH3 |
| 2627 | Me | Et | H | CH(CH3)cPr |
| 2628 | Me | Et | H | CH(CH3)CF3 |
| 2629 | Me | Et | H | CH(CH3)(CH2)2CH3 |
| 2630 | Me | Et | H | CH(CH3)(CH2)4CH3 |
| 2631 | Me | Et | H | CH(CH3)(CH2)5CH3 |
| 2632 | Me | Et | H | CH(CH2CH3)(CH2)3CH3 |
| 2633 | Me | Et | H | CH(CH3)CH2CH(CH3)2 |
| 2634 | Me | Et | H | CH(CH3)C(CH3)3 |
| 2635 | Me | Et | H | CH(CH3)CH(CH3)2 |
| 2636 | Me | Et | H | CH(CH3)CH2CH2CH(CH3)2 |
| 2637 | Me | Et | H | CH(CH2CH3)2 |
| 2638 | Me | Et | H | C(CH3)2CH2CH3 |
| 2639 | Me | Et | H | C(CH3)2CH2C(CH3)3 |
| 2640 | Me | Et | H | CH2—CH(OMe)2 |
| 2641 | Me | Et | H | CH2—CH(OEt)2 |
| 2642 | Me | Et | H | CH2CH2—OH |
| 2643 | Me | Et | H | CH2CH2—OMe |
| 2644 | Me | Et | H | CH2CH2—OEt |
| 2645 | Me | Et | H | CH2CH2—SMe |
| 2646 | Me | Et | H | CH2CH2—CN |
| 2647 | Me | Et | H | CH2CH2—NMe2 |
| 2648 | Me | Et | H | CH2CH2-Morpholin-4-yl |
| 2649 | Me | Et | H | CH(CH3)CH2—OMe |
| 2650 | Me | Et | H | CH(CH3)CH2—NMe2 |
| 2651 | Me | Et | H | CH2CH2CH2—OMe |
| 2652 | Me | Et | H | CH2CH2CH2—SMe |
| 2653 | Me | Et | H | CH2CH2CH2—OEt |
| 2654 | Me | Et | H | CH2CH2CH2—OiPr |
| 2655 | Me | Et | H | CH2CH2CH2—OBu |
| 2656 | Me | Et | H | CH2—COOMe |
| 2657 | Me | Et | H | CH2—COOH |
| 2658 | Me | Et | H | CH(CH3)COOMe |
| 2659 | Me | Et | H | CH(CH3)COOEt |
| 2660 | Me | Et | H | CH2CH2—COOMe |
| 2661 | Me | Et | H | CH(COOMe)2 |
| 2662 | Me | Et | H | CH(COOEt)CH2—CH(CH3)2 |
| 2663 | Me | Et | H | CH(COOMe)CH(CH3)2 |
| 2664 | Me | Et | H | COCH2OMe |
| 2665 | Me | Et | H | CH(cPr)2 |
| 2666 | Me | Et | H | Ph |
| 2667 | Me | Et | H | CH2PH |
| 2668 | Me | Et | H | CH2-Pyridin-3-yl |
| 2669 | Me | Et | H | CH2—6-Cl-Pyridin-3-yl |
| 2670 | Me | Et | H | CH(CH3)Ph |
| 2671 | Me | Et | H | CH2CH2—Ph |
| 2672 | Me | Et | H | CH2—2-CF3—Ph |
| 2673 | Me | Et | H | CH2CH2CHPh2 |
| 2674 | Me | Et | H | O—CH2CH3 |
| 2675 | Me | Et | H | O—CH3 |
| 2676 | Me | Et | H | O—CH2CH═CH2 |
| 2677 | Me | Et | H | O—tBu |
| 2678 | Me | Et | H | O—Pr |
| 2679 | Me | Et | H | O—CH2cPr |
| 2680 | Me | Et | H | O—CH2CH(CH3)2 |
| 2681 | Me | Et | H | O—CH2CF3 |
| 2682 | Me | Et | H | O—CH(CH3)cPr |
| 2683 | Me | Et | H | O—CH2CH2Cl |
| 2684 | Me | Et | H | O—CH2C≡CH |
| 2685 | Me | Et | H | O—CH2C≡CCH3 |
| 2686 | Me | Et | H | O—CH(CH3)C≡CH |
| 2687 | Me | Et | H | Indan-1-yl |
| 2688 | Me | Et | H | Tetrahydrofuran-2-ylmethyl |
| 2689 | Me | Et | Morpholin-4-yl | |
| 2690 | Me | Et | 2-(Methoxycarbonyl)piperidin-1-yl | |
| 2691 | Me | Et | 2-(Ethoxycarbonyl)piperidin-1-yl | |
| 2692 | Me | Et | Thiazolidin-3-yl | |
| 2693 | Me | Et | Pyrrolidin-1-yl | |
| 2694 | Me | Et | 2-Methylpyrrolidin-1-yl | |
| 2695 | Me | Et | ═CH—N(CH3)2 | |
| 2696 | Me | Et | ═C(CH3)N(CH3)2 | |
| 2697 | Me | Et | ═CH—N(C2H5)2 | |
| 2698 | Me | Et | ═C(CH3)N(C2H5)2 | |
| 2699 | Me | Et | ═CH-Piperidine | |
| 2700 | Me | Et | ═CH-Morpholine | |
| 2701 | Me | Et | ═CH-Pyrrolidine | |
| 2702 | Me | Pr | Me | Me |
| 2703 | Me | Pr | Me | Et |
| 2704 | Me | Pr | Et | Me |
| 2705 | Me | Pr | Et | Et |
| 2706 | Me | Pr | Me | Ph |
| 2707 | Me | Pr | Me | CH2CO2Me |
| 2708 | Me | Pr | H | Me |
| 2709 | Me | Pr | H | Et |
| 2710 | Me | Pr | H | Pr |
| 2711 | Me | Pr | H | iPr |
| 2712 | Me | Pr | H | cPr |
| 2713 | Me | Pr | H | Bu |
| 2714 | Me | Pr | H | cBu |
| 2715 | Me | Pr | H | tBu |
| 2716 | Me | Pr | H | cPentyl |
| 2717 | Me | Pr | H | cHexyl |
| 2718 | Me | Pr | H | CH2(CH2)3CH3 |
| 2719 | Me | Pr | H | CH2(CH2)4CH3 |
| 2720 | Me | Pr | H | CH2—cPr |
| 2721 | Me | Pr | H | CH2—CN |
| 2722 | Me | Pr | H | CH2—C(CH3)3 |
| 2723 | Me | Pr | H | CH2CF2CF3 |
| 2724 | Me | Pr | H | CH2CF3 |
| 2725 | Me | Pr | H | CH2(CF2)2CF3 |
| 2726 | Me | Pr | H | CH2CH(CH3)CH2CH3 |
| 2727 | Me | Pr | H | CH2C(CH3)2CH2F |
| 2728 | Me | Pr | H | CH2CH(CH3)2 |
| 2729 | Me | Pr | H | CH2CH(CH2CH3)2 |
| 2730 | Me | Pr | H | CH2CH2CH(CH3)2 |
| 2731 | Me | Pr | H | CH2CH2C(CH3)3 |
| 2732 | Me | Pr | H | CH2CH═CH2 |
| 2733 | Me | Pr | H | CH2CH═CHCH3 |
| 2734 | Me | Pr | H | CH2—C(CH3)═CH2 |
| 2735 | Me | Pr | H | CH2—C≡CH |
| 2736 | Me | Pr | H | CH(CH3)CH2CH3 |
| 2737 | Me | Pr | H | CH(CH3)cPr |
| 2738 | Me | Pr | H | CH(CH3)CF3 |
| 2739 | Me | Pr | H | CH(CH3)(CH2)2CH3 |
| 2740 | Me | Pr | H | CH(CH3)(CH2)4CH3 |
| 2741 | Me | Pr | H | CH(CH3)(CH2)5CH3 |
| 2742 | Me | Pr | H | CH(CH2CH3)(CH2)3CH3 |
| 2743 | Me | Pr | H | CH(CH3)CH2CH(CH3)2 |
| 2744 | Me | Pr | H | CH(CH3)C(CH3)3 |
| 2745 | Me | Pr | H | CH(CH3)CH(CH3)2 |
| 2746 | Me | Pr | H | CH(CH3)CH2CH2CH(CH3)2 |
| 2747 | Me | Pr | H | CH(CH2CH3)2 |
| 2748 | Me | Pr | H | C(CH3)2CH2CH3 |
| 2749 | Me | Pr | H | C(CH3)2CH2C(CH3)3 |
| 2750 | Me | Pr | H | CH2—CH(OMe)2 |
| 2751 | Me | Pr | H | CH2—CH(OEt)2 |
| 2752 | Me | Pr | H | CH2CH2—OH |
| 2753 | Me | Pr | H | CH2CH2—OMe |
| 2754 | Me | Pr | H | CH2CH2—OEt |
| 2755 | Me | Pr | H | CH2CH2—SMe |
| 2756 | Me | Pr | H | CH2CH2—CN |
| 2757 | Me | Pr | H | CH2CH2—NMe2 |
| 2758 | Me | Pr | H | CH2CH2-Morpholin-4-yl |
| 2759 | Me | Pr | H | CH(CH3)CH2—OMe |
| 2760 | Me | Pr | H | CH(CH3)CH2—NMe2 |
| 2761 | Me | Pr | H | CH2CH2CH2—OMe |
| 2762 | Me | Pr | H | CH2CH2CH2—SMe |
| 2763 | Me | Pr | H | CH2CH2CH2—OEt |
| 2764 | Me | Pr | H | CH2CH2CH2—OiPr |
| 2765 | Me | Pr | H | CH2CH2CH2—OBu |
| 2766 | Me | Pr | H | CH2—COOMe |
| 2767 | Me | Pr | H | CH2—COOH |
| 2768 | Me | Pr | H | CH(CH3)COOMe |
| 2769 | Me | Pr | H | CH(CH3)COOEt |
| 2770 | Me | Pr | H | CH2CH2—COOMe |
| 2771 | Me | Pr | H | CH(COOMe)2 |
| 2772 | Me | Pr | H | CH(COOEt)CH2—CH(CH3)2 |
| 2773 | Me | Pr | H | CH(COOMe)CH(CH3)2 |
| 2774 | Me | Pr | H | COCH2OMe |
| 2775 | Me | Pr | H | CH(cPr)2 |
| 2776 | Me | Pr | H | Ph |
| 2777 | Me | Pr | H | CH2PH |
| 2778 | Me | Pr | H | CH2-Pyridin-3-yl |
| 2779 | Me | Pr | H | CH2—6-Cl-Pyridin-3-yl |
| 2780 | Me | Pr | H | CH(CH3)Ph |
| 2781 | Me | Pr | H | CH2CH2—Ph |
| 2782 | Me | Pr | H | CH2—2-CF3—Ph |
| 2783 | Me | Pr | H | CH2CH2CHPh2 |
| 2784 | Me | Pr | H | O—CH2CH3 |
| 2785 | Me | Pr | H | O—CH3 |
| 2786 | Me | Pr | H | O—CH2CH═CH2 |
| 2787 | Me | Pr | H | O—tBu |
| 2788 | Me | Pr | H | O—Pr |
| 2789 | Me | Pr | H | O—CH2cPr |
| 2790 | Me | Pr | H | O—CH2CH(CH3)2 |
| 2791 | Me | Pr | H | O—CH2CF3 |
| 2792 | Me | Pr | H | O—CH(CH3)cPr |
| 2793 | Me | Pr | H | O—CH2CH2Cl |
| 2794 | Me | Pr | H | O—CH2C≡CH |
| 2795 | Me | Pr | H | O—CH2C≡CCH3 |
| 2796 | Me | Pr | H | O—CH(CH3)C≡CH |
| 2797 | Me | Pr | H | Indan-1-yl |
| 2798 | Me | Pr | H | Tetrahydrofuran-2-ylmethyl |
| 2799 | Me | Pr | Morpholin-4-yl | |
| 2800 | Me | Pr | 2-(Methoxycarbonyl)piperidin-1-yl | |
| 2801 | Me | Pr | 2-(Ethoxycarbonyl)piperidin-1-yl | |
| 2802 | Me | Pr | Thiazolidin-3-yl | |
| 2803 | Me | Pr | Pyrrolidin-1-yl | |
| 2804 | Me | Pr | 2-Methylpyrrolidin-1-yl | |
| 2805 | Me | Pr | ═CH—N(CH3)2 | |
| 2806 | Me | Pr | ═C(CH3)N(CH3)2 | |
| 2807 | Me | Pr | ═CH—N(C2H5)2 | |
| 2808 | Me | Pr | ═C(CH3)N(C2H5)2 | |
| 2809 | Me | Pr | ═CH-Piperidine | |
| 2810 | Me | Pr | ═CH-Morpholine | |
| 2811 | Me | Pr | ═CH-Pyrrolidine | |
| 2812 | Et | Me | Me | Me |
| 2813 | Et | Me | Me | Et |
| 2814 | Et | Me | Et | Me |
| 2815 | Et | Me | Et | Et |
| 2816 | Et | Me | Me | Ph |
| 2817 | Et | Me | Me | CH2CO2Me |
| 2818 | Et | Me | H | Me |
| 2819 | Et | Me | H | Et |
| 2820 | Et | Me | H | Pr |
| 2821 | Et | Me | H | iPr |
| 2822 | Et | Me | H | cPr |
| 2823 | Et | Me | H | Bu |
| 2824 | Et | Me | H | cBu |
| 2825 | Et | Me | H | tBu |
| 2826 | Et | Me | H | cPentyl |
| 2827 | Et | Me | H | cHexyl |
| 2828 | Et | Me | H | CH2(CH2)3CH3 |
| 2829 | Et | Me | H | CH2(CH2)4CH3 |
| 2830 | Et | Me | H | CH2—cPr |
| 2831 | Et | Me | H | CH2—CN |
| 2832 | Et | Me | H | CH2—C(CH3)3 |
| 2833 | Et | Me | H | CH2CF2CF3 |
| 2834 | Et | Me | H | CH2CF3 |
| 2835 | Et | Me | H | CH2(CF2)2CF3 |
| 2836 | Et | Me | H | CH2CH(CH3)CH2CH3 |
| 2837 | Et | Me | H | CH2C(CH3)2CH2F |
| 2838 | Et | Me | H | CH2CH(CH3)2 |
| 2839 | Et | Me | H | CH2CH(CH2CH3)2 |
| 2840 | Et | Me | H | CH2CH2CH(CH3)2 |
| 2841 | Et | Me | H | CH2CH2C(CH3)3 |
| 2842 | Et | Me | H | CH2CH═CH2 |
| 2843 | Et | Me | H | CH2CH═CHCH3 |
| 2844 | Et | Me | H | CH2—C(CH3)═CH2 |
| 2845 | Et | Me | H | CH2—C≡CH |
| 2846 | Et | Me | H | CH(CH3)CH2CH3 |
| 2847 | Et | Me | H | CH(CH3)cPr |
| 2848 | Et | Me | H | CH(CH3)CF3 |
| 2849 | Et | Me | H | CH(CH3)(CH2)2CH3 |
| 2850 | Et | Me | H | CH(CH3)(CH2)4CH3 |
| 2851 | Et | Me | H | CH(CH3)(CH2)5CH3 |
| 2852 | Et | Me | H | CH(CH2CH3)(CH2)3CH3 |
| 2853 | Et | Me | H | CH(CH3)CH2CH(CH3)2 |
| 2854 | Et | Me | H | CH(CH3)C(CH3)3 |
| 2855 | Et | Me | H | CH(CH3)CH(CH3)2 |
| 2856 | Et | Me | H | CH(CH3)CH2CH2CH(CH3)2 |
| 2857 | Et | Me | H | CH(CH2CH3)2 |
| 2858 | Et | Me | H | C(CH3)2CH2CH3 |
| 2859 | Et | Me | H | C(CH3)2CH2C(CH3)3 |
| 2860 | Et | Me | H | CH2—CH(OMe)2 |
| 2861 | Et | Me | H | CH2—CH(OEt)2 |
| 2862 | Et | Me | H | CH2CH2—OH |
| 2863 | Et | Me | H | CH2CH2—OMe |
| 2864 | Et | Me | H | CH2CH2—OEt |
| 2865 | Et | Me | H | CH2CH2—SMe |
| 2866 | Et | Me | H | CH2CH2—CN |
| 2867 | Et | Me | H | CH2CH2—NMe2 |
| 2868 | Et | Me | H | CH2CH2-Morpholin-4-yl |
| 2869 | Et | Me | H | CH(CH3)CH2—OMe |
| 2870 | Et | Me | H | CH(CH3)CH2—NMe2 |
| 2871 | Et | Me | H | CH2CH2CH2—OMe |
| 2872 | Et | Me | H | CH2CH2CH2—SMe |
| 2873 | Et | Me | H | CH2CH2CH2—OEt |
| 2874 | Et | Me | H | CH2CH2CH2—OiPr |
| 2875 | Et | Me | H | CH2CH2CH2—OBu |
| 2876 | Et | Me | H | CH2—COOMe |
| 2877 | Et | Me | H | CH2—COOH |
| 2878 | Et | Me | H | CH(CH3)COOMe |
| 2879 | Et | Me | H | CH(CH3)COOEt |
| 2880 | Et | Me | H | CH2CH2—COOMe |
| 2881 | Et | Me | H | CH(COOMe)2 |
| 2882 | Et | Me | H | CH(COOEt)CH2—CH(CH3)2 |
| 2883 | Et | Me | H | CH(COOMe)CH(CH3)2 |
| 2884 | Et | Me | H | COCH2OMe |
| 2885 | Et | Me | H | CH(cPr)2 |
| 2886 | Et | Me | H | Ph |
| 2887 | Et | Me | H | CH2PH |
| 2888 | Et | Me | H | CH2-Pyridin-3-yl |
| 2889 | Et | Me | H | CH2—6-Cl-Pyridin-3-yl |
| 2890 | Et | Me | H | CH(CH3)Ph |
| 2891 | Et | Me | H | CH2CH2—Ph |
| 2892 | Et | Me | H | CH2—2-CF3—Ph |
| 2893 | Et | Me | H | CH2CH2CHPh2 |
| 2894 | Et | Me | H | O—CH2CH3 |
| 2895 | Et | Me | H | O—CH3 |
| 2896 | Et | Me | H | O—CH2CH═CH2 |
| 2897 | Et | Me | H | O—tBu |
| 2898 | Et | Me | H | O—Pr |
| 2899 | Et | Me | H | O—CH2cPr |
| 2900 | Et | Me | H | O—CH2CH(CH3)2 |
| 2901 | Et | Me | H | O—CH2CF3 |
| 2902 | Et | Me | H | O—CH(CH3)cPr |
| 2903 | Et | Me | H | O—CH2CH2Cl |
| 2904 | Et | Me | H | O—CH2C≡CH |
| 2905 | Et | Me | H | O—CH2C≡CCH3 |
| 2906 | Et | Me | H | O—CH(CH3)C≡CH |
| 2907 | Et | Me | H | Indan-1-yl |
| 2908 | Et | Me | H | Tetrahydrofuran-2-ylmethyl |
| 2909 | Et | Me | Morpholin-4-yl | |
| 2910 | Et | Me | 2-(Methoxycarbonyl)piperidin-1-yl | |
| 2911 | Et | Me | 2-(Ethoxycarbonyl)piperidin-1-yl | |
| 2912 | Et | Me | Thiazolidin-3-yl | |
| 2913 | Et | Me | Pyrrolidin-1-yl | |
| 2914 | Et | Me | 2-Methylpyrrolidin-1-yl | |
| 2915 | Et | Me | ═CH—N(CH3)2 | |
| 2916 | Et | Me | ═C(CH3)N(CH3)2 | |
| 2917 | Et | Me | ═CH—N(C2H5)2 | |
| 2918 | Et | Me | ═C(CH3)N(C2H5)2 | |
| 2919 | Et | Me | ═CH-Piperidine | |
| 2920 | Et | Me | ═CH-Morpholine | |
| 2921 | Et | Me | ═CH-Pyrrolidine | |
| 2922 | Et | Et | Me | Me |
| 2923 | Et | Et | Me | Et |
| 2924 | Et | Et | Et | Me |
| 2925 | Et | Et | Et | Et |
| 2926 | Et | Et | Me | Ph |
| 2927 | Et | Et | Me | CH2CO2Me |
| 2928 | Et | Et | H | Me |
| 2929 | Et | Et | H | Et |
| 2930 | Et | Et | H | Pr |
| 2931 | Et | Et | H | iPr |
| 2932 | Et | Et | H | cPr |
| 2933 | Et | Et | H | Bu |
| 2934 | Et | Et | H | cBu |
| 2935 | Et | Et | H | tBu |
| 2936 | Et | Et | H | cPentyl |
| 2937 | Et | Et | H | cHexyl |
| 2938 | Et | Et | H | CH2(CH2)3CH3 |
| 2939 | Et | Et | H | CH2(CH2)4CH3 |
| 2940 | Et | Et | H | CH2—cPr |
| 2941 | Et | Et | H | CH2—CN |
| 2942 | Et | Et | H | CH2—C(CH3)3 |
| 2943 | Et | Et | H | CH2CF2CF3 |
| 2944 | Et | Et | H | CH2CF3 |
| 2945 | Et | Et | H | CH2(CF2)2CF3 |
| 2946 | Et | Et | H | CH2CH(CH3)CH2CH3 |
| 2947 | Et | Et | H | CH2C(CH3)2CH2F |
| 2948 | Et | Et | H | CH2CH(CH3)2 |
| 2949 | Et | Et | H | CH2CH(CH2CH3)2 |
| 2950 | Et | Et | H | CH2CH2CH(CH3)2 |
| 2951 | Et | Et | H | CH2CH2C(CH3)3 |
| 2952 | Et | Et | H | CH2CH═CH2 |
| 2953 | Et | Et | H | CH2CH═CHCH3 |
| 2954 | Et | Et | H | CH2—C(CH3)═CH2 |
| 2955 | Et | Et | H | CH2—C≡CH |
| 2956 | Et | Et | H | CH(CH3)CH2CH3 |
| 2957 | Et | Et | H | CH(CH3)cPr |
| 2958 | Et | Et | H | CH(CH3)CF3 |
| 2959 | Et | Et | H | CH(CH3)(CH2)2CH3 |
| 2960 | Et | Et | H | CH(CH3)(CH2)4CH3 |
| 2961 | Et | Et | H | CH(CH3)(CH2)5CH3 |
| 2962 | Et | Et | H | CH(CH2CH3)(CH2)3CH3 |
| 2963 | Et | Et | H | CH(CH3)CH2CH(CH3)2 |
| 2964 | Et | Et | H | CH(CH3)C(CH3)3 |
| 2965 | Et | Et | H | CH(CH3)CH(CH3)2 |
| 2966 | Et | Et | H | CH(CH3)CH2CH2CH(CH3)2 |
| 2967 | Et | Et | H | CH(CH2CH3)2 |
| 2968 | Et | Et | H | C(CH3)2CH2CH3 |
| 2969 | Et | Et | H | C(CH3)2CH2C(CH3)3 |
| 2970 | Et | Et | H | CH2—CH(OMe)2 |
| 2971 | Et | Et | H | CH2—CH(OEt)2 |
| 2972 | Et | Et | H | CH2CH2—OH |
| 2973 | Et | Et | H | CH2CH2—OMe |
| 2974 | Et | Et | H | CH2CH2—OEt |
| 2975 | Et | Et | H | CH2CH2—SMe |
| 2976 | Et | Et | H | CH2CH2—CN |
| 2977 | Et | Et | H | CH2CH2—NMe2 |
| 2978 | Et | Et | H | CH2CH2-Morpholin-4-yl |
| 2979 | Et | Et | H | CH(CH3)CH2—OMe |
| 2980 | Et | Et | H | CH(CH3)CH2—NMe2 |
| 2981 | Et | Et | H | CH2CH2CH2—OMe |
| 2982 | Et | Et | H | CH2CH2CH2—SMe |
| 2983 | Et | Et | H | CH2CH2CH2—OEt |
| 2984 | Et | Et | H | CH2CH2CH2—OiPr |
| 2985 | Et | Et | H | CH2CH2CH2—OBu |
| 2986 | Et | Et | H | CH2—COOMe |
| 2987 | Et | Et | H | CH2—COOH |
| 2988 | Et | Et | H | CH(CH3)COOMe |
| 2989 | Et | Et | H | CH(CH3)COOEt |
| 2990 | Et | Et | H | CH2CH2—COOMe |
| 2991 | Et | Et | H | CH(COOMe)2 |
| 2992 | Et | Et | H | CH(COOEt)CH2—CH(CH3)2 |
| 2993 | Et | Et | H | CH(COOMe)CH(CH3)2 |
| 2994 | Et | Et | H | COCH2OMe |
| 2995 | Et | Et | H | CH(cPr)2 |
| 2996 | Et | Et | H | Ph |
| 2997 | Et | Et | H | CH2PH |
| 2998 | Et | Et | H | CH2-Pyridin-3-yl |
| 2999 | Et | Et | H | CH2—6-Cl-Pyridin-3-yl |
| 3000 | Et | Et | H | CH(CH3)Ph |
| 3001 | Et | Et | H | CH2CH2—Ph |
| 3002 | Et | Et | H | CH2—2-CF3—Ph |
| 3003 | Et | Et | H | CH2CH2CHPh2 |
| 3004 | Et | Et | H | O—CH2CH3 |
| 3005 | Et | Et | H | O—CH3 |
| 3006 | Et | Et | H | O—CH2CH═CH2 |
| 3007 | Et | Et | H | O—tBu |
| 3008 | Et | Et | H | O—Pr |
| 3009 | Et | Et | H | O—CH2cPr |
| 3010 | Et | Et | H | O—CH2CH(CH3)2 |
| 3011 | Et | Et | H | O—CH2CF3 |
| 3012 | Et | Et | H | O—CH(CH3)cPr |
| 3013 | Et | Et | H | O—CH2CH2Cl |
| 3014 | Et | Et | H | O—CH2C≡CH |
| 3015 | Et | Et | H | O—CH2C≡CCH3 |
| 3016 | Et | Et | H | O—CH(CH3)C≡CH |
| 3017 | Et | Et | H | Indan-1-yl |
| 3018 | Et | Et | H | Tetrahydrofuran-2-ylmethyl |
| 3019 | Et | Et | Morpholin-4-yl | |
| 3020 | Et | Et | 2-(Methoxycarbonyl)piperidin-1-yl | |
| 3021 | Et | Et | 2-(Ethoxycarbonyl)piperidin-1-yl | |
| 3022 | Et | Et | Thiazolidin-3-yl | |
| 3023 | Et | Et | Pyrrolidin-1-yl | |
| 3024 | Et | Et | 2-Methylpyrrolidin-1-yl | |
| 3025 | Et | Et | ═CH—N(CH3)2 | |
| 3026 | Et | Et | ═C(CH3)N(CH3)2 | |
| 3027 | Et | Et | ═CH—N(C2H5)2 | |
| 3028 | Et | Et | ═C(CH3)N(C2H5)2 | |
| 3029 | Et | Et | ═CH-Piperidine | |
| 3030 | Et | Et | ═CH-Morpholine | |
| 3031 | Et | Et | ═CH-Pyrrolidine | |
| 3032 | Et | Pr | Me | Me |
| 3033 | Et | Pr | Me | Et |
| 3034 | Et | Pr | Et | Me |
| 3035 | Et | Pr | Et | Et |
| 3036 | Et | Pr | Me | Ph |
| 3037 | Et | Pr | Me | CH2CO2Me |
| 3038 | Et | Pr | H | Me |
| 3039 | Et | Pr | H | Et |
| 3040 | Et | Pr | H | Pr |
| 3041 | Et | Pr | H | iPr |
| 3042 | Et | Pr | H | cPr |
| 3043 | Et | Pr | H | Bu |
| 3044 | Et | Pr | H | cBu |
| 3045 | Et | Pr | H | tBu |
| 3046 | Et | Pr | H | cPentyl |
| 3047 | Et | Pr | H | cHexyl |
| 3048 | Et | Pr | H | CH2(CH2)3CH3 |
| 3049 | Et | Pr | H | CH2(CH2)4CH3 |
| 3050 | Et | Pr | H | CH2—cPr |
| 3051 | Et | Pr | H | CH2—CN |
| 3052 | Et | Pr | H | CH2—C(CH3)3 |
| 3053 | Et | Pr | H | CH2CF2CF3 |
| 3054 | Et | Pr | H | CH2CF3 |
| 3055 | Et | Pr | H | CH2(CF2)2CF3 |
| 3056 | Et | Pr | H | CH2CH(CH3)CH2CH3 |
| 3057 | Et | Pr | H | CH2C(CH3)2CH2F |
| 3058 | Et | Pr | H | CH2CH(CH3)2 |
| 3059 | Et | Pr | H | CH2CH(CH2CH3)2 |
| 3060 | Et | Pr | H | CH2CH2CH(CH3)2 |
| 3061 | Et | Pr | H | CH2CH2C(CH3)3 |
| 3062 | Et | Pr | H | CH2CH═CH2 |
| 3063 | Et | Pr | H | CH2CH═CHCH3 |
| 3064 | Et | Pr | H | CH2—C(CH3)═CH2 |
| 3065 | Et | Pr | H | CH2—C≡CH |
| 3066 | Et | Pr | H | CH(CH3)CH2CH3 |
| 3067 | Et | Pr | H | CH(CH3)cPr |
| 3068 | Et | Pr | H | CH(CH3)CF3 |
| 3069 | Et | Pr | H | CH(CH3)(CH2)2CH3 |
| 3070 | Et | Pr | H | CH(CH3)(CH2)4CH3 |
| 3071 | Et | Pr | H | CH(CH3)(CH2)5CH3 |
| 3072 | Et | Pr | H | CH(CH2CH3)(CH2)3CH3 |
| 3073 | Et | Pr | H | CH(CH3)CH2CH(CH3)2 |
| 3074 | Et | Pr | H | CH(CH3)C(CH3)3 |
| 3075 | Et | Pr | H | CH(CH3)CH(CH3)2 |
| 3076 | Et | Pr | H | CH(CH3)CH2CH2CH(CH3)2 |
| 3077 | Et | Pr | H | CH(CH2CH3)2 |
| 3078 | Et | Pr | H | C(CH3)2CH2CH3 |
| 3079 | Et | Pr | H | C(CH3)2CH2C(CH3)3 |
| 3080 | Et | Pr | H | CH2—CH(OMe)2 |
| 3081 | Et | Pr | H | CH2—CH(OEt)2 |
| 3082 | Et | Pr | H | CH2CH2—OH |
| 3083 | Et | Pr | H | CH2CH2—OMe |
| 3084 | Et | Pr | H | CH2CH2—OEt |
| 3085 | Et | Pr | H | CH2CH2—SMe |
| 3086 | Et | Pr | H | CH2CH2—CN |
| 3087 | Et | Pr | H | CH2CH2—NMe2 |
| 3088 | Et | Pr | H | CH2CH2-Morpholin-4-yl |
| 3089 | Et | Pr | H | CH(CH3)CH2—OMe |
| 3090 | Et | Pr | H | CH(CH3)CH2—NMe2 |
| 3091 | Et | Pr | H | CH2CH2CH2—OMe |
| 3092 | Et | Pr | H | CH2CH2CH2—SMe |
| 3093 | Et | Pr | H | CH2CH2CH2—OEt |
| 3094 | Et | Pr | H | CH2CH2CH2—OiPr |
| 3095 | Et | Pr | H | CH2CH2CH2—OBu |
| 3096 | Et | Pr | H | CH2—COOMe |
| 3097 | Et | Pr | H | CH2—COOH |
| 3098 | Et | Pr | H | CH(CH3)COOMe |
| 3099 | Et | Pr | H | CH(CH3)COOEt |
| 3100 | Et | Pr | H | CH2CH2—COOMe |
| 3101 | Et | Pr | H | CH(COOMe)2 |
| 3102 | Et | Pr | H | CH(COOEt)CH2—CH(CH3)2 |
| 3103 | Et | Pr | H | CH(COOMe)CH(CH3)2 |
| 3104 | Et | Pr | H | COCH2OMe |
| 3105 | Et | Pr | H | CH(cPr)2 |
| 3106 | Et | Pr | H | Ph |
| 3107 | Et | Pr | H | CH2PH |
| 3108 | Et | Pr | H | CH2-Pyridin-3-yl |
| 3109 | Et | Pr | H | CH2—6-Cl-Pyridin-3-yl |
| 3110 | Et | Pr | H | CH(CH3)Ph |
| 3111 | Et | Pr | H | CH2CH2—Ph |
| 3112 | Et | Pr | H | CH2—2-CF3—Ph |
| 3113 | Et | Pr | H | CH2CH2CHPh2 |
| 3114 | Et | Pr | H | O—CH2CH3 |
| 3115 | Et | Pr | H | O—CH3 |
| 3116 | Et | Pr | H | O—CH2CH═CH2 |
| 3117 | Et | Pr | H | O—tBu |
| 3118 | Et | Pr | H | O—Pr |
| 3119 | Et | Pr | H | O—CH2cPr |
| 3120 | Et | Pr | H | O—CH2CH(CH3)2 |
| 3121 | Et | Pr | H | O—CH2CF3 |
| 3122 | Et | Pr | H | O—CH(CH3)cPr |
| 3123 | Et | Pr | H | O—CH2CH2Cl |
| 3124 | Et | Pr | H | O—CH2C≡CH |
| 3125 | Et | Pr | H | O—CH2C≡CCH3 |
| 3126 | Et | Pr | H | O—CH(CH3)C≡CH |
| 3127 | Et | Pr | H | Indan-1-yl |
| 3128 | Et | Pr | H | Tetrahydrofuran-2-ylmethyl |
| 3129 | Et | Pr | Morpholin-4-yl | |
| 3130 | Et | Pr | 2-(Methoxycarbonyl)piperidin-1-yl | |
| 3131 | Et | Pr | 2-(Ethoxycarbonyl)piperidin-1-yl | |
| 3132 | Et | Pr | Thiazolidin-3-yl | |
| 3133 | Et | Pr | Pyrrolidin-1-yl | |
| 3134 | Et | Pr | 2-Methylpyrrolidin-1-yl | |
| 3135 | Et | Pr | ═CH—N(CH3)2 | |
| 3136 | Et | Pr | ═C(CH3)N(CH3)2 | |
| 3137 | Et | Pr | ═CH—N(C2H5)2 | |
| 3138 | Et | Pr | ═C(CH3)N(C2H5)2 | |
| 3139 | Et | Pr | ═CH-Piperidine | |
| 3140 | Et | Pr | ═CH-Morpholine | |
| 3141 | Et | Pr | ═CH-Pyrrolidine | |
| 3142 | Et | Cl | Me | Me |
| 3143 | Et | Cl | Me | Et |
| 3144 | Et | Cl | Et | Me |
| 3145 | Et | Cl | Et | Et |
| 3146 | Et | Cl | Me | Ph |
| 3147 | Et | Cl | Me | CH2CO2Me |
| 3148 | Et | Cl | H | Me |
| 3149 | Et | Cl | H | Et |
| 3150 | Et | Cl | H | Pr |
| 3151 | Et | Cl | H | iPr |
| 3152 | Et | Cl | H | cPr |
| 3153 | Et | Cl | H | Bu |
| 3154 | Et | Cl | H | cBu |
| 3155 | Et | Cl | H | tBu |
| 3156 | Et | Cl | H | cPentyl |
| 3157 | Et | Cl | H | cHexyl |
| 3158 | Et | Cl | H | CH2(CH2)3CH3 |
| 3159 | Et | Cl | H | CH2(CH2)4CH3 |
| 3160 | Et | Cl | H | CH2—cPr |
| 3161 | Et | Cl | H | CH2—CN |
| 3162 | Et | Cl | H | CH2—C(CH3)3 |
| 3163 | Et | Cl | H | CH2CF2CF3 |
| 3164 | Et | Cl | H | CH2CF3 |
| 3165 | Et | Cl | H | CH2(CF2)2CF3 |
| 3166 | Et | Cl | H | CH2CH(CH3)CH2CH3 |
| 3167 | Et | Cl | H | CH2C(CH3)2CH2F |
| 3168 | Et | Cl | H | CH2CH(CH3)2 |
| 3169 | Et | Cl | H | CH2CH(CH2CH3)2 |
| 3170 | Et | Cl | H | CH2CH2CH(CH3)2 |
| 3171 | Et | Cl | H | CH2CH2C(CH3)3 |
| 3172 | Et | Cl | H | CH2CH═CH2 |
| 3173 | Et | Cl | H | CH2CH═CHCH3 |
| 3174 | Et | Cl | H | CH2—C(CH3)═CH2 |
| 3175 | Et | Cl | H | CH2—C≡CH |
| 3176 | Et | Cl | H | CH(CH3)CH2CH3 |
| 3177 | Et | Cl | H | CH(CH3)cPr |
| 3178 | Et | Cl | H | CH(CH3)CF3 |
| 3179 | Et | Cl | H | CH(CH3)(CH2)2CH3 |
| 3180 | Et | Cl | H | CH(CH3)(CH2)4CH3 |
| 3181 | Et | Cl | H | CH(CH3)(CH2)5CH3 |
| 3182 | Et | Cl | H | CH(CH2CH3)(CH2)3CH3 |
| 3183 | Et | Cl | H | CH(CH3)CH2CH(CH3)2 |
| 3184 | Et | Cl | H | CH(CH3)C(CH3)3 |
| 3185 | Et | Cl | H | CH(CH3)CH(CH3)2 |
| 3186 | Et | Cl | H | CH(CH3)CH2CH2CH(CH3)2 |
| 3187 | Et | Cl | H | CH(CH2CH3)2 |
| 3188 | Et | Cl | H | C(CH3)2CH2CH3 |
| 3189 | Et | Cl | H | C(CH3)2CH2C(CH3)3 |
| 3190 | Et | Cl | H | CH2—CH(OMe)2 |
| 3191 | Et | Cl | H | CH2—CH(OEt)2 |
| 3192 | Et | Cl | H | CH2CH2—OH |
| 3193 | Et | Cl | H | CH2CH2—OMe |
| 3194 | Et | Cl | H | CH2CH2—OEt |
| 3195 | Et | Cl | H | CH2CH2—SMe |
| 3196 | Et | Cl | H | CH2CH2—CN |
| 3197 | Et | Cl | H | CH2CH2—NMe2 |
| 3198 | Et | Cl | H | CH2CH2-Morpholin-4-yl |
| 3199 | Et | Cl | H | CH(CH3)CH2—OMe |
| 3200 | Et | Cl | H | CH(CH3)CH2—NMe2 |
| 3201 | Et | Cl | H | CH2CH2CH2—OMe |
| 3202 | Et | Cl | H | CH2CH2CH2—SMe |
| 3203 | Et | Cl | H | CH2CH2CH2—OEt |
| 3204 | Et | Cl | H | CH2CH2CH2—OiPr |
| 3205 | Et | Cl | H | CH2CH2CH2—OBu |
| 3206 | Et | Cl | H | CH2—COOMe |
| 3207 | Et | Cl | H | CH2—COOH |
| 3208 | Et | Cl | H | CH(CH3)COOMe |
| 3209 | Et | Cl | H | CH(CH3)COOEt |
| 3210 | Et | Cl | H | CH2CH2—COOMe |
| 3211 | Et | Cl | H | CH(COOMe)2 |
| 3212 | Et | Cl | H | CH(COOEt)CH2—CH(CH3)2 |
| 3213 | Et | Cl | H | CH(COOMe)CH(CH3)2 |
| 3214 | Et | Cl | H | COCH2OMe |
| 3215 | Et | Cl | H | CH(cPr)2 |
| 3216 | Et | Cl | H | Ph |
| 3217 | Et | Cl | H | CH2PH |
| 3218 | Et | Cl | H | CH2-Pyridin-3-yl |
| 3219 | Et | Cl | H | CH2—6-Cl-Pyridin-3-yl |
| 3220 | Et | Cl | H | CH(CH3)Ph |
| 3221 | Et | Cl | H | CH2CH2—Ph |
| 3222 | Et | Cl | H | CH2—2-CF3—Ph |
| 3223 | Et | Cl | H | CH2CH2CHPh2 |
| 3224 | Et | Cl | H | O—CH2CH3 |
| 3225 | Et | Cl | H | O—CH3 |
| 3226 | Et | Cl | H | O—CH2CH═CH2 |
| 3227 | Et | Cl | H | O—tBu |
| 3228 | Et | Cl | H | O—Pr |
| 3229 | Et | Cl | H | O—CH2cPr |
| 3230 | Et | Cl | H | O—CH2CH(CH3)2 |
| 3231 | Et | Cl | H | O—CH2CF3 |
| 3232 | Et | Cl | H | O—CH(CH3)cPr |
| 3233 | Et | Cl | H | O—CH2CH2Cl |
| 3234 | Et | Cl | H | O—CH2C≡CH |
| 3235 | Et | Cl | H | O—CH2C≡CCH3 |
| 3236 | Et | Cl | H | O—CH(CH3)C≡CH |
| 3237 | Et | Cl | H | Indan-1-yl |
| 3238 | Et | Cl | H | Tetrahydrofuran-2-ylmethyl |
| 3239 | Et | Cl | Morpholin-4-yl | |
| 3240 | Et | Cl | 2-(Methoxycarbonyl)piperidin-1-yl | |
| 3241 | Et | Cl | 2-(Ethoxycarbonyl)piperidin-1-yl | |
| 3242 | Et | Cl | Thiazolidin-3-yl | |
| 3243 | Et | Cl | Pyrrolidin-1-yl | |
| 3244 | Et | Cl | 2-Methylpyrrolidin-1-yl | |
| 3245 | Et | Cl | ═CH—N(CH3)2 | |
| 3246 | Et | Cl | ═C(CH3)N(CH3)2 | |
| 3247 | Et | Cl | ═CH—N(C2H5)2 | |
| 3248 | Et | Cl | ═C(CH3)N(C2H5)2 | |
| 3249 | Et | Cl | ═CH-Piperidine | |
| 3250 | Et | Cl | ═CH-Morpholine | |
| 3251 | Et | Cl | ═CH-Pyrrolidine | |
| 3252 | Et | Br | Me | Me |
| 3253 | Et | Br | Me | Et |
| 3254 | Et | Br | Et | Me |
| 3255 | Et | Br | Et | Et |
| 3256 | Et | Br | Me | Ph |
| 3257 | Et | Br | Me | CH2CO2Me |
| 3258 | Et | Br | H | Me |
| 3259 | Et | Br | H | Et |
| 3260 | Et | Br | H | Pr |
| 3261 | Et | Br | H | iPr |
| 3262 | Et | Br | H | cPr |
| 3263 | Et | Br | H | Bu |
| 3264 | Et | Br | H | cBu |
| 3265 | Et | Br | H | tBu |
| 3266 | Et | Br | H | cPentyl |
| 3267 | Et | Br | H | cHexyl |
| 3268 | Et | Br | H | CH2(CH2)3CH3 |
| 3269 | Et | Br | H | CH2(CH2)4CH3 |
| 3270 | Et | Br | H | CH2—cPr |
| 3271 | Et | Br | H | CH2—CN |
| 3272 | Et | Br | H | CH2—C(CH3)3 |
| 3273 | Et | Br | H | CH2CF2CF3 |
| 3274 | Et | Br | H | CH2CF3 |
| 3275 | Et | Br | H | CH2(CF2)2CF3 |
| 3276 | Et | Br | H | CH2CH(CH3)CH2CH3 |
| 3277 | Et | Br | H | CH2C(CH3)2CH2F |
| 3278 | Et | Br | H | CH2CH(CH3)2 |
| 3279 | Et | Br | H | CH2CH(CH2CH3)2 |
| 3280 | Et | Br | H | CH2CH2CH(CH3)2 |
| 3281 | Et | Br | H | CH2CH2C(CH3)3 |
| 3282 | Et | Br | H | CH2CH═CH2 |
| 3283 | Et | Br | H | CH2CH═CHCH3 |
| 3284 | Et | Br | H | CH2—C(CH3)═CH2 |
| 3285 | Et | Br | H | CH2—C≡CH |
| 3286 | Et | Br | H | CH(CH3)CH2CH3 |
| 3287 | Et | Br | H | CH(CH3)cPr |
| 3288 | Et | Br | H | CH(CH3)CF3 |
| 3289 | Et | Br | H | CH(CH3)(CH2)2CH3 |
| 3290 | Et | Br | H | CH(CH3)(CH2)4CH3 |
| 3291 | Et | Br | H | CH(CH3)(CH2)5CH3 |
| 3292 | Et | Br | H | CH(CH2CH3)(CH2)3CH3 |
| 3293 | Et | Br | H | CH(CH3)CH2CH(CH3)2 |
| 3294 | Et | Br | H | CH(CH3)C(CH3)3 |
| 3295 | Et | Br | H | CH(CH3)CH(CH3)2 |
| 3296 | Et | Br | H | CH(CH3)CH2CH2CH(CH3)2 |
| 3297 | Et | Br | H | CH(CH2CH3)2 |
| 3298 | Et | Br | H | C(CH3)2CH2CH3 |
| 3299 | Et | Br | H | C(CH3)2CH2C(CH3)3 |
| 3300 | Et | Br | H | CH2—CH(OMe)2 |
| 3301 | Et | Br | H | CH2—CH(OEt)2 |
| 3302 | Et | Br | H | CH2CH2—OH |
| 3303 | Et | Br | H | CH2CH2—OMe |
| 3304 | Et | Br | H | CH2CH2—OEt |
| 3305 | Et | Br | H | CH2CH2—SMe |
| 3306 | Et | Br | H | CH2CH2—CN |
| 3307 | Et | Br | H | CH2CH2—NMe2 |
| 3308 | Et | Br | H | CH2CH2-Morpholin-4-yl |
| 3309 | Et | Br | H | CH(CH3)CH2—OMe |
| 3310 | Et | Br | H | CH(CH3)CH2—NMe2 |
| 3311 | Et | Br | H | CH2CH2CH2—OMe |
| 3312 | Et | Br | H | CH2CH2CH2—SMe |
| 3313 | Et | Br | H | CH2CH2CH2—OEt |
| 3314 | Et | Br | H | CH2CH2CH2—OiPr |
| 3315 | Et | Br | H | CH2CH2CH2—OBu |
| 3316 | Et | Br | H | CH2—COOMe |
| 3317 | Et | Br | H | CH2—COOH |
| 3318 | Et | Br | H | CH(CH3)COOMe |
| 3319 | Et | Br | H | CH(CH3)COOEt |
| 3320 | Et | Br | H | CH2CH2—COOMe |
| 3321 | Et | Br | H | CH(COOMe)2 |
| 3322 | Et | Br | H | CH(COOEt)CH2—CH(CH3)2 |
| 3323 | Et | Br | H | CH(COOMe)CH(CH3)2 |
| 3324 | Et | Br | H | COCH2OMe |
| 3325 | Et | Br | H | CH(cPr)2 |
| 3326 | Et | Br | H | Ph |
| 3327 | Et | Br | H | CH2PH |
| 3328 | Et | Br | H | CH2-Pyridin-3-yl |
| 3329 | Et | Br | H | CH2—6-Cl-Pyridin-3-yl |
| 3330 | Et | Br | H | CH(CH3)Ph |
| 3331 | Et | Br | H | CH2CH2—Ph |
| 3332 | Et | Br | H | CH2—2-CF3—Ph |
| 3333 | Et | Br | H | CH2CH2CHPh2 |
| 3334 | Et | Br | H | O—CH2CH3 |
| 3335 | Et | Br | H | O—CH3 |
| 3336 | Et | Br | H | O—CH2CH═CH2 |
| 3337 | Et | Br | H | O—tBu |
| 3338 | Et | Br | H | O—Pr |
| 3339 | Et | Br | H | O—CH2cPr |
| 3340 | Et | Br | H | O—CH2CH(CH3)2 |
| 3341 | Et | Br | H | O—CH2CF3 |
| 3342 | Et | Br | H | O—CH(CH3)cPr |
| 3343 | Et | Br | H | O—CH2CH2Cl |
| 3344 | Et | Br | H | O—CH2C≡CH |
| 3345 | Et | Br | H | O—CH2C≡CCH3 |
| 3346 | Et | Br | H | O—CH(CH3)C≡CH |
| 3347 | Et | Br | H | Indan-1-yl |
| 3348 | Et | Br | H | Tetrahydrofuran-2-ylmethyl |
| 3349 | Et | Br | Morpholin-4-yl | |
| 3350 | Et | Br | 2-(Methoxycarbonyl)piperidin-1-yl | |
| 3351 | Et | Br | 2-(Ethoxycarbonyl)piperidin-1-yl | |
| 3352 | Et | Br | Thiazolidin-3-yl | |
| 3353 | Et | Br | Pyrrolidin-1-yl | |
| 3354 | Et | Br | 2-Methylpyrrolidin-1-yl | |
| 3355 | Et | Br | ═CH—N(CH3)2 | |
| 3356 | Et | Br | ═C(CH3)N(CH3)2 | |
| 3357 | Et | Br | ═CH—N(C2H5)2 | |
| 3358 | Et | Br | ═C(CH3)N(C2H5)2 | |
| 3359 | Et | Br | ═CH-Piperidine | |
| 3360 | Et | Br | ═CH-Morpholine | |
| 3361 | Et | Br | ═CH-Pyrrolidine | |
| 3362 | Et | I | Me | Me |
| 3363 | Et | I | Me | Et |
| 3364 | Et | I | Et | Me |
| 3365 | Et | I | Et | Et |
| 3366 | Et | I | Me | Ph |
| 3367 | Et | I | Me | CH2CO2Me |
| 3368 | Et | I | H | Me |
| 3369 | Et | I | H | Et |
| 3370 | Et | I | H | Pr |
| 3371 | Et | I | H | iPr |
| 3372 | Et | I | H | cPr |
| 3373 | Et | I | H | Bu |
| 3374 | Et | I | H | cBu |
| 3375 | Et | I | H | tBu |
| 3376 | Et | I | H | cPentyl |
| 3377 | Et | I | H | cHexyl |
| 3378 | Et | I | H | CH2(CH2)3CH3 |
| 3379 | Et | I | H | CH2(CH2)4CH3 |
| 3380 | Et | I | H | CH2—cPr |
| 3381 | Et | I | H | CH2—CN |
| 3382 | Et | I | H | CH2—C(CH3)3 |
| 3383 | Et | I | H | CH2CF2CF3 |
| 3384 | Et | I | H | CH2CF3 |
| 3385 | Et | I | H | CH2(CF2)2CF3 |
| 3386 | Et | I | H | CH2CH(CH3)CH2CH3 |
| 3387 | Et | I | H | CH2C(CH3)2CH2F |
| 3388 | Et | I | H | CH2CH(CH3)2 |
| 3389 | Et | I | H | CH2CH(CH2CH3)2 |
| 3390 | Et | I | H | CH2CH2CH(CH3)2 |
| 3391 | Et | I | H | CH2CH2C(CH3)3 |
| 3392 | Et | I | H | CH2CH═CH2 |
| 3393 | Et | I | H | CH2CH═CHCH3 |
| 3394 | Et | I | H | CH2—C(CH3)═CH2 |
| 3395 | Et | I | H | CH2—C≡CH |
| 3396 | Et | I | H | CH(CH3)CH2CH3 |
| 3397 | Et | I | H | CH(CH3)cPr |
| 3398 | Et | I | H | CH(CH3)CF3 |
| 3399 | Et | I | H | CH(CH3)(CH2)2CH3 |
| 3400 | Et | I | H | CH(CH3)(CH2)4CH3 |
| 3401 | Et | I | H | CH(CH3)(CH2)5CH3 |
| 3402 | Et | I | H | CH(CH2CH3)(CH2)3CH3 |
| 3403 | Et | I | H | CH(CH3)CH2CH(CH3)2 |
| 3404 | Et | I | H | CH(CH3)C(CH3)3 |
| 3405 | Et | I | H | CH(CH3)CH(CH3)2 |
| 3406 | Et | I | H | CH(CH3)CH2CH2CH(CH3)2 |
| 3407 | Et | I | H | CH(CH2CH3)2 |
| 3408 | Et | I | H | C(CH3)2CH2CH3 |
| 3409 | Et | I | H | C(CH3)2CH2C(CH3)3 |
| 3410 | Et | I | H | CH2—CH(OMe)2 |
| 3411 | Et | I | H | CH2—CH(OEt)2 |
| 3412 | Et | I | H | CH2CH2—OH |
| 3413 | Et | I | H | CH2CH2—OMe |
| 3414 | Et | I | H | CH2CH2—OEt |
| 3415 | Et | I | H | CH2CH2—SMe |
| 3416 | Et | I | H | CH2CH2—CN |
| 3417 | Et | I | H | CH2CH2—NMe2 |
| 3418 | Et | I | H | CH2CH2-Morpholin-4-yl |
| 3419 | Et | I | H | CH(CH3)CH2—OMe |
| 3420 | Et | I | H | CH(CH3)CH2—NMe2 |
| 3421 | Et | I | H | CH2CH2CH2—OMe |
| 3422 | Et | I | H | CH2CH2CH2—SMe |
| 3423 | Et | I | H | CH2CH2CH2—OEt |
| 3424 | Et | I | H | CH2CH2CH2—OiPr |
| 3425 | Et | I | H | CH2CH2CH2—OBu |
| 3426 | Et | I | H | CH2—COOMe |
| 3427 | Et | I | H | CH2—COOH |
| 3428 | Et | I | H | CH(CH3)COOMe |
| 3429 | Et | I | H | CH(CH3)COOEt |
| 3430 | Et | I | H | CH2CH2—COOMe |
| 3431 | Et | I | H | CH(COOMe)2 |
| 3432 | Et | I | H | CH(COOEt)CH2—CH(CH3)2 |
| 3433 | Et | I | H | CH(COOMe)CH(CH3)2 |
| 3434 | Et | I | H | COCH2OMe |
| 3435 | Et | I | H | CH(cPr)2 |
| 3436 | Et | I | H | Ph |
| 3437 | Et | I | H | CH2PH |
| 3438 | Et | I | H | CH2-Pyridin-3-yl |
| 3439 | Et | I | H | CH2—6-Cl-Pyridin-3-yl |
| 3440 | Et | I | H | CH(CH3)Ph |
| 3441 | Et | I | H | CH2CH2—Ph |
| 3442 | Et | I | H | CH2—2-CF3—Ph |
| 3443 | Et | I | H | CH2CH2CHPh2 |
| 3444 | Et | I | H | O—CH2CH3 |
| 3445 | Et | I | H | O—CH3 |
| 3446 | Et | I | H | O—CH2CH═CH2 |
| 3447 | Et | I | H | O—tBu |
| 3448 | Et | I | H | O—Pr |
| 3449 | Et | I | H | O—CH2cPr |
| 3450 | Et | I | H | O—CH2CH(CH3)2 |
| 3451 | Et | I | H | O—CH2CF3 |
| 3452 | Et | I | H | O—CH(CH3)cPr |
| 3453 | Et | I | H | O—CH2CH2Cl |
| 3454 | Et | I | H | O—CH2C≡CH |
| 3455 | Et | I | H | O—CH2C≡CCH3 |
| 3456 | Et | I | H | O—CH(CH3)C≡CH |
| 3457 | Et | I | H | Indan-1-yl |
| 3458 | Et | I | H | Tetrahydrofuran-2-ylmethyl |
| 3459 | Et | I | Morpholin-4-yl | |
| 3460 | Et | I | 2-(Methoxycarbonyl)piperidin-1-yl | |
| 3461 | Et | I | 2-(Ethoxycarbonyl)piperidin-1-yl | |
| 3462 | Et | I | Thiazolidin-3-yl | |
| 3463 | Et | I | Pyrrolidin-1-yl | |
| 3464 | Et | I | 2-Methylpyrrolidin-1-yl | |
| 3465 | Et | I | ═CH—N(CH3)2 | |
| 3466 | Et | I | ═C(CH3)N(CH3)2 | |
| 3467 | Et | I | ═CH—N(C2H5)2 | |
| 3468 | Et | I | ═C(CH3)N(C2H5)2 | |
| 3469 | Et | I | ═CH-Piperidine | |
| 3470 | Et | I | ═CH-Morpholine | |
| 3471 | Et | I | ═CH-Pyrrolidine | |
| 3472 | Pr | Me | Me | Me |
| 3473 | Pr | Me | Me | Et |
| 3474 | Pr | Me | Et | Me |
| 3475 | Pr | Me | Et | Et |
| 3476 | Pr | Me | Me | Ph |
| 3477 | Pr | Me | Me | CH2CO2Me |
| 3478 | Pr | Me | H | Me |
| 3479 | Pr | Me | H | Et |
| 3480 | Pr | Me | H | Pr |
| 3481 | Pr | Me | H | iPr |
| 3482 | Pr | Me | H | cPr |
| 3483 | Pr | Me | H | Bu |
| 3484 | Pr | Me | H | cBu |
| 3485 | Pr | Me | H | tBu |
| 3486 | Pr | Me | H | cPentyl |
| 3487 | Pr | Me | H | cHexyl |
| 3488 | Pr | Me | H | CH2(CH2)3CH3 |
| 3489 | Pr | Me | H | CH2(CH2)4CH3 |
| 3490 | Pr | Me | H | CH2—cPr |
| 3491 | Pr | Me | H | CH2—CN |
| 3492 | Pr | Me | H | CH2—C(CH3)3 |
| 3493 | Pr | Me | H | CH2CF2CF3 |
| 3494 | Pr | Me | H | CH2CF3 |
| 3495 | Pr | Me | H | CH2(CF2)2CF3 |
| 3496 | Pr | Me | H | CH2CH(CH3)CH2CH3 |
| 3497 | Pr | Me | H | CH2C(CH3)2CH2F |
| 3498 | Pr | Me | H | CH2CH(CH3)2 |
| 3499 | Pr | Me | H | CH2CH(CH2CH3)2 |
| 3500 | Pr | Me | H | CH2CH2CH(CH3)2 |
| 3501 | Pr | Me | H | CH2CH2C(CH3)3 |
| 3502 | Pr | Me | H | CH2CH═CH2 |
| 3503 | Pr | Me | H | CH2CH═CHCH3 |
| 3504 | Pr | Me | H | CH2—C(CH3)═CH2 |
| 3505 | Pr | Me | H | CH2—C≡CH |
| 3506 | Pr | Me | H | CH(CH3)CH2CH3 |
| 3507 | Pr | Me | H | CH(CH3)cPr |
| 3508 | Pr | Me | H | CH(CH3)CF3 |
| 3509 | Pr | Me | H | CH(CH3)(CH2)2CH3 |
| 3510 | Pr | Me | H | CH(CH3)(CH2)4CH3 |
| 3511 | Pr | Me | H | CH(CH3)(CH2)5CH3 |
| 3512 | Pr | Me | H | CH(CH2CH3)(CH2)3CH3 |
| 3513 | Pr | Me | H | CH(CH3)CH2CH(CH3)2 |
| 3514 | Pr | Me | H | CH(CH3)C(CH3)3 |
| 3515 | Pr | Me | H | CH(CH3)CH(CH3)2 |
| 3516 | Pr | Me | H | CH(CH3)CH2CH2CH(CH3)2 |
| 3517 | Pr | Me | H | CH(CH2CH3)2 |
| 3518 | Pr | Me | H | C(CH3)2CH2CH3 |
| 3519 | Pr | Me | H | C(CH3)2CH2C(CH3)3 |
| 3520 | Pr | Me | H | CH2—CH(OMe)2 |
| 3521 | Pr | Me | H | CH2—CH(OEt)2 |
| 3522 | Pr | Me | H | CH2CH2—OH |
| 3523 | Pr | Me | H | CH2CH2—OMe |
| 3524 | Pr | Me | H | CH2CH2—OEt |
| 3525 | Pr | Me | H | CH2CH2—SMe |
| 3526 | Pr | Me | H | CH2CH2—CN |
| 3527 | Pr | Me | H | CH2CH2—NMe2 |
| 3528 | Pr | Me | H | CH2CH2-Morpholin-4-yl |
| 3529 | Pr | Me | H | CH(CH3)CH2—OMe |
| 3530 | Pr | Me | H | CH(CH3)CH2—NMe2 |
| 3531 | Pr | Me | H | CH2CH2CH2—OMe |
| 3532 | Pr | Me | H | CH2CH2CH2—SMe |
| 3533 | Pr | Me | H | CH2CH2CH2—OEt |
| 3534 | Pr | Me | H | CH2CH2CH2—OiPr |
| 3535 | Pr | Me | H | CH2CH2CH2—OBu |
| 3536 | Pr | Me | H | CH2—COOMe |
| 3537 | Pr | Me | H | CH2—COOH |
| 3538 | Pr | Me | H | CH(CH3)COOMe |
| 3539 | Pr | Me | H | CH(CH3)COOEt |
| 3540 | Pr | Me | H | CH2CH2—COOMe |
| 3541 | Pr | Me | H | CH(COOMe)2 |
| 3542 | Pr | Me | H | CH(COOEt)CH2—CH(CH3)2 |
| 3543 | Pr | Me | H | CH(COOMe)CH(CH3)2 |
| 3544 | Pr | Me | H | COCH2OMe |
| 3545 | Pr | Me | H | CH(cPr)2 |
| 3546 | Pr | Me | H | Ph |
| 3547 | Pr | Me | H | CH2PH |
| 3548 | Pr | Me | H | CH2-Pyridin-3-yl |
| 3549 | Pr | Me | H | CH2—6-Cl-Pyridin-3-yl |
| 3550 | Pr | Me | H | CH(CH3)Ph |
| 3551 | Pr | Me | H | CH2CH2—Ph |
| 3552 | Pr | Me | H | CH2—2-CF3—Ph |
| 3553 | Pr | Me | H | CH2CH2CHPh2 |
| 3554 | Pr | Me | H | O—CH2CH3 |
| 3555 | Pr | Me | H | O—CH3 |
| 3556 | Pr | Me | H | O—CH2CH═CH2 |
| 3557 | Pr | Me | H | O—tBu |
| 3558 | Pr | Me | H | O—Pr |
| 3559 | Pr | Me | H | O—CH2cPr |
| 3560 | Pr | Me | H | O—CH2CH(CH3)2 |
| 3561 | Pr | Me | H | O—CH2CF3 |
| 3562 | Pr | Me | H | O—CH(CH3)cPr |
| 3563 | Pr | Me | H | O—CH2CH2Cl |
| 3564 | Pr | Me | H | O—CH2C≡CH |
| 3565 | Pr | Me | H | O—CH2C≡CCH3 |
| 3566 | Pr | Me | H | O—CH(CH3)C≡CH |
| 3567 | Pr | Me | H | Indan-1-yl |
| 3568 | Pr | Me | H | Tetrahydrofuran-2-ylmethyl |
| 3569 | Pr | Me | Morpholin-4-yl | |
| 3570 | Pr | Me | 2-(Methoxycarbonyl)piperidin-1-yl | |
| 3571 | Pr | Me | 2-(Ethoxycarbonyl)piperidin-1-yl | |
| 3572 | Pr | Me | Thiazolidin-3-yl | |
| 3573 | Pr | Me | Pyrrolidin-1-yl | |
| 3574 | Pr | Me | 2-Methylpyrrolidin-1-yl | |
| 3575 | Pr | Me | ═CH—N(CH3)2 | |
| 3576 | Pr | Me | ═C(CH3)N(CH3)2 | |
| 3577 | Pr | Me | ═CH—N(C2H5)2 | |
| 3578 | Pr | Me | ═C(CH3)N(C2H5)2 | |
| 3579 | Pr | Me | ═CH-Piperidine | |
| 3580 | Pr | Me | ═CH-Morpholine | |
| 3581 | Pr | Me | ═CH-Pyrrolidine | |
| 3582 | Pr | Et | Me | Me |
| 3583 | Pr | Et | Me | Et |
| 3584 | Pr | Et | Et | Me |
| 3585 | Pr | Et | Et | Et |
| 3586 | Pr | Et | Me | Ph |
| 3587 | Pr | Et | Me | CH2CO2Me |
| 3588 | Pr | Et | H | Me |
| 3589 | Pr | Et | H | Et |
| 3590 | Pr | Et | H | Pr |
| 3591 | Pr | Et | H | iPr |
| 3592 | Pr | Et | H | cPr |
| 3593 | Pr | Et | H | Bu |
| 3594 | Pr | Et | H | cBu |
| 3595 | Pr | Et | H | tBu |
| 3596 | Pr | Et | H | cPentyl |
| 3597 | Pr | Et | H | cHexyl |
| 3598 | Pr | Et | H | CH2(CH2)3CH3 |
| 3599 | Pr | Et | H | CH2(CH2)4CH3 |
| 3600 | Pr | Et | H | CH2—cPr |
| 3601 | Pr | Et | H | CH2—CN |
| 3602 | Pr | Et | H | CH2—C(CH3)3 |
| 3603 | Pr | Et | H | CH2CF2CF3 |
| 3604 | Pr | Et | H | CH2CF3 |
| 3605 | Pr | Et | H | CH2(CF2)2CF3 |
| 3606 | Pr | Et | H | CH2CH(CH3)CH2CH3 |
| 3607 | Pr | Et | H | CH2C(CH3)2CH2F |
| 3608 | Pr | Et | H | CH2CH(CH3)2 |
| 3609 | Pr | Et | H | CH2CH(CH2CH3)2 |
| 3610 | Pr | Et | H | CH2CH2CH(CH3)2 |
| 3611 | Pr | Et | H | CH2CH2C(CH3)3 |
| 3612 | Pr | Et | H | CH2CH═CH2 |
| 3613 | Pr | Et | H | CH2CH═CHCH3 |
| 3614 | Pr | Et | H | CH2—C(CH3)═CH2 |
| 3615 | Pr | Et | H | CH2—C≡CH |
| 3616 | Pr | Et | H | CH(CH3)CH2CH3 |
| 3617 | Pr | Et | H | CH(CH3)cPr |
| 3618 | Pr | Et | H | CH(CH3)CF3 |
| 3619 | Pr | Et | H | CH(CH3)(CH2)2CH3 |
| 3620 | Pr | Et | H | CH(CH3)(CH2)4CH3 |
| 3621 | Pr | Et | H | CH(CH3)(CH2)5CH3 |
| 3622 | Pr | Et | H | CH(CH2CH3)(CH2)3CH3 |
| 3623 | Pr | Et | H | CH(CH3)CH2CH(CH3)2 |
| 3624 | Pr | Et | H | CH(CH3)C(CH3)3 |
| 3625 | Pr | Et | H | CH(CH3)CH(CH3)2 |
| 3626 | Pr | Et | H | CH(CH3)CH2CH2CH(CH3)2 |
| 3627 | Pr | Et | H | CH(CH2CH3)2 |
| 3628 | Pr | Et | H | C(CH3)2CH2CH3 |
| 3629 | Pr | Et | H | C(CH3)2CH2C(CH3)3 |
| 3630 | Pr | Et | H | CH2—CH(OMe)2 |
| 3631 | Pr | Et | H | CH2—CH(OEt)2 |
| 3632 | Pr | Et | H | CH2CH2—OH |
| 3633 | Pr | Et | H | CH2CH2—OMe |
| 3634 | Pr | Et | H | CH2CH2—OEt |
| 3635 | Pr | Et | H | CH2CH2—SMe |
| 3636 | Pr | Et | H | CH2CH2—CN |
| 3637 | Pr | Et | H | CH2CH2—NMe2 |
| 3638 | Pr | Et | H | CH2CH2-Morpholin-4-yl |
| 3639 | Pr | Et | H | CH(CH3)CH2—OMe |
| 3640 | Pr | Et | H | CH(CH3)CH2—NMe2 |
| 3641 | Pr | Et | H | CH2CH2CH2—OMe |
| 3642 | Pr | Et | H | CH2CH2CH2—SMe |
| 3643 | Pr | Et | H | CH2CH2CH2—OEt |
| 3644 | Pr | Et | H | CH2CH2CH2—OiPr |
| 3645 | Pr | Et | H | CH2CH2CH2—OBu |
| 3646 | Pr | Et | H | CH2—COOMe |
| 3647 | Pr | Et | H | CH2—COOH |
| 3648 | Pr | Et | H | CH(CH3)COOMe |
| 3649 | Pr | Et | H | CH(CH3)COOEt |
| 3650 | Pr | Et | H | CH2CH2—COOMe |
| 3651 | Pr | Et | H | CH(COOMe)2 |
| 3652 | Pr | Et | H | CH(COOEt)CH2—CH(CH3)2 |
| 3653 | Pr | Et | H | CH(COOMe)CH(CH3)2 |
| 3654 | Pr | Et | H | COCH2OMe |
| 3655 | Pr | Et | H | CH(cPr)2 |
| 3656 | Pr | Et | H | Ph |
| 3657 | Pr | Et | H | CH2PH |
| 3658 | Pr | Et | H | CH2-Pyridin-3-yl |
| 3659 | Pr | Et | H | CH2—6-Cl-Pyridin-3-yl |
| 3660 | Pr | Et | H | CH(CH3)Ph |
| 3661 | Pr | Et | H | CH2CH2—Ph |
| 3662 | Pr | Et | H | CH2—2-CF3—Ph |
| 3663 | Pr | Et | H | CH2CH2CHPh2 |
| 3664 | Pr | Et | H | O—CH2CH3 |
| 3665 | Pr | Et | H | O—CH3 |
| 3666 | Pr | Et | H | O—CH2CH═CH2 |
| 3667 | Pr | Et | H | O—tBu |
| 3668 | Pr | Et | H | O—Pr |
| 3669 | Pr | Et | H | O—CH2cPr |
| 3670 | Pr | Et | H | O—CH2CH(CH3)2 |
| 3671 | Pr | Et | H | O—CH2CF3 |
| 3672 | Pr | Et | H | O—CH(CH3)cPr |
| 3673 | Pr | Et | H | O—CH2CH2Cl |
| 3674 | Pr | Et | H | O—CH2C≡CH |
| 3675 | Pr | Et | H | O—CH2C≡CCH3 |
| 3676 | Pr | Et | H | O—CH(CH3)C≡CH |
| 3677 | Pr | Et | H | Indan-1-yl |
| 3678 | Pr | Et | H | Tetrahydrofuran-2-ylmethyl |
| 3679 | Pr | Et | Morpholin-4-yl | |
| 3680 | Pr | Et | 2-(Methoxycarbonyl)piperidin-1-yl | |
| 3681 | Pr | Et | 2-(Ethoxycarbonyl)piperidin-1-yl | |
| 3682 | Pr | Et | Thiazolidin-3-yl | |
| 3683 | Pr | Et | Pyrrolidin-1-yl | |
| 3684 | Pr | Et | 2-Methylpyrrolidin-1-yl | |
| 3685 | Pr | Et | ═CH—N(CH3)2 | |
| 3686 | Pr | Et | ═C(CH3)N(CH3)2 | |
| 3687 | Pr | Et | ═CH—N(C2H5)2 | |
| 3688 | Pr | Et | ═C(CH3)N(C2H5)2 | |
| 3689 | Pr | Et | ═CH-Piperidine | |
| 3690 | Pr | Et | ═CH-Morpholine | |
| 3691 | Pr | Et | ═CH-Pyrrolidine | |
| 3692 | Pr | Cl | Me | Me |
| 3693 | Pr | Cl | Me | Et |
| 3694 | Pr | Cl | Et | Me |
| 3695 | Pr | Cl | Et | Et |
| 3696 | Pr | Cl | Me | Ph |
| 3697 | Pr | Cl | Me | CH2CO2Me |
| 3698 | Pr | Cl | H | Me |
| 3699 | Pr | Cl | H | Et |
| 3700 | Pr | Cl | H | Pr |
| 3701 | Pr | Cl | H | iPr |
| 3702 | Pr | Cl | H | cPr |
| 3703 | Pr | Cl | H | Bu |
| 3704 | Pr | Cl | H | cBu |
| 3705 | Pr | Cl | H | tBu |
| 3706 | Pr | Cl | H | cPentyl |
| 3707 | Pr | Cl | H | cHexyl |
| 3708 | Pr | Cl | H | CH2(CH2)3CH3 |
| 3709 | Pr | Cl | H | CH2(CH2)4CH3 |
| 3710 | Pr | Cl | H | CH2—cPr |
| 3711 | Pr | Cl | H | CH2—CN |
| 3712 | Pr | Cl | H | CH2—C(CH3)3 |
| 3713 | Pr | Cl | H | CH2CF2CF3 |
| 3714 | Pr | Cl | H | CH2CF3 |
| 3715 | Pr | Cl | H | CH2(CF2)2CF3 |
| 3716 | Pr | Cl | H | CH2CH(CH3)CH2CH3 |
| 3717 | Pr | Cl | H | CH2C(CH3)2CH2F |
| 3718 | Pr | Cl | H | CH2CH(CH3)2 |
| 3719 | Pr | Cl | H | CH2CH(CH2CH3)2 |
| 3720 | Pr | Cl | H | CH2CH2CH(CH3)2 |
| 3721 | Pr | Cl | H | CH2CH2C(CH3)3 |
| 3722 | Pr | Cl | H | CH2CH═CH2 |
| 3723 | Pr | Cl | H | CH2CH═CHCH3 |
| 3724 | Pr | Cl | H | CH2—C(CH3)═CH2 |
| 3725 | Pr | Cl | H | CH2—C≡CH |
| 3726 | Pr | Cl | H | CH(CH3)CH2CH3 |
| 3727 | Pr | Cl | H | CH(CH3)cPr |
| 3728 | Pr | Cl | H | CH(CH3)CF3 |
| 3729 | Pr | Cl | H | CH(CH3)(CH2)2CH3 |
| 3730 | Pr | Cl | H | CH(CH3)(CH2)4CH3 |
| 3731 | Pr | Cl | H | CH(CH3)(CH2)5CH3 |
| 3732 | Pr | Cl | H | CH(CH2CH3)(CH2)3CH3 |
| 3733 | Pr | Cl | H | CH(CH3)CH2CH(CH3)2 |
| 3734 | Pr | Cl | H | CH(CH3)C(CH3)3 |
| 3735 | Pr | Cl | H | CH(CH3)CH(CH3)2 |
| 3736 | Pr | Cl | H | CH(CH3)CH2CH2CH(CH3)2 |
| 3737 | Pr | Cl | H | CH(CH2CH3)2 |
| 3738 | Pr | Cl | H | C(CH3)2CH2CH3 |
| 3739 | Pr | Cl | H | C(CH3)2CH2C(CH3)3 |
| 3740 | Pr | Cl | H | CH2—CH(OMe)2 |
| 3741 | Pr | Cl | H | CH2—CH(OEt)2 |
| 3742 | Pr | Cl | H | CH2CH2—OH |
| 3743 | Pr | Cl | H | CH2CH2—OMe |
| 3744 | Pr | Cl | H | CH2CH2—OEt |
| 3745 | Pr | Cl | H | CH2CH2—SMe |
| 3746 | Pr | Cl | H | CH2CH2—CN |
| 3747 | Pr | Cl | H | CH2CH2—NMe2 |
| 3748 | Pr | Cl | H | CH2CH2-Morpholin-4-yl |
| 3749 | Pr | Cl | H | CH(CH3)CH2—OMe |
| 3750 | Pr | Cl | H | CH(CH3)CH2—NMe2 |
| 3751 | Pr | Cl | H | CH2CH2CH2—OMe |
| 3752 | Pr | Cl | H | CH2CH2CH2—SMe |
| 3753 | Pr | Cl | H | CH2CH2CH2—OEt |
| 3754 | Pr | Cl | H | CH2CH2CH2—OiPr |
| 3755 | Pr | Cl | H | CH2CH2CH2—OBu |
| 3756 | Pr | Cl | H | CH2—COOMe |
| 3757 | Pr | Cl | H | CH2—COOH |
| 3758 | Pr | Cl | H | CH(CH3)COOMe |
| 3759 | Pr | Cl | H | CH(CH3)COOEt |
| 3760 | Pr | Cl | H | CH2CH2—COOMe |
| 3761 | Pr | Cl | H | CH(COOMe)2 |
| 3762 | Pr | Cl | H | CH(COOEt)CH2—CH(CH3)2 |
| 3763 | Pr | Cl | H | CH(COOMe)CH(CH3)2 |
| 3764 | Pr | Cl | H | COCH2OMe |
| 3765 | Pr | Cl | H | CH(cPr)2 |
| 3766 | Pr | Cl | H | Ph |
| 3767 | Pr | Cl | H | CH2PH |
| 3768 | Pr | Cl | H | CH2-Pyridin-3-yl |
| 3769 | Pr | Cl | H | CH2—6-Cl-Pyridin-3-yl |
| 3770 | Pr | Cl | H | CH(CH3)Ph |
| 3771 | Pr | Cl | H | CH2CH2—Ph |
| 3772 | Pr | Cl | H | CH2—2-CF3—Ph |
| 3773 | Pr | Cl | H | CH2CH2CHPh2 |
| 3774 | Pr | Cl | H | O—CH2CH3 |
| 3775 | Pr | Cl | H | O—CH3 |
| 3776 | Pr | Cl | H | O—CH2CH═CH2 |
| 3777 | Pr | Cl | H | O—tBu |
| 3778 | Pr | Cl | H | O—Pr |
| 3779 | Pr | Cl | H | O—CH2cPr |
| 3780 | Pr | Cl | H | O—CH2CH(CH3)2 |
| 3781 | Pr | Cl | H | O—CH2CF3 |
| 3782 | Pr | Cl | H | O—CH(CH3)cPr |
| 3783 | Pr | Cl | H | O—CH2CH2Cl |
| 3784 | Pr | Cl | H | O—CH2C≡CH |
| 3785 | Pr | Cl | H | O—CH2C≡CCH3 |
| 3786 | Pr | Cl | H | O—CH(CH3)C≡CH |
| 3787 | Pr | Cl | H | Indan-1-yl |
| 3788 | Pr | Cl | H | Tetrahydrofuran-2-ylmethyl |
| 3789 | Pr | Cl | Morpholin-4-yl | |
| 3790 | Pr | Cl | 2-(Methoxycarbonyl)piperidin-1-yl | |
| 3791 | Pr | Cl | 2-(Ethoxycarbonyl)piperidin-1-yl | |
| 3792 | Pr | Cl | Thiazolidin-3-yl | |
| 3793 | Pr | Cl | Pyrrolidin-1-yl | |
| 3794 | Pr | Cl | 2-Methylpyrrolidin-1-yl | |
| 3795 | Pr | Cl | ═CH—N(CH3)2 | |
| 3796 | Pr | Cl | ═C(CH3)N(CH3)2 | |
| 3797 | Pr | Cl | ═CH—N(C2H5)2 | |
| 3798 | Pr | Cl | ═C(CH3)N(C2H5)2 | |
| 3799 | Pr | Cl | ═CH-Piperidine | |
| 3800 | Pr | Cl | ═CH-Morpholine | |
| 3801 | Pr | Cl | ═CH-Pyrrolidine | |
| 3802 | Pr | Br | Me | Me |
| 3803 | Pr | Br | Me | Et |
| 3804 | Pr | Br | Et | Me |
| 3805 | Pr | Br | Et | Et |
| 3806 | Pr | Br | Me | Ph |
| 3807 | Pr | Br | Me | CH2CO2Me |
| 3808 | Pr | Br | H | Me |
| 3809 | Pr | Br | H | Et |
| 3810 | Pr | Br | H | Pr |
| 3811 | Pr | Br | H | iPr |
| 3812 | Pr | Br | H | cPr |
| 3813 | Pr | Br | H | Bu |
| 3814 | Pr | Br | H | cBu |
| 3815 | Pr | Br | H | tBu |
| 3816 | Pr | Br | H | cPentyl |
| 3817 | Pr | Br | H | cHexyl |
| 3818 | Pr | Br | H | CH2(CH2)3CH3 |
| 3819 | Pr | Br | H | CH2(CH2)4CH3 |
| 3820 | Pr | Br | H | CH2—cPr |
| 3821 | Pr | Br | H | CH2—CN |
| 3822 | Pr | Br | H | CH2—C(CH3)3 |
| 3823 | Pr | Br | H | CH2CF2CF3 |
| 3824 | Pr | Br | H | CH2CF3 |
| 3825 | Pr | Br | H | CH2(CF2)2CF3 |
| 3826 | Pr | Br | H | CH2CH(CH3)CH2CH3 |
| 3827 | Pr | Br | H | CH2C(CH3)2CH2F |
| 3828 | Pr | Br | H | CH2CH(CH3)2 |
| 3829 | Pr | Br | H | CH2CH(CH2CH3)2 |
| 3830 | Pr | Br | H | CH2CH2CH(CH3)2 |
| 3831 | Pr | Br | H | CH2CH2C(CH3)3 |
| 3832 | Pr | Br | H | CH2CH═CH2 |
| 3833 | Pr | Br | H | CH2CH═CHCH3 |
| 3834 | Pr | Br | H | CH2—C(CH3)═CH2 |
| 3835 | Pr | Br | H | CH2—C≡CH |
| 3836 | Pr | Br | H | CH(CH3)CH2CH3 |
| 3837 | Pr | Br | H | CH(CH3)cPr |
| 3838 | Pr | Br | H | CH(CH3)CF3 |
| 3839 | Pr | Br | H | CH(CH3)(CH2)2CH3 |
| 3840 | Pr | Br | H | CH(CH3)(CH2)4CH3 |
| 3841 | Pr | Br | H | CH(CH3)(CH2)5CH3 |
| 3842 | Pr | Br | H | CH(CH2CH3)(CH2)3CH3 |
| 3843 | Pr | Br | H | CH(CH3)CH2CH(CH3)2 |
| 3844 | Pr | Br | H | CH(CH3)C(CH3)3 |
| 3845 | Pr | Br | H | CH(CH3)CH(CH3)2 |
| 3846 | Pr | Br | H | CH(CH3)CH2CH2CH(CH3)2 |
| 3847 | Pr | Br | H | CH(CH2CH3)2 |
| 3848 | Pr | Br | H | C(CH3)2CH2CH3 |
| 3849 | Pr | Br | H | C(CH3)2CH2C(CH3)3 |
| 3850 | Pr | Br | H | CH2—CH(OMe)2 |
| 3851 | Pr | Br | H | CH2—CH(OEt)2 |
| 3852 | Pr | Br | H | CH2CH2—OH |
| 3853 | Pr | Br | H | CH2CH2—OMe |
| 3854 | Pr | Br | H | CH2CH2—OEt |
| 3855 | Pr | Br | H | CH2CH2—SMe |
| 3856 | Pr | Br | H | CH2CH2—CN |
| 3857 | Pr | Br | H | CH2CH2—NMe2 |
| 3858 | Pr | Br | H | CH2CH2-Morpholin-4-yl |
| 3859 | Pr | Br | H | CH(CH3)CH2—OMe |
| 3860 | Pr | Br | H | CH(CH3)CH2—NMe2 |
| 3861 | Pr | Br | H | CH2CH2CH2—OMe |
| 3862 | Pr | Br | H | CH2CH2CH2—SMe |
| 3863 | Pr | Br | H | CH2CH2CH2—OEt |
| 3864 | Pr | Br | H | CH2CH2CH2—OiPr |
| 3865 | Pr | Br | H | CH2CH2CH2—OBu |
| 3866 | Pr | Br | H | CH2—COOMe |
| 3867 | Pr | Br | H | CH2—COOH |
| 3868 | Pr | Br | H | CH(CH3)COOMe |
| 3869 | Pr | Br | H | CH(CH3)COOEt |
| 3870 | Pr | Br | H | CH2CH2—COOMe |
| 3871 | Pr | Br | H | CH(COOMe)2 |
| 3872 | Pr | Br | H | CH(COOEt)CH2—CH(CH3)2 |
| 3873 | Pr | Br | H | CH(COOMe)CH(CH3)2 |
| 3874 | Pr | Br | H | COCH2OMe |
| 3875 | Pr | Br | H | CH(cPr)2 |
| 3876 | Pr | Br | H | Ph |
| 3877 | Pr | Br | H | CH2PH |
| 3878 | Pr | Br | H | CH2-Pyridin-3-yl |
| 3879 | Pr | Br | H | CH2—6-Cl-Pyridin-3-yl |
| 3880 | Pr | Br | H | CH(CH3)Ph |
| 3881 | Pr | Br | H | CH2CH2—Ph |
| 3882 | Pr | Br | H | CH2—2-CF3—Ph |
| 3883 | Pr | Br | H | CH2CH2CHPh2 |
| 3884 | Pr | Br | H | O—CH2CH3 |
| 3885 | Pr | Br | H | O—CH3 |
| 3886 | Pr | Br | H | O—CH2CH═CH2 |
| 3887 | Pr | Br | H | O—tBu |
| 3888 | Pr | Br | H | O—Pr |
| 3889 | Pr | Br | H | O—CH2cPr |
| 3890 | Pr | Br | H | O—CH2CH(CH3)2 |
| 3891 | Pr | Br | H | O—CH2CF3 |
| 3892 | Pr | Br | H | O—CH(CH3)cPr |
| 3893 | Pr | Br | H | O—CH2CH2Cl |
| 3894 | Pr | Br | H | O—CH2C≡CH |
| 3895 | Pr | Br | H | O—CH2C≡CCH3 |
| 3896 | Pr | Br | H | O—CH(CH3)C≡CH |
| 3897 | Pr | Br | H | Indan-1-yl |
| 3898 | Pr | Br | H | Tetrahydrofuran-2-ylmethyl |
| 3899 | Pr | Br | Morpholin-4-yl | |
| 3900 | Pr | Br | 2-(Methoxycarbonyl)piperidin-1-yl | |
| 3901 | Pr | Br | 2-(Ethoxycarbonyl)piperidin-1-yl | |
| 3902 | Pr | Br | Thiazolidin-3-yl | |
| 3903 | Pr | Br | Pyrrolidin-1-yl | |
| 3904 | Pr | Br | 2-Methylpyrrolidin-1-yl | |
| 3905 | Pr | Br | ═CH—N(CH3)2 | |
| 3906 | Pr | Br | ═C(CH3)N(CH3)2 | |
| 3907 | Pr | Br | ═CH—N(C2H5)2 | |
| 3908 | Pr | Br | ═C(CH3)N(C2H5)2 | |
| 3909 | Pr | Br | ═CH-Piperidine | |
| 3910 | Pr | Br | ═CH-Morpholine | |
| 3911 | Pr | Br | ═CH-Pyrrolidine | |
| 3912 | Pr | I | Me | Me |
| 3913 | Pr | I | Me | Et |
| 3914 | Pr | I | Et | Me |
| 3915 | Pr | I | Et | Et |
| 3916 | Pr | I | Me | Ph |
| 3917 | Pr | I | Me | CH2CO2Me |
| 3918 | Pr | I | H | Me |
| 3919 | Pr | I | H | Et |
| 3920 | Pr | I | H | Pr |
| 3921 | Pr | I | H | iPr |
| 3922 | Pr | I | H | cPr |
| 3923 | Pr | I | H | Bu |
| 3924 | Pr | I | H | cBu |
| 3925 | Pr | I | H | tBu |
| 3926 | Pr | I | H | cPentyl |
| 3927 | Pr | I | H | cHexyl |
| 3928 | Pr | I | H | CH2(CH2)3CH3 |
| 3929 | Pr | I | H | CH2(CH2)4CH3 |
| 3930 | Pr | I | H | CH2—cPr |
| 3931 | Pr | I | H | CH2—CN |
| 3932 | Pr | I | H | CH2—C(CH3)3 |
| 3933 | Pr | I | H | CH2CF2CF3 |
| 3934 | Pr | I | H | CH2CF3 |
| 3935 | Pr | I | H | CH2(CF2)2CF3 |
| 3936 | Pr | I | H | CH2CH(CH3)CH2CH3 |
| 3937 | Pr | I | H | CH2C(CH3)2CH2F |
| 3938 | Pr | I | H | CH2CH(CH3)2 |
| 3939 | Pr | I | H | CH2CH(CH2CH3)2 |
| 3940 | Pr | I | H | CH2CH2CH(CH3)2 |
| 3941 | Pr | I | H | CH2CH2C(CH3)3 |
| 3942 | Pr | I | H | CH2CH═CH2 |
| 3943 | Pr | I | H | CH2CH═CHCH3 |
| 3944 | Pr | I | H | CH2—C(CH3)═CH2 |
| 3945 | Pr | I | H | CH2—C≡CH |
| 3946 | Pr | I | H | CH(CH3)CH2CH3 |
| 3947 | Pr | I | H | CH(CH3)cPr |
| 3948 | Pr | I | H | CH(CH3)CF3 |
| 3949 | Pr | I | H | CH(CH3)(CH2)2CH3 |
| 3950 | Pr | I | H | CH(CH3)(CH2)4CH3 |
| 3951 | Pr | I | H | CH(CH3)(CH2)5CH3 |
| 3952 | Pr | I | H | CH(CH2CH3)(CH2)3CH3 |
| 3953 | Pr | I | H | CH(CH3)CH2CH(CH3)2 |
| 3954 | Pr | I | H | CH(CH3)C(CH3)3 |
| 3955 | Pr | I | H | CH(CH3)CH(CH3)2 |
| 3956 | Pr | I | H | CH(CH3)CH2CH2CH(CH3)2 |
| 3957 | Pr | I | H | CH(CH2CH3)2 |
| 3958 | Pr | I | H | C(CH3)2CH2CH3 |
| 3959 | Pr | I | H | C(CH3)2CH2C(CH3)3 |
| 3960 | Pr | I | H | CH2—CH(OMe)2 |
| 3961 | Pr | I | H | CH2—CH(OEt)2 |
| 3962 | Pr | I | H | CH2CH2—OH |
| 3963 | Pr | I | H | CH2CH2—OMe |
| 3964 | Pr | I | H | CH2CH2—OEt |
| 3965 | Pr | I | H | CH2CH2—SMe |
| 3966 | Pr | I | H | CH2CH2—CN |
| 3967 | Pr | I | H | CH2CH2—NMe2 |
| 3968 | Pr | I | H | CH2CH2-Morpholin-4-yl |
| 3969 | Pr | I | H | CH(CH3)CH2—OMe |
| 3970 | Pr | I | H | CH(CH3)CH2—NMe2 |
| 3971 | Pr | I | H | CH2CH2CH2—OMe |
| 3972 | Pr | I | H | CH2CH2CH2—SMe |
| 3973 | Pr | I | H | CH2CH2CH2—OEt |
| 3974 | Pr | I | H | CH2CH2CH2—OiPr |
| 3975 | Pr | I | H | CH2CH2CH2—OBu |
| 3976 | Pr | I | H | CH2—COOMe |
| 3977 | Pr | I | H | CH2—COOH |
| 3978 | Pr | I | H | CH(CH3)COOMe |
| 3979 | Pr | I | H | CH(CH3)COOEt |
| 3980 | Pr | I | H | CH2CH2—COOMe |
| 3981 | Pr | I | H | CH(COOMe)2 |
| 3982 | Pr | I | H | CH(COOEt)CH2—CH(CH3)2 |
| 3983 | Pr | I | H | CH(COOMe)CH(CH3)2 |
| 3984 | Pr | I | H | COCH2OMe |
| 3985 | Pr | I | H | CH(cPr)2 |
| 3986 | Pr | I | H | Ph |
| 3987 | Pr | I | H | CH2PH |
| 3988 | Pr | I | H | CH2-Pyridin-3-yl |
| 3989 | Pr | I | H | CH2—6-Cl-Pyridin-3-yl |
| 3990 | Pr | I | H | CH(CH3)Ph |
| 3991 | Pr | I | H | CH2CH2—Ph |
| 3992 | Pr | I | H | CH2—2-CF3—Ph |
| 3993 | Pr | I | H | CH2CH2CHPh2 |
| 3994 | Pr | I | H | O—CH2CH3 |
| 3995 | Pr | I | H | O—CH3 |
| 3996 | Pr | I | H | O—CH2CH═CH2 |
| 3997 | Pr | I | H | O—tBu |
| 3998 | Pr | I | H | O—Pr |
| 3999 | Pr | I | H | O—CH2cPr |
| 4000 | Pr | I | H | O—CH2CH(CH3)2 |
| 4001 | Pr | I | H | O—CH2CF3 |
| 4002 | Pr | I | H | O—CH(CH3)cPr |
| 4003 | Pr | I | H | O—CH2CH2Cl |
| 4004 | Pr | I | H | O—CH2C≡CH |
| 4005 | Pr | I | H | O—CH2C≡CCH3 |
| 4006 | Pr | I | H | O—CH(CH3)C≡CH |
| 4007 | Pr | I | H | Indan-1-yl |
| 4008 | Pr | I | H | Tetrahydrofuran-2-ylmethyl |
| 4009 | Pr | I | Morpholin-4-yl | |
| 4010 | Pr | I | 2-(Methoxycarbonyl)piperidin-1-yl | |
| 4011 | Pr | I | 2-(Ethoxycarbonyl)piperidin-1-yl | |
| 4012 | Pr | I | Thiazolidin-3-yl | |
| 4013 | Pr | I | Pyrrolidin-1-yl | |
| 4014 | Pr | I | 2-Methylpyrrolidin-1-yl | |
| 4015 | Pr | I | ═CH—N(CH3)2 | |
| 4016 | Pr | I | ═C(CH3)N(CH3)2 | |
| 4017 | Pr | I | ═CH—N(C2H5)2 | |
| 4018 | Pr | I | ═C(CH3)N(C2H5)2 | |
| 4019 | Pr | I | ═CH-Piperidine | |
| 4020 | Pr | I | ═CH-Morpholine | |
| 4021 | Pr | I | ═CH-Pyrrolidine | |
| 4022 | Me | Me | Me | Me |
| 4023 | Me | Me | Me | Et |
| 4024 | Me | Me | Et | Me |
| 4025 | Me | Me | Et | Et |
| 4026 | Me | Me | Me | Ph |
| 4027 | Me | Me | Me | CH2CO2Me |
| 4028 | Me | Me | Morpholin-4-yl | |
| 4029 | Me | Me | 2-(Methoxycarbonyl)piperidin-1-yl | |
| 4030 | Me | Me | 2-(Ethoxycarbonyl)piperidin-1-yl | |
| 4031 | Me | Me | Thiazolidin-3-yl | |
| 4032 | Me | Me | Pyrrolidin-1-yl | |
| 4033 | Me | Me | 2-Methylpyrrolidin-1-yl | |
| 4034 | Me | Me | ═CH—N(CH3)2 | |
| 4035 | Me | Me | ═C(CH3)N(CH3)2 | |
| 4036 | Me | Me | ═CH—N(C2H5)2 | |
| 4037 | Me | Me | ═C(CH3)N(C2H5)2 | |
| 4038 | Me | Me | ═CH-Piperidine | |
| 4039 | Me | Me | ═CH-Morpholine | |
| 4040 | Me | Me | ═CH-Pyrrolidine | |
| 4041 | Me | Et | Me | Me |
| 4042 | Me | Et | Me | Et |
| 4043 | Me | Et | Et | Me |
| 4044 | Me | Et | Et | Et |
| 4045 | Me | Et | Me | Ph |
| 4046 | Me | Et | Me | CH2CO2Me |
| 4047 | Me | Et | Morpholin-4-yl | |
| 4048 | Me | Et | 2-(Methoxycarbonyl)piperidin-1-yl | |
| 4049 | Me | Et | 2-(Ethoxycarbonyl)piperidin-1-yl | |
| 4050 | Me | Et | Thiazolidin-3-yl | |
| 4051 | Me | Et | Pyrrolidin-1-yl | |
| 4052 | Me | Et | 2-Methylpyrrolidin-1-yl | |
| 4053 | Me | Et | ═CH—N(CH3)2 | |
| 4054 | Me | Et | ═C(CH3)N(CH3)2 | |
| 4055 | Me | Et | ═CH—N(C2H5)2 | |
| 4056 | Me | Et | ═C(CH3)N(C2H5)2 | |
| 4057 | Me | Et | ═CH-Piperidine | |
| 4058 | Me | Et | ═CH-Morpholine | |
| 4059 | Me | Et | ═CH-Pyrrolidine | |
| 4060 | Me | Pr | Me | Me |
| 4061 | Me | Pr | Me | Et |
| 4062 | Me | Pr | Et | Me |
| 4063 | Me | Pr | Et | Et |
| 4064 | Me | Pr | Me | Ph |
| 4065 | Me | Pr | Me | CH2CO2Me |
| 4066 | Me | Pr | Morpholin-4-yl | |
| 4067 | Me | Pr | 2-(Methoxycarbonyl)piperidin-1-yl | |
| 4068 | Me | Pr | 2-(Ethoxycarbonyl)piperidin-1-yl | |
| 4069 | Me | Pr | Thiazolidin-3-yl | |
| 4070 | Me | Pr | Pyrrolidin-1-yl | |
| 4071 | Me | Pr | 2-Methylpyrrolidin-1-yl | |
| 4072 | Me | Pr | ═CH—N(CH3)2 | |
| 4073 | Me | Pr | ═C(CH3)N(CH3)2 | |
| 4074 | Me | Pr | ═CH—N(C2H5)2 | |
| 4075 | Me | Pr | ═C(CH3)N(C2H5)2 | |
| 4076 | Me | Pr | ═CH-Piperidine | |
| 4077 | Me | Pr | ═CH-Morpholine | |
| 4078 | Me | Pr | ═CH-Pyrrolidine | |
| 4079 | Me | Cl | Me | Me |
| 4080 | Me | Cl | Me | Et |
| 4081 | Me | Cl | Et | Me |
| 4082 | Me | Cl | Et | Et |
| 4083 | Me | Cl | Me | Ph |
| 4084 | Me | Cl | Me | CH2CO2Me |
| 4085 | Me | Cl | Morpholin-4-yl | |
| 4086 | Me | Cl | 2-(Methoxycarbonyl)piperidin-1-yl | |
| 4087 | Me | Cl | 2-(Ethoxycarbonyl)piperidin-1-yl | |
| 4088 | Me | Cl | Thiazolidin-3-yl | |
| 4089 | Me | Cl | Pyrrolidin-1-yl | |
| 4090 | Me | Cl | 2-Methylpyrrolidin-1-yl | |
| 4091 | Me | Cl | ═CH—N(CH3)2 | |
| 4092 | Me | Cl | ═C(CH3)N(CH3)2 | |
| 4093 | Me | Cl | ═CH—N(C2H5)2 | |
| 4094 | Me | Cl | ═C(CH3)N(C2H5)2 | |
| 4095 | Me | Cl | ═CH-Piperidine | |
| 4096 | Me | Cl | ═CH-Morpholine | |
| 4097 | Me | Cl | ═CH-Pyrrolidine | |
| 4098 | Me | Br | Me | Me |
| 4099 | Me | Br | Me | Et |
| 4100 | Me | Br | Et | Me |
| 4101 | Me | Br | Et | Et |
| 4102 | Me | Br | Me | Ph |
| 4103 | Me | Br | Me | CH2CO2Me |
| 4104 | Me | Br | Morpholin-4-yl | |
| 4105 | Me | Br | 2-(Methoxycarbonyl)piperidin-1-yl | |
| 4106 | Me | Br | 2-(Ethoxycarbonyl)piperidin-1-yl | |
| 4107 | Me | Br | Thiazolidin-3-yl | |
| 4108 | Me | Br | Pyrrolidin-1-yl | |
| 4109 | Me | Br | 2-Methylpyrrolidin-1-yl | |
| 4110 | Me | Br | ═CH—N(CH3)2 | |
| 4111 | Me | Br | ═C(CH3)N(CH3)2 | |
| 4112 | Me | Br | ═CH—N(C2H5)2 | |
| 4113 | Me | Br | ═C(CH3)N(C2H5)2 | |
| 4114 | Me | Br | ═CH-Piperidine | |
| 4115 | Me | Br | ═CH-Morpholine | |
| 4116 | Me | Br | ═CH-Pyrrolidine | |
| 4117 | Me | I | Me | Me |
| 4118 | Me | I | Me | Et |
| 4119 | Me | I | Et | Me |
| 4120 | Me | I | Et | Et |
| 4121 | Me | I | Me | Ph |
| 4122 | Me | I | Me | CH2CO2Me |
| 4123 | Me | I | Morpholin-4-yl | |
| 4124 | Me | I | 2-(Methoxycarbonyl)piperidin-1-yl | |
| 4125 | Me | I | 2-(Ethoxycarbonyl)piperidin-1-yl | |
| 4126 | Me | I | Thiazolidin-3-yl | |
| 4127 | Me | I | Pyrrolidin-1-yl | |
| 4128 | Me | I | 2-Methylpyrrolidin-1-yl | |
| 4129 | Me | I | ═CH—N(CH3)2 | |
| 4130 | Me | I | ═C(CH3)N(CH3)2 | |
| 4131 | Me | I | ═CH—N(C2H5)2 | |
| 4132 | Me | I | ═C(CH3)N(C2H5)2 | |
| 4133 | Me | I | ═CH-Piperidine | |
| 4134 | Me | I | ═CH-Morpholine | |
| 4135 | Me | I | ═CH-Pyrrolidine | |
| 4136 | CF3 | Me | Me | Me |
| 4137 | CF3 | Me | Me | Et |
| 4138 | CF3 | Me | Et | Me |
| 4139 | CF3 | Me | Et | Et |
| 4140 | CF3 | Me | Me | Ph |
| 4141 | CF3 | Me | Me | CH2CO2Me |
| 4142 | CF3 | Me | Morpholin-4-yl | |
| 4143 | CF3 | Me | 2-(Methoxycarbonyl)piperidin-1-yl | |
| 4144 | CF3 | Me | 2-(Ethoxycarbonyl)piperidin-1-yl | |
| 4145 | CF3 | Me | Thiazolidin-3-yl | |
| 4146 | CF3 | Me | Pyrrolidin-1-yl | |
| 4147 | CF3 | Me | 2-Methylpyrrolidin-1-yl | |
| 4148 | CF3 | Me | ═CH—N(CH3)2 | |
| 4149 | CF3 | Me | ═C(CH3)N(CH3)2 | |
| 4150 | CF3 | Me | ═CH—N(C2H5)2 | |
| 4151 | CF3 | Me | ═C(CH3)N(C2H5)2 | |
| 4152 | CF3 | Me | ═CH-Piperidine | |
| 4153 | CF3 | Me | ═CH-Morpholine | |
| 4154 | CF3 | Me | ═CH-Pyrrolidine | |
| 4155 | CF2CF3 | Me | Me | Me |
| 4156 | CF2CF3 | Me | Me | Et |
| 4157 | CF2CF3 | Me | Et | Me |
| 4158 | CF2CF3 | Me | Et | Et |
| 4159 | CF2CF3 | Me | Me | Ph |
| 4160 | CF2CF3 | Me | Me | CH2CO2Me |
| 4161 | CF2CF3 | Me | Morpholin-4-yl | |
| 4162 | CF2CF3 | Me | 2-(Methoxycarbonyl)piperidin-1-yl | |
| 4163 | CF2CF3 | Me | 2-(Ethoxycarbonyl)piperidin-1-yl | |
| 4164 | CF2CF3 | Me | Thiazolidin-3-yl | |
| 4165 | CF2CF3 | Me | Pyrrolidin-1-yl | |
| 4166 | CF2CF3 | Me | 2-Methylpyrrolidin-1-yl | |
| 4167 | CF2CF3 | Me | ═CH—N(CH3)2 | |
| 4168 | CF2CF3 | Me | ═C(CH3)N(CH3)2 | |
| 4169 | CF2CF3 | Me | ═CH—N(C2H5)2 | |
| 4170 | CF2CF3 | Me | ═C(CH3)N(C2H5)2 | |
| 4171 | CF2CF3 | Me | ═CH-Piperidine | |
| 4172 | CF2CF3 | Me | ═CH-Morpholine | |
| 4173 | CF2CF3 | Me | ═CH-Pyrrolidine | |
| 4174 | CF3 | Et | Me | Me |
| 4175 | CF3 | Et | Me | Et |
| 4176 | CF3 | Et | Et | Me |
| 4177 | CF3 | Et | Et | Et |
| 4178 | CF3 | Et | Me | Ph |
| 4179 | CF3 | Et | Me | CH2CO2Me |

| | | | |
|---|---|---|---|
| 4180 | CF3 | Et | Morpholin-4-yl |
| 4181 | CF3 | Et | 2-(Methoxycarbonyl)piperidin-1-yl |
| 4182 | CF3 | Et | 2-(Ethoxycarbonyl)piperidin-1-yl |
| 4183 | CF3 | Et | Thiazolidin-3-yl |
| 4184 | CF3 | Et | Pyrrolidin-1-yl |
| 4185 | CF3 | Et | 2-Methylpyrrolidin-1-yl |
| 4186 | CF3 | Et | ═CH—N(CH3)2 |
| 4187 | CF3 | Et | ═C(CH3)N(CH3)2 |
| 4188 | CF3 | Et | ═CH—N(C2H5)2 |
| 4189 | CF3 | Et | ═C(CH3)N(C2H5)2 |
| 4190 | CF3 | Et | ═CH-Piperidine |
| 4191 | CF3 | Et | ═CH-Morpholine |
| 4192 | CF3 | Et | ═CH-Pyrrolidine |

NMR data of selected compounds mentioned in table A above

The NMR data of selected compounds mentioned in table A are listed either in conventional form (δ values, number of hydrogen atoms, multiplet splitting) or as NMR peak lists. The compounds mentioned in table A are assigned via the example numbers according to table A.

When the ¹H NMR data of selected examples are noted in the form of ¹H NMR peak lists, for each signal peak, first the δ value in ppm and then the signal intensity in round brackets are listed. The pairs of δ value-signal intensity numbers for different signal peaks are listed with separation from one another by semicolons.

The peak list therefore has the following form:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

Calibration of the chemical shift of ¹H NMR spectra is accomplished using tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which likewise form part of the subject matter of the invention, and/or peaks of impurities.

In the reporting of compound signals within the delta range of solvents and/or water, the lists of ¹H NMR peaks show the standard solvent peaks, for example peaks of DMSO in DMSO and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

A person skilled in the art calculating the peaks of the target compounds by known methods (MestreC, ACD simulation, but also with empirically evaluated expected values) can, if required, isolate the peaks of the target compounds, optionally using additional intensity filters. This isolation is similar to the peak picking in question in conventional ¹H NMR interpretation.

Compound No. 1212, solvent: [DMSO], 400 MHz

8.7623 (2.03); 8.434 (12.64); 8.433 (13.22); 7.1577 (0.37); 3.3649 (3.75); 3.3507 (5.06); 3.3469 (9.73); 3.3329 (10.53); 3.3289 (10.54); 3.3191 (4.46); 3.3148 (9.66); 3.311 (4.89); 3.3011 (2.24); 3.2968 (3.67); 2.8418 (0.53); 2.8273 (0.55); 2.8236 (0.53); 2.8091 (0.53); 2.673 (0.31); 2.5266 (0.53); 2.5219 (0.73); 2.5132 (15.56); 2.5086 (35.14); 2.504 (50); 2.4994 (35.33); 2.4948 (15.84); 2.0873 (1.5); 2.0752 (0.57); 1.2806 (0.31); 1.1984 (0.47); 1.195 (0.31); 1.1601 (2.01); 1.1529 (16.88); 1.142 (4.22); 1.1349 (36.51); 1.1238 (2.23); 1.1168 (16.39); 0.6742 (0.35); −0.0002 (5.99);

Compound No. 2202, solvent: [DMSO], 400 MHz

13.4417 (0.37); 9.0046 (0.5); 8.4124 (8.33); 7.206 (5.24); 7.0745 (13.79); 6.9429 (6.22); 3.4828 (0.56); 3.3619 (2.96); 3.3477 (3.4); 3.3439 (9.47); 3.3297 (9.62); 3.3258 (10.14); 3.3117 (9.62); 3.3079 (3.94); 3.2937 (3.16); 2.5138 (11.84); 2.5092 (28.43); 2.5046 (41.41); 2.5 (30.07); 2.4953 (13.75); 1.1466 (22); 1.1286 (50); 1.1105 (21.47); −0.0002 (7.09);

Compound No. 2209, solvent: [CDCl3], 400 MHz

8.8346 (0.69); 8.6969 (8.07); 7.2663 (0.34); 7.262 (46.18); 5.3002 (2.24); 4.302 (0.56); 4.2856 (1.55); 4.2691 (2.23); 4.2663 (1.8); 4.2526 (1.81); 4.2498 (2.27); 4.2334 (1.68); 4.2171 (0.64); 1.2632 (50); 1.2467 (49.93); 0.9211 (0.33); 0.0697 (0.52); 0.0081 (0.58); −0.0002 (23.9); −0.0085 (0.78);

Compound No. 2223, solvent: [DMSO], 400 MHz

8.6658 (4.79); 8.6259 (0.75); 3.3465 (8.69); 3.1458 (4.55); 3.1302 (4.53); 2.6728 (0.3); 2.555 (0.31); 2.5131 (16.61); 2.5086 (36.23); 2.5041 (50); 2.4996 (35.61); 2.4951 (16.09); 2.4534 (0.32); 2.0875 (3.81); 0.955 (0.4); 0.9451 (4.23); 0.904 (49.74); 0.8532 (0.37); −0.0002 (3.76);

Compound No. 1565, solvent: [DMSO], 400 MHz

8.8649 (1.47); 8.851 (2.71); 8.8375 (1.59); 8.3737 (13.08); 5.9356 (0.79); 5.9226 (1.74); 5.9098 (1.67); 5.8968 (2.07); 5.8817 (1.9); 5.8797 (2.16); 5.8668 (1.91); 5.8538 (2.13); 5.841 (1.01); 5.2502 (3.77); 5.2484 (3.81); 5.2462 (3.79); 5.2071 (3.33); 5.2053 (3.36); 5.2031 (3.34); 5.1354 (4.06); 5.1317 (3.83); 5.1097 (3.81); 5.1083 (3.71); 5.1059 (3.62); 3.9556 (4.13); 3.9427 (7.68); 3.9284 (4.13); 3.9109 (0.84); 3.3412 (22.6); 2.6734 (0.32); 2.5066 (46.89); 2.5047 (50); 2.0767 (0.77); 2.0746 (1.11); 1.234 (0.49); 0.0019 (0.84); −0.0002 (1.09);

Compound No. 2224, solvent: [DMSO], 400 MHz

9.0276 (0.38); 9.0137 (0.77); 8.9996 (0.41); 8.7018 (2.82); 8.5105 (0.81); 4.1208 (2.6); 4.1066 (2.6); 3.837 (0.31); 3.6645 (10.33); 3.4379 (6.63); 2.6718 (0.34); 2.5023 (50); 2.0729 (0.43); 1.356 (1.07); 0.0017 (4.72); −0.0002 (4.79);

Compound No. 2225, solvent: [DMSO], 400 MHz

8.6055 (3.29); 3.9028 (0.93); 3.8857 (1.08); 3.8798 (0.99); 3.8626 (0.92); 3.3173 (7.37); 2.5057 (35.73); 2.5018 (50); 2.4975 (37.71); 2.0738 (1.06); 1.3553 (0.47); 1.2334 (0.61); 1.0762 (8.19); 1.0592 (8.18); 0.8972 (45.37); 0.8579 (0.53); 0.8484 (0.42); −0.0002 (5.77); −0.0017 (4.97);

Compound No. 1679, solvent: [DMSO], 400 MHz

8.3725 (0.53); 3.6269 (50); 2.5107 (2.79); 2.5075 (3.65); 2.5036 (2.74); 1.5003 (0.32); 1.1429 (0.91); 1.1264 (0.9); 0.884 (0.46); 0.8656 (0.92); 0.847 (0.4);

Compound No. 1688, solvent: [DMSO], 400 MHz

8.4737 (1.39); 8.4531 (1.41); 8.3891 (8.91); 3.8839 (0.99); 3.8676 (1.63); 3.8467 (1.61); 3.8304 (1); 3.3364 (6.91); 2.5428 (0.32); 2.5072 (36.58); 2.5032 (50); 2.499 (37.24); 2.3298 (0.32); 2.0747 (0.45); 1.7996 (0.4); 1.7828 (1.15); 1.7662 (1.95); 1.7495 (1.99); 1.7331 (1.23); 1.7161 (0.45); 1.1126 (13.68); 1.0958 (13.57); 0.9013 (22.11); 0.8843 (21.5); −0.0002 (5.66); −0.0015 (5.39);

Compound No. 2001, solvent: [CDCl3], 400 MHz

8.5005 (6.96); 8.118 (0.35); 7.2608 (50); 3.3225 (5.44); 3.3074 (6.42); 3.3056 (6.96); 3.2904 (5.61); 2.009 (0.74); 1.9488 (0.91); 1.932 (1.84); 1.9151 (2.33); 1.8983 (1.9); 1.8815 (0.99); 1.8647 (0.31); 1.0717 (0.76); 1.0549 (0.72); 0.9931 (37.46); 0.9764 (36.24); 0.008 (0.67); −0.0002 (25.44); −0.0085 (0.68);

Compound No. 2007, solvent: [CDCl3], 400 MHz

8.5485 (10.65); 8.2909 (0.56); 7.2608 (50); 4.9202 (5.67); 4.9173 (10.78); 4.9142 (10.55); 4.9112 (5.67); 4.0378 (5.8); 4.023 (5.79); 2.0089 (6.04); 1.8713 (0.39); 1.7932 (20.42); 0.9913 (1.15); 0.9745 (1.11); 0.008 (0.59); −0.0002 (25.86); −0.0085 (0.77);

Compound No. 2009, solvent: [CDCl3], 400 MHz

8.4585 (5.62); 7.7568 (0.34); 7.3115 (0.74); 7.2608 (50); 7.2108 (1.19); 4.1531 (0.68); 4.1365 (1.48); 4.1197 (1.68); 4.1159 (1.53); 4.0993 (1.48); 4.0827 (0.72); 2.0089 (0.47); 1.6682 (0.31); 1.6505 (0.35); 1.6352 (1.24); 1.6166 (4.09); 1.5993 (5.66); 1.581 (4.61); 1.5627 (1.48); 1.3992 (0.42); 1.383 (0.4); 1.3071 (0.36); 1.256 (15.88); 1.2395 (15.64); 1.2058 (0.45); 1.1893 (0.44); 1.0382 (0.47); 1.0151 (0.33); 0.9826 (7.57); 0.9641 (15.59); 0.9454 (6.85); 0.9139 (0.43); 0.0504 (0.32); 0.0079 (0.68); −0.0002 (21.18); −0.0086 (0.75); −0.0503 (0.5);

Compound No. 2016, solvent: [CDCl3], 400 MHz

8.4341 (7.93); 7.6601 (2.26); 7.3047 (18.35); 7.2858 (24.53); 7.2606 (50); 5.2997 (3.97); 4.2928 (5.23); 1.6585 (8.93); 1.511 (9.14); 1.3936 (8.21); 1.2486 (25.99); 1.2325 (18.68); 0.9459 (49.4); 0.9328 (34.96); −0.0002 (28.64);

Compound No. 2020, solvent: [CDCl3], 400 MHz 0.146 (0.45); 0.0502 (0.54); 0.011 (0.57); 0.0101 (0.66); 0.0093 (0.9); 0.0082 (4.03); 0.0069 (1.59); 0.0061 (1.83); 0.0053 (1.43); 0.0044 (1.48); 0.0036 (2.31); 0.0028 (4.61); 0.0019 (7.29); 0 (179.38); −0.0019 (45.97); −0.0048 (2.9); −0.0057 (2.05); −0.0065 (1.71); −0.0083 (5.22); −0.1496 (0.5);

Compound No. 2023, solvent: [CDCl3], 400 MHz

8.4635 (5.14); 8.0225 (0.4); 7.2625 (14.19); 4.5184 (1.49); 4.5059 (3.37); 4.4934 (1.53); 3.6466 (2.71); 3.6325 (3.88); 3.6195 (2.47); 3.4415 (50); 3.3907 (0.37); 3.3893 (0.39); 2.0107 (1.38); −0.0002 (7.96);

Compound No. 1755, solvent: [DMSO], 400 MHz

12.5538 (1.28); 8.1739 (0.53); 8.0175 (8.7); 3.6181 (0.6); 3.6015 (1.23); 3.5954 (0.64); 3.5851 (0.77); 3.4502 (2.44); 3.001 (1.04); 2.9711 (30.11); 2.8456 (1.14); 2.8285 (31.56); 2.8078 (0.42); 2.6713 (0.31); 2.5247 (0.6); 2.5201 (0.81); 2.5112 (15.67); 2.5067 (35.26); 2.5022 (50); 2.4976 (35.64); 2.493 (16.2); 2.0736 (1.04); 1.7767 (0.37); 1.7689 (0.39); 1.7601 (1.15); 1.7514 (0.4); 1.7437 (0.4); 1.3558 (0.98); −0.0002 (3.81);

Compound No. 1269, solvent: [DMSO], 400 MHz

9.0311 (0.45); 9.017 (0.87); 9.0026 (0.46); 8.4052 (3.78); 8.4038 (4.12); 4.6207 (0.6); 4.2824 (0.68); 4.1307 (4.33); 4.1226 (1.01); 4.1163 (4.33); 3.85 (0.32); 3.7271 (0.47); 3.6793 (1.69); 3.6666 (21.41); 3.3406 (4.06); 2.6708 (0.31); 2.5243 (0.62); 2.5196 (0.85); 2.5108 (15.64); 2.5063 (35.39); 2.5017 (50); 2.497 (35.26); 2.4924 (15.57); 2.0732 (1.41); 1.3556 (1.89); −0.0002 (4.95);

Compound No. 2085, solvent: [CDCl3], 400 MHz

7.9751 (5.08); 7.2659 (0.33); 7.2651 (0.37); 7.2642 (0.55); 7.2634 (0.89); 7.2603 (50); 7.2567 (1); 7.2558 (0.73); 7.255 (0.53); 7.2542 (0.36); 3.1275 (29.16); 1.5851 (1.02); 0.0079 (0.5); −0.0002 (18.81); −0.0083 (0.57);

Compound No. 1988, solvent: [CDCl3], 400 MHz

8.3303 (0.95); 7.5185 (0.31); 7.2598 (50); 2.0062 (0.33); 1.5742 (0.43); 1.5531 (0.45); 1.474 (28.84); 1.3148 (0.32); 0.8819 (0.37); 0.008 (0.58); −0.0002 (22.15); −0.0085 (0.66);

Compound No. 1687, solvent: [CDCl3], 400 MHz

9.0593 (0.64); 9.0378 (0.67); 8.6077 (3.78); 7.2647 (0.48); 7.2615 (33.25); 7.2571 (0.65); 7.2563 (0.47); 7.2554 (0.37); 4.1269 (0.3); 4.11 (1.01); 4.0929 (1.12); 4.0862 (1.13); 4.0691 (1.04); 4.052 (0.34); 1.3213 (0.41); 1.3039 (0.8); 1.2866 (1); 1.2642 (3.74); 1.1826 (8.34); 1.1656 (8.33); 1.1185 (0.35); 1.0138 (0.32); 0.9656 (50); 0.9256 (1.04); 0.8987 (2.23); 0.8879 (1.24); 0.882 (6.95); 0.8642 (2.84); 0.8484 (0.39); 0.8369 (0.31); 0.8076 (0.38); 0.0081 (0.43); −0.0002 (18.19); −0.0014 (7.13); −0.0085 (0.55);

Compound No. 1883, solvent: [CDCl3], 400 MHz

9.0338 (0.93); 8.655 (7.86); 7.2629 (50); 6.5319 (0.55); 6.5199 (1.16); 6.5077 (0.58); 6.4001 (1.15); 6.388 (2.43); 6.3758 (1.22); 6.268 (0.57); 6.256 (1.24); 6.2438 (0.64); 3.3392 (5.33); 3.3255 (5.78); 3.3214 (6.05); 3.3077 (5.49); 2.1185 (5.02); 2.048 (0.94); 2.0114 (0.44); 1.2786 (0.39); 1.2607 (0.77); 1.2534 (0.74); 1.2428 (0.38); 1.1001 (0.41); 1.0925 (0.57); 1.0804 (1.15); 1.0741 (0.98); 1.0703 (0.77); 1.0622 (1.92); 1.0544 (0.82); 1.0501 (1.06); 1.0424 (1.32); 1.0304 (0.7); 1.0243 (0.5); 0.593 (1.51); 0.581 (4.28); 0.5783 (4.96); 0.5735 (2.28); 0.5666 (2.12); 0.5611 (4.76); 0.5583 (4.22); 0.5467 (1.89); 0.2974 (1.72); 0.2858 (5.57); 0.2739 (4.43); 0.2712 (5.82); 0.2591 (1.38); 0.008 (0.54); −0.0002 (22.1); −0.0085 (0.78);

Compound No. 1924, solvent: [CDCl3], 400 MHz

8.8401 (0.86); 8.4303 (4.54); 7.3135 (0.42); 7.2633 (35.51); 6.5831 (0.62); 6.571 (1.25); 6.5588 (0.63); 6.4511 (1.27); 6.439 (2.58); 6.4268 (1.3); 6.3192 (0.64); 6.307 (1.31); 6.2948 (0.66); 3.5955 (3.08); 3.58 (6.11); 3.5635 (11.29); 3.5489 (14.61); 3.5341 (6.59); 3.4464 (0.65); 3.3958 (50); 3.348 (0.37); 1.9355 (1.68); 1.9205 (5.03); 1.905 (6.44); 1.8893 (4.77); 1.8742 (1.52); 0.0079 (0.72); 0.0008 (14.44); −0.0002 (18.78); −0.0072 (0.45); −0.0086 (0.5);

Compound No. 2215, solvent: [CDCl3], 400 MHz

8.7826 (0.49); 8.2616 (2.5); 7.262 (50); 6.5476 (0.46); 6.5356 (0.92); 6.5235 (0.45); 6.4155 (0.92); 6.4036 (1.89); 6.3915 (0.95); 6.2835 (0.46); 6.2715 (0.97); 6.2595 (0.5); 3.5986 (2.3); 3.5831 (4.19); 3.5683 (8.85); 3.5536 (11.04); 3.5393 (5.06); 3.3984 (43.07); 3.3694 (0.31); 1.9357 (1.23); 1.9208 (3.42); 1.9044 (4.29); 1.8895 (3.35); 1.8747 (1.18); 0.0079 (0.84); −0.0002 (30.48); −0.0086 (0.86);

Compound No. 2220, solvent: [CDCl3], 400 MHz

0.1464 (0.7); 0.0697 (1.57); 0.0524 (0.42); 0.019 (0.42); 0.0174 (0.47); 0.0157 (0.52); 0.0149 (0.54); 0.0141 (0.57); 0.0133 (0.62); 0.0124 (0.68); 0.0116 (0.79); 0.0108 (1.03); 0.0083 (5.31); 0.0059 (2.36); 0.0051 (2.85); 0 (174.28); −0.005 (1.82); −0.0058 (1.31); −0.0083 (4.88); −0.0515 (0.43); −0.149 (0.66);

Compound No. 2219, solvent: [CDCl3], 400 MHz

0.1461 (0.39); 0.0539 (0.28); 0.0105 (0.44); 0.0081 (2.8); 0.0056 (1.13); 0.0022 (11.59); 0 (102.98); −0.0053 (2.22); −0.0061 (1.79); −0.0085 (3.22); −0.0094 (2.01); −0.0119 (0.68); −0.0127 (0.55); −0.0135 (0.5); −0.0151 (0.42); −0.0159 (0.4); −0.0168 (0.36); −0.0184 (0.33); −0.0477 (0.39); −0.0526 (0.47); −0.1494 (0.4);

Compound No. 2096, solvent: [CDCl3], 400 MHz

12.3751 (0.33); 9.3183 (1.64); 8.6946 (11.42); 8.6919 (11.21); 7.2667 (0.37); 7.2659 (0.52); 7.2612 (50); 7.2575 (1.05); 7.2566 (0.76); 7.2558 (0.58); 7.2549 (0.46); 7.2541 (0.38); 7.2533 (0.31); 6.1695 (0.83); 6.1559 (2.78); 6.1423 (2.96); 6.1285 (1); 6.0627 (0.81); 6.0492 (2.76); 6.0355 (3.02); 6.0218 (1.05); 3.4904 (0.43); 3.4746 (2.02); 3.4665 (1.98); 3.4568 (4.35); 3.4526 (2.75); 3.4488 (4.08); 3.4426 (4.11); 3.4386 (2.82); 3.4348 (4.38); 3.4246 (2.08); 3.4171 (2.13); 3.401 (0.46); 1.6257 (1.57); 1.6078 (4.87); 1.6016 (1.79); 1.5896 (6.36); 1.5838 (3.29); 1.5706 (5.41); 1.5522 (2.38); 1.4549 (1.29); 1.4364 (3.95); 1.4227 (2.22); 1.4174 (5.42); 1.4043 (2.32); 1.3982 (5.55); 1.3861 (1.02); 1.3802 (3.48); 1.3621 (1.09); 0.9653 (15.68); 0.947 (32.38); 0.9286 (13.57); 0.0078 (0.71); −0.0002 (28.52); −0.0057 (0.52); −0.0065 (0.44); −0.0083 (0.95); −0.0089 (0.93);

Compound No. 2126, solvent: [DMSO], 400 MHz

8.4268 (1.22); 6.63 (0.3); 6.5251 (0.31); 3.3633 (0.42); 3.3171 (18.18); 3.1688 (0.37); 2.55 (0.46); 2.5456 (0.58); 2.5411 (0.42); 2.5237 (0.66); 2.519 (0.86); 2.5103 (15.4); 2.5057 (35.25); 2.5011 (50); 2.4964 (35.48); 2.4918 (16.07); 2.4624 (0.49); 2.458 (0.45); 1.4655 (0.36); 1.4435 (0.32); 1.3112 (0.31); 1.2972 (0.35); 1.2767 (0.49); 1.145 (2.45); 1.1286 (2.41); 0.8863 (5.06); 0.8698 (5.14); −0.0002 (4.67);

Compound No. 2230, solvent: [DMSO], 400 MHz

8.5233 (0.92); 3.3137 (35.51); 3.1685 (0.41); 2.8574 (0.36); 2.8471 (0.37); 2.5234 (0.65); 2.5187 (0.85); 2.51 (15.41); 2.5054 (35.21); 2.5008 (50); 2.4961 (35.18); 2.4915 (15.65); 2.0721 (0.63); 0.7534 (0.63); 0.7479 (0.88); 0.7355 (0.88); 0.7298 (0.66); 0.7178 (0.3); 0.5698 (0.69); 0.5632 (0.65); 0.5588 (0.6); 0.5535 (0.6); −0.0002 (4.73)

Compound No. 2231, solvent: [DMSO], 400 MHz

8.5564 (1.06); 6.5033 (0.33); 6.488 (0.34); 6.3985 (0.32); 6.3833 (0.36); 5.7526 (1.26); 3.9721 (0.34); 3.317 (16.71); 3.1691 (0.46); 2.5239 (0.55); 2.5193 (0.74); 2.5106 (15.16); 2.506 (34.94); 2.5013 (50); 2.4967 (35.25); 2.4921 (15.87); 2.4531 (0.32); 1.4701 (0.5); 1.4516 (0.31); 1.2783 (1.05); 1.2707 (1.18); 1.2614 (1.68); 1.2347 (0.51); 1.146 (2.68); 1.1303 (2.7); 0.8682 (0.83); 0.8517 (3.11); 0.8345 (1.03); −0.0002 (10.3); −0.0085 (0.32);

Compound No. 2232, solvent: [DMSO], 400 MHz

8.5637 (0.77); 6.3904 (0.31); 3.4784 (0.46); 3.4674 (0.82); 3.4575 (2.17); 3.4509 (1.13); 3.3143 (20.1); 3.2703 (10.86); 3.1685 (0.38); 2.5234 (0.58); 2.5187 (0.77); 2.51 (15.25); 2.5054 (34.86); 2.5007 (50); 2.4961 (35.24); 2.4915 (15.71); −0.0002 (1.61);

Compound No. 2233, solvent: [DMSO], 400 MHz

8.5551 (1.32); 6.5024 (0.47); 6.4872 (0.52); 6.3976 (0.46); 6.3823 (0.52); 3.3998 (0.79); 3.3951 (1.79); 3.3848 (1.53); 3.3796 (4.04); 3.3735 (0.89); 3.3698 (1.11); 3.3641 (2.08); 3.3567 (1.71); 3.3417 (1.94); 3.3166 (17.14); 3.2834 (0.34); 3.2381 (21.77); 3.1687 (0.42); 2.5239 (0.7); 2.5192 (0.92); 2.5105 (15.37); 2.5059 (35.13); 2.5013 (50); 2.4966 (35.35); 2.492 (15.92); 1.7895 (0.33); 1.7727 (0.62); 1.7563 (1.36); 1.7403 (1.92); 1.7242 (1.15); 1.7078 (0.33); −0.0002 (2.14);

Compound No. 2128, solvent: [DMSO], 400 MHz

8.412 (1.92); 8.4095 (1.93); 6.6529 (0.36); 6.6374 (0.38); 6.548 (0.35); 6.5328 (0.37); 5.7527 (2.06); 3.8704 (0.42); 3.8494 (0.42); 3.8341 (0.33); 3.3306 (5.07); 3.1691 (0.57); 2.5239 (0.58); 2.5192 (0.77); 2.5105 (15.19); 2.5059 (35.08); 2.5013 (50); 2.4966 (35.41); 2.492 (15.88); 1.7788 (0.35); 1.762 (0.55); 1.7465 (0.56); 1.7297 (0.39); 1.1076 (3.08); 1.1052 (3.24); 1.0907 (3.1); 1.0883 (3.21); 0.9007 (4.65); 0.8948 (4.35); 0.8837 (4.62); 0.8779 (4.18); −0.0002 (12.11); −0.0085 (0.35);

Compound No. 2234, solvent: [DMSO], 400 MHz

3.3126 (40.61); 2.5233 (0.63); 2.5187 (0.86); 2.51 (15.25); 2.5054 (35.14); 2.5007 (50); 2.4961 (35.27); 2.4915 (15.71); 2.0721 (0.35); 1.5188 (0.46); 1.5005 (0.65); 1.483 (0.45); 1.154 (0.37); 1.1421 (1.44); 1.1401 (1.53); 1.1256 (1.42); 1.1236 (1.45); 0.8941 (0.47); 0.8894 (0.91); 0.8707 (1.8); 0.8524 (0.77); −0.0002 (4.93);

Compound No. 2235, solvent: [DMSO], 400 MHz

8.5434 (1.4); 6.5103 (0.31); 6.495 (0.33); 6.4057 (0.31); 6.3906 (0.31); 3.8671 (0.37); 3.846 (0.35); 3.3268 (3.6); 2.524 (0.57); 2.5194 (0.74); 2.5107 (15.16); 2.5061 (34.98); 2.5014 (50); 2.4968 (35.24); 2.4921 (15.63); 1.7731 (0.32); 1.7562 (0.47); 1.7411 (0.5); 1.7241 (0.34); 1.1017 (2.59); 1.0992 (2.9); 1.0848 (2.62); 1.0823 (2.85); 0.8973 (3.95); 0.8903 (3.5); 0.8803 (3.94); 0.8733 (3.32); −0.0002 (3.98);

Compound No. 2127, solvent: [DMSO], 400 MHz

8.3884 (3.9); 6.6602 (0.61); 6.6476 (0.62); 6.6448 (0.63); 6.5587 (0.62); 6.5559 (0.63); 6.5434 (0.69); 6.5404 (0.66); 5.7528 (0.86); 3.9199 (0.3); 3.9028 (1.07); 3.897 (0.39); 3.8857 (1.14); 3.8797 (1.12); 3.8687 (0.44); 3.8626 (1.09); 3.8455 (0.33); 3.8353 (0.33); 3.3824 (3.34); 3.256 (0.54); 3.24 (0.46); 3.1705 (0.84); 2.5252 (0.33); 2.5205 (0.45); 2.5119 (13.2); 2.5073 (30.48); 2.5026 (43.96); 2.498 (31.34); 2.4934 (14.21); 2.0983 (0.47); 2.0733 (0.39); 1.7122 (0.42); 1.083 (5.45); 1.0794 (5.88); 1.0693 (2.41); 1.066 (5.6); 1.0624 (5.88); 1.0567 (1.78); 1.0092 (0.34); 0.9752 (0.82); 0.9711 (0.41); 0.9593 (0.83); 0.954 (0.61); 0.9512 (0.48); 0.9188 (1.34); 0.9031 (50); 0.8697 (0.51); 0.865 (0.51); 0.8532 (0.63); 0.8432 (0.51); 0.8374 (2.08); 0.824 (0.61); 0.8177 (0.61); 0.8033 (4.27); −0.0002 (8.79);

Compound No. 1445, solvent: [CDCl3], 400 MHz

9.3978 (0.45); 8.6158 (3.29); 8.613 (3.22); 7.2609 (21.23); 6.1627 (0.82); 6.1491 (0.87); 6.0558 (0.82); 6.0421 (0.89); 6.0284 (0.31); 3.339 (0.77); 3.3228 (0.79); 3.3059 (1.73); 3.2897 (1.69); 3.2568 (1.67); 3.2415 (1.74); 3.2237 (0.79); 3.2084 (0.76); 1.0976 (0.52); 0.9727 (50); −0.0002 (10.36); −0.0082 (0.33);

Compound No. 1489, solvent: [CDCl3], 400 MHz

9.7362 (0.94); 8.5617 (4.87); 8.5592 (4.88); 7.2621 (50); 6.1699 (0.38); 6.1564 (1.3); 6.1428 (1.38); 6.1293 (0.47); 6.0628 (0.39); 6.0494 (1.3); 6.0358 (1.4); 6.0221 (0.48); 4.2457 (4.03); 4.2418 (4.02); 4.2317 (4.14); 4.2278 (4.13); 4.0384 (0.66); 4.0261 (1.1); 3.7822 (28.47); 3.7554 (2.68); 3.7504 (0.73); 3.7356 (0.47); 3.7182 (0.96); 1.8554 (0.75); 1.8467 (0.31); 1.5889 (0.48); 1.4322 (2.32); 1.2539 (0.63); 0.008 (0.74); −0.0002 (27.06); −0.0085 (0.99);

Compound No. 1462, solvent: [CDCl3], 400 MHz

9.2378 (1.17); 9.2175 (1.2); 8.5984 (7.02); 8.5962 (7.34); 7.3115 (0.33); 7.261 (50); 6.1988 (0.56); 6.185 (1.83); 6.1713 (1.92); 6.1576 (0.66); 6.0917 (0.55); 6.0782 (1.81); 6.0645 (1.97); 6.0508 (0.7); 5.2986 (2.24); 4.2107 (0.67); 4.194 (1.33); 4.1737 (1.59); 4.1568 (1.4); 4.14 (0.72); 4.0216 (0.94); 1.5612 (1.02); 1.5502 (1.35); 1.5452 (1.37); 1.5309 (2.46); 1.5109 (3.93); 1.4939 (3.95); 1.478 (2.27); 1.4603 (0.92); 1.4456 (0.78); 1.4306 (0.83); 1.4082 (1.8); 1.3917 (2.69); 1.384 (2.1); 1.3764 (2.3); 1.3669 (1.95); 1.3582 (1.38); 1.3498 (1.23); 1.2559 (0.5); 1.222 (10.2); 1.2179 (10.37); 1.2056 (10.37); 1.2015 (10.26); 0.941 (10.07); 0.9228 (19.94); 0.9049 (8.24); 0.008 (0.57); −0.0002 (22.8); −0.0084 (0.88);

Compound No. 1975, solvent: [CDCl3], 400 MHz

7.9409 (2.4); 7.2595 (50); 6.6218 (0.35); 6.5029 (0.35); 6.4897 (0.73); 6.4765 (0.37); 6.3576 (0.34); 3.1367 (21.54); 1.5504 (2.88); 0.008 (0.73); −0.0002 (24.64); −0.0086 (1.05);

Compound No. 2239, solvent: [DMSO], 400 MHz

8.7524 (6.37); 8.6706 (0.95); 8.649 (0.95); 7.0736 (0.53); 6.9576 (0.55); 6.944 (1.12); 6.9302 (0.55); 6.8143 (0.58); 6.8011 (0.31); 4.0309 (0.35); 4.0125 (0.76); 3.9956 (1.01); 3.9784 (0.75); 3.9606 (0.36); 3.343 (50); 3.1721 (0.4); 2.5132 (15.5); 2.5089 (34.58); 2.5044 (49.22); 2.4999 (35.88); 2.4955 (16.96); 1.5072 (0.89); 1.4912 (1.4); 1.4733 (1.22); 1.4567 (0.63); 1.2811 (3.22); 1.2656 (4.78); 1.16 (8.54); 1.1435 (8.48); 0.8706 (2.29); 0.8544 (6.77); 0.8372 (2.51); −0.0002 (8.13);

Compound No. 1790, solvent: [CDCl3], 400 MHz

8.8072 (0.88); 8.6436 (7.69); 7.5188 (0.46); 7.26 (82.13); 6.996 (0.46); 3.6152 (1.05); 3.5957 (2.03); 3.5786 (2.06); 3.5588 (1.16); 1.659 (0.35); 1.6518 (0.35); 1.644 (0.36); 1.6379 (0.37); 1.6241 (0.38); 1.6126 (0.4); 1.587 (0.39); 1.5785 (0.4); 1.5442 (0.38); 1.5373 (0.36); 1.5244 (0.35); 1.4783 (0.39); 1.4618 (0.34); 1.3222 (16); 1.3057 (16); 0.9802 (0.33); 0.9674 (0.65); 0.9591 (1); 0.9469 (1.94); 0.9352 (1.36); 0.9264 (2.08); 0.9141 (1.19); 0.906 (0.85); 0.8938 (0.46); 0.6013 (0.49); 0.588 (0.78); 0.5782 (1.44); 0.566 (1.92); 0.5551 (1.82); 0.5446 (1.28); 0.5337 (1.07); 0.5267 (1.02); 0.5138 (1.57); 0.5021 (1.43); 0.4937 (2.06); 0.4806 (1.42); 0.4714 (0.83); 0.4595 (0.68); 0.4208 (0.73); 0.408 (1.51); 0.397 (1.98); 0.3848 (2.59); 0.3723 (2.05); 0.36 (0.78); 0.3083 (0.92); 0.296 (2.09); 0.2843 (2.49); 0.2723 (2.16); 0.2607 (1.33); 0.2491 (0.53); 0.0076 (0.86); 0 (33.52);

Compound No. 1460, solvent: [DMSO], 400 MHz

8.0921 (1.4); 8.0899 (1.43); 6.4804 (0.37); 6.4649 (0.39); 6.3757 (0.39); 6.3601 (0.39); 3.2543 (0.36); 3.2354 (0.37); 3.0772 (1.58); 2.2679 (4.99); 2.2634 (11.3); 2.2588 (16); 2.2541 (11.79); 2.2496 (5.7); 1.1134 (1.46); 0.9853 (2.3); 0.9834 (2.42); 0.9687 (2.31); 0.9667 (2.37); 0.7505 (0.43); 0.7302 (0.46); 0.2359 (0.34); 0.2267 (0.34); 0.2227 (0.35); 0.2147 (0.49); 0.169 (0.39); 0.1586 (0.39); 0.1477 (0.39); 0.039 (0.47); 0.0272 (0.44); 0.0229 (0.35); 0.0142 (0.36); 0.0094 (0.48); 0 (0.44);

Compound No. 1901, solvent: [DMSO], 400 MHz

9.0841 (0.79); 8.3959 (10.95); 7.7958 (0.34); 7.0784 (0.61); 7.065 (1.23); 7.0514 (0.64); 6.9488 (1.25); 6.9355 (2.6); 6.9219 (1.28); 6.8194 (0.62); 6.806 (1.33); 6.7925 (0.66); 4.8724 (0.35); 4.855 (0.93); 4.836 (1.52); 4.8162 (1.46); 4.8041 (0.79); 4.7963 (0.94); 4.7846 (0.32); 4.7775 (0.37); 3.3496 (5.6); 3.1652 (1.17); 2.6756 (1.57); 2.6711 (2.19); 2.6667 (1.5); 2.5497 (0.53); 2.5451 (1.33); 2.5411 (1.72); 2.5246 (6.07); 2.5199 (8.36); 2.511 (124.4); 2.5066 (268.94); 2.5021 (367.92); 2.4975 (261.2); 2.4931 (120.09); 2.3334 (1.58); 2.3289 (2.12); 2.3244 (1.48); 2.0587 (1.92); 1.3517 (16); 1.3342 (15.89); 0.1462 (0.61); 0.0481 (0.4); 0.0082 (4.28);

Compound No. 1828, solvent: [CDCl3], 400 MHz

8.9958 (1.96); 8.978 (1.98); 8.6874 (16); 8.6861 (15.52); 7.5199 (1.05); 7.3106 (1.28); 7.3079 (0.43); 7.3072 (0.37); 7.281 (0.33); 7.2802 (0.39); 7.2794 (0.41); 7.2786 (0.42); 7.2778 (0.49); 7.2769 (0.55); 7.2761 (0.58); 7.2753 (0.64); 7.2745 (0.7); 7.2737 (0.76); 7.2729 (0.86); 7.272 (0.94); 7.2712 (1.07); 7.2704 (1.23); 7.2696 (1.49); 7.2687 (1.82); 7.2679 (2.33); 7.2671 (2.94); 7.2662 (3.74); 7.2654 (5.06); 7.2611 (192.25); 7.2547 (3.81); 7.2538 (3.23); 7.253 (2.83); 7.2522 (2.43); 7.2514 (2.11); 7.2505 (1.87); 7.2497 (1.63); 7.2489 (1.46); 7.2481 (1.34); 7.2473 (1.19); 7.2464 (1.08); 7.2456 (0.98); 7.2448 (0.9); 7.244 (0.85); 7.2432 (0.8); 7.2424 (0.74); 7.2415 (0.69); 7.2407 (0.66); 7.2399 (0.64); 7.2391 (0.6); 7.2383 (0.57); 7.2375 (0.53); 7.2367 (0.45); 7.2359 (0.42); 7.235 (0.4); 7.2342 (0.37); 7.2334 (0.36); 6.9972 (1.04); 3.4954 (0.62); 3.3334 (2.82); 3.3134 (7.05); 3.2924 (7.11); 3.2724 (2.95); 2.0065 (4.84); 1.0509 (1.6); 1.0382 (3.54); 1.0308 (5); 1.0258 (2.89); 1.0181 (9.87); 1.0108 (6.1); 1.0056 (6.36); 0.998 (10.45); 0.991 (2.94); 0.9855 (6); 0.978 (4.34); 0.9654 (2.19); 0.6018 (2.95); 0.5931 (3.92); 0.5896 (4.37); 0.5806 (10.36); 0.5718 (7.82); 0.5688 (7.26); 0.5673 (7.47); 0.5595 (8.59); 0.5575 (7.86); 0.5514 (5); 0.5467 (5.83); 0.5368 (5.23); 0.4868 (3.8); 0.476 (6.02); 0.473 (5.9); 0.4666 (4.43); 0.4622 (7.93); 0.4557 (7.7); 0.4528 (7.61); 0.4508 (5.62); 0.4416 (11.14); 0.4383 (6.12); 0.4349 (4.86); 0.4304 (4.6); 0.4246 (6.85); 0.421 (6.58); 0.415 (8.63); 0.4109 (4.81); 0.4022 (13.33); 0.3918 (11.99); 0.3891 (8.96); 0.3849 (8.61); 0.3792 (7.43); 0.376 (8.08); 0.3721 (11.26); 0.3629 (10.11); 0.3588 (5.94); 0.3498 (7.54); 0.3403 (4.09); 0.327 (2.11); 0.0495 (0.38); 0.0082 (1.65); 0 (60.44);

Compound No. 1760, solvent: [DMSO], 400 MHz

12.6667 (1.42); 12.6321 (0.74); 7.9501 (3.49); 7.8416 (2.08); 4.2547 (6.73); 4.0786 (3.51); 3.692 (16); 3.6253 (9.73); 3.3395 (7.36); 3.1678 (0.83); 2.9968 (8.75); 2.9052 (15.5); 2.6703 (0.59); 2.5237 (1.54); 2.519 (2.35); 2.5104 (32.37); 2.5058 (70.23); 2.5011 (97.68); 2.4965 (68.11); 2.4919 (30.19); 2.328 (0.58); −0.0002 (5.74);

Compound No. 1744, solvent: [DMSO], 400 MHz

12.6022 (2.04); 12.5494 (1.06); 7.9287 (3.24); 7.8172 (1.13); 5.2344 (1.23); 5.2224 (1.11); 4.365 (0.75); 4.353 (0.78); 4.203 (1.09); 4.199 (1.03); 4.1852 (3.67); 4.1813 (3.21); 4.1763 (0.74); 4.1674 (3.9); 4.1636 (3.13); 4.1582 (1.48); 4.1495 (1.33); 4.146 (1.04); 4.1403 (1.43); 4.1365 (0.78); 4.1224 (0.55); 4.1185 (1.52); 4.1007 (1.38); 4.0916 (0.65); 4.0739 (0.66); 3.3452 (49.88); 3.3109 (1.59); 3.1808 (0.57); 3.1678 (0.89); 3.1563 (0.79); 3.1492 (0.8); 2.675 (0.72); 2.6703 (0.98); 2.6657 (0.7); 2.5238 (2.88); 2.5191 (4.05); 2.5104 (53.76); 2.5058 (116.61); 2.5012 (162.08); 2.4966 (113.24); 2.492 (50.12); 2.4718 (0.61); 2.4672 (0.99); 2.4624 (1.02); 2.4558 (1.44); 2.4512 (1.68); 2.4466 (1.14); 2.4421 (0.52); 2.3327 (0.69); 2.328 (0.97); 2.3234 (0.68); 2.1858 (0.67); 2.1556 (0.68); 2.0735 (0.63); 1.6969 (1.61); 1.6719 (2.09); 1.6439 (0.73); 1.5403 (0.77); 1.4344 (0.53); 1.4024 (0.61); 1.3904 (0.54); 1.3652 (0.71); 1.3381 (0.63); 1.3208 (0.56); 1.303 (0.64); 1.2852 (0.68); 1.2819 (0.68); 1.251 (7.56); 1.2333 (16); 1.2155 (7.23); 1.2089 (4.57); 1.1911 (9.37); 1.1734 (4.2); −0.0002 (12.43);

Compound No. 1759, solvent: [DMSO], 400 MHz

12.3685 (6.98); 8.0101 (8.23); 7.4186 (0.79); 7.2856 (7.55); 7.2666 (16); 7.2191 (3.15); 7.2018 (3.77); 3.3721 (0.87); 3.345 (27.53); 3.3183 (275.19); 3.2846 (1.77); 3.2677 (1.76); 3.169 (0.93); 2.6752 (1.48); 2.6705 (2.03); 2.6659 (1.42); 2.5326 (0.71); 2.524 (5.3); 2.5193 (8.13); 2.5106 (121.54); 2.506 (265.32); 2.5014 (371.76); 2.4968 (257.76); 2.4922 (112.48); 2.4723 (1.26); 2.4677 (1.5); 2.4632 (0.85); 2.4555 (0.94); 2.4507 (1.6); 2.4462 (1.49); 2.3329 (1.45); 2.3282 (2.05); 2.3237 (1.64); 2.0735 (1.31); 0.0079 (0.73); −0.0002 (26.08); −0.0085 (0.74);

Compound No. 1429, solvent: [DMSO], 400 MHz

12.3898 (4.34); 8.0289 (5.63); 7.4321 (0.59); 7.3011 (1.81); 7.2808 (5.98); 7.2643 (16); 7.2167 (2.19); 7.2009 (2.51); 3.349 (22.41); 3.3181 (110.81); 3.1894 (0.54); 2.6755 (0.75); 2.6709 (1.06); 2.6663 (0.76); 2.5411 (0.61); 2.5243 (3.17); 2.5196 (4.85); 2.5109 (58.69); 2.5064 (124.7); 2.5018 (171.94); 2.4972 (120.81); 2.4927 (54.65); 2.4691 (0.61); 2.4567 (0.57); 2.4519 (0.56); 2.3332 (0.74); 2.3286 (1.04); 2.324 (0.74); 2.0738 (6.83); 0.008 (1.39); −0.0002 (41.66); −0.0086 (1.25);

Compound No. 1414, solvent: [DMSO], 400 MHz

12.5934 (0.87); 7.9437 (2.85); 7.826 (1); 5.2375 (1.3); 5.2264 (1.15); 4.41 (0.51); 4.374 (0.76); 4.3627 (0.83); 4.2059 (1.03); 4.2017 (1.07); 4.188 (3.41); 4.184 (3.27); 4.1701 (3.64); 4.1663 (3.27); 4.1632 (1.73); 4.157 (0.53); 4.1523 (1.25); 4.1485 (1.13); 4.1452 (1.48); 4.1405 (0.68); 4.1272 (0.53); 4.1229 (1.51); 4.1052 (1.4); 4.0961 (0.67); 4.0783 (0.68); 3.3226 (40.15); 3.1853 (0.58); 3.16 (0.82); 3.1532 (0.82); 2.5257 (0.66); 2.521 (0.97); 2.5123 (12.82); 2.5078 (27.71); 2.5032 (38.53); 2.4986 (26.92); 2.494 (11.99); 2.189 (0.7); 2.1559 (0.7); 2.0746 (0.94); 1.7023 (1.76); 1.6906 (1.49); 1.6766 (2.23); 1.6579 (1.06); 1.6481 (0.73); 1.6428 (0.79); 1.634 (0.5); 1.5772 (0.51); 1.5452 (0.78); 1.436 (0.58); 1.4036 (0.7); 1.3938 (0.65); 1.3708 (0.81); 1.3627 (0.74); 1.3454 (0.65); 1.3208 (0.53); 1.2863 (0.71); 1.2534 (7.71); 1.2357 (16); 1.2179 (7.31); 1.2105 (4.6); 1.1927 (9.44); 1.175 (4.24); −0.0002 (12.05);

Compound No. 1430, solvent: [DMSO], 400 MHz

12.6611 (1.57); 12.62 (0.81); 7.9667 (2.97); 7.8518 (1.72); 4.2585 (6.39); 4.0837 (3.19); 3.6932 (16); 3.6262 (9.49); 3.321 (36.53); 2.997 (8.57); 2.9043 (14.86); 2.5241 (1.23); 2.5193 (1.86); 2.5107 (27.45); 2.5061 (60.41); 2.5015 (84.12); 2.4969 (58.33); 2.4923 (25.83); 0.0081 (0.77); −0.0002 (26.39); −0.0085 (0.75);

Compound No. 1304, solvent: [DMSO], 400 MHz

12.6305 (2.24); 12.5773 (1.45); 7.95 (2.75); 7.8344 (1.01); 5.2351 (1.31); 4.444 (0.58); 4.359 (0.91); 4.2031 (1.11); 4.1853 (3.71); 4.1812 (3.07); 4.1675 (3.95); 4.1636 (2.93); 4.1586 (1.5); 4.1499 (1.34); 4.1408 (1.34); 4.1154 (1.43); 4.0978 (1.51); 4.0881 (0.68); 4.0709 (0.73); 3.3746 (1.5); 3.3233 (286.83); 3.2736 (2.27); 3.1672 (3.68); 3.1507 (0.83); 2.6747 (1.4); 2.6701 (1.88); 2.6654 (1.49); 2.5566 (1.19); 2.5519 (1.24); 2.5404 (0.96); 2.5237 (4.12); 2.5189 (6.1); 2.5102 (108.85); 2.5056 (240.21); 2.501 (335.73); 2.4964 (234.64); 2.4918 (103.77); 2.4556 (2.09); 2.451 (2.81); 2.4462 (2.1); 2.3326 (1.42); 2.3279 (1.91); 2.3232 (1.39); 2.1885 (0.71); 2.1499 (0.74); 2.0733 (1.16); 1.6973 (1.63); 1.6709 (2.06); 1.5328 (0.82); 1.3992 (0.72); 1.3634 (0.84); 1.2844 (0.79); 1.2507 (7.45); 1.233 (16); 1.2152 (7.19); 1.2092 (4.89); 1.1914 (9.87); 1.1736 (4.39); 0.008 (3.05); −0.0002 (112.88); −0.0085 (3.45); −0.0503 (1.04);

Compound No. 112, solvent: [DMSO], 400 MHz

13.0274 (1.41); 8.2688 (10.45); 3.3672 (2.14); 3.3528 (2.87); 3.3491 (6.77); 3.3391 (3.28); 3.335 (7.71); 3.3311 (7.95); 3.3212 (3.67); 3.317 (7.61); 3.3133 (3.59); 3.3032 (1.48); 3.299 (2.6); 2.525 (0.43); 2.5204 (0.61); 2.5116 (15.72); 2.507 (35.22); 2.5024 (50); 2.4978 (35.4); 2.4933 (16.12); 2.3568 (12.34); 2.3513 (12.48); 2.3294 (0.63); 2.0864 (0.41); 1.2335 (0.37); 1.1581 (1.09); 1.151 (14.93); 1.1399 (2.35); 1.133 (31.76); 1.1217 (1.41); 1.115 (14.55); −0.0002 (0.91);

Compound No. 552, solvent: [DMSO], 400 MHz

8.3079 (1.3); 3.409 (0.4); 3.3612 (50); 3.3393 (1.6); 3.3355 (1.38); 3.3211 (1.03); 2.7 (0.42); 2.6797 (0.45); 2.5138 (3.11); 2.5094 (6.86); 2.5048 (9.58); 2.5003 (6.8); 2.4958 (3.05); 1.1932 (1.03); 1.1745 (2.2); 1.1545 (2.35); 1.1362 (3.39); 1.1181 (1.54); −0.0002 (0.7);

Compound No. 445, solvent: [CDCl3], 400 MHz

9.4566 (0.52); 8.4679 (3.52); 7.2604 (47.18); 7.2597 (50); 3.0201 (0.5); 3.0101 (0.59); 3.0019 (1.03); 2.9918 (1.07); 2.9836 (0.59); 2.9736 (0.52); 2.3725 (3.77); 2.3665 (7.99); 2.3603 (4.12); 1.552 (0.83); 0.8935 (0.69); 0.8795 (1.65); 0.8758 (2.34); 0.8626 (2.28); 0.8578 (1.84); 0.8448 (0.92); 0.6536 (0.82); 0.6435 (1.66); 0.6406 (1.76); 0.6369 (1.71); 0.6307 (1.76); 0.6269 (1.65); 0.6133 (0.64); 0.0005 (11.23); −0.0002 (11.34); −0.0077 (0.36);

Compound No. 476, solvent: [DMSO], 400 MHz

13.0388 (0.79); 8.2434 (5.53); 4.1403 (0.31); 4.119 (0.63); 4.1037 (0.88); 4.0879 (0.67); 4.0668 (0.32); 3.3383 (40.8); 2.5261 (0.44); 2.5125 (16.29); 2.5082 (36.06); 2.5037 (50); 2.4993 (35.83); 2.4951 (16.59); 2.3753 (6); 2.3305 (0.37); 1.652 (0.38); 1.6355 (0.71); 1.6158 (0.88); 1.5995 (0.85); 1.583 (0.54); 1.5067 (0.67); 1.492 (0.56); 1.4841 (0.7); 1.4724 (1.11); 1.4583 (0.81); 1.4504 (0.94); 1.4358 (0.75); 1.3126 (0.98); 1.2988 (1.06); 1.2918 (0.95); 1.2783 (1.57); 1.2648 (0.75); 1.258 (0.72); 1.2441 (0.66); 1.1485 (9.94); 1.1321 (9.88); 0.8876 (19.3); 0.8711 (19.03); 0.0008 (5.94); −0.0002 (6.66);

Compound No. 221, solvent: [DMSO], 400 MHz

12.968 (0.34); 8.7347 (0.35); 8.5995 (0.64); 8.2534 (5.03); 3.678 (1.81); 3.464 (1.9); 2.8409 (8.22); 2.8316 (8.26); 2.8292 (8.62); 2.6721 (0.32); 2.5531 (0.37); 2.5059 (43.69); 2.5031 (50); 2.499 (35.9); 2.3773 (7.2); 2.3303 (0.34); 2.076 (0.46); 0.0025 (0.64); −0.0002 (0.87);

Compound No. 771, solvent: [DMSO], 400 MHz

13.0386 (0.83); 8.2817 (6.7); 3.3375 (18.09); 2.8459 (10.28); 2.8346 (10.85); 2.7386 (2.31); 2.7203 (2.37); 2.6723 (0.43); 2.5033 (50); 2.0761 (0.58); 1.2097 (4.54); 1.191 (9.28); 1.1724 (4.47); 0.002 (2.91); −0.0002 (3.26);

Compound No. 777, solvent: [DMSO], 400 MHz

13.0215 (0.62); 8.2677 (5.39); 4.4369 (0.57); 4.4165 (1.12); 4.3971 (1.13); 4.3765 (0.59); 3.3317 (50); 2.7322 (1.67); 2.7145 (1.72); 2.6958 (0.69); 2.6721 (0.33); 2.5027 (46.72); 2.4988 (36.24); 2.2947 (0.38); 2.2675 (1.39); 2.258 (1.33); 2.2488 (1.47); 2.2283 (0.7); 2.2211 (0.57); 2.076 (1.12); 2.0535 (1.3); 2.0301 (1.72); 2.003 (1.15); 1.9793 (0.36); 1.7411 (0.83); 1.7274 (1.35); 1.7172 (1.5); 1.7065 (1.61); 1.7008 (1.86); 1.6828 (1.12); 1.6751 (0.93); 1.6566 (0.37); 1.2095 (3.39); 1.191 (7.1); 1.1723 (3.34); −0.0002 (3.77); −0.0019 (3.46);

Compound No. 783, solvent: [DMSO], 400 MHz

12.8482 (0.77); 8.0788 (8.67); 3.1459 (0.34); 3.0965 (42.3); 2.9793 (4.08); 2.9636 (6.42); 2.948 (4.2); 2.5036 (2.59); 2.4853 (2.66); 2.4676 (1.19); 2.4545 (0.71); 2.4355 (0.88); 2.2657 (50); 2.2621 (38.93); 1.8375 (0.65); 0.9742 (5.55); 0.9556 (11.57); 0.9369 (5.47); 0.8331 (0.52); 0.8208 (1.04); 0.8132 (0.95); 0.8018 (1.57); 0.7898 (0.99); 0.7835 (1.12); 0.7709 (0.63); 0.7482 (0.3); 0.3076 (0.68); 0.2918 (0.64); 0.2405 (1.26); 0.2296 (4.26); 0.2256 (4.47); 0.2152 (1.92); 0.2091 (4.28); 0.2059 (4.23); 0.1959 (1.5); 0.0774 (0.82); 0.0627 (0.92); 0.0507 (0.32); 0.025 (1.52); 0.0129 (5.49); −0.0002 (5.24); −0.0118 (1.16); −0.2377 (3.96); −0.2395 (4);

Compound No. 793, solvent: [DMSO], 400 MHz

13.0433 (0.44); 8.2789 (3.27); 3.3815 (1.21); 3.3559 (0.73); 3.3286 (50); 3.3069 (1.38); 3.2781 (1.55); 2.7363 (1.15); 2.7179 (1.2); 2.6715 (0.4); 2.5577 (1.11); 2.5026 (49.8); 2.4523 (1.51); 2.0755 (0.58); 1.6506 (0.33); 1.6335 (0.68); 1.617 (0.91); 1.6 (0.81); 1.5838 (0.47); 1.4527 (0.91); 1.435 (2.18); 1.4168 (2.12); 1.3995 (0.84); 1.2093 (2.17); 1.1907 (4.53); 1.1721 (2.18); 0.9664 (0.34); 0.9132 (12.44); 0.8967 (11.9); 0.874 (0.87); 0.863 (0.39); 0.8465 (0.39); 0.0022 (2.22); −0.0002 (2.91);

Compound No. 795, solvent: [DMSO], 400 MHz

9.0032 (0.34); 8.3124 (7.83); 8.1074 (0.66); 5.9531 (0.51); 5.9403 (1.12); 5.9273 (1.08); 5.9188 (0.71); 5.9145 (1.28); 5.9128 (1.29); 5.8955 (1.59); 5.8843 (1.23); 5.8773 (1.09); 5.8715 (1.38); 5.8587 (0.74); 5.8508 (0.78); 5.8358 (0.37); 5.4019 (1.55); 5.4006 (1.53); 5.3586 (1.31); 5.326 (1.52); 5.3241 (1.71); 5.2998 (1.41); 5.2979 (1.59); 5.2315 (2.3); 5.229 (2.37); 5.1902 (1.91); 5.1884 (2.01); 5.186 (2.06); 5.1342 (2.24); 5.1324 (2.48); 5.1303 (2.39); 5.1084 (2.1); 5.1067 (2.3); 5.1045 (2.24); 3.9707 (2.59); 3.9573 (4.89); 3.9441 (2.65); 3.6691 (13.41); 3.473 (0.62); 3.4582 (1.65); 3.4436 (2.38); 3.4292 (1.63); 3.4146 (0.52); 2.7587 (0.93); 2.7398 (2.78); 2.7213 (2.86); 2.7029 (1.04); 2.6741 (0.32); 2.5083 (38.65); 2.5055 (50); 2.5014 (38.46); 2.3315 (0.3); 2.0771 (0.48); 2.0753 (0.44); 1.7615 (1.1); 1.7599 (1.08); 1.2106 (5.53); 1.192 (11.74); 1.1733 (5.48); −0.0002 (2.42); −0.0019 (2.08);

Compound No. 798, solvent: [DMSO], 400 MHz

12.9867 (0.35); 8.2831 (3.28); 4.1211 (1.84); 4.1148 (2.15); 4.1073 (2.24); 4.1013 (1.91); 3.3266 (48.45); 3.2756 (0.34); 3.1791 (1.04); 3.1741 (2.25); 3.1678 (1.14); 2.7396 (1.13); 2.7212 (1.16); 2.703 (0.46); 2.6712 (0.32); 2.5051 (36.99); 2.5017 (50); 2.4977 (38.47); 2.4467 (0.31); 2.0862 (0.46); 2.0751 (0.51); 1.2047 (2.44); 1.1862 (5.17); 1.1675 (2.41); −0.0002 (3.28); −0.0018 (2.87);

Compound No. 222, solvent: [DMSO], 400 MHz

8.2559 (1.03); 3.3364 (27.06); 2.5511 (0.36); 2.5062 (37.48); 2.5029 (50); 2.499 (38.89); 2.3777 (1.27); 2.3301 (0.31); 1.1486 (1.01); 1.1306 (1.99); 1.1126 (0.95); 0.0013 (2.83); −0.0002 (2.87);

Compound No. 222, solvent: [DMSO], 400 MHz

8.2487 (0.88); 3.6615 (0.34); 3.4433 (50); 3.3638 (0.53); 3.3461 (0.79); 3.3287 (0.82); 3.3138 (0.65); 2.5078 (9.99); 2.3821 (0.86); 2.3749 (1.44); 2.3665 (0.74); 2.071 (0.66); 1.1483 (0.96); 1.1303 (1.95); 1.1122 (0.89);

Compound No. 223, solvent: [DMSO], 400 MHz

8.2515 (1.84); 3.6604 (0.37); 3.375 (50); 3.2947 (1.27); 3.2779 (1.67); 3.2618 (1.56); 3.2452 (0.8); 2.5046 (31.98); 2.3776 (2.61); 2.0747 (0.57); 1.5642 (0.58); 1.5463 (1.17); 1.5282 (1.21); 1.5101 (0.63); 1.081 (0.43); 0.915 (1.78); 0.8965 (3.5); 0.878 (1.55); 0.0016 (1.8); −0.0002 (2.07);

Compound No. 224, solvent: [DMSO], 400 MHz

13.0023 (0.37); 8.6127 (0.33); 8.5562 (1.59); 8.275 (6.18); 8.1862 (0.37); 4.1384 (0.34); 4.1216 (0.93); 4.1051 (1.42); 4.0865 (1.48); 4.07 (0.99); 4.0533 (0.37); 3.8559 (2.28); 3.61 (3.3); 3.3468 (13.49); 2.5301 (0.49); 2.5165 (15.05); 2.512 (34.71); 2.5074 (50); 2.5028 (36.11); 2.4982 (16.7); 2.3893 (4.39); 2.3812 (8.23); 2.3725 (4.44); 2.3343 (0.33); 2.0782 (0.89); 1.1942 (29.69); 1.1874 (10.73); 1.1777 (29.95); 1.1711 (10.29);

Compound No. 225, solvent: [DMSO], 400 MHz

11.133 (0.61); 7.8959 (6.09); 3.33 (2.14); 2.832 (0.6); 2.8222 (0.88); 2.8139 (1.35); 2.8038 (1.4); 2.7953 (0.96); 2.7856 (0.7); 2.5465 (0.75); 2.5354 (1.08); 2.5285 (1.23); 2.5271 (1.31); 2.5187 (3.25); 2.511 (15.64); 2.5064 (34.22); 2.5017 (50); 2.4971 (35.68); 2.4925 (17.34); 2.3286 (0.31); 2.2192 (5.41); 2.2101 (11.43); 2.2009 (6.18); 2.0728 (0.96); 0.7214 (1.28); 0.709 (3.51); 0.704 (4.48); 0.6914 (4.34); 0.6863 (3.86); 0.6744 (2.21); 0.6574 (1.73); 0.6546 (3.46); 0.6519 (2.7); 0.6484 (3.68); 0.6439 (2.84); 0.6368 (3.53); 0.6312 (7.49); 0.6272 (6.48); 0.6239 (3.25); 0.6206 (6.04); 0.6167 (5.46); 0.6146 (3.33); 0.6112 (3.67); 0.6008 (0.78); 0.5897 (0.47); 0.452 (1.5); 0.4406 (4.05); 0.4355 (3.87); 0.4303 (3.79); 0.4255 (4.02); 0.4135 (1.43);

Compound No. 226, solvent: [DMSO], 400 MHz

12.992 (0.31); 8.8052 (0.33); 8.5871 (0.46); 8.2755 (5.66); 7.8157 (0.59); 3.6542 (1.38); 3.4157 (4.58); 3.3311 (2.21); 3.3139 (3.94); 3.2988 (3.81); 3.2818 (1.85); 3.1687 (0.35); 2.7867 (0.8); 2.7711 (1.26); 2.75 (1.29); 2.7349 (0.88); 2.5089 (36.4); 2.5048 (50); 2.5005 (37.53); 2.3842 (3.97); 2.3762 (7.43); 2.3678 (4.2); 2.3316 (0.34); 2.0877 (1.3); 2.0775 (0.71); 1.5545 (0.51); 1.5368 (1.31); 1.5273 (2.31); 1.5169 (2.45); 1.5095 (3.66); 1.4915 (2.79); 1.4789 (0.9); 1.4722 (0.98); 1.376 (0.52); 1.3703 (0.47); 1.3572 (1.95); 1.3522 (1.69); 1.3377 (3.43); 1.3192 (3.4); 1.3015 (1.82); 1.296 (1.38); 1.2839 (0.48); 1.2776 (0.37); 0.918 (6.19); 0.8998 (12.67); 0.8859 (9.32); 0.8816 (6.14); 0.8676 (3.86); −0.0002 (7.56);

Compound No. 227, solvent: [DMSO], 400 MHz

8.6259 (0.33); 8.2521 (2.54); 8.2216 (1.54); 4.434 (0.34); 4.4141 (0.63); 4.3939 (0.64); 4.3728 (0.37); 3.717 (1.7); 3.5604 (2.26); 2.6725 (0.31); 2.5516 (0.82); 2.5032 (50); 2.4992 (39.34); 2.4593 (0.83); 2.3977 (1.24); 2.3888 (2.72); 2.3806 (2.82); 2.3747 (3.53); 2.3659 (1.9); 2.3295 (0.37); 2.2608 (0.72); 2.2416 (0.77); 2.2218 (0.41); 2.179 (0.39); 2.1559 (0.51); 2.0845 (0.61); 2.0757 (0.68); 2.0592 (1.08); 2.0338 (1.21); 2.0113 (0.7); 1.786 (0.42); 1.7579 (0.45); 1.7367 (0.58); 1.724 (0.63); 1.7133 (0.82); 1.7036 (0.84); 1.6965 (0.96); 1.679 (0.54); 1.6708 (0.48); −0.0002 (5.17); −0.0019 (4.75);

Compound No. 233, solvent: [DMSO], 400 MHz

12.7621 (0.64); 8.0452 (5.6); 5.5192 (0.32); 3.1408 (0.32); 3.087 (15.43); 2.9787 (3.2); 2.962 (4.05); 2.9474 (3.28); 2.4355 (0.31); 2.322 (0.36); 2.3175 (0.31); 2.289 (0.34); 2.2844 (0.51); 2.2756 (15.38); 2.271 (35.18); 2.2664 (50); 2.2618 (35.59); 2.2572 (16.07); 2.221 (0.38); 2.2164 (0.53); 2.2118 (0.41); 2.154 (3.39); 2.1457 (6.5); 2.137 (3.61); 2.0977 (0.33); 2.0934 (0.36); 1.8383 (0.5); 0.829 (0.31); 0.8169 (0.65); 0.8116 (0.63); 0.8077 (0.51); 0.7996 (1.22); 0.7914 (0.51); 0.7874 (0.68); 0.7824 (0.66); 0.7797 (0.74); 0.7677 (0.39); 0.7624 (0.3); 0.24 (1.08); 0.2293 (3.15); 0.225 (3.34); 0.2201 (1.38); 0.2148 (1.56); 0.2092 (3.43); 0.2046 (3.11); 0.1948 (1.39); 0.0259 (1.3); 0.0155 (3.48); 0.0118 (3.58); 0.0037 (3.15); −0.0002 (3.75); −0.0112 (0.94); −0.236 (1.47);

Compound No. 243, solvent: [DMSO], 400 MHz

12.9731 (0.41); 8.251 (3.21); 3.6158 (0.41); 3.363 (3.77); 3.352 (3.83); 3.3357 (3.14); 3.3175 (2.36); 3.3008 (1.31); 2.5069 (38.1); 2.5032 (50); 2.4997 (38.49); 2.3839 (2.12); 2.3761 (3.99); 2.3675 (2.38); 2.33 (0.34); 1.6514 (0.38); 1.6345 (0.81); 1.6178 (1.07); 1.6012 (0.94); 1.5844 (0.53); 1.449 (0.99); 1.4313 (2.3); 1.413 (2.23); 1.3957 (0.82); 0.9105 (12.69); 0.8938 (12.64); 0.8734 (2.91); 0.0015 (7.1); 0.0004 (6.65); −0.0002 (7.27);

Compound No. 245, solvent: [DMSO], 400 MHz

12.9256 (0.6); 8.8875 (0.57); 8.264 (11.72); 8.1541 (0.47); 5.9471 (1.08); 5.9341 (2.44); 5.9213 (2.29); 5.9083 (2.75); 5.9041 (1.44); 5.8954 (1.33); 5.8911 (2.96); 5.8782 (2.69); 5.8654 (2.97); 5.8525 (1.36); 5.756 (0.57); 5.2409 (1.47); 5.2366 (4.54); 5.2323 (4.79); 5.228 (1.64); 5.1979 (1.31); 5.1936 (4.05); 5.1893 (4.21); 5.185 (1.45); 5.1374 (1.63); 5.1334 (4.89); 5.1294 (4.71); 5.1255 (1.53); 5.1116 (1.51); 5.1077 (4.56); 5.1037 (4.46); 5.0997 (1.45); 3.9716 (2.45); 3.9675 (4.5); 3.9632 (3.04); 3.9574 (4.93); 3.9537 (7.66); 3.9444 (3.2); 3.9401 (4.62); 3.9359 (2.41); 3.4657 (0.78); 3.3667 (3.05); 2.6735 (0.31); 2.527 (0.58); 2.5223 (0.79); 2.5136 (15.95); 2.5091 (35.67); 2.5045 (50); 2.4999 (35.19); 2.4953 (15.63); 2.3882 (7.8); 2.3796 (15.38); 2.3707 (8.48); 2.3314 (0.32); 2.0875 (0.99); 2.0757 (0.67); −0.0002 (1.47);

Compound No. 247, solvent: [DMSO], 400 MHz

12.9086 (0.38); 8.8968 (0.51); 8.6098 (1.87); 8.2584 (10.84); 4.8631 (5.16); 4.8612 (5.55); 4.8593 (4.93); 4.826 (4.36); 4.8223 (5.58); 4.8186 (4.21); 3.8865 (6.34); 3.872 (6.58); 3.6943 (4.81); 3.4504 (2.83); 3.148 (0.33); 2.673 (0.33); 2.5266 (0.67); 2.5218 (0.95); 2.5131 (15.95); 2.5086 (35.36); 2.5039 (50); 2.4993 (34.98); 2.4947 (15.22); 2.4474 (0.43); 2.4428 (0.45); 2.3885 (7.4); 2.38 (14.34); 2.3713 (7.54); 2.0749 (0.55); 1.7604 (0.6); 1.7256 (23.96); 1.7245 (24.57); 0.9096 (1.48); 0.8929 (1.41); −0.0002 (0.72);

Compound No. 248, solvent: [DMSO], 400 MHz

8.3011 (1.32); 8.0237 (0.69); 7.6598 (0.51); 7.5785 (0.55); 7.5139 (0.43); 7.4915 (0.51); 7.3044 (0.62); 7.2819 (0.45); 7.2582 (2.44); 7.2385 (5.15); 7.2189 (3.28); 7.1995 (0.63); 7.1765 (0.34); 7.0748 (5.12); 7.0693 (5.19); 7.0638 (4.5); 7.0446 (3.12); 7.0108 (0.32); 6.8625 (2.64); 6.8588 (2.62); 6.8442 (2.38); 6.8384 (2.41); 4.0574 (0.94); 4.0396 (2.89); 4.0218 (2.92); 4.004 (0.99); 3.8377 (0.32); 3.7943 (0.38); 3.745 (4.68); 3.7019 (5.62); 3.68 (0.44); 3.6358 (0.47); 3.4015 (0.64); 3.3576 (1.1); 3.3317 (6.83); 3.2886 (5.58); 3.1638 (0.54); 3.1196 (0.44); 2.6727 (0.31); 2.5082 (37.45); 2.5038 (50); 2.4993 (36.11); 2.3305 (0.33); 1.9873 (12.41); 1.9091 (6.68); 1.5881 (2.39); 1.569 (2.38); 1.5524 (28.49); 1.2371 (0.58); 1.1964 (3.35); 1.1786 (6.69); 1.1608 (3.29); 0.9141 (0.35); 0.9055 (0.43); 0.8887 (0.47); 0.8274 (0.39); 0.8214 (0.38); 0.8047 (0.35); −0.0002 (2.99);

Compound No. 249, solvent: [DMSO], 400 MHz

8.5933 (0.78); 8.2694 (3.47); 3.974 (0.31); 3.9574 (0.65); 3.9405 (0.73); 3.9206 (0.67); 3.9035 (0.32); 3.8559 (1.09); 3.6684 (1.68); 3.3741 (9.33); 2.5164 (15.24); 2.5119 (34.89); 2.5072 (50); 2.5026 (36.47); 2.498 (17.07); 2.3922 (2.29); 2.3837 (4.45); 2.3751 (2.49); 2.3342 (0.31); 2.0783 (0.71); 1.5997 (0.32); 1.5605 (0.51); 1.5418 (1.87); 1.5238 (2.57); 1.5062 (2.02); 1.488 (0.56); 1.4615 (0.42); 1.4427 (0.3); 1.1625 (8.17); 1.1459 (8.14); 0.9187 (1.43); 0.9041 (3.79); 0.9 (3.59); 0.8857 (7.88); 0.867 (3.28);

Compound No. 256, solvent: [DMSO], 400 MHz

8.5969 (0.93); 8.2677 (3.34); 4.1141 (0.55); 4.0988 (0.43); 3.6729 (2.07); 3.3883 (8.21); 2.5569 (0.4); 2.5159 (17.58); 2.5114 (37.24); 2.5068 (50); 2.5022 (34.86); 2.4977 (15.48); 2.4575 (0.35); 2.382 (4.22); 2.3734 (2.33); 2.3339 (0.31); 2.0779 (0.89); 1.6602 (0.49); 1.6434 (0.61); 1.6246 (0.66); 1.6077 (0.56); 1.5924 (0.34); 1.5205 (0.44); 1.5059 (0.37); 1.4985 (0.46); 1.4867 (0.73); 1.4721 (0.54); 1.4642 (0.61); 1.4496 (0.53); 1.4203 (0.38); 1.4053 (0.34); 1.3396 (0.31); 1.3179 (0.83); 1.3035 (0.93); 1.2965 (0.63); 1.2832 (1.02); 1.2693 (0.61); 1.2628 (0.47); 1.2487 (0.43); 1.1723 (2.76); 1.1559 (8.97); 1.1395 (6.27); 0.9068 (3.09); 0.8928 (15.35); 0.8764 (14.08); 0.8711 (4.01); 0.8546 (2.95);

Compound No. 258, solvent: [DMSO], 400 MHz

8.5998 (0.82); 8.2564 (3.86); 3.9055 (0.57); 3.8892 (0.82); 3.884 (0.66); 3.8676 (0.81); 3.8562 (1.41); 3.8519 (0.64); 3.6789 (1.82); 3.3864 (5); 2.53 (0.44); 2.5253 (0.61); 2.5165 (14.91); 2.512 (34.65); 2.5074 (50); 2.5027 (36.15); 2.4981 (16.68); 2.3897 (2.76); 2.3818 (4.99); 2.3733 (2.72); 2.3343 (0.3); 2.0784 (0.73); 1.7904 (0.61); 1.7737 (1); 1.7574 (1.03); 1.741 (0.66); 1.1623 (0.63); 1.1458 (0.68); 1.1189 (8.63); 1.102 (8.58); 0.9268 (0.64); 0.9097 (9.6); 0.907 (9.63); 0.8996 (1.85); 0.8927 (8.98); 0.8901 (9.23); 0.8677 (0.33);

Compound No. 266, solvent: [DMSO], 400 MHz

8.2867 (3.35); 3.6241 (0.44); 3.5031 (0.46); 3.4919 (1.37); 3.4812 (2.22); 3.4713 (5.9); 3.4635 (2.84); 3.4516 (0.69); 3.3971 (0.48); 3.3409 (35.55); 3.274 (14.91); 3.2725 (14.52); 2.6705 (0.31); 2.5053 (37.53); 2.5019 (50); 2.498 (38.45); 2.3849 (2.23); 2.3772 (4.11); 2.3688 (2.32); 2.3286 (0.31); −0.0002 (3.49); −0.002 (3.24);

Compound No. 272, solvent: [DMSO], 400 MHz

12.9871 (0.54); 8.2768 (3.79); 4.2082 (0.53); 4.1914 (0.66); 4.1719 (0.52); 3.4269 (1.05); 3.4129 (1.06); 3.4028 (2.03); 3.3888 (1.96); 3.3662 (0.39); 3.3573 (1.8); 3.3443 (2.01); 3.333 (1.65); 3.3148 (31.37); 3.2814 (25.74); 3.2647 (0.53); 3.2314 (0.36); 2.6708 (0.31); 2.5617 (0.35); 2.5573 (0.49); 2.5527 (0.45); 2.5243 (0.3); 2.5196 (0.47); 2.5109 (15.37); 2.5064 (35.17); 2.5017 (50); 2.4971 (35.56); 2.4926 (16.03); 2.4564 (0.47); 2.4518 (0.67); 2.4472 (0.51); 2.386 (2.27); 2.3777 (4.36); 2.3691 (2.43); 2.3287 (0.37); 2.0738 (0.73); 1.1664 (8.48); 1.1495 (8.47); −0.0002 (2.42);

Compound No. 274, solvent: [DMSO], 400 MHz

12.9397 (0.33); 8.2519 (2.85); 3.6289 (0.38); 3.4035 (2.13); 3.3879 (4.42); 3.3717 (4.63); 3.3455 (29.66); 3.2435 (9.67); 3.2412 (13.02); 2.6718 (0.31); 2.5027 (50); 2.4476 (0.48); 2.3783 (3.84); 2.3293 (0.34); 2.0864 (0.35); 2.075 (0.43); 1.7849 (0.39); 1.7692 (1.44); 1.7526 (2.12); 1.7368 (1.39); 1.7202 (0.36); 0.0023 (3.29); −0.0002 (4.22);

Compound No. 661, solvent: [DMSO], 400 MHz

13.0033 (0.79); 8.7891 (0.41); 8.303 (6.25); 3.3367 (5.33); 2.8494 (11.19); 2.8377 (11.05); 2.7178 (2.13); 2.6996 (2.15); 2.5119 (21.59); 2.5075 (40.38); 2.503 (50); 2.4984 (34.59); 2.0866 (0.45); 2.0744 (0.4); 1.2029 (4.42); 1.1843 (9.35); 1.1657 (4.28); −0.0002 (6.37);

Compound No. 662, solvent: [DMSO], 400 MHz

13.0264 (0.52); 8.3123 (3.14); 3.3726 (0.83); 3.3551 (1.56); 3.337 (2.22); 3.3164 (26.5); 3.2666 (0.41); 2.7174 (1.06); 2.699 (1.07); 2.5568 (0.96); 2.5524 (0.99); 2.5058 (39.26); 2.5015 (50); 2.4972 (36.04); 2.4511 (0.64); 1.2046 (2.18); 1.1862 (4.6); 1.1673 (2.24); 1.1572 (3.17); 1.1392 (6.35); 1.1212 (2.98); −0.0002 (4.68);

Compound No. 663, solvent: [DMSO], 400 MHz

13.0064 (0.5); 8.3049 (3.28); 3.3181 (22.78); 3.285 (1.91); 3.2701 (1.84); 3.2525 (0.94); 2.7167 (1.03); 2.6988 (1.06); 2.5109 (20.03); 2.5065 (38.97); 2.5019 (50); 2.4973 (35.14); 2.4929 (16.17); 2.0736 (0.3); 1.5728 (0.96); 1.5544 (1.87); 1.5367 (1.9); 1.5185 (1.08); 1.2036 (2.25); 1.185 (4.8); 1.1665 (2.18); 0.9204 (3.45); 0.902 (7.2); 0.8834 (3.04); −0.0002 (5.97);

Compound No. 664, solvent: [DMSO], 400 MHz

8.3088 (1.27); 4.1058 (0.32); 4.0891 (0.32); 3.3946 (50); 2.7128 (0.49); 2.6946 (0.49); 2.5106 (9.3); 2.5063 (11.76); 2.5023 (8.46); 2.07 (0.35); 1.1962 (4.53); 1.1798 (4.69); 1.1664 (0.98);

Compound No. 665, solvent: [DMSO], 400 MHz

12.9601 (1.09); 8.8178 (0.69); 8.2676 (9.4); 3.3309 (8.3); 2.8946 (1.19); 2.8848 (1.59); 2.8764 (2.56); 2.8664 (2.56); 2.8581 (1.67); 2.8483 (1.22); 2.8384 (0.45); 2.7072 (3.31); 2.6884 (3.36); 2.5134 (20.06); 2.509 (39.01); 2.5044 (50); 2.4999 (35.2); 2.4955 (16.28); 2.0876 (0.4); 1.1999 (6.86); 1.1813 (14.69); 1.1626 (6.66); 0.7817 (1.5); 0.7691 (4.68); 0.7637 (5.92); 0.7512 (6.07); 0.7457 (4.52); 0.7339 (2.02); 0.7142 (0.56); 0.6901 (0.73); 0.6781 (0.63); 0.6093 (1.94); 0.5988 (5.49); 0.592 (5.31); 0.5829 (4.5); 0.5703 (1.39); −0.0002 (7.95);

Compound No. 666, solvent: [DMSO], 400 MHz

13.0076 (0.57); 8.3039 (4.37); 3.321 (22.36); 3.2897 (1.51); 2.7162 (1.4); 2.6981 (1.42); 2.5115 (20.6); 2.5071 (39.52); 2.5025 (50); 2.498 (34.87); 2.4936 (15.98); 2.0865 (0.44); 2.0741 (0.39); 1.5528 (0.47); 1.5347 (1.42); 1.5171 (2.17); 1.4987 (1.73); 1.4801 (0.68); 1.3823 (0.39); 1.3641 (1.33); 1.3449 (1.96); 1.3261 (1.88); 1.3084 (1.13); 1.2035 (3.02); 1.185 (6.4); 1.1662 (2.91); 0.9227 (4.56); 0.9043 (9.56); 0.886 (3.94); −0.0002 (5.02);

Compound No. 667, solvent: [DMSO], 400 MHz

12.9695 (1.15); 8.9642 (0.58); 8.2934 (9.63); 4.4423 (0.98); 4.4222 (1.94); 4.4025 (1.89); 4.3816 (1.02); 3.3303 (6.99); 2.7153 (3.04); 2.6967 (3.13); 2.5133 (19.76); 2.5088 (38.74); 2.5043 (50); 2.4997 (35.02); 2.4953 (15.86); 2.3311 (0.33); 2.2998 (0.78); 2.2733 (2.47); 2.2667 (2.42); 2.2618 (2.44); 2.2545 (2.6); 2.2436 (2.15); 2.2332 (1.26); 2.2253 (1.03); 2.0948 (0.69); 2.0876 (1.01); 2.0712 (2.23); 2.0464 (3.08); 2.0237 (2.16); 2.0176 (2.14); 1.9948 (0.69); 1.7485 (1.35); 1.7341 (2.21); 1.7247 (2.6); 1.715 (3); 1.708 (3.45); 1.6904 (1.88); 1.6824 (1.67); 1.6637 (0.7); 1.207 (6.51); 1.1884 (13.95); 1.1697 (6.28); −0.0002 (8.28);

Compound No. 685, solvent: [DMSO], 400 MHz 12.9591 (0.79); 8.9431 (0.41); 8.3183 (7.09); 5.9517 (0.61); 5.9387 (1.4); 5.9259 (1.22); 5.9129 (1.58); 5.9087 (0.84); 5.9 (0.97); 5.8956 (1.67); 5.8828 (1.39); 5.8699 (1.63); 5.857 (0.74); 5.2441 (2.72); 5.2398 (2.64); 5.201 (2.42); 5.1968 (2.34); 5.1388 (2.93); 5.1348 (2.68); 5.1131 (2.7); 5.109 (2.46); 3.9738 (2.59); 3.9604 (4.41); 3.9508 (2.21); 3.9465 (2.55); 3.3279 (13.67); 2.7205 (2.28); 2.7019 (2.34); 2.5127 (20.18); 2.5081 (39.26); 2.5035 (50); 2.499 (35.07); 2.4945 (15.82); 2.087 (0.43); 2.0749 (0.32); 1.2041 (4.97); 1.1855 (10.72); 1.1668 (4.82); −0.0002 (13.59);

Compound No. 688, solvent: [DMSO], 400 MHz

12.8955 (0.36); 9.0916 (0.56); 8.305 (6.79); 4.1254 (4.3); 4.1192 (4.79); 4.1115 (4.95); 4.1054 (4.04); 3.7405 (0.45); 3.6084 (0.62); 3.326 (15.72); 3.1821 (3.21); 3.1758 (6.24); 3.1696 (2.75); 2.7197 (2.35); 2.7011 (2.38); 2.5126 (21.19); 2.5082 (40.02); 2.5037 (50); 2.4991 (35.01); 2.4948 (16.22); 2.3309 (0.3); 2.087 (1.32); 2.075 (0.49); 1.1993 (4.82); 1.1807 (10.26); 1.1622 (4.6); −0.0002 (7.4);

Compound No. 689, solvent: [DMSO], 400 MHz

13.0222 (0.71); 8.3117 (6.54); 3.9765 (0.5); 3.9596 (1.16); 3.9427 (1.38); 3.9234 (1.1); 3.9071 (0.49); 3.3278 (16.62); 2.7169 (2.13); 2.6985 (2.17); 2.5081 (39.01); 2.5036 (50); 2.4991 (35.82); 2.0872 (0.46); 2.0753 (0.42); 1.5627 (0.79); 1.5444 (2.93); 1.5265 (4.32); 1.5087 (3.19); 1.4906 (0.91); 1.2064 (4.46); 1.1878 (9.51); 1.1659 (13.17); 1.1493 (11.55); 0.9048 (5.73); 0.8863 (11.73); 0.8677 (5.04); −0.0002 (6.35);

Compound No. 712, solvent: [DMSO], 400 MHz

8.3262 (1.58); 4.1961 (0.38); 3.4336 (0.73); 3.3652 (50); 3.2838 (5.76); 2.7133 (0.69); 2.6961 (0.7); 2.5034 (21.58); 2.0704 (0.38); 1.1987 (1.21); 1.1742 (3.04); 1.1576 (2.97); −0.0002 (0.36);

Compound No. 714, solvent: [DMSO], 400 MHz

12.9731 (0.58); 8.3074 (4.05); 3.4084 (2.5); 3.3929 (6.15); 3.3774 (4.49); 3.3607 (2.71); 3.3434 (2.03); 3.3224 (20.38); 3.2444 (24.3); 3.2399 (3.8); 2.718 (1.24); 2.6996 (1.3); 2.5115 (20.18); 2.507 (39.1); 2.5024 (50); 2.4978 (34.84); 2.4934 (15.68); 2.074 (0.32); 1.7938 (0.56); 1.7781 (1.88); 1.7614 (2.83); 1.7454 (1.77); 1.7287 (0.52); 1.2023 (2.77); 1.1836 (5.91); 1.1649 (2.67); −0.0002 (6.59);

Compound No. 700, solvent: [DMSO], 400 MHz

13.0035 (1.16); 8.3017 (4.29); 3.8286 (2.12); 3.3573 (18.18); 2.7196 (4.64); 2.5777 (4.04); 2.508 (50); 1.5765 (4.17); 1.4738 (4.2); 1.2065 (7.34); 1.1891 (8.58); 0.8912 (15.33); 0.8737 (17.75); 0.8557 (8.83); −0.0002 (3.72);

Compound No. 697, solvent: [DMSO], 400 MHz

13.0051 (1.24); 8.3008 (3.99); 3.889 (2.2); 3.3374 (24.01); 2.7128 (4.65); 2.5075 (50); 1.7743 (2.07); 1.182 (8.92); 1.1206 (10.37); 1.1041 (7.4); 0.9071 (18.7); 0.8915 (12.96); −0.0002 (3.57);

Compound No. 696, solvent: [DMSO], 400 MHz

13.0419 (2.04); 8.5883 (1.28); 8.3079 (6.12); 4.1323 (2.81); 3.3359 (25.49); 2.7164 (6.74); 2.5091 (50); 1.6274 (2.85); 1.4976 (3.09); 1.2913 (3.82); 1.1882 (18.08); 1.161 (16.92); 1.1445 (11.47); 0.8934 (30.47); 0.8773 (21.45); −0.0002 (6.23);

Compound No. 265, solvent: [DMSO], 400 MHz

13.0208 (0.34); 8.5755 (0.35); 8.3064 (5.08); 3.6445 (0.87); 3.5729 (0.35); 3.5594 (0.33); 3.5373 (2.23); 3.5226 (5.72); 3.5085 (3.62); 3.4086 (2.81); 3.3948 (5.59); 3.3806 (5.65); 3.3658 (3.68); 2.5249 (0.4); 2.5202 (0.57); 2.5114 (14.84); 2.5069 (34.81); 2.5023 (50); 2.4976 (35.84); 2.493 (16.17); 2.3889 (3.01); 2.3807 (5.74); 2.3721 (3.22); 2.3291 (0.33); 2.0738 (0.38); −0.0002 (4.93);

Compound No. 807, solvent: [DMSO], 400 MHz

13.0396 (0.37); 8.285 (4.75); 3.9244 (0.84); 3.9074 (1); 3.9014 (0.88); 3.8906 (0.47); 3.8842 (0.83); 3.3172 (4.64); 2.7516 (0.46); 2.7335 (1.34); 2.7149 (1.4); 2.6967 (0.54); 2.5134 (5.87); 2.5089 (13.58); 2.5042 (19.63); 2.4996 (14.07); 2.495 (6.37); 1.2024 (3.42); 1.1838 (7.61); 1.1651 (3.42); 1.0884 (9.3); 1.0714 (9.29); 0.9602 (0.56); 0.9093 (50); 0.8594 (0.57); −0.0002 (2.84);

Compound No. 325, solvent: [DMSO], 400 MHz 11.8151 (0.65); 7.7169 (1.31); 3.3315 (50); 2.9705 (5.95); 2.8053 (6.17); 2.5118 (3.81); 2.5073 (8.52); 2.5027 (12.06); 2.4981 (8.52); 2.4935 (3.75); 2.3595 (1.15); 2.3508 (2.23); 2.3419 (1.2); −0.0002 (0.38);

Compound No. 328, solvent: [DMSO], 400 MHz

11.7329 (1.26); 7.6992 (4.23); 3.4525 (1.05); 3.4349 (3.46); 3.4172 (3.54); 3.3995 (1.23); 3.3879 (0.58); 3.3368 (50); 3.2869 (0.64); 3.1247 (0.95); 3.107 (3.06); 3.0892 (3.13); 3.0716 (1.01); 2.5123 (7.61); 2.5078 (17.32); 2.5032 (24.58); 2.4986 (17.63); 2.494 (7.99); 2.3617 (3.55); 2.353 (7.09); 2.3439 (3.83); 2.3303 (0.31); 2.0743 (0.42); 1.1432 (3.77); 1.1256 (8.41); 1.1078 (3.76); 1.0931 (0.33); 1.0329 (3.54); 1.0153 (7.94); 0.9975 (3.46);

Compound No. 875, solvent: [DMSO], 400 MHz

11.8949 (1.33); 7.7346 (4.5); 3.6678 (0.38); 3.6016 (0.56); 3.3792 (0.36); 3.3308 (50); 2.9721 (15.73); 2.8083 (16.27); 2.7422 (0.64); 2.7235 (1.93); 2.7049 (2); 2.6864 (0.73); 2.5209 (0.33); 2.5121 (7.02); 2.5076 (15.92); 2.503 (22.59); 2.4984 (16.08); 2.4939 (7.25); 1.76 (0.69); 1.3556 (2.02); 1.1907 (4.55); 1.1721 (9.98); 1.1534 (4.45); −0.0002 (1.15);

Compound No. 883, solvent: [DMSO], 400 MHz

12.9766 (0.98); 8.2908 (5.01); 3.7814 (0.42); 3.3361 (4.49); 3.2996 (1.43); 3.2826 (2.83); 3.2672 (2.82); 3.25 (1.42); 2.7306 (0.39); 2.7157 (0.7); 2.698 (1.26); 2.6778 (1.38); 2.526 (0.56); 2.5211 (0.88); 2.5124 (16.85); 2.508 (36.46); 2.5035 (50); 2.499 (36.38); 2.4946 (17.42); 2.4522 (0.38); 2.4478 (0.51); 2.3304 (0.3); 1.5859 (0.34); 1.5674 (1.54); 1.5492 (2.97); 1.5315 (3.08); 1.5134 (1.69); 1.4953 (0.4); 1.1918 (3.06); 1.1733 (6.49); 1.1546 (2.96); 0.918 (5.34); 0.8995 (10.98); 0.8809 (4.73); 0.0079 (0.6); −0.0002 (19.78); −0.0085 (0.87);

Compound No. 885, solvent: [DMSO], 400 MHz

8.2255 (3.8); 3.7782 (0.41); 3.333 (22.2); 2.8834 (0.51); 2.8737 (0.7); 2.8652 (1.13); 2.8552 (1.14); 2.8467 (0.72); 2.837 (0.55); 2.6811 (1.45); 2.6627 (1.49); 2.5536 (0.44); 2.5477 (0.38); 2.5258 (0.77); 2.521 (1.07); 2.5123 (16.43); 2.5078 (36.11); 2.5032 (50); 2.4987 (36.29); 2.4942 (17.11); 1.1829 (3.09); 1.1644 (6.73); 1.1458 (2.99); 0.7734 (0.69); 0.7608 (1.97); 0.7555 (2.72); 0.743 (2.68); 0.7374 (2.08); 0.7257 (0.95); 0.7115 (0.32); 0.6868 (0.35); 0.6676 (0.44); 0.6643 (0.38); 0.5894 (0.82); 0.5786 (2.32); 0.5724 (2.24); 0.5681 (2.07); 0.5628 (2.07); 0.5505 (0.66); 0.008 (0.34); −0.0002 (12.95); −0.0086 (0.56);

Compound No. 908, solvent: [DMSO], 400 MHz

9.2675 (0.32); 8.2686 (6.28); 4.1214 (3.79); 4.1153 (4.25); 4.1077 (4.24); 4.1016 (3.8); 3.7801 (0.61); 3.7547 (0.46); 3.7484 (0.51); 3.6088 (0.32); 3.336 (10.88); 3.1789 (2.67); 3.1727 (5.58); 3.1665 (2.63); 2.6928 (2.26); 2.6738 (2.53); 2.5265 (0.9); 2.5129 (17.64); 2.5086 (36.99); 2.5041 (50); 2.4996 (36.17); 2.4952 (17.15); 2.331 (0.31); 1.1833 (5.04); 1.1648 (10.74); 1.1461 (4.76); 0.0077 (0.32); −0.0002 (9.55); −0.0084 (0.42);

Compound No. 926, solvent: [DMSO], 400 MHz

12.9978 (0.38); 8.3225 (7.85); 3.7821 (0.67); 3.5171 (0.48); 3.5077 (0.85); 3.4984 (3.01); 3.4871 (4.18); 3.4837 (3.95); 3.4762 (10.19); 3.4666 (5.99); 3.4544 (1.37); 3.3412 (25.55); 3.3068 (1.18); 3.2769 (44.06); 3.1703 (0.43); 2.6993 (1.83); 2.682 (1.95); 2.5269 (0.67); 2.5222 (0.97); 2.5134 (16.63); 2.509 (36.22); 2.5045 (50); 2.4999 (36.32); 2.4954 (17.24); 2.3313 (0.31); 1.1878 (4.66); 1.1693 (9.99); 1.1507 (4.49); 1.0451 (0.32); −0.0002 (6.42);

Compound No. 912, solvent: [DMSO], 400 MHz

12.9886 (0.36); 8.2881 (7.65); 5.7588 (0.31); 4.0632 (0.48); 4.0447 (1.02); 4.0281 (1.42); 4.0116 (1.06); 3.9926 (0.5); 3.7831 (0.64); 3.7045 (0.31); 3.3558 (8.62); 3.1711 (0.4); 2.6978 (2.08); 2.6791 (2.27); 2.561 (0.46); 2.5566 (0.48); 2.5277 (0.64); 2.5141 (17.01); 2.5098 (36.54); 2.5053 (50); 2.5009 (36.26); 2.4966 (17.22); 2.4552 (0.42); 1.5437 (0.3); 1.5352 (0.35); 1.525 (0.86); 1.5106 (0.91); 1.5016 (1.31); 1.4961 (1.02); 1.487 (1.35); 1.4819 (1.76); 1.475 (1.25); 1.4675 (1.3); 1.4634 (1.52); 1.4595 (1.68); 1.4434 (1.32); 1.4259 (0.63); 1.4108 (0.52); 1.3924 (0.31); 1.3779 (0.54); 1.36 (1.24); 1.3456 (1.84); 1.3414 (1.71); 1.328 (2.17); 1.3232 (1.89); 1.3111 (1.48); 1.3055 (1.78); 1.2878 (1.05); 1.2722 (0.49); 1.2545 (0.31); 1.2342 (0.36); 1.1944 (4.85); 1.1759 (10.55); 1.1628 (14.54); 1.1579 (6.57); 1.1464 (13.25); 1.0462 (0.34); 0.9029 (7.31); 0.8846 (15.05); 0.8665 (6.21); 0.0079 (0.41); −0.0002 (13.75);

Compound No. 331, solvent: [DMSO], 400 MHz

8.23 (3.8); 3.3432 (8.53); 3.1705 (0.49); 2.8409 (9.79); 2.829 (9.8); 2.6714 (0.3); 2.5251 (0.63); 2.5204 (0.86); 2.5117 (15.63); 2.5071 (35.55); 2.5025 (50); 2.4978 (35.63); 2.4932 (15.96); 2.3841 (0.99); 2.3688 (4.93); 2.3599 (2.6); 2.3294 (0.34); 2.0861 (0.86); −0.0002 (10.56); −0.0086 (0.34);

Compound No. 353, solvent: [DMSO], 400 MHz

12.9211 (0.31); 8.225 (4.04); 3.3659 (0.35); 3.3508 (1.38); 3.3325 (2.94); 3.3167 (50); 3.3006 (2.35); 2.5246 (0.62); 2.5198 (0.83); 2.5112 (15.37); 2.5066 (34.52); 2.5021 (48.79); 2.4975 (34.88); 2.493 (16.13); 2.367 (4.1); 2.3587 (2.42); 2.3289 (0.34); 1.6499 (0.44); 1.6332 (0.91); 1.6165 (1.22); 1.5997 (1.06); 1.5831 (0.59); 1.4473 (1.39); 1.4297 (2.79); 1.4111 (2.8); 1.3938 (1.13); 0.9099 (21.69); 0.8934 (20.74); −0.0002 (11.13); −0.0085 (0.35);

Compound No. 354, solvent: [DMSO], 400 MHz

8.243 (3.1); 5.9459 (0.38); 5.9329 (0.86); 5.9201 (0.75); 5.9072 (0.94); 5.9028 (0.49); 5.8942 (0.44); 5.8899 (1.02); 5.877 (0.88); 5.8641 (1.01); 5.8512 (0.43); 5.2362 (0.49); 5.2318 (1.48); 5.2275 (1.53); 5.2231 (0.53); 5.1931 (0.42); 5.1887 (1.29); 5.1845 (1.36); 5.18 (0.48); 5.1344 (0.53); 5.1304 (1.63); 5.1263 (1.56); 5.1224 (0.5); 5.1086 (0.49); 5.1046 (1.52); 5.1006 (1.49); 5.0966 (0.48); 3.9698 (0.73); 3.9655 (1.32); 3.9612 (0.87); 3.9519 (2.11); 3.9423 (0.89); 3.9381 (1.33); 3.9339 (0.73); 3.3193 (50); 2.5248 (0.54); 2.52 (0.73); 2.5114 (14.1); 2.5068 (32.37); 2.5022 (45.89); 2.4975 (32.74); 2.4929 (14.7); 2.3788 (1.85); 2.3704 (3.46); 2.3617 (1.98); −0.0002 (7.45);

Compound No. 338, solvent: [DMSO], 400 MHz

8.2123 (3.64); 3.3202 (20.81); 2.5249 (0.53); 2.5111 (13.12); 2.5069 (26.66); 2.5024 (35.37); 2.4978 (24.88); 2.4933 (11.13); 2.3525 (3.9); 2.3294 (0.41); 1.3744 (50); 1.3563 (0.97); 1.3401 (0.31); 1.2565 (2.08); 1.2472 (1.66); 1.1882 (1.07); 0.008 (0.35); 0.0025 (2.48); −0.0002 (11.42); −0.0086 (0.38);

Compound No. 365, solvent: [DMSO], 400 MHz 8.2314 (3.59); 4.12 (0.42); 4.1038 (0.59); 4.0884 (0.48); 3.37 (0.47); 3.3172 (42.77); 3.266 (0.45); 2.5245 (0.45); 2.5107 (16); 2.5064 (35.34); 2.5019 (50); 2.4973 (36.96); 2.4928 (17.97); 2.4465 (0.32); 2.3687 (3.83); 2.3289 (0.38); 1.6391 (0.47); 1.6187 (0.59); 1.6027 (0.58); 1.5865 (0.36); 1.5086 (0.45); 1.4942 (0.39); 1.4862 (0.49); 1.4744 (0.76); 1.46 (0.57); 1.4523 (0.63); 1.4378 (0.53); 1.3129 (0.66); 1.2991 (0.73); 1.292 (0.64); 1.2785 (1.05); 1.2651 (0.5); 1.2583 (0.48); 1.2441 (0.42); 1.1492 (6.71); 1.1329 (6.7); 0.8881 (14.62); 0.8716 (14.33); −0.0002 (4.23)

Compound No. 2253, solvent: [DMSO], 400 MHz

11.4283 (0.67); 8.0816 (3.1); 3.3318 (1.11); 2.6537 (1.19); 2.6358 (2.06); 2.6163 (1.31); 2.5054 (34.64); 2.501 (50); 2.4965 (37.14); 2.3408 (0.96); 2.0719 (1.61); 1.5837 (1.09); 1.5644 (1.96); 1.5455 (1.99); 1.5268 (1.19); 0.9647 (0.63); 0.9543 (1.3); 0.9468 (3.07); 0.9334 (5.88); 0.927 (3.18); 0.915 (9.55); 0.8964 (6.63); 0.8852 (3.05); 0.8775 (2.08); 0.8655 (0.74); −0.0002 (14.49);

Compound No. 1095, solvent: [DMSO], 400 MHz

11.894 (0.52); 7.7305 (1.31); 3.3021 (20.16); 3.2789 (0.6); 2.9697 (5.86); 2.7998 (6); 2.6687 (0.32); 2.646 (0.47); 2.6263 (0.82); 2.6089 (0.51); 2.5547 (0.32); 2.5502 (0.41); 2.5219 (0.61); 2.508 (16.4); 2.5041 (35.23); 2.4998 (50); 2.4953 (36.77); 2.491 (17.71); 2.0848 (0.88); 1.5817 (0.46); 1.5629 (0.83); 1.5439 (0.83); 1.5252 (0.5); 0.9214 (1.97); 0.9032 (3.92); 0.8848 (1.72); 0.008 (0.56); −0.0002 (18.87); −0.0015 (15.77); −0.0086 (0.65);

Compound No. 1049, solvent: [CDCl3], 400 MHz

8.4693 (3.46); 7.2594 (50); 5.298 (1.51); 4.7897 (10.69); 3.8092 (18.82); 3.7458 (0.59); 2.8934 (0.98); 2.8735 (1.47); 2.8538 (1.07); 1.8524 (0.71); 1.752 (0.9); 1.733 (1.49); 1.7134 (1.56); 1.6946 (1.04); 1.676 (0.36); 1.5739 (0.37); 1.4318 (1.43); 1.0623 (3.97); 1.0441 (8.13); 1.0257 (3.79); 0.008 (0.62); −0.0002 (27.42); −0.0085 (0.92);

Compound No. 1096, solvent: [DMSO], 400 MHz

11.8221 (0.58); 7.7137 (1.84); 3.4505 (0.51); 3.433 (1.63); 3.4155 (1.64); 3.3977 (0.54); 3.3037 (13.35); 3.1137 (0.5); 3.0958 (1.47); 3.078 (1.51); 3.0609 (0.49); 2.6317 (1.09); 2.5002 (50); 2.4958 (37.61); 1.5795 (0.59); 1.5604 (1.05); 1.5418 (1.08); 1.5223 (0.64); 1.3554 (0.64); 1.1448 (1.73); 1.1273 (3.71); 1.1098 (1.74); 1.018 (1.65); 1.0005 (3.54); 0.9829 (1.64); 0.9176 (2.33); 0.8995 (4.59); 0.881 (2.05); −0.0002 (17.38);

Compound No. 991, solvent: [DMSO], 400 MHz

13.0543 (0.43); 8.2854 (3.8); 3.3201 (3.61); 2.8461 (8.19); 2.8343 (8.32); 2.6597 (0.9); 2.6405 (1.37); 2.5512 (0.7); 2.5103 (15.31); 2.5058 (34.75); 2.5012 (50); 2.4967 (36.48); 2.4921 (17.14); 2.4511 (0.6); 2.3832 (0.39); 2.368 (0.38); 1.5974 (0.77); 1.5781 (1.35); 1.5592 (1.44); 1.5405 (0.84); 0.939 (3.67); 0.9207 (7.56); 0.9023 (3.34); −0.0002 (9.28);

Compound No. 992, solvent: [DMSO], 400 MHz

13.0808 (0.51); 8.2936 (4.46); 3.3715 (0.99); 3.3534 (2.98); 3.3391 (3.64); 3.3353 (4.12); 3.3211 (5.78); 2.661 (1.01); 2.6413 (1.62); 2.6228 (1.05); 2.5243 (0.6); 2.5109 (15.13); 2.5063 (34.7); 2.5017 (50); 2.4972 (36.67); 2.4926 (17.4); 1.599 (0.92); 1.5803 (1.64); 1.561 (1.69); 1.5423 (1.02); 1.1552 (5.5); 1.1372 (11.98); 1.1191 (5.4); 0.9421 (4.31); 0.9239 (8.96); 0.9055 (3.93); −0.0002 (12.13);

Compound No. 993, solvent: [DMSO], 400 MHz

13.0638 (0.3); 8.2871 (1.95); 3.3087 (9.08); 3.2827 (1.55); 3.2675 (1.32); 3.2505 (0.67); 2.6403 (0.7); 2.5097 (14.94); 2.5051 (34.45); 2.5006 (50); 2.496 (36.74); 2.4914 (17.45); 1.5971 (0.42); 1.5774 (0.75); 1.5687 (0.88); 1.5503 (1.51); 1.5322 (1.42); 1.5147 (0.75); 0.9415 (1.9); 0.9234 (4.09); 0.9175 (2.73); 0.9048 (2.09); 0.8991 (5.11); 0.8804 (2.14); −0.0002 (7.98);

Compound No. 994, solvent: [DMSO], 400 MHz

13.0946 (0.42); 8.2966 (3.91); 4.1151 (0.55); 4.0985 (0.86); 4.0804 (0.9); 4.0638 (0.59); 3.3136 (6.17); 3.17 (0.32); 2.6702 (0.38); 2.656 (0.82); 2.6367 (1.36); 2.6186 (0.88); 2.5238 (0.56); 2.5192 (0.78); 2.5104 (14.98); 2.5058 (34.58); 2.5012 (50); 2.4966 (36.75); 2.4921 (17.43); 1.5973 (0.79); 1.5784 (1.39); 1.5591 (1.43); 1.5404 (0.85); 1.1947 (18.13); 1.1782 (17.98); 0.9439 (3.7); 0.9257 (7.68); 0.9073 (3.34); 0.0078 (0.33); −0.0002 (13.26); −0.0084 (0.46);

Compound No. 995, solvent: [DMSO], 400 MHz 13.0165 (0.51); 8.2464 (3.96); 3.3195 (2.88); 2.889 (0.54); 2.8797 (0.8); 2.871 (1.18); 2.8611 (1.27); 2.8518 (0.77); 2.8427 (0.63); 2.648 (1); 2.6297 (1.61); 2.6116 (1.05); 2.5104 (15.25); 2.5059 (34.71); 2.5014 (50); 2.4969 (36.85); 2.4925 (17.69); 1.5926 (0.91); 1.5737 (1.59); 1.5543 (1.65); 1.5355 (1.01); 0.9393 (4.01); 0.9211 (8.22); 0.9026 (3.62); 0.7777 (0.68); 0.7648 (2.02); 0.7599 (2.77); 0.7473 (2.74); 0.7419 (2.08); 0.73 (0.95); 0.5978 (0.84); 0.5868 (2.43); 0.5804 (2.29); 0.5712 (2.1); 0.559 (0.63); −0.0002 (13.52);

Compound No. 996, solvent: [DMSO], 400 MHz

13.0634 (0.69); 8.2845 (4.88); 3.3368 (2.15); 3.319 (7.02); 3.3055 (6.49); 3.2877 (2.14); 2.6589 (1.11); 2.6386 (1.81); 2.6206 (1.18); 2.523 (0.73); 2.5096 (16.94); 2.5053 (35.88); 2.5011 (50); 2.4967 (37.03); 2.4923 (18.13); 2.3278 (0.3); 1.5963 (1.03); 1.5774 (1.82); 1.5582 (1.88); 1.5482 (0.97); 1.539 (1.22); 1.531 (1.93); 1.5128 (2.66); 1.4944 (1.99); 1.4758 (0.84); 1.3788 (0.45); 1.3603 (1.55); 1.3411 (2.41); 1.3223 (2.39); 1.3044 (1.37); 1.2866 (0.39); 0.9413 (4.46); 0.9223 (12.04); 0.9031 (13.23); 0.8843 (4.74); 0.0067 (0.4); −0.0002 (11.78); −0.0015 (11.52); −0.0085 (0.5);

Compound No. 997, solvent: [DMSO], 400 MHz

13.0434 (0.3); 8.2701 (2.22); 4.4164 (0.49); 4.3964 (0.5); 3.3117 (6.58); 2.6343 (0.77); 2.5232 (0.47); 2.5094 (15.05); 2.505 (34.53); 2.5004 (50); 2.4958 (36.59); 2.4913 (17.32); 2.2509 (0.58); 2.0558 (0.47); 2.0315 (0.64); 2.0016 (0.5); 1.7294 (0.52); 1.7193 (0.61); 1.7095 (0.68); 1.7031 (0.83); 1.6856 (0.45); 1.6772 (0.44); 1.597 (0.44); 1.5779 (0.75); 1.5594 (0.79); 1.5396 (0.48); 0.9416 (2); 0.9234 (4.15); 0.905 (1.84); −0.0002 (12.2);

Compound No. 1005, solvent: [DMSO], 400 MHz

13.0546 (0.43); 8.2891 (3.92); 3.3169 (4.48); 3.1669 (4.89); 3.1514 (4.87); 2.6418 (1.47); 2.5109 (14.43); 2.5065 (32.75); 2.5019 (46.9); 2.4974 (34.24); 2.4929 (16.21); 1.5907 (0.85); 1.5721 (1.51); 1.5526 (1.57); 1.5341 (0.92); 0.9414 (3.91); 0.9231 (8.57); 0.913 (50); −0.0002 (8.26);

Compound No. 1013, solvent: [DMSO], 400 MHz 8.2814 (3.36); 3.3559 (1.35); 3.3218 (8.4); 3.3057 (2.45); 2.6696 (0.41); 2.6596 (0.73); 2.6381 (1.19); 2.619 (0.75); 2.5234 (0.57); 2.5186 (0.77); 2.5097 (15.65); 2.5052 (34.9); 2.5008 (50); 2.4963 (36.65); 2.4918 (17.44); 1.6505 (0.35); 1.6337 (0.74); 1.6169 (1.09); 1.6 (1.15); 1.5771 (1.26); 1.5579 (1.26); 1.5391 (0.75); 1.4543 (1.11); 1.4367 (2.19); 1.4179 (2.21); 1.4006 (0.92); 0.941 (3.19); 0.9228 (6.65); 0.9131 (16.57); 0.9044 (3.5); 0.8966 (15.84); 0.008 (0.33); −0.0002 (12.3); −0.0012 (10.71); −0.0086 (0.41);

Compound No. 1017, solvent: [DMSO], 400 MHz

13.0219 (0.32); 8.2898 (2.47); 4.8574 (1.36); 4.8243 (1.36); 3.8927 (1.58); 3.8783 (1.62); 3.3167 (5.35); 3.1696 (0.44); 2.6646 (0.74); 2.6439 (0.96); 2.5505 (0.47); 2.5096 (15.22); 2.5052 (34.65); 2.5006 (50); 2.4961 (36.83); 2.4916 (17.58); 2.4506 (0.45); 1.7251 (6); 1.597 (0.57); 1.5775 (0.97); 1.5588 (1.02); 1.541 (0.59); 0.9421 (2.5); 0.9238 (5.07); 0.9054 (2.26); −0.0002 (12.99);

Compound No. 1018, solvent: [DMSO], 400 MHz

8.2858 (1.3); 4.1228 (0.88); 4.1165 (0.95); 4.109 (0.97); 4.1028 (0.93); 3.3138 (2.7); 3.1688 (0.65); 3.1626 (1.32); 3.1564 (0.61); 2.6646 (0.5); 2.6435 (0.53); 2.509 (14.98); 2.5046 (34.39); 2.5 (50); 2.4955 (37.05); 2.4909 (17.83); 1.5737 (0.52); 1.5547 (0.52); 0.9379 (1.26); 0.9197 (2.65); 0.9013 (1.18); −0.0002 (14.68);

Compound No. 1022, solvent: [DMSO], 400 MHz

13.081 (0.51); 8.293 (3.85); 4.0418 (0.58); 4.0255 (0.75); 4.0076 (0.57); 3.3349 (0.6); 2.6388 (1.32); 2.6198 (0.91); 2.5104 (15.3); 2.5059 (34.71); 2.5014 (50); 2.4969 (36.74); 2.4924 (17.54); 2.3283 (0.3); 1.5975 (0.78); 1.5783 (1.36); 1.5598 (1.5); 1.5407 (1.03); 1.526 (0.65); 1.5029 (0.78); 1.4825 (0.86); 1.4601 (0.92); 1.4441 (0.77); 1.4261 (0.38); 1.3557 (0.75); 1.338 (0.97); 1.3204 (1.16); 1.3031 (1.07); 1.2853 (0.57); 1.1633 (8.54); 1.1468 (8.49); 0.9452 (3.4); 0.927 (7.1); 0.9085 (3.36); 0.9021 (4.64); 0.8838 (8.83); 0.8656 (3.62); 0.008 (0.63); −0.0002 (24.62); −0.0086 (0.8);

Compound No. 1026, solvent: [DMSO], 400 MHz

8.2867 (1.58); 5.7516 (1.13); 3.3207 (2.93); 2.6366 (0.57); 2.5184 (0.69); 2.5096 (15.09); 2.5051 (34.59); 2.5005 (50); 2.496 (36.45); 2.4914 (17.07); 1.5782 (0.59); 1.5585 (0.59); 1.5403 (0.37); 1.4753 (0.33); 1.4538 (0.33); 1.3113 (0.36); 1.291 (0.47); 1.156 (3.11); 1.1396 (3.1); 0.9451 (1.49); 0.927 (3.12); 0.9086 (1.39); 0.8907 (5.84); 0.8743 (5.81); −0.0002 (10.92); −0.0084 (0.44);

Compound No. 1033, solvent: [DMSO], 400 MHz

8.3138 (1.32); 4.5158 (0.42); 4.5026 (0.94); 4.4895 (0.42); 3.4595 (0.78); 3.4453 (1.4); 3.4314 (0.79); 3.3089 (26.55); 2.6646 (0.52); 2.5091 (15.51); 2.5046 (34.9); 2.5001 (50); 2.4955 (36.23); 2.491 (16.99); 1.571 (0.49); 1.5514 (0.5); 0.9375 (1.27); 0.9192 (2.59); 0.9009 (1.11); −0.0002 (9.45);

Compound No. 1035, solvent: [DMSO], 400 MHz

8.3345 (2.84); 5.7513 (0.59); 3.5417 (1.14); 3.5273 (2.85); 3.5131 (1.78); 3.4116 (1.07); 3.3977 (2.54); 3.3834 (2.33); 3.3692 (1.12); 3.3227 (1.77); 2.6652 (0.79); 2.6454 (1.01); 2.5097 (15.15); 2.5053 (34.66); 2.5007 (50); 2.4962 (36.95); 2.4917 (17.7); 1.5965 (0.59); 1.5782 (1.04); 1.5587 (1.08); 1.5395 (0.62); 0.9405 (2.7); 0.9223 (5.58); 0.9038 (2.44); −0.0002 (10.11);

Compound No. 1042, solvent: [DMSO], 400 MHz

13.0901 (0.4); 8.3131 (4.57); 5.7519 (0.6); 4.2077 (0.6); 4.1898 (0.79); 4.172 (0.65); 3.4325 (1.23); 3.4189 (1.23); 3.4084 (2.26); 3.3948 (2.27); 3.3669 (2.69); 3.3541 (3.11); 3.3428 (2.64); 3.3301 (2.64); 3.2856 (24.01); 3.2717 (2.55); 3.2306 (0.42); 2.641 (1.71); 2.5524 (0.33); 2.5109 (15.31); 2.5064 (34.83); 2.5019 (50); 2.4974 (36.68); 2.493 (17.55); 2.3351 (0.31); 1.5932 (0.97); 1.5747 (1.7); 1.5551 (1.79); 1.5364 (1.06); 1.1736 (8.57); 1.1567 (8.5); 1.1019 (0.69); 1.0868 (0.65); 0.9417 (4.13); 0.9234 (8.46); 0.905 (3.72); −0.0002 (5.98);

Compound No. 1028, solvent: [DMSO], 400 MHz

8.2826 (1.85); 3.8881 (0.37); 3.8673 (0.39); 3.3044 (22.59); 2.6696 (0.35); 2.6596 (0.42); 2.6365 (0.66); 2.6184 (0.43); 2.5231 (0.56); 2.5185 (0.77); 2.5097 (15.29); 2.5052 (34.79); 2.5006 (50); 2.496 (36.36); 2.4915 (17.03); 1.7713 (0.48); 1.7555 (0.49); 1.7388 (0.32); 1.5917 (0.39); 1.5726 (0.69); 1.5535 (0.72); 1.5348 (0.43); 1.1156 (4.03); 1.0987 (4.02); 0.9427 (1.81); 0.9245 (3.82); 0.9058 (5.71); 0.9007 (4.45); 0.8887 (4.06); 0.8837 (4.12); 0.0081 (0.65); −0.0002 (25.56); −0.0085 (0.83);

Compound No. 1030, solvent: [DMSO], 400 MHz

8.2847 (2.19); 3.8261 (0.42); 3.8058 (0.44); 3.3235 (19.85); 2.6415 (0.8); 2.5103 (14.9); 2.5059 (34.34); 2.5013 (50); 2.4968 (37.48); 2.4924 (18.24); 1.5727 (1.2); 1.5596 (1.49); 1.5408 (1.2); 1.5088 (0.41); 1.4905 (0.76); 1.4716 (1.08); 1.4522 (0.91); 1.4369 (0.68); 1.4176 (0.47); 0.9478 (1.99); 0.9296 (4.1); 0.9196 (0.89); 0.9113 (1.83); 0.9008 (1.48); 0.8869 (4.24); 0.8684 (8.9); 0.8498 (3.74); −0.0002 (15.55);

Compound No. 1027, solvent: [DMSO], 400 MHz

13.0701 (0.32); 8.2918 (4.85); 8.1664 (0.41); 7.7617 (0.5); 3.9238 (1.05); 3.9016 (1.25); 3.8837 (1.08); 3.4342 (0.37); 2.966 (0.46); 2.6381 (2.59); 2.5041 (39.74); 1.5873 (1.26); 1.5684 (2.29); 1.5499 (2.31); 1.5316 (1.37); 1.1349 (3.41); 1.1181 (3.51); 1.0901 (8.74); 1.0731 (8.77); 0.9413 (5.06); 0.9227 (30.86); 0.9091 (50); −0.0002 (9.65);

Compound No. 1128, solvent: [DMSO], 400 MHz

12.904 (0.37); 9.0849 (0.41); 8.2887 (5.63); 4.1237 (3.29); 4.1174 (3.58); 4.1097 (3.53); 4.1035 (3.39); 3.6042 (0.44); 3.322 (21.37); 3.1755 (2.2); 3.1693 (5.58); 3.163 (2.17); 2.6545 (1.73); 2.5104 (16.03); 2.5059 (35.72); 2.5013 (50); 2.4967 (36.1); 2.4922 (16.93); 2.3282 (0.31); 1.6021 (1); 1.5833 (1.75); 1.564 (1.8); 1.5451 (1.09); 0.9491 (4.44); 0.9309 (9.34); 0.9125 (4.04); −0.0002 (6.87);

Compound No. 1140, solvent: [DMSO], 400 MHz

8.2881 (1.53); 3.3056 (50); 2.6695 (0.48); 2.5096 (12.05); 2.505 (26.93); 2.5004 (38.05); 2.4958 (27.02); 2.4912 (12.4); 1.6079 (0.47); 1.5894 (0.72); 1.574 (0.79); 1.5611 (0.61); 1.5554 (0.64); 1.5423 (0.5); 1.4928 (0.43); 1.4741 (0.6); 1.4541 (0.53); 1.4385 (0.38); 0.9598 (1.23); 0.9416 (2.63); 0.9233 (1.16); 0.9001 (0.4); 0.8897 (2.68); 0.8712 (5.89); 0.8526 (2.4); −0.0002 (5.22);

Compound No. 1129, solvent: [DMSO], 400 MHz

13.0277 (0.39); 8.2973 (3.53); 3.9581 (0.59); 3.9406 (0.63); 3.9214 (0.58); 3.3097 (37.17); 3.1695 (0.58); 2.6694 (0.82); 2.6479 (0.98); 2.51 (15.76); 2.5054 (35.27); 2.5008 (50); 2.4962 (35.75); 2.4916 (16.49); 1.6072 (0.59); 1.5874 (1.03); 1.5686 (1.1); 1.5429 (1.75); 1.5251 (2.21); 1.5071 (1.73); 1.489 (0.46); 1.164 (6.56); 1.1474 (6.55); 0.9576 (2.73); 0.9394 (5.75); 0.921 (2.47); 0.9023 (3.17); 0.8839 (6.76); 0.8652 (2.8); −0.0002 (7.22);

Compound No. 1105, solvent: [DMSO], 400 MHz

12.9697 (0.48); 8.8126 (0.47); 8.2562 (7.03); 5.7507 (0.38); 3.3148 (50); 3.1703 (1.54); 2.893 (0.82); 2.8832 (1.1); 2.8748 (1.78); 2.8647 (1.85); 2.8564 (1.08); 2.8466 (0.87); 2.8366 (0.32); 2.6583 (1.32); 2.639 (2.12); 2.6196 (1.41); 2.5243 (0.48); 2.511 (15.45); 2.5064 (34.87); 2.5018 (49.64); 2.4972 (35.7); 2.4926 (16.64); 1.6196 (0.31); 1.6017 (1.23); 1.5825 (2.1); 1.5631 (2.16); 1.5443 (1.34); 1.5264 (0.37); 0.9499 (5.65); 0.9316 (11.94); 0.9132 (5.14); 0.7795 (1.06); 0.7672 (2.85); 0.7617 (4.06); 0.7493 (4); 0.7436 (3.03); 0.7318 (1.5); 0.7126 (0.61); 0.7063 (0.56); 0.6887 (1.11); 0.6847 (0.95); 0.6775 (0.92); 0.6738 (0.82); 0.6077 (1.33); 0.5969 (3.49); 0.5906 (3.26); 0.5864 (3.06); 0.5809 (3.06); 0.5686 (1); −0.0002 (6.94);

Compound No. 2245, solvent: [CDCl3], 400 MHz

9.2422 (0.61); 8.393 (6.07); 7.26 (50); 5.298 (0.46); 4.0312 (0.45); 3.7153 (0.39); 2.9852 (0.85); 2.9759 (1.18); 2.9669 (1.95); 2.9572 (1.94); 2.9486 (1.27); 2.939 (0.93); 2.9291 (0.32); 2.3944 (9.85); 2.3832 (10.07); 2.004 (0.84); 1.5632 (0.36); 1.2562 (1.18); 0.8868 (1.16); 0.8692 (4.22); 0.8556 (4.25); 0.8513 (3.3); 0.8381 (1.51); 0.8165 (0.31); 0.6448 (1.34); 0.6317 (3.31); 0.6276 (3.38); 0.6218 (3.3); 0.618 (3.27); 0.6044 (1.16); −0.0002 (7.26);

Compound No. 2, solvent: [DMSO], 400 MHz

9.7378 (0.47); 8.1072 (3.3); 3.4458 (0.35); 3.391 (50); 3.332 (0.76); 3.3135 (1.43); 3.2992 (1.45); 3.2954 (1.51); 3.2812 (1.4); 3.2632 (0.44); 3.1763 (0.76); 2.5168 (9.37); 2.5124 (20.54); 2.5079 (29.03); 2.5033 (20.64); 2.4988 (9.29); 2.2478 (7.94); 2.054 (8.09); 1.127 (3.03); 1.109 (6.35); 1.0909 (2.93); 0.0064 (0.47);

Compound No. 3, solvent: [DMSO], 400 MHz

12.3016 (0.49); 9.8105 (0.53); 9.7967 (0.96); 9.7832 (0.53); 8.1013 (5.7); 7.9524 (0.35); 3.4415 (50); 3.2631 (1.49); 3.246 (2.95); 3.2312 (2.94); 3.2139 (1.53); 3.17 (1.31); 2.9214 (0.34); 2.8915 (2.92); 2.7324 (2.57); 2.5245 (0.47); 2.5197 (0.64); 2.5112 (13.61); 2.5067 (30.05); 2.5021 (42.36); 2.4976 (30.25); 2.4932 (14.29); 2.244 (13.85); 2.0713 (0.52); 2.049 (14.16); 1.5419 (0.33); 1.5236 (1.58); 1.5056 (3.09); 1.4877 (3.23); 1.4697 (1.76); 1.4516 (0.4); 0.9058 (5.33); 0.8874 (11); 0.8689 (4.79); 0.8387 (0.45); 0.82 (0.89); 0.8015 (0.39); 0.0009 (1.2); −0.0002 (1.21);

Compound No. 5, solvent: [DMSO], 400 MHz

12.3151 (0.47); 9.7875 (1.1); 9.7771 (1.11); 8.0972 (5.74); 3.4683 (50); 3.383 (0.51); 3.1699 (1.68); 2.8369 (0.56); 2.8269 (0.73); 2.8188 (1.21); 2.8085 (1.28); 2.8003 (0.71); 2.7903 (0.63); 2.5241 (0.46); 2.5195 (0.66); 2.5106 (13.6); 2.5062 (30.78); 2.5016 (44.25); 2.497 (32.42); 2.4925 (15.59); 2.3945 (0.36); 2.2393 (14.08); 2.0713 (0.52); 2.0486 (14.43); 0.7415 (0.83); 0.7289 (2.31); 0.7241 (2.94); 0.7113 (2.94); 0.7064 (2.35); 0.6943 (1.08); 0.4885 (1.02); 0.477 (2.73); 0.4718 (2.51); 0.4667 (2.42); 0.4618 (2.56); 0.4495 (0.85); −0.0002 (3.82);

Compound No. 108, solvent: [DMSO], 400 MHz

11.838 (1.63); 7.2251 (7.56); 3.3826 (0.98); 3.3651 (2.9); 3.3474 (3.28); 3.315 (23.35); 3.1755 (0.97); 3.1698 (0.9); 3.158 (2.76); 3.1403 (2.78); 3.1229 (0.97); 2.5105 (8.42); 2.5062 (17.58); 2.5017 (24.22); 2.4973 (17.97); 2.4931 (9.01); 2.3375 (0.39); 2.1487 (16.82); 2.1079 (0.4); 1.9641 (17.1); 1.9083 (50); 1.103 (3.13); 1.0855 (6.59); 1.0679 (3.2); 1.0337 (3.18); 1.0161 (6.56); 0.9985 (3.03); −0.0002 (4.64);

Compound No. 2246, solvent: [CDCl3], 400 MHz

8.4115 (7.55); 7.2629 (44.63); 7.2618 (50); 5.2995 (1.39); 5.2985 (1.57); 4.2867 (1.21); 4.2709 (1.75); 4.2551 (1.29); 4.2497 (1.02); 4.0504 (1.11); 3.0949 (2.25); 2.3973 (12.81); 2.3861 (13.11); 1.9871 (1.39); 1.9717 (1.41); 1.6883 (1.34); 1.67 (1.74); 1.6535 (1.66); 1.6369 (1.14); 1.5277 (1.14); 1.5122 (1.08); 1.5061 (1.22); 1.4927 (2.21); 1.478 (1.51); 1.4722 (1.83); 1.4567 (1.47); 1.368 (2.02); 1.3531 (1.96); 1.3476 (1.9); 1.3335 (2.65); 1.319 (1.22); 1.3137 (1.27); 1.2988 (1.08); 1.2617 (2.25); 1.2453 (2.12); 1.2261 (14.13); 1.2098 (14.11); 1.1371 (2.26); 1.1311 (2.31); 1.1207 (2.66); 1.115 (2.23); 0.9532 (1.59); 0.9432 (19.63); 0.9314 (16.52); 0.927 (21.05); 0.9149 (18.01); 0.9056 (6.38); 0.8976 (4.18); 0.889 (4.6); 0.001 (18.35); 0 (20.43);

Compound No. 7, solvent: [DMSO], 400 MHz 12.3177 (0.39); 9.9824 (1.56); 9.9628 (1.69); 8.077 (10.85); 4.4017 (0.96); 4.3817 (1.77); 4.3605 (1.84); 4.3411 (1.03); 4.044 (1.28); 4.0235 (3.26); 4.0021 (4.89); 3.9824 (3.64); 3.9609 (1.59); 3.7602 (1.92); 3.1704 (1.69); 2.5123 (14.7); 2.5077 (34.35); 2.5031 (50); 2.4985 (36.44); 2.4939 (17.36); 2.2927 (0.73); 2.2849 (0.96); 2.2742 (1.49); 2.2665 (2.33); 2.2454 (27.28); 2.2264 (1.52); 2.2191 (1.17); 2.1832 (0.4); 2.1416 (1.87); 2.1348 (2.61); 2.1283 (2.04); 2.1228 (4.3); 2.1158 (6.22); 2.1095 (4.19); 2.1069 (3.54); 2.1039 (3.48); 2.1014 (3.99); 2.0943 (7.14); 2.0881 (5.31); 2.0825 (2.89); 2.0752 (3.3); 2.0724 (2.29); 2.0692 (2.84); 2.0422 (25.74); 1.9543 (0.55); 1.9306 (1.78); 1.9096 (21.9); 1.8986 (2.77); 1.8826 (2.26); 1.8761 (2.37); 1.8606 (0.68); 1.8552 (0.7); 1.7917 (1.02); 1.7839 (0.94); 1.7681 (3.71); 1.7644 (3.47); 1.7613 (3.23); 1.7576 (2.3); 1.7459 (4.58); 1.7421 (5.85); 1.7355 (5.71); 1.7198 (5.54); 1.7129 (5.63); 1.7014 (2.46); 1.6977 (2.69); 1.6898 (2.82); 1.6754 (2.82); 1.6665 (1.26); 1.6559 (1.28); 1.6498 (1.37); 1.6313 (0.68); 1.5888 (0.82); 1.5684 (2.18); 1.564 (2.86); 1.5616 (3.11); 1.5588 (3.29); 1.5471 (2.39); 1.5401 (6.51); 1.5344 (3.82); 1.5205 (2.53); 1.5137 (5.86); 1.5067 (2.06); 1.4937 (2.11); 1.4878 (3.66); 1.4689 (1.66); 1.4613 (0.89); 1.4421 (0.44); 1.3562 (2.41); −0.0002 (5.08);

Compound No. 8, solvent: [CDCl3], 400 MHz

9.5162 (1.07); 8.3966 (4.67); 7.2654 (15.89); 3.4883 (22.76); 2.356 (13.32); 2.1341 (13.29); 2.1066 (19.4); 2.0052 (45.5); 1.9928 (0.84); 1.906 (0.31); 1.4488 (50); 1.3498 (0.33); 1.315 (1.88); −0.0002 (2.58);

Compound No. 4, solvent: [DMSO], 400 MHz

9.6938 (0.55); 9.6746 (0.54); 8.0945 (3.72); 4.0361 (0.49); 4.0191 (0.7); 4.0009 (0.77); 3.9842 (0.54); 3.6656 (0.78); 3.6494 (1.61); 3.6333 (2.11); 3.617 (1.74); 3.6006 (1); 3.4322 (4.21); 3.1693 (2.13); 2.5104 (15.13); 2.5058 (34.79); 2.5012 (50); 2.4966 (36.18); 2.492 (16.82); 2.3283 (0.31); 2.2427 (8.61); 2.0712 (1.48); 2.0454 (8.82); 1.9084 (16.91); 1.3556 (0.77); 1.1536 (13.42); 1.1372 (13.34); 1.0096 (29.31); 0.9933 (29.32); −0.0002 (4.88);

Compound No. 25, solvent: [CDCl3], 400 MHz

12.8088 (0.94); 9.744 (1.83); 8.4046 (8.45); 7.313 (0.4); 7.2674 (15.84); 7.2627 (50); 6.0119 (0.8); 5.9986 (1.71); 5.9857 (1.57); 5.9728 (2.16); 5.9691 (1.1); 5.9596 (1.59); 5.9556 (2.05); 5.9428 (1.79); 5.9298 (2.06); 5.9171 (1.66); 5.9039 (1.79); 5.8916 (1.42); 5.8783 (2.28); 5.8647 (1.68); 5.8611 (2.15); 5.8487 (1.61); 5.8354 (2.15); 5.8219 (1.03); 5.3047 (2.17); 5.3006 (3.97); 5.2967 (3.52); 5.2617 (1.93); 5.2577 (3.55); 5.2538 (3.11); 5.2372 (4.19); 5.2334 (3.71); 5.1942 (3.71); 5.1905 (3.28); 5.1629 (4.23); 5.1592 (3.62); 5.1452 (4.94); 5.1415 (5.6); 5.1373 (4.91); 5.1335 (3.55); 5.1196 (4.2); 5.1161 (3.55); 4.1097 (3.82); 4.1 (5.82); 4.0962 (7.17); 4.0868 (3.37); 4.0828 (3.67); 3.8339 (6.52); 3.83 (8.73); 3.8259 (5.7); 3.8204 (6.9); 3.8164 (8.65); 3.8125 (4.51); 3.489 (0.37); 2.3609 (29.47); 2.3123 (0.36); 2.1994 (0.35); 2.1494 (25.76); 2.0046 (0.38); 1.4318 (1.44); 0.0045 (2.02); −0.0002 (7.16);

Compound No. 29, solvent: [CDCl3], 400 MHz

13.0704 (0.74); 9.6106 (2.67); 9.5912 (2.74); 8.3941 (16.84); 7.2686 (26.66); 7.2661 (48.06); 4.3314 (0.45); 4.1493 (0.54); 4.1324 (1.55); 4.1159 (3.22); 4.0991 (3.94); 4.0804 (3.11); 4.0638 (1.57); 4.0472 (0.49); 3.7461 (0.52); 3.6971 (0.72); 3.6811 (3.29); 3.6648 (6.66); 3.6485 (6.8); 3.6323 (3.55); 3.6164 (0.84); 3.4883 (0.38); 2.3767 (47.61); 2.3062 (0.3); 2.1482 (48.49); 1.8535 (0.47); 1.6181 (1.72); 1.5992 (6.14); 1.5802 (9.03); 1.5644 (5.74); 1.5615 (6.09); 1.546 (2.23); 1.5336 (2.38); 1.5277 (2.12); 1.5149 (6.97); 1.5094 (6.45); 1.4974 (10.15); 1.4925 (9.73); 1.4789 (8.1); 1.4748 (7.82); 1.46 (2.93); 1.4569 (2.9); 1.432 (1.73); 1.3182 (0.34); 1.2322 (30.89); 1.2157 (30.72); 1.1623 (50); 1.146 (49.35); 1.0991 (0.32); 1.0822 (0.33); 1.0028 (0.39); 0.9865 (15.29); 0.9679 (30.74); 0.9482 (22.43); 0.9457 (25.24); 0.9271 (44.18); 0.9084 (20.17); 0.0022 (4.39); −0.0002 (7.73);

Compound No. 32, solvent: [CDCl3], 400 MHz

13.039 (0.4); 9.5932 (1.96); 9.5739 (2.04); 8.3979 (12.41); 7.3178 (0.53); 7.2675 (35.16); 7.2656 (50); 7.218 (0.53); 5.3012 (1.19); 5.2993 (1.64); 4.8022 (0.31); 4.1957 (0.89); 4.1791 (1.94); 4.1618 (2.45); 4.1441 (2.07); 4.1271 (1.03); 3.7559 (1.93); 3.74 (3.86); 3.7241 (4.09); 3.7079 (2.31); 3.6918 (0.64); 2.4303 (0.45); 2.379 (37.4); 2.3312 (0.39); 2.2014 (0.46); 2.15 (37.81); 2.1025 (0.4); 1.5577 (1.34); 1.5502 (1.59); 1.5418 (3.01); 1.5334 (4.69); 1.525 (3.12); 1.5165 (6.68); 1.4991 (5.31); 1.4851 (3.96); 1.4683 (4.02); 1.4547 (6.06); 1.4376 (8.32); 1.4212 (6.45); 1.4039 (4.97); 1.3978 (6.94); 1.3797 (7.28); 1.3715 (3.7); 1.3641 (5.48); 1.3481 (3.91); 1.3315 (2.33); 1.3151 (0.96); 1.2991 (0.4); 1.2877 (0.41); 1.2718 (0.35); 1.2364 (24.1); 1.22 (23.94); 1.1839 (35.23); 1.1677 (34.89); 1.1362 (0.63); 1.1199 (0.44); 0.986 (0.36); 0.9809 (0.35); 0.9682 (0.59); 0.9476 (12.58); 0.9352 (21.16); 0.9292 (26.46); 0.9171 (35.97); 0.8997 (13.41); 0.8695 (0.45); 0.0017 (6.65); −0.0002 (9.45);

Compound No. 34, solvent: [CDCl3], 400 MHz

12.9296 (0.66); 9.5825 (1.45); 9.5624 (1.51); 8.3892 (9.88); 7.264 (50); 5.2986 (2.12); 4.1807 (0.69); 4.1644 (1.44); 4.1478 (1.61); 4.1446 (1.7); 4.128 (1.55); 4.1115 (0.78); 3.7287 (0.32); 3.7103 (1.34); 3.6946 (2.74); 3.6785 (2.93); 3.6621 (1.63); 3.6482 (0.47); 2.3636 (24.2); 2.1439 (25.17); 1.5616 (0.74); 1.5548 (0.83); 1.5451 (1.87); 1.5358 (2.03); 1.5285 (1.61); 1.5191 (3.35); 1.5034 (2.41); 1.4442 (1.07); 1.4318 (2.87); 1.4284 (2.57); 1.41 (4.61); 1.394 (5.81); 1.3847 (3.98); 1.3789 (3.93); 1.3574 (3); 1.3428 (3.98); 1.3257 (5.41); 1.3215 (5.08); 1.3079 (10.88); 1.29 (13.4); 1.284 (15.4); 1.2308 (18.18); 1.2144 (17.72); 1.1955 (0.65); 1.1802 (0.44); 1.1315 (29.71); 1.1153 (29.94); 0.8931 (9.68); 0.8784 (26.35); 0.8608 (12.49); −0.0002 (10.14); −0.0085 (0.49);

Compound No. 13, solvent: [CDCl3], 400 MHz

12.9993 (1.15); 9.5691 (3.43); 8.2084 (17.89); 7.0722 (39.47); 3.5957 (0.37); 3.2942 (0.5); 3.1555 (8.49); 3.139 (12.03); 3.125 (8.89); 2.8762 (22.79); 2.8587 (23.36); 2.2109 (49.29); 2.1169 (0.33); 1.9589 (50); 1.8121 (0.31); 1.7974 (0.3); 1.2376 (1.56); 0.9041 (1.05); 0.892 (2.21); 0.8863 (1.9); 0.8741 (3.33); 0.8622 (2.13); 0.8546 (2.48); 0.8424 (1.3); 0.8209 (0.97); 0.8132 (1.36); 0.801 (2.77); 0.7957 (2.37); 0.7833 (4.13); 0.7713 (2.63); 0.7637 (3.18); 0.7515 (1.74); 0.7318 (0.73); 0.3572 (2.39); 0.3429 (10.78); 0.3372 (7.57); 0.3221 (23.01); 0.3104 (7.63); 0.3045 (11.5); 0.3019 (12.95); 0.2903 (4.13); 0.2733 (0.76); 0.2536 (0.42); 0.0991 (2.87); 0.0858 (12.53); 0.0735 (12.47); 0.0615 (2.79); 0.0256 (3.7); 0.0121 (15.64); −0.0002 (15.71); −0.0124 (3.25); −0.1942 (5.9);

Compound No. 15, solvent: [CDCl3], 400 MHz

8.4189 (3.59); 7.2647 (13.05); 7.263 (14.52); 3.2913 (3.67); 3.2765 (3.64); 2.9856 (5.37); 2.3763 (9.39); 2.1485 (9.52); 0.9932 (33.21); 0.9922 (35.3); 0.9142 (45.96); 0.9128 (50); 0.0017 (1.85); 0 (2.21);

Compound No. 21, solvent: [CDCl3], 400 MHz

9.7388 (1.04); 8.4032 (6.57); 7.2661 (20.9); 3.3173 (3.46); 3.3022 (5.47); 3.2865 (3.57); 3.042 (13.44); 3.0249 (13.87); 2.381 (18.56); 2.1507 (18.89); 1.9217 (0.72); 1.905 (1.47); 1.8883 (1.89); 1.8716 (1.56); 1.8551 (0.9); 1.8427 (1.31); 1.8259 (2.56); 1.8092 (3.25); 1.7924 (2.67); 1.7756 (1.37); 1.759 (0.41); 1.432 (1.01); 1.0013 (26.66); 0.9846 (25.77); 0.9447 (50); 0.928 (48.17); −0.0002 (4.49);

Compound No. 23, solvent: [CDCl3], 400 MHz

9.6164 (0.9); 8.4008 (5.87); 7.2652 (38.73); 7.2153 (0.48); 5.2991 (2.8); 3.7725 (0.35); 3.7705 (0.35); 3.7662 (0.89); 3.7625 (4.54); 3.7605 (2.77); 3.7562 (2.66); 3.7522 (3.62); 3.7459 (11.05); 3.7398 (3.86); 3.7377 (2.61); 3.7356 (2.69); 3.7309 (3.35); 3.7292 (4.8); 3.7256 (1.2); 3.4986 (1.33); 3.481 (2.76); 3.4674 (2.77); 3.4497 (1.48); 3.2253 (4.34); 3.2069 (5.71); 3.1881 (4.65); 2.3775 (16.95); 2.1525 (17.14); 1.8879 (0.35); 1.8744 (0.83); 1.87 (4.62); 1.8623 (4.32); 1.8534 (13.01); 1.8485 (3.12); 1.8448 (4.22); 1.8368 (4.46); 1.8203 (0.52); 1.7593 (0.59); 1.7425 (1.21); 1.7257 (1.61); 1.7088 (1.41); 1.6922 (0.89); 1.6784 (1.01); 1.6617 (1.9); 1.645 (2.49); 1.6283 (2.18); 1.6116 (1.25); 1.5951 (0.46); 1.5322 (2.05); 1.5146 (4.95); 1.4966 (4.75); 1.4791 (1.78); 1.4652 (2.76); 1.4477 (5.04); 1.4317 (3.87); 1.4282 (5.11); 1.4109 (2.26); 0.9759 (0.35); 0.9572 (32.09); 0.9407 (30.84); 0.9246 (50); 0.9081 (47.57); 0.8909 (0.89); 0.8748 (0.74); 0.8582 (0.64); −0.0002 (7.32);

Compound No. 40, solvent: [CDCl3], 400 MHz

13.1285 (0.7); 9.5733 (1.15); 9.5517 (1.18); 8.398 (8.23); 7.2655 (23.81); 7.2649 (23.5); 4.033 (0.56); 4.0277 (0.68); 4.014 (1.22); 3.9935 (1.25); 3.9799 (0.7); 3.9743 (0.6); 3.9607 (0.3); 3.5238 (0.55); 3.5053 (1.46); 3.4916 (2.42); 3.4778 (1.52); 3.4597 (0.64); 2.3654 (21.39); 2.1436 (22.03); 2.0052 (0.33); 1.6918 (0.32); 1.6732 (1.16); 1.6583 (1.59); 1.655 (1.56); 1.6392 (2.84); 1.625 (2.21); 1.6203 (2.45); 1.6066 (2.06); 1.5939 (0.82); 1.5882 (0.77); 1.5752 (2.28); 1.5616 (3.06); 1.557 (3.05); 1.5437 (5.44); 1.5414 (5.39); 1.5269 (7.78); 1.5226 (4.89); 1.5087 (6.98); 1.4909 (3.51); 1.4737 (1.67); 1.4554 (0.47); 1.4319 (1.23); 1.4303 (1.25); 1.4098 (4.08); 1.3913 (6.69); 1.3732 (5.85); 1.3565 (4.76); 1.3381 (3.08); 1.3197 (0.86); 0.9677 (13.18); 0.9493 (27.32); 0.9308 (36.6); 0.9125 (50); 0.8938 (21.92); 0.7538 (0.31); 0.0004 (3.01); −0.0002 (3.01); −0.0017 (2.38);

Compound No. 43, solvent: [CDCl3], 400 MHz

13.1238 (0.35); 9.7995 (0.36); 9.7859 (0.66); 9.7727 (0.38); 9.7537 (0.74); 8.3893 (4.09); 8.3806 (0.49); 7.261 (42.51); 6.3043 (0.48); 6.2985 (0.45); 6.2654 (0.39); 6.2593 (0.46); 4.5206 (1.32); 4.5164 (0.43); 4.5072 (3.12); 4.5031 (0.88); 4.4939 (1.43); 4.49 (0.44); 4.4026 (0.52); 4.3904 (0.51); 3.7425 (1.27); 3.7292 (1.28); 3.6445 (2.53); 3.6306 (4.43); 3.6168 (2.5); 3.4899 (0.31); 3.4194 (13.62); 3.4171 (50); 3.4 (0.34); 3.3922 (0.95); 3.367 (0.35); 2.4348 (0.98); 2.4132 (9.77); 2.1609 (1.16); 2.1501 (10.71); 2.0041 (0.62); 1.6154 (0.48); 1.4319 (1.84); −0.0002 (5.54);

Compound No. 52, solvent: [CDCl3], 400 MHz

13.1021 (0.32); 9.6407 (0.83); 9.6216 (0.85); 8.4323 (6.07); 7.2627 (50); 5.3016 (1.06); 4.3793 (0.35); 4.3678 (0.73); 4.3509 (0.87); 4.3481 (0.9); 4.3312 (0.79); 4.3198 (0.39); 3.4636 (7.06); 3.4521 (7); 3.4191 (0.35); 3.3869 (29.76); 2.4129 (15.67); 2.1668 (15.93); 1.2883 (10.92); 1.2715 (10.96); 0.008 (0.44); −0.0002 (18.44); −0.0085 (0.69);

Compound No. 54, solvent: [CDCl3], 400 MHz

12.7486 (0.39); 9.5584 (1.94); 8.4212 (9.85); 7.2705 (11.68); 7.2674 (23.68); 3.5564 (2.59); 3.5403 (6.31); 3.5251 (6.77); 3.5076 (3.69); 3.5015 (5.71); 3.486 (11.35); 3.4707 (5.61); 3.4494 (0.7); 3.4325 (0.5); 3.4177 (1.26); 3.3847 (4.24); 3.3571 (40.1); 2.394 (0.6); 2.3583 (27.12); 2.2708 (0.31); 2.1954 (0.48); 2.1489 (29.81); 2.111 (50); 1.9064 (1.42); 1.8905 (4.92); 1.8744 (7.12); 1.8583 (4.61); 1.8421 (1.22); 1.2774 (1.77); 1.2604 (1.93); −0.0002 (2.72);

Compound No. 36, solvent: [CDCl3], 400 MHz

12.682 (0.3); 9.3598 (1.36); 9.3396 (1.39); 8.4146 (9.36); 7.2607 (50); 4.2592 (0.54); 4.2383 (1.15); 4.2227 (1.61); 4.207 (1.14); 4.1862 (0.59); 2.3525 (25.84); 2.142 (25.58); 2.1135 (29.73); 2.0039 (0.35); 1.7339 (0.69); 1.7172 (1.24); 1.6979 (1.6); 1.6813 (1.52); 1.6649 (0.98); 1.648 (0.42); 1.5157 (1.21); 1.5005 (1.11); 1.4942 (1.24); 1.4805 (2.02); 1.4665 (1.57); 1.4601 (1.74); 1.4449 (1.44); 1.3511 (1.57); 1.3365 (1.69); 1.3309 (1.62); 1.3165 (2.46); 1.3024 (1.15); 1.2966 (1.14); 1.2821 (1.01); 1.2207 (15.14); 1.2045 (15.14); 0.9341 (16.43); 0.9288 (17.72); 0.9178 (16.31); 0.9122 (16.86); −0.0002 (5.64);

Compound No. 37, solvent: [CDCl3], 400 MHz

8.4096 (2.36); 7.2605 (31.03); 7.2594 (50); 2.365 (6.08); 2.1438 (6.37); 1.5424 (2.69); 1.1652 (4.24); 1.1483 (4.21); 0.9751 (24.03); 0.0011 (3.62); 0 (5.97);

Compound No. 38, solvent: [CDCl3], 400 MHz

9.4461 (0.54); 9.426 (0.56); 8.434 (5.09); 7.264 (15.25); 7.2625 (11.66); 4.0822 (0.55); 4.0651 (0.73); 4.0606 (0.62); 4.0517 (0.61); 4.0472 (0.75); 4.0441 (0.67); 4.0302 (0.56); 2.3524 (14.02); 2.1472 (13.69); 2.1072 (50); 2.106 (39.89); 2.0984 (0.98); 1.8236 (0.62); 1.8066 (0.93); 1.7928 (0.9); 1.7759 (0.66); 1.1686 (8.9); 1.1519 (8.96); 0.9621 (8.64); 0.9458 (13.41); 0.9303 (8.51); −0.0002 (2.25); −0.0016 (1.66);

Compound No. 45, solvent: [DMSO], 400 MHz

12.2949 (1.07); 9.8598 (1.82); 8.1067 (9.54); 3.4985 (3.89); 3.484 (8.91); 3.4699 (6.29); 3.3829 (50); 3.3539 (14.97); 3.3396 (9.64); 3.3251 (4.19); 3.1707 (1.54); 2.5121 (12.08); 2.5077 (27.13); 2.5032 (39.16); 2.4986 (28.67); 2.494 (13.78); 2.2439 (21.96); 2.0711 (0.81); 2.0486 (22.52); −0.0002 (2.02);

Compound No. 46, solvent: [CDCl3], 400 MHz

13.0823 (1.02); 9.8798 (1.77); 8.3944 (9.57); 7.3098 (0.62); 7.2628 (47.69); 7.2605 (50); 3.6825 (1.85); 3.6691 (5.61); 3.6562 (6.74); 3.6435 (3.18); 3.6323 (0.33); 3.5855 (6.44); 3.573 (8.08); 3.5597 (3.53); 3.4924 (0.3); 3.4634 (0.4); 3.4364 (0.43); 3.3894 (34.13); 3.3872 (34.6); 3.3595 (1.65); 3.3573 (1.6); 2.405 (26.14); 2.1979 (0.37); 2.1512 (26.89); 2.004 (0.65); 1.5872 (2.89); 0.0021 (6.22); −0.0002 (6.64);

Compound No. 59, solvent: [DMSO], 400 MHz

12.4036 (0.93); 10.0757 (0.8); 10.0614 (1.66); 10.0469 (0.89); 9.0939 (0.51); 8.1033 (7.72); 7.6968 (1.15); 7.6937 (1.14); 4.1149 (7.43); 4.1004 (7.51); 3.748 (0.33); 3.6936 (0.37); 3.6437 (32.78); 3.4572 (0.48); 3.3683 (48.13); 3.1691 (7.28); 3.0981 (0.66); 3.086 (0.66); 3.0799 (0.68); 3.0679 (0.66); 2.5513 (0.35); 2.524 (0.54); 2.5192 (0.74); 2.5107 (15.78); 2.5061 (35.24); 2.5015 (50); 2.4969 (35.83); 2.4924 (16.86); 2.2596 (17.82); 2.0713 (1.15); 2.0554 (19.11); 1.9085 (5.06); 1.2046 (1.43); 1.1864 (2.98); 1.1681 (1.38); −0.0002 (2.02);

Compound No. 67, solvent: [CDCl3], 400 MHz

13.0683 (0.47); 12.5279 (1.36); 8.4287 (6.22); 8.4216 (0.6); 7.2611 (50); 7.2094 (0.41); 5.1639 (0.38); 4.5331 (0.69); 4.5161 (0.4); 4.3687 (11.48); 4.2536 (0.69); 4.1879 (0.53); 4.1791 (0.84); 4.0932 (1.34); 4.0873 (0.5); 3.8932 (0.35); 3.7173 (1.21); 3.666 (0.78); 3.5098 (1.86); 3.5042 (29.69); 3.4998 (3.12); 3.4909 (0.78); 3.4807 (1.09); 3.4684 (0.75); 3.4355 (0.81); 3.4272 (1.26); 3.3817 (1.31); 3.3687 (0.7); 3.3063 (0.82); 3.1316 (0.46); 3.1132 (0.47); 2.4989 (0.42); 2.4682 (16.41); 2.4254 (0.99); 2.417 (0.71); 2.202 (0.54); 2.1884 (16.9); 2.1634 (1.18); 2.1171 (0.3); 2.0045 (1.35); 1.5731 (0.35); 1.4232 (0.59); 1.4049 (1.12); 1.3865 (0.55); −0.0002 (5.43);

Compound No. 359, solvent: [DMSO], 400 MHz

12.9647 (0.77); 8.2528 (8.24); 3.5306 (0.94); 3.5104 (1.82); 3.4937 (1.9); 3.4735 (0.97); 3.3209 (3.11); 2.6758 (0.34); 2.6711 (0.48); 2.6665 (0.35); 2.5246 (1.3); 2.5199 (1.83); 2.5112 (28.79); 2.5067 (62.05); 2.5021 (85.33); 2.4975 (60.2); 2.493 (27.42); 2.3718 (7.82); 2.3384 (0.36); 2.3335 (0.48); 2.329 (0.58); 2.3245 (0.42); 1.3569 (1.38); 1.232 (16); 1.2153 (15.85); 1.1489 (0.33); 1.0294 (0.35); 1.0172 (0.7); 1.0091 (1.02); 1.0052 (0.58); 0.9968 (1.98); 0.9887 (1.23); 0.9846 (1.27); 0.9765 (2.08); 0.9681 (0.63); 0.9641 (1.15); 0.9561 (0.77); 0.9439 (0.39); 0.502 (0.48); 0.4938 (0.61); 0.4899 (0.68); 0.4812 (1.69); 0.4717 (1.56); 0.4682 (1.55); 0.4597 (2.14); 0.4511 (1.07); 0.4469 (1.21); 0.4384 (1.12); 0.4321 (1.09); 0.4228 (1.29); 0.4197 (1.28); 0.4099 (1.76); 0.3999 (1.77); 0.3895 (1.85); 0.3808 (0.67); 0.3773 (0.71); 0.3683 (0.67); 0.3193 (0.6); 0.307 (1.05); 0.2971 (1.65); 0.2844 (2.41); 0.2748 (2.3); 0.2655 (1.61); 0.2627 (1.51); 0.2572 (1.53); 0.2535 (2.35); 0.2447 (2.11); 0.2319 (1.37); 0.222 (0.94); 0.2186 (0.57); 0.2096 (0.47); 0.0082 (0.79);

Compound No. 250, solvent: [DMSO], 400 MHz

18.1302 (0.33); 17.8361 (0.38); 17.7326 (0.33); 16.8803 (0.36); 16.8757 (0.32); 8.7145 (0.34); 8.2739 (7.49); 3.8925 (0.32); 3.5322 (1.04); 3.5121 (1.71); 3.4953 (1.83); 3.4751 (0.87); 3.4583 (0.36); 3.4406 (0.41); 3.3971 (1.38); 3.342 (91.18); 3.198 (0.38); 3.1708 (0.38); 3.1501 (0.36); 3.1457 (0.51); 3.1412 (0.59); 3.1366 (0.56); 3.132 (0.4); 3.1021 (0.33); 3.0547 (0.34); 2.8005 (0.45); 2.7518 (0.35); 2.7199 (0.37); 2.6768 (0.4); 2.6723 (0.52); 2.6677 (0.34); 2.5632 (0.47); 2.5586 (0.65); 2.5539 (0.48); 2.5391 (0.39); 2.5256 (1.89); 2.5209 (2.87); 2.5124 (36.97); 2.5078 (78.21); 2.5032 (105.6); 2.4986 (71.37); 2.494 (29.64); 2.3891 (4.93); 2.3807 (9.32); 2.3719 (4.76); 2.3346 (0.43); 2.3301 (0.64); 2.3255 (0.49); 1.9663 (0.37); 1.3567 (1.08); 1.235 (16); 1.2183 (15.53); 1.0331 (0.35); 1.021 (0.77); 1.0129 (1.03); 1.0087 (0.53); 1.0007 (1.92); 0.9924 (1.14); 0.9884 (1.18); 0.9803 (2.04); 0.9721 (0.57); 0.968 (1.1); 0.9599 (0.74); 0.9477 (0.33); 0.505 (0.51); 0.497 (0.65); 0.493 (0.69); 0.4843 (1.65); 0.4747 (1.44); 0.471 (1.34); 0.4627 (1.94); 0.4541 (0.94); 0.4499 (1.1); 0.4414 (1.03); 0.4332 (1.02); 0.424 (1.26); 0.4207 (1.21); 0.4109 (1.64); 0.4039 (1.27); 0.401 (1.53); 0.3906 (1.75); 0.382 (0.56); 0.3784 (0.57); 0.3695 (0.61); 0.3217 (0.58); 0.3092 (1.04); 0.2993 (1.6); 0.2865 (2.3); 0.2771 (2.23); 0.2737 (1.28); 0.2682 (1.4); 0.2649 (1.26); 0.2599 (1.33); 0.2561 (2.13); 0.2474 (1.87); 0.2346 (1.16); 0.2245 (0.79); 0.2123 (0.37); 0.0082 (1.29); 0 (36.67);

Compound No. 360, solvent: [DMSO], 400 MHz

12.7674 (0.53); 8.1683 (6.25); 6.5471 (0.56); 6.524 (0.6); 6.509 (0.58); 6.486 (0.54); 4.9013 (0.33); 4.8827 (0.96); 4.8641 (1.5); 4.8453 (1.46); 4.8237 (0.95); 4.8051 (0.36); 4.4416 (0.5); 4.4241 (0.52); 4.4079 (0.47); 3.3066 (6.28); 2.6756 (0.42); 2.6712 (0.57); 2.6666 (0.4); 2.5867 (0.37); 2.5244 (1.41); 2.5193 (2.12); 2.5109 (35.71); 2.5065 (74.62); 2.502 (100); 2.4976 (70.67); 2.4933 (32.78); 2.3769 (6.55); 2.3687 (12.54); 2.3601 (6.87); 2.3335 (0.6); 2.3291 (0.69); 2.3246 (0.5); 1.3564 (16); 1.3387 (14.98); 1.2262 (4.52); 1.2231 (4.37); 1.2085 (4.54); 1.2055 (4.36); 0.0081 (0.95); 0 (31);

Compound No. 1131, solvent: [DMSO], 400 MHz

12.7693 (0.37); 9.0635 (0.44); 9.0546 (0.45); 8.2073 (7.59); 4.8855 (0.81); 4.8666 (1.26); 4.8461 (1.24); 4.8265 (0.85); 4.8073 (0.33); 3.3239 (7.1); 2.667 (2.22); 2.6615 (1.85); 2.6482 (3); 2.6354 (1.76); 2.6289 (2.01); 2.5562 (0.33); 2.5515 (0.43); 2.5244 (1.33); 2.5196 (1.9); 2.511 (35.73); 2.5065 (77.26); 2.502 (106.95); 2.4974 (76.79); 2.4929 (36.28); 2.4584 (0.45); 2.3333 (0.48); 2.3288 (0.66); 2.3243 (0.48); 2.086 (2.19); 1.613 (0.44); 1.5946 (1.72); 1.5757 (2.95); 1.5564 (3.03); 1.5376 (1.86); 1.5196 (0.53); 1.3617 (13.04); 1.3441 (13.01); 0.9511 (7.63); 0.9329 (16); 0.9145 (6.91); 0 (7.62);

Compound No. 141, solvent: [DMSO], 400 MHz

9.0978 (0.34); 8.2144 (7.36); 4.9072 (0.42); 4.8886 (1.13); 4.8695 (1.71); 4.8488 (1.64); 4.8294 (1.12); 4.8111 (0.43); 3.325 (7.02); 3.2556 (0.94); 3.2339 (0.57); 3.1982 (0.33); 3.1687 (0.46); 3.0473 (0.35); 2.6763 (0.42); 2.6717 (0.56); 2.6672 (0.41); 2.5413 (0.37); 2.5251 (1.84); 2.5201 (2.79); 2.5117 (35.87); 2.5072 (73.7); 2.5027 (98.89); 2.4982 (70.52); 2.4937 (33); 2.3578 (13.28); 2.3521 (13.35); 2.3467 (5.6); 2.3342 (0.81); 2.3297 (0.82); 2.3254 (0.61); 2.0863 (1.36); 1.3615 (16); 1.344 (15.87); 0.0081 (0.61);

Compound No. 251, solvent: [DMSO], 400 MHz

12.7416 (0.43); 12.726 (0.42); 9.0169 (0.79); 8.608 (1.55); 8.1698 (8.83); 4.9009 (0.42); 4.882 (1.17); 4.8628 (1.89); 4.8435 (1.9); 4.824 (1.24); 4.8046 (0.48); 3.696 (4.2); 3.6662 (0.44); 3.6492 (0.5); 3.5101 (2.05); 3.3366 (0.36); 2.6714 (0.54); 2.5562 (1.03); 2.5531 (1.07); 2.5027 (92.18); 2.466 (1.33); 2.3855 (8.3); 2.3775 (15.74); 2.369 (9.12); 2.3302 (0.73); 1.3568 (15.88); 1.3393 (16); 0.0011 (16.75); 0 (17.8);

Compound No. 169, solvent: [CDCl3], 400 MHz

9.7704 (0.39); 8.4552 (2.34); 7.5182 (1.07); 7.3093 (0.35); 7.2718 (0.34); 7.2675 (0.68); 7.2668 (0.72); 7.2594 (185.1); 6.9955 (1.04); 4.2454 (4.29); 4.2314 (4.3); 3.7823 (16); 3.491 (0.37); 2.3413 (1.61); 2.3363 (4.77); 2.331 (4.95); 2.3258 (1.83); 1.6366 (0.41); 1.5526 (2.26); 0.0082 (1.97); 0 (71.54);

Compound No. 2251, solvent: [CDCl3], 400 MHz

9.7332 (2.06); 9.7107 (2.06); 8.5156 (10.08); 7.5192 (0.55); 7.2627 (56.85); 7.2604 (101.3); 6.9964 (0.54); 4.9462 (0.52); 4.9277 (1.37); 4.9096 (2.05); 4.8914 (1.87); 4.8867 (1.73); 4.869 (1.24); 4.8513 (0.52); 4.6822 (0.62); 4.6631 (0.81); 4.5911 (1.94); 4.5746 (2.15); 4.5569 (2.05); 4.5391 (1.36); 4.5336 (1.1); 4.5158 (0.69); 3.748 (0.37); 2.5652 (0.33); 2.3595 (15.51); 2.0054 (0.85); 1.8551 (0.48); 1.6895 (0.4); 1.5785 (2.32); 1.4322 (2.33); 1.4188 (16); 1.4013 (15.76); 1.3298 (11.69); 1.3134 (9.68); 0.0024 (13.37); 0 (23.26)

Compound No. 397, solvent: [DMSO], 400 MHz

12.9636 (3.26); 8.7957 (0.33); 8.7874 (0.36); 8.7731 (0.35); 8.7685 (0.36); 8.759 (0.35); 8.7446 (0.37); 8.7226 (0.35); 8.2482 (15.56); 5.5334 (0.49); 5.5108 (0.53); 3.2999 (77.15); 3.1678 (1.99); 3.1475 (4.73); 3.127 (4.82); 3.1067 (1.88); 2.9072 (0.51); 2.8845 (0.5); 2.6741 (0.88); 2.6697 (1.18); 2.6649 (0.89); 2.5397 (0.94); 2.5228 (2.55); 2.5096 (64.36); 2.5051 (138.28); 2.5006 (193.4); 2.4961 (143.28); 2.4917 (71.27); 2.3708 (16); 2.3317 (1.14); 2.3276 (1.39); 2.3229 (1.05); 2.0716 (0.59); 1.247 (0.48); 1.1017 (1.08); 1.0893 (2.53); 1.0818 (3.64); 1.0692 (6.63); 1.0625 (4.27); 1.0569 (4.87); 1.0491 (7.06); 1.0368 (4.14); 1.0292 (2.97); 1.0166 (1.38); 0.8947 (0.36); 0.8765 (0.85); 0.8584 (1.12); 0.8369 (0.95); 0.8247 (0.52); 0.8188 (0.78); 0.8055 (0.43); 0.799 (0.4); 0.5361 (0.32); 0.517 (1.99); 0.5108 (3.12); 0.5067 (3.04); 0.4974 (5.96); 0.4925 (5.12); 0.4851 (5.99); 0.4763 (8.98); 0.464 (3.74); 0.4555 (3.73); 0.4053 (2.75); 0.3967 (3.63); 0.393 (4.65); 0.3845 (8.14); 0.3766 (4.28); 0.372 (6.87); 0.3646 (10.55); 0.3603 (6.35); 0.3476 (7.58); 0.3445 (7.74); 0.3409 (8.81); 0.328 (8.81); 0.3184 (7.94); 0.306 (4.02); 0.2973 (5.69); 0.2882 (5.3); 0.2838 (6.47); 0.2754 (8.57); 0.2623 (7.68); 0.2548 (3.69); 0.2486 (2.82); 0.2393 (0.74); 0.2288 (0.69); 0.2162 (0.66); 0.2039 (1.2); 0.1987 (1.5); 0.1947 (1.37); 0.1862 (1.55); 0.1826 (1.19); 0.17 (0.55); 0.1594 (0.37); 0.1468 (0.37); 0.0081 (1.88);

Compound No. 219, solvent: [DMSO], 400 MHz

11.6353 (2.84); 7.6671 (5.21); 7.2155 (11.77); 3.3635 (53.54); 3.3401 (25.69); 3.1649 (5.72); 2.6746 (3.58); 2.67 (5.03); 2.6653 (3.65); 2.5381 (1.99); 2.5333 (4.78); 2.5235 (15.86); 2.5188 (21.26); 2.5101 (305.53); 2.5055 (667.42); 2.5009 (930.02); 2.4963 (645.02); 2.4917 (282.72); 2.4663 (2.81); 2.4499 (3.19); 2.3323 (4.72); 2.3275 (6.07); 2.323 (4.74); 2.2028 (16); 2.0732 (1.9); 0.008 (7.46); −0.0002 (299.19); −0.0086 (8.02);

Compound No. 204, solvent: [DMSO], 400 MHz

11.909 (1.22); 7.67 (2.17); 7.6179 (1.06); 5.2481 (1.12); 5.2371 (1); 4.2026 (1.47); 4.2004 (1.42); 4.1848 (3.41); 4.1827 (3.4); 4.1669 (3.69); 4.165 (3.39); 4.1537 (0.5); 4.149 (1.32); 4.1359 (1.13); 4.1181 (1.16); 4.1076 (1.15); 4.0898 (1.07); 3.3298 (34.63); 3.2933 (0.74); 3.2784 (0.55); 3.26 (0.81); 3.1656 (1.08); 3.1566 (0.56); 3.1316 (0.77); 3.1246 (0.75); 2.6747 (0.67); 2.6701 (0.92); 2.6656 (0.66); 2.5452 (0.61); 2.5404 (1.07); 2.5379 (0.97); 2.5328 (1.3); 2.5236 (3.66); 2.5189 (4.88); 2.5103 (54.44); 2.5057 (116.52); 2.501 (160.76); 2.4964 (110.01); 2.4918 (47.65); 2.455 (0.68); 2.4503 (1.26); 2.4457 (1.18); 2.4412 (0.84); 2.3543 (4.72); 2.349 (4.72); 2.3324 (1.21); 2.3277 (2.01); 2.3225 (2.61); 2.3159 (2.37); 2.1892 (0.67); 2.1529 (0.64); 2.0734 (1.27); 1.7002 (1.5); 1.6857 (1.43); 1.6741 (1.68); 1.6599 (1.46); 1.6413 (0.63); 1.626 (0.5); 1.5372 (0.74); 1.3954 (0.55); 1.3542 (0.54); 1.3264 (0.51); 1.2708 (0.64); 1.253 (7.13); 1.2352 (16); 1.2174 (6.92); 1.19 (3.24); 1.1722 (6.98); 1.1545 (3.12); 0.008 (1.37); 0.0057 (0.51); 0.0048 (0.62); 0.004 (0.81); −0.0002 (42.99); −0.0085 (1.07);

Compound No. 220, solvent: [DMSO], 400 MHz

11.9778 (0.5); 7.7003 (2.04); 7.6231 (1.06); 4.2494 (6.39); 4.0227 (2.92); 3.6922 (16); 3.6812 (0.54); 3.6046 (8.06); 3.3596 (1.18); 3.0045 (7.15); 2.8773 (15.21); 2.5244 (0.82); 2.5196 (1.17); 2.511 (17.21); 2.5064 (37.74); 2.5018 (52.89); 2.4971 (36.74); 2.4926 (16.22); 2.3583 (3.5); 2.3529 (3.67); 2.3189 (1.86); 2.3133 (1.81); −0.0002 (2.24);

Compound No. 329, solvent: [DMSO], 400 MHz

11.5835 (4.4); 7.6627 (6.33); 7.4239 (1); 7.2468 (9.63); 7.2188 (14.78); 7.1689 (4.88); 3.3446 (24.36); 3.3242 (138.5); 3.3014 (8.54); 3.2652 (0.98); 3.2619 (1.01); 3.2396 (0.75); 3.177 (0.94); 2.6767 (0.6); 2.6721 (0.84); 2.6674 (0.6); 2.5423 (0.88); 2.5374 (0.57); 2.5326 (0.56); 2.5255 (2.03); 2.5208 (3.18); 2.5122 (45.06); 2.5076 (98.48); 2.503 (137.52); 2.4983 (96.02); 2.4938 (42.71); 2.3735 (0.87); 2.3391 (0.9); 2.3344 (1.22); 2.3298 (1.45); 2.3251 (1.2); 2.2153 (16); 2.0739 (0.77); 0.008 (1.42); 0.0056 (0.56); −0.0002 (45.6); −0.0085 (1.19);

Compound No. 330, solvent: [DMSO], 400 MHz

11.912 (0.51); 8.1919 (1.03); 7.7006 (2.31); 7.6204 (1.16); 4.2524 (6.67); 4.0242 (2.91); 3.6932 (16); 3.6022 (7.85); 3.5061 (0.73); 3.4438 (0.79); 3.0071 (6.81); 2.8778 (15.08); 2.5245 (0.76); 2.5198 (1.16); 2.5111 (18.17); 2.5066 (40.02); 2.5019 (56.21); 2.4973 (39.58); 2.4927 (17.85); 2.3886 (1.23); 2.3805 (3.81); 2.3725 (4.77); 2.364 (2.31); 2.3404 (1.14); 2.3321 (2.14); 2.3237 (1.3); −0.0002 (15.03);

Compound No. 2252, solvent: [DMSO], 400 MHz

7.682 (3.24); 7.5992 (1.9); 4.148 (7.07); 3.9115 (3.67); 3.3215 (108.24); 2.9899 (8.76); 2.8594 (16); 2.6705 (0.64); 2.5407 (0.65); 2.524 (1.35); 2.5193 (1.98); 2.5106 (33.34); 2.506 (73.45); 2.5014 (102.9); 2.4968 (72.2); 2.4922 (32.23); 2.3787 (2.44); 2.3701 (4.84); 2.3612 (2.64); 2.3327 (1.9); 2.3237 (3.29); 2.3148 (1.54); −0.0002 (10.57);

Compound No. 2, solvent [DMSO], 400 MHz

9.738 (0.5); 8.107 (3.3); 3.446 (0.4); 3.391 (50.0); 3.332 (0.8); 3.314 (1.4); 3.299 (1.5); 3.295 (1.5); 3.281 (1.4); 3.263 (0.4); 3.176 (0.8); 2.517 (9.4); 2.512 (20.5); 2.508 (29.0); 2.503 (20.6); 2.499 (9.3); 2.248 (7.9); 2.054 (8.1); 1.127 (3.0); 1.109 (6.4); 1.091 (2.9); 0.006 (0.5)

Compound No. 23, solvent [CDCl3], 400 MHz

9.403 (1.1); 8.426 (7.8); 7.2642 (16.9); 7.2635 (16.2); 3.475 (2.1); 3.457 (3.3); 3.443 (3.1); 3.439 (3.3); 3.425 (2.2); 2.351 (21.4); 2.148 (21.5); 2.108 (50.0); 1.722 (0.7); 1.706 (1.5); 1.689 (1.9); 1.672 (1.7); 1.655 (1.0); 1.639 (0.3); 1.522 (2.4); 1.504 (5.0); 1.486 (4.9); 1.468 (1.9); 0.979 (0.6); 0.962 (0.7); 0.949 (30.0); 0.932 (28.7); 0.922 (1.7); 0.906 (1.2); 0.000 (4.3)

Compound No. 59, solvent [DMSO], 400 MHz

12.414 (0.5); 10.063 (0.7); 8.104 (3.5); 7.670 (1.1); 7.667 (1.1); 4.115 (3.4); 4.100 (3.4); 3.643 (16.0); 3.324 (11.7); 2.523 (1.0); 2.519 (1.5); 2.510 (21.2); 2.506 (45.0); 2.501 (61.5); 2.496 (42.8); 2.492 (19.2); 2.259 (7.7); 2.055 (8.4); 0.008 (1.2); 0.000 (38.9); −0.009 (1.1)

Compound No. 610, solvent [DMSO], 400 MHz

8.339 (3.4); 8.221 (1.6); 4.137 (3.8); 4.123 (3.8); 3.664 (16.0); 2.711 (1.9); 2.693 (1.9); 2.672 (1.0); 2.506 (58.3); 2.502 (75.3); 2.497 (55.0); 2.329 (0.5); 1.189 (2.8); 1.182 (2.0); 1.170 (5.9); 1.163 (3.8); 1.151 (2.9); 1.144 (1.7); 0.000 (20.5)

Compound No. 645, solvent [DMSO], 400 MHz

11.950 (1.0); 7.696 (5.8); 7.639 (2.9); 5.255 (2.5); 5.246 (2.4); 4.466 (0.9); 4.433 (0.9); 4.204 (3.0); 4.188 (5.6); 4.170 (5.3); 4.152 (2.5); 4.133 (1.9); 4.116 (2.2); 4.112 (2.2); 4.095 (1.8); 4.077 (0.6); 4.068 (0.6); 3.546 (2.9); 3.282 (1.1); 3.250 (1.8); 3.162 (1.1); 3.131 (1.6); 3.104 (0.8); 2.765 (0.7); 2.706 (3.4); 2.688 (3.9); 2.672 (3.2); 2.508 (52.1); 2.503 (67.2); 2.499 (53.7); 2.191 (1.4); 2.157 (1.5); 2.087 (0.6); 2.061 (0.9); 2.029 (0.9); 1.669 (4.1); 1.571 (1.1); 1.539 (1.7); 1.433 (1.1); 1.399 (1.3); 1.359 (1.4); 1.331 (1.3); 1.275 (1.4); 1.256 (7.8); 1.239 (16.0); 1.221 (7.5); 1.192 (5.1); 1.174 (11.6); 1.159 (13.3); 1.146 (6.1); 1.141 (7.1); 1.128 (6.9); 1.109 (3.3); 0.000 (9.2)

Compound No. 719, solvent [DMSO], 400 MHz

13.007 (1.0); 8.339 (3.8); 4.137 (3.7); 4.122 (3.9); 3.666 (16.0); 3.317 (13.7); 2.728 (1.4); 2.709 (1.5); 2.506 (43.2); 2.502 (58.0); 2.497 (47.2); 1.197 (2.7); 1.178 (5.7); 1.159 (2.7); 0.000 (17.2)

Compound No. 754, solvent [DMSO], 400 MHz

11.863 (5.0); 7.702 (5.8); 7.640 (2.8); 5.253 (2.8); 4.462 (1.0); 4.428 (1.0); 4.203 (2.8); 4.186 (6.7); 4.169 (5.7); 4.151 (2.6); 4.133 (2.1); 4.115 (2.8); 4.099 (2.0); 3.336 (75.3); 3.271 (1.4); 3.235 (2.1); 3.169 (1.5); 3.136 (1.8); 3.109 (0.8); 2.771 (0.9); 2.713 (3.2); 2.697 (3.8); 2.677 (3.5); 2.502 (164.0); 2.498 (137.5); 2.448 (1.0); 2.329 (1.0); 2.189 (1.6); 2.161 (1.7); 2.071 (0.9); 2.034 (0.9); 1.672 (4.2); 1.583 (1.2); 1.550 (1.9); 1.431 (1.3); 1.400 (1.6); 1.358 (1.6); 1.336 (1.4); 1.310 (1.1); 1.257 (8.0); 1.239 (16.0); 1.221 (7.6); 1.188 (4.7); 1.180 (6.7); 1.171 (10.1); 1.162 (12.9); 1.153 (7.1); 1.143 (8.5); 1.127 (6.6); 1.109 (3.2); 0.000 (33.8)

Compound No. 770, solvent [DMSO], 400 MHz

11.919 (0.7); 7.733 (2.9); 7.634 (1.6); 4.257 (6.8); 4.016 (3.4); 3.696 (16.0); 3.680 (0.6); 3.599 (9.1); 3.407 (3.9); 3.017 (8.1); 2.878 (14.9); 2.719 (1.0); 2.701 (1.2); 2.680 (0.8); 2.675 (1.0); 2.671 (0.8); 2.666 (0.6); 2.661 (0.6); 2.656 (0.6); 2.524 (0.7); 2.520 (1.0); 2.511 (12.9); 2.507 (27.9); 2.502 (38.9); 2.497 (27.0); 2.493 (12.0); 1.185 (2.5); 1.166 (5.5); 1.147 (2.5); 1.139 (1.6); 1.121 (3.2); 1.102 (1.4); 0.000 (7.0)

Compound No. 1049, solvent [DMSO], 400 MHz

8.319 (2.6); 4.136 (3.0); 4.121 (3.0); 3.663 (16.0); 3.316 (45.4); 2.675 (1.0); 2.670 (1.2); 2.665 (0.9); 2.661 (0.8); 2.655 (0.9); 2.635 (0.5); 2.523 (1.7); 2.519 (2.5); 2.510 (41.4); 2.506 (89.0); 2.501 (122.2); 2.496 (85.2); 2.492 (38.3); 2.332 (0.5); 2.328 (0.7); 2.323 (0.5); 1.591 (0.5); 1.572 (0.9); 1.553 (0.9); 1.534 (0.6); 0.938 (2.6); 0.920 (5.5); 0.902 (2.3); 0.008 (1.0); 0.000 (35.7); −0.009 (1.1)

Compound No. 1084, solvent [DMSO], 400 MHz

11.952 (0.6); 7.671 (4.5); 7.606 (2.1); 5.253 (1.6); 5.242 (1.4); 4.463 (0.5); 4.429 (0.5); 4.205 (1.9); 4.202 (2.0); 4.197 (1.3); 4.187 (4.1); 4.185 (4.0); 4.169 (4.0); 4.167 (3.9); 4.150 (1.7); 4.141 (0.7); 4.133 (0.5); 4.123 (2.0); 4.106 (3.0); 4.088 (2.0); 3.561 (1.3); 3.277 (0.7); 3.246 (1.0); 3.162 (0.6); 3.155 (0.7); 3.130 (1.0); 3.123 (1.0); 3.098 (0.5); 2.668 (1.5); 2.648 (2.3); 2.633 (2.1); 2.618 (1.6); 2.597 (0.9); 2.526 (0.5); 2.521 (0.8); 2.512 (15.0); 2.508 (32.2); 2.503 (44.1); 2.499 (31.4); 2.494 (14.7); 2.190 (0.9); 2.159 (0.9); 2.087 (0.6); 2.075 (0.8); 2.033 (0.5); 1.697 (1.9); 1.686 (2.2); 1.679 (2.3); 1.667 (2.3); 1.653 (1.3); 1.644 (0.8); 1.638 (0.7); 1.580 (1.6); 1.562 (2.7); 1.542 (3.4); 1.524 (2.4); 1.507 (1.2); 1.489 (0.5); 1.426 (0.7); 1.417 (0.6); 1.404 (0.6); 1.394 (0.9); 1.386 (0.7); 1.353 (0.9); 1.325 (0.7); 1.309 (0.5); 1.301 (0.5); 1.274 (0.9); 1.255 (7.6); 1.237 (16.0); 1.219 (7.2); 1.190 (4.1); 1.172 (8.6); 1.154 (4.0); 0.931 (5.2); 0.913 (11.0); 0.894 (5.7); 0.879 (6.2); 0.861 (2.7); 0.000 (13.2)

Compound No. 1100, solvent [DMSO], 400 MHz

12.009 (0.9); 7.699 (1.7); 7.596 (0.9); 4.250 (6.4); 4.018 (3.3); 3.693 (16.0); 3.592 (9.7); 3.309 (183.4); 3.007 (8.2); 2.872 (14.1); 2.674 (1.4); 2.670 (1.9); 2.665 (1.4); 2.660 (1.0); 2.650 (1.0); 2.631 (1.0); 2.608 (0.7); 2.523 (3.7); 2.518 (5.2); 2.510 (68.2); 2.505 (148.3); 2.500 (206.6); 2.496 (143.0); 2.491 (63.5); 2.332 (0.9); 2.327 (1.3); 2.323 (0.9); 1.583 (0.6); 1.564 (1.1); 1.544 (1.3); 1.525 (1.2); 1.504 (0.8); 0.932 (3.0); 0.914 (6.4); 0.895 (3.8); 0.875 (3.9); 0.857 (1.6); 0.008 (1.5); 0.000 (55.6); −0.006 (0.7); −0.007 (0.6); −0.009 (1.7)

Compound No. 1159, solvent [DMSO], 400 MHz

13.014 (0.9); 8.324 (3.9); 4.136 (3.7); 4.122 (3.9); 3.666 (16.0); 3.322 (9.5); 2.663 (1.7); 2.507 (34.2); 2.502 (46.4); 2.498 (38.2); 2.074 (0.8); 1.598 (0.8); 1.578 (1.5); 1.560 (1.6); 1.541 (0.9); 0.949 (3.0); 0.931 (6.2); 0.912 (2.9); 0.000 (14.8)

Compound No. 1194, solvent [DMSO], 400 MHz

11.862 (3.8); 7.681 (4.9); 7.612 (2.3); 5.254 (2.2); 5.243 (2.0); 4.463 (0.8); 4.430 (0.8); 4.202 (2.5); 4.188 (5.3); 4.185 (5.0); 4.170 (4.6); 4.152 (1.8); 4.141 (0.9); 4.123 (1.7); 4.111 (1.9); 4.105 (1.9); 4.093 (1.7); 4.076 (0.6); 3.368 (12.7); 3.266 (1.0); 3.233 (1.5); 3.162 (1.1); 3.136 (1.4); 3.105 (0.7); 2.763 (0.6); 2.671 (2.3); 2.653 (2.9); 2.507 (86.9); 2.502 (111.7); 2.498 (81.3); 2.451 (0.5); 2.329 (0.7); 2.191 (1.3); 2.157 (1.3); 2.086 (0.7); 2.074 (1.2); 2.041 (0.7); 1.682 (3.2); 1.581 (2.4); 1.561 (3.5); 1.542 (4.2); 1.523 (2.6); 1.502 (1.4); 1.426 (1.0); 1.394 (1.3); 1.359 (1.3); 1.328 (1.0); 1.309 (0.8); 1.273 (1.3); 1.256 (7.8); 1.238 (16.0); 1.220 (7.3); 1.187 (4.1); 1.169 (8.2); 1.151 (3.9); 0.937 (5.6); 0.919 (11.4); 0.901 (7.6); 0.884 (6.3); 0.865 (2.8); 0.000 (33.0)

Compound No. 1210, solvent [DMSO], 400 MHz

11.935 (0.8); 11.916 (1.2); 7.712 (2.5); 7.605 (1.5); 4.255 (6.7); 4.020 (3.4); 3.695 (16.0); 3.591 (9.5); 3.313 (36.7); 3.011 (8.4); 2.873 (14.4); 2.675 (0.8); 2.671 (0.9); 2.666 (0.9); 2.655 (1.0); 2.636 (1.0); 2.616 (0.7); 2.524 (0.8); 2.519 (1.2); 2.511 (17.0); 2.506 (37.2); 2.501 (51.9); 2.497 (36.2); 2.492 (16.2); 2.086 (1.3); 2.073 (2.7); 1.585 (0.6); 1.566 (1.1); 1.547 (1.2); 1.528 (0.9); 1.522 (0.9); 1.502 (0.8); 0.939 (2.9); 0.921 (6.2); 0.902 (3.5); 0.882 (3.8); 0.864 (1.6); 0.000 (15.6)

Compound No. 2379, solvent [CDCl3], 400 MHz

13.084 (0.7); 9.304 (1.5); 8.663 (15.1); 8.643 (0.6); 7.263 (32.2); 3.518 (1.5); 3.504 (1.9); 3.500 (5.1); 3.486 (5.3); 3.482 (5.4); 3.468 (5.2); 3.464 (2.0); 3.450 (1.6); 2.621 (1.6); 2.528 (44.8); 1.671 (0.6); 1.428 (0.9); 1.260 (12.5); 1.242 (26.5); 1.224 (12.1); 0.008 (0.5); 0.000 (18.4); −0.002 (7.2)

Compound No. 2380, solvent [CDCl3], 400 MHz

13.239 (1.2); 9.364 (1.8); 8.669 (17.1); 7.264 (22.9); 7.263 (27.5); 7.260 (0.7); 7.259 (0.5); 3.451 (3.6); 3.434 (7.8); 3.420 (7.9); 3.402 (3.8); 2.525 (48.4); 1.676 (0.9); 1.658 (4.1); 1.640 (8.4); 1.622 (9.7); 1.604 (4.6); 1.586 (1.0); 1.058 (0.5); 1.019 (13.5); 1.000 (27.3); 0.982 (11.9); 0.000 (17.2); −0.008 (0.5)

Compound No. 2400, solvent [CDCl3], 400 MHz

12.980 (0.5); 9.305 (1.9); 8.669 (1.8); 7.260 (4.8); 3.502 (0.5); 3.482 (1.4); 3.462 (1.4); 3.422 (0.5); 2.501 (5.7); 1.749 (0.2); 1.719 (0.5); 1.699 (0.7); 1.879 (0.5); 1.659 (0.2); 1.585 (4.2); 1.522 (0.5); 1.502 (1.4); 1.482 (1.4); 1.462 (0.5); 0.979 (6.3); 0.921 (6.3); −0.002 (2.5)

Compound No. 2423, solvent [DMSO], 400 MHz

12.873 (1.0); 9.650 (1.0); 8.252 (7.7); 3.482 (0.4); 3.473 (0.7); 3.464 (2.3); 3.453 (3.1); 3.450 (2.7); 3.441 (7.5); 3.431 (4.1); 3.419 (0.9); 3.413 (0.5); 3.317 (45.4); 3.305 (1.8); 3.268 (30.6); 2.525 (0.4); 2.511 (8.9); 2.507 (19.4); 2.502 (27.3); 2.498 (19.4); 2.493 (8.7); 2.371 (19.7); 2.074 (0.3); 0.000 (1.5)

Compound No. 3915, solvent [DMSO], 400 MHz

11.838 (1.6); 7.225 (7.6); 3.383 (1.0); 3.365 (2.9); 3.347 (3.3); 3.315 (23.4); 3.176 (1.0); 3.170 (0.9); 3.158 (2.8); 3.140 (2.8); 3.123 (1.0); 2.511 (8.4); 2.506 (17.6); 2.502 (24.2); 2.497 (18.0); 2.493 (9.0); 2.338 (0.4); 2.149 (16.8); 2.108 (0.4); 1.964 (17.1); 1.908 (50.0); 1.103 (3.1); 1.086 (6.6); 1.068 (3.2); 1.034 (3.2); 1.016 (6.6); 0.999 (3.0); 0.000 (4.6)

Compound No. 3917, solvent [DMSO], 400 MHz

8.395 (0.6); 7.702 (0.7); 7.699 (0.7); 7.626 (0.7); 7.316 (2.6); 7.299 (1.8); 7.208 (1.6); 4.252 (8.8); 4.152 (3.3); 4.075 (2.8); 3.760 (2.2); 3.701 (16.0); 3.669 (10.7); 3.655 (1.6); 3.580 (9.6); 3.168 (0.8); 3.098 (7.2); 2.943 (6.4); 2.914 (9.8); 2.674 (0.6); 2.670 (0.9); 2.665 (0.6); 2.523 (2.5); 2.518 (3.8); 2.510 (50.5); 2.505 (109.6); 2.501 (152.9); 2.496 (106.2); 2.491 (47.2); 2.332 (0.7); 2.327 (1.0); 2.323 (0.7); 2.173 (5.4); 2.159 (4.1); 1.987 (5.7); 1.958 (4.0); 0.008 (0.9); 0.000 (31.4); −0.009 (0.9)

Compound No. 2599, solvent [DMSO], 400 MHz

12.289 (0.7); 9.730 (0.9); 8.132 (6.6); 7.918 (1.1); 3.710 (2.6); 3.431 (0.6); 3.317 (171.5); 3.309 (4.1); 3.295 (3.0); 3.291 (3.0); 3.277 (2.7); 3.259 (0.9); 2.670 (0.7); 2.524 (3.3); 2.519 (4.8); 2.510 (41.3); 2.506 (86.5); 2.501 (119.0); 2.497 (83.2); 2.492 (37.3); 2.448 (1.4); 2.429 (3.6); 2.410 (3.6); 2.391 (1.2); 2.370 (0.6); 2.333 (0.5); 2.328 (0.7); 2.324 (0.5); 2.271 (16.0); 2.229 (1.8); 1.120 (5.9); 1.102 (13.5); 1.086 (6.3); 1.084 (7.3); 1.068 (12.6); 1.049 (5.5); 1.028 (0.7); 0.008 (1.1); 0.000 (34.3); −0.009 (1.0)

Compound No. 2620, solvent [DMSO], 400 MHz

12.268 (0.6); 9.756 (0.7); 8.128 (4.2); 3.317 (71.3); 3.307 (1.7); 3.290 (1.4); 3.272 (0.8); 2.524 (2.0); 2.511 (20.4); 2.506 (42.3); 2.501 (58.0); 2.497 (40.9); 2.492 (18.6); 2.448 (0.8); 2.429 (2.2); 2.410 (2.2); 2.391 (0.7); 2.272 (9.7); 1.614 (0.6); 1.598 (0.8); 1.581 (0.7); 1.410 (1.0); 1.393 (1.9); 1.374 (1.9); 1.357 (0.8); 1.086 (3.0); 1.068 (7.2); 1.049 (2.9); 0.902 (16.0); 0.886 (15.1); 0.008 (0.8); 0.000 (21.5); −0.009 (0.7)

Compound No. 2643, solvent [DMSO], 400 MHz

12.279 (0.9); 9.849 (1.0); 9.836 (0.5); 8.137 (6.5); 3.463 (0.6); 3.453 (2.0); 3.442 (2.8); 3.438 (2.8); 3.431 (6.4); 3.423 (3.5); 3.410 (0.8); 3.368 (0.6); 3.350 (0.5); 3.318 (138.4); 3.269 (1.0); 3.265 (35.4); 2.524 (2.1); 2.519 (3.1); 2.511 (25.1); 2.506 (51.3); 2.501 (69.8); 2.497 (49.0); 2.492 (21.9); 2.450 (1.1); 2.431 (3.4); 2.412 (3.5); 2.393 (1.2); 2.276 (16.0); 1.087 (4.9); 1.068 (11.8); 1.049 (4.7); 0.008 (1.2); 0.000 (33.4); −0.009 (1.0)

Compound No. 2595, solvent [DMSO], 400 MHz

11.681 (0.7); 7.242 (6.7); 3.384 (0.8); 3.366 (2.5); 3.349 (2.7); 3.325 (21.0); 3.167 (0.8); 3.149 (2.2); 3.132 (2.2); 3.114 (0.7); 2.524 (1.2); 2.511 (11.9); 2.506 (24.6); 2.501 (33.8); 2.497 (23.8); 2.492 (10.8); 2.363 (1.0); 2.344 (3.1); 2.325 (3.2); 2.306 (1.0); 2.176 (16.0); 1.106 (2.7); 1.088 (5.7); 1.071 (2.7); 1.048 (4.9); 1.029 (12.5); 1.014 (6.1); 1.010 (5.9); 0.997 (2.5); 0.008 (0.5); 0.000 (13.2)

Compound No. 2597, solvent [DMSO], 400 MHz

12.388 (0.7); 10.055 (0.9); 8.132 (4.4); 4.114 (3.5); 4.100 (3.5); 3.643 (16.0); 3.317 (146.7); 3.205 (0.6); 2.670 (0.7); 2.523 (1.9); 2.510 (37.7); 2.506 (79.7); 2.501 (109.1); 2.496 (76.7); 2.492 (34.4); 2.456 (0.8); 2.438 (2.4); 2.419 (2.4); 2.401 (0.9); 2.328 (0.6); 2.289 (10.1); 1.090 (3.1); 1.072 (7.1); 1.053 (3.0); 0.008 (1.3); 0.000 (39.7); −0.009 (1.1)

Compound No. 2485, solvent [DMSO], 400 MHz

12.262 (1.1); 7.568 (6.4); 3.394 (19.8); 3.361 (7.6); 3.343 (6.9); 3.325 (2.3); 3.168 (0.9); 3.162 (1.8); 3.144 (5.8); 3.126 (5.9); 3.109 (1.8); 2.675 (0.9); 2.670 (1.2); 2.665 (0.8); 2.523 (3.1); 2.519 (4.6); 2.510 (70.5); 2.506 (151.0); 2.501 (207.0); 2.496 (144.9); 2.492 (65.4); 2.325 (25.8); 1.100 (7.2); 1.082 (16.0); 1.065 (7.1); 1.041 (6.8); 1.024 (15.1); 1.006 (6.5); 0.008 (2.2); 0.000 (77.3); −0.009 (2.3)

Compound No. 2600, solvent [DMSO], 400 MHz

3.501 (16.0); 2.522 (1.3); 2.517 (1.8); 2.514 (1.4); 2.283 (0.6)

Compound No. 2471, solvent [DMSO], 400 MHz

12.359 (1.2); 7.467 (4.9); 7.451 (4.8); 5.153 (2.6); 4.373 (1.2); 4.307 (2.1); 4.185 (2.6); 4.167 (7.6); 4.149 (7.8); 4.132 (3.1); 4.124 (2.4); 4.094 (1.8); 4.076 (1.8); 3.573 (9.9); 3.416 (2.0); 3.382 (2.2); 3.137 (1.2); 3.111 (1.9); 2.725 (1.4); 2.670 (1.8); 2.604 (0.8); 2.505 (221.2); 2.501 (300.1); 2.497 (245.7); 2.445 (1.7); 2.301 (16.4); 2.284 (13.8); 2.151 (1.6); 2.120 (1.8); 2.057 (1.2); 2.025 (1.4); 1.682 (4.6); 1.654 (5.0); 1.525 (2.3); 1.482 (1.5); 1.323 (1.6); 1.292 (2.1); 1.241 (8.1); 1.224 (16.0); 1.206 (8.3); 1.195 (6.0); 1.178 (10.4); 1.160 (5.1); 0.000 (72.6)

Compound No. 2489, solvent [DMSO], 400 MHz

14.204 (0.9); 12.828 (2.5); 9.511 (2.8); 9.022 (2.5); 8.382 (9.3); 7.672 (6.2); 3.323 (114.9); 3.280 (5.8); 3.236 (1.1); 2.670 (3.7); 2.505 (469.2); 2.501 (482.1); 2.403 (25.6); 2.328 (3.0); 1.120 (8.5); 1.101 (16.0); 1.084 (7.5); 0.002 (67.4); 0.000 (86.3)

Compound No. 2581, solvent [DMSO], 400 MHz

12.329 (1.1); 7.539 (7.2); 5.145 (1.6); 5.133 (1.5); 4.370 (0.8); 4.335 (0.8); 4.302 (1.2); 4.291 (1.2); 4.184 (2.1); 4.166 (6.7); 4.148 (6.9); 4.142 (2.1); 4.130 (2.3); 4.124 (1.8); 4.107 (0.7); 4.092 (1.6); 4.074 (1.6); 4.065 (0.9); 4.056 (0.5); 4.047 (0.8); 3.466 (7.1); 3.413 (1.6); 3.374 (1.3); 3.120 (0.6); 3.096 (1.0); 3.090 (1.0); 3.065 (0.5); 2.715 (0.7); 2.679 (0.6); 2.675 (0.7); 2.670 (0.9); 2.666 (0.6); 2.524 (2.1); 2.519 (3.3); 2.510 (50.2); 2.506 (106.7); 2.501 (145.6); 2.496 (102.7); 2.492 (47.1); 2.341 (15.2); 2.324 (12.7); 2.149 (0.9); 2.113 (0.9); 2.053 (0.7); 2.019 (0.8); 1.680 (2.7); 1.651 (2.9); 1.624 (0.7); 1.608 (0.5); 1.558 (0.6); 1.526 (1.2); 1.480 (0.8); 1.448 (0.6); 1.319 (0.9); 1.289 (1.1); 1.242 (7.9); 1.224 (16.0); 1.207 (7.9); 1.201 (5.7); 1.183 (10.4); 1.165 (4.8); 0.008 (1.5); 0.000 (51.5); −0.009 (1.7)

Compound No. 2375, solvent [DMSO], 400 MHz

12.282 (0.5); 7.515 (4.8); 3.462 (3.8); 3.386 (1.7); 3.369 (4.4); 3.351 (4.4); 3.333 (1.6); 3.168 (1.4); 3.151 (4.0); 3.133 (4.1); 3.116 (1.4); 2.670 (0.6); 2.506 (82.5); 2.501 (107.7); 2.497 (78.9); 2.449 (0.7); 2.328 (0.7); 2.286 (16.0); 1.105 (4.7); 1.087 (10.1); 1.069 (4.8); 1.046 (4.7); 1.028 (9.7); 1.011 (4.4); 0.000 (30.2)

Compound No. 2533, solvent [DMSO], 400 MHz

14.151 (2.1); 12.819 (4.2); 9.641 (3.9); 8.954 (3.0); 8.387 (16.0); 7.640 (8.5); 3.434 (23.1); 3.425 (12.0); 3.372 (4.3); 3.315 (862.0); 3.264 (60.7); 2.669 (14.3); 2.590 (3.3); 2.555 (11.7); 2.505 (2167.4); 2.501 (2396.5); 2.496 (1632.8); 2.407 (44.8); 2.327 (14.6); 2.073 (2.0); 0.146 (3.1); 0.000 (750.3); −0.009 (51.0); −0.150 (3.8)

Compound No. 2490, solvent [DMSO], 400 MHz

12.824 (2.2); 9.573 (2.3); 8.382 (9.7); 7.654 (1.2); 3.317 (99.1); 3.267 (2.3); 3.251 (5.3); 3.236 (5.7); 3.218 (2.5); 2.675 (1.5); 2.670 (1.7); 2.505 (253.6); 2.501 (277.2); 2.496 (184.9); 2.404 (25.2); 2.328 (1.8); 1.543 (0.6); 1.524 (2.5); 1.507 (4.9); 1.489 (5.1); 1.471 (2.8); 1.453 (0.7); 0.900 (8.0); 0.881 (16.0); 0.863 (7.2); 0.000 (92.4); −0.009 (5.9)

Compound No. 2510, solvent [DMSO], 400 MHz

12.807 (0.6); 9.534 (0.6); 8.378 (4.6); 3.324 (1.0); 3.314 (22.0); 3.292 (1.2); 3.289 (1.2); 3.274 (0.8); 2.524 (0.5); 2.519 (0.8); 2.511 (12.6); 2.506 (27.1); 2.501 (37.3); 2.497 (26.1); 2.492 (11.8); 2.403 (10.1); 1.606 (0.6); 1.590 (0.8); 1.573 (0.7); 1.411 (0.9); 1.394 (1.8); 1.375 (1.8); 1.358 (0.8); 0.899 (16.0); 0.883 (15.1); 0.008 (0.7); 0.000 (22.1); −0.009 (0.6)

Compound No. 2546, solvent [DMSO], 400 MHz

9.843 (0.7); 8.971 (1.2); 8.382 (5.2); 7.648 (3.5); 7.645 (3.4); 4.166 (0.5); 4.119 (3.1); 4.105 (3.1); 3.795 (1.2); 3.780 (1.3); 3.723 (1.0); 3.673 (1.1); 3.646 (16.0); 3.615 (5.6); 3.413 (0.6); 3.369 (0.6); 3.351 (0.8); 3.317 (85.5); 3.267 (0.6); 3.131 (0.5); 2.679 (0.6); 2.674 (1.3); 2.670 (2.0); 2.665 (1.4); 2.660 (0.6); 2.604 (0.7); 2.599 (2.0); 2.595 (2.0); 2.590 (1.5); 2.561 (1.0); 2.556 (1.1); 2.552 (0.9); 2.534 (0.5); 2.523 (3.7); 2.518 (6.1); 2.510 (110.1); 2.505 (239.2); 2.501 (330.6); 2.496 (230.8); 2.491 (104.2); 2.474 (2.0); 2.470 (1.6); 2.465 (1.0); 2.460 (0.9); 2.455 (1.1); 2.451 (1.5); 2.446 (1.0); 2.441 (0.6); 2.418 (11.0); 2.337 (0.7); 2.332 (1.4); 2.327 (2.0); 2.323 (1.4); 2.318 (0.6); 2.073 (0.5); 1.176 (1.0); 0.146 (0.6); 0.099 (1.3); 0.008 (5.5); 0.000 (214.5); −0.009 (6.6); −0.031 (0.7); −0.050 (0.9); −0.150 (0.7)

Compound No. 2290, solvent [DMSO], 400 MHz

12.851 (0.5); 9.563 (0.6); 8.153 (4.1); 3.332 (1.2); 3.314 (138.1); 3.300 (1.8); 3.282 (0.9); 2.674 (0.7); 2.670 (1.1); 2.665 (0.7); 2.523 (2.4); 2.519 (3.5); 2.510 (61.6); 2.505 (133.0); 2.501 (183.0); 2.496 (129.0); 2.492 (59.2); 2.455 (0.6); 2.450 (0.7); 2.446 (0.6); 2.349 (10.1); 2.337 (0.5); 2.332 (0.9); 2.328 (1.1); 2.323 (0.8); 1.611 (0.6); 1.594 (0.8); 1.577 (0.7); 1.418 (1.0); 1.400 (1.9); 1.382 (1.9); 1.364 (0.8); 0.902 (16.0); 0.885 (15.2); 0.008 (1.5); 0.000 (58.5); −0.009 (1.9)

Compound No. 2265, solvent [DMSO], 400 MHz

7.455 (5.5); 3.389 (1.7); 3.371 (4.3); 3.354 (4.5); 3.336 (2.0); 3.316 (43.2); 3.170 (1.3); 3.152 (3.8); 3.134 (3.9); 3.117 (1.4); 2.538 (1.8); 2.524 (2.5); 2.519 (2.8); 2.510 (22.4); 2.506 (46.4); 2.501 (63.5); 2.497 (46.9); 2.492 (24.3); 2.333 (0.5); 2.328 (0.6); 2.324 (0.5); 2.267 (16.0); 1.106 (4.7); 1.089 (9.8); 1.071 (5.0); 1.046 (4.6); 1.029 (9.2); 1.011 (4.4); 0.008 (0.5); 0.000 (14.9); −0.009 (0.8)

Compound No. 2270, solvent [DMSO], 400 MHz

12.865 (1.0); 9.601 (1.1); 8.157 (8.7); 3.315 (32.7); 3.276 (1.9); 3.259 (3.7); 3.244 (3.7); 3.227 (2.0); 2.524 (0.8); 2.519 (1.4); 2.511 (18.2); 2.506 (38.1); 2.502 (51.7); 2.497 (37.4); 2.493 (18.1); 2.350 (23.2); 2.333 (0.5); 1.532 (2.1); 1.514 (3.9); 1.496 (3.9); 1.478 (2.3); 1.460 (0.5); 0.905 (7.5); 0.886 (16.0); 0.868 (6.7); 0.008 (0.6); 0.000 (17.3); −0.005 (0.7); −0.006 (0.6); −0.009 (0.8)

Compound No. 2313, solvent [DMSO], 400 MHz

12.857 (2.1); 9.670 (2.1); 8.163 (16.0); 3.484 (1.4); 3.478 (1.8); 3.474 (2.0); 3.466 (5.2); 3.454 (6.8); 3.450 (6.0); 3.443 (14.8); 3.433 (9.1); 3.420 (2.7); 3.414 (1.6); 3.373 (1.1); 3.367 (1.4); 3.315 (68.0); 3.268 (72.6); 3.236 (0.6); 3.218 (0.6); 3.169 (0.6); 2.671 (0.7); 2.666 (0.5); 2.557 (3.9); 2.552 (4.0); 2.548 (3.6); 2.543 (3.6); 2.524 (5.1); 2.519 (5.6); 2.511 (39.1); 2.506 (81.0); 2.501 (111.4); 2.497 (82.7); 2.492 (43.4); 2.461 (1.2); 2.457 (1.3); 2.452 (1.2); 2.447 (0.6); 2.427 (0.8); 2.412 (0.7); 2.405 (0.7); 2.353 (38.1); 2.333 (1.4); 2.328 (1.2); 2.324 (0.7); 0.008 (0.9); 0.000 (26.0); −0.009 (1.4)

Compound No. 2269, solvent [DMSO], 400 MHz

12.865 (1.1); 9.540 (1.3); 8.157 (8.6); 3.366 (0.6); 3.339 (1.1); 3.321 (6.8); 3.314 (55.7); 3.307 (7.8); 3.303 (5.8); 3.289 (4.0); 3.271 (1.5); 3.265 (1.1); 2.561 (0.8); 2.557 (0.9); 2.552 (0.7); 2.524 (0.8); 2.510 (30.8); 2.506 (63.7); 2.501 (85.9); 2.497 (64.2); 2.492 (32.9); 2.456 (1.4); 2.452 (1.4); 2.447 (0.9); 2.348 (23.4); 2.328 (0.8); 2.324 (0.6); 1.126 (7.4); 1.108 (16.0); 1.090 (7.4); 0.008 (0.9); 0.000 (21.8); −0.008 (1.3)

Compound No. 4069, solvent [DMSO], 400 MHz

12.004 (0.7); 8.182 (0.7); 7.724 (2.2); 7.630 (1.3); 4.252 (6.7); 4.013 (3.4); 3.694 (16.0); 3.615 (0.9); 3.600 (9.5); 3.353 (5.0); 3.169 (1.0); 3.013 (8.4); 2.876 (14.9); 2.712 (1.1); 2.693 (1.4); 2.679 (1.2); 2.674 (1.7); 2.670 (2.0); 2.665 (1.6); 2.645 (0.6); 2.533 (0.7); 2.523 (3.5); 2.519 (5.0); 2.510 (64.7); 2.505 (140.6); 2.501 (195.7); 2.496 (135.4); 2.491 (60.1); 2.332 (0.9); 2.327 (1.2); 2.323 (0.8); 1.180 (2.6); 1.162 (5.7); 1.143 (2.7); 1.139 (2.6); 1.120 (3.3); 1.101 (1.4); 0.008 (1.6); 0.006 (0.5); 0.005 (0.6); 0.004 (0.8); 0.000 (50.9); −0.009 (1.4)

Compound No. 3993, solvent [DMSO], 400 MHz

7.511 (1.7); 7.473 (1.7); 4.171 (4.3); 4.070 (3.3); 3.674 (12.9); 3.607 (9.4); 3.470 (16.0); 3.168 (2.2); 2.946 (7.8); 2.918 (11.7); 2.674 (0.9); 2.670 (1.2); 2.665 (1.0); 2.523 (4.0); 2.518 (5.8); 2.510 (78.8); 2.505 (170.8); 2.500 (237.1); 2.496 (164.0); 2.491 (72.4); 2.332 (1.0); 2.327 (1.5); 2.322 (1.3); 2.309 (4.8); 2.288 (3.8); 2.072 (1.1); 0.008 (1.2); 0.000 (42.8); −0.009 (1.3)

Compound No. 3977, solvent [DMSO], 400 MHz

12.308 (0.9); 7.403 (3.8); 7.382 (3.7); 5.158 (1.7); 5.146 (1.5); 4.383 (0.8); 4.350 (0.8); 4.319 (1.2); 4.308 (1.2); 4.185 (2.2); 4.167 (7.0); 4.157 (1.1); 4.149 (7.3); 4.139 (2.1); 4.132 (2.5); 4.121 (2.0); 4.113 (0.6); 4.104 (0.7); 4.096 (1.6); 4.078 (1.6); 4.069 (0.8); 4.060 (0.6); 4.051 (0.8); 3.417 (0.9); 3.385 (1.1); 3.313 (125.6); 3.150 (0.6); 3.141 (0.7); 3.110 (1.1); 3.086 (0.6); 2.730 (0.7); 2.675 (0.7); 2.670 (1.0); 2.665 (0.7); 2.523 (2.7); 2.518 (4.1); 2.510 (54.3); 2.505 (116.1); 2.501 (160.8); 2.496 (112.8); 2.492 (51.2); 2.450 (0.5); 2.332 (0.8); 2.328 (1.1); 2.323 (0.8); 2.283 (12.2); 2.266 (10.2); 2.152 (1.0); 2.117 (1.0); 2.072 (0.6); 2.057 (0.7); 2.023 (0.9); 1.686 (2.9); 1.674 (2.4); 1.655 (3.1); 1.627 (0.7); 1.612 (0.6); 1.558 (0.6); 1.527 (1.4); 1.484 (0.8); 1.453 (0.7); 1.324 (1.0); 1.296 (1.3); 1.257 (1.2); 1.242 (7.6); 1.224 (16.0); 1.206 (7.5); 1.193 (5.2); 1.175 (10.1); 1.157 (4.7); 0.008 (1.9); 0.000 (63.2); −0.009 (2.0)

Compound No. 2840, solvent [DMSO], 400 MHz

12.248 (0.5); 9.762 (0.6); 8.108 (3.6); 3.333 (17.4); 3.305 (1.5); 3.290 (1.3); 3.288 (1.3); 3.272 (0.9); 2.588 (0.6); 2.569 (1.9); 2.550 (2.0); 2.531 (0.7); 2.524 (0.7); 2.519 (0.9); 2.510 (11.5); 2.506 (24.4); 2.501 (33.7); 2.497 (23.6); 2.492 (10.6); 2.086 (10.8); 1.615 (0.6); 1.598 (0.8); 1.581 (0.7); 1.408 (1.0); 1.391 (1.9); 1.372 (1.9); 1.355 (0.8); 1.129 (2.4); 1.111 (5.8); 1.092 (2.4); 0.902 (16.0); 0.885 (15.2); 0.000 (13.8)

Compound No. 2911, solvent [DMSO], 400 MHz

7.279 (3.6); 7.263 (6.1); 5.154 (1.2); 5.142 (1.2); 4.388 (0.5); 4.297 (0.9); 4.285 (0.8); 4.180 (1.5); 4.162 (4.9); 4.144 (5.4); 4.135 (0.7); 4.127 (2.2); 4.118 (1.3); 4.100 (1.4); 4.084 (1.4); 4.066 (1.2); 4.057 (0.6); 4.039 (0.6); 3.792 (1.3); 3.425 (0.7); 3.392 (0.9); 3.169 (1.1); 3.091 (0.7); 3.083 (0.7); 2.761 (0.5); 2.524 (1.0); 2.511 (16.3); 2.506 (34.6); 2.501 (48.5); 2.497 (36.1); 2.492 (16.5); 2.479 (3.6); 2.469 (2.0); 2.461 (1.4); 2.449 (0.5); 2.150 (0.8); 2.113 (0.7); 2.047 (0.5); 2.031 (0.6); 2.017 (16.0); 1.993 (9.6); 1.681 (1.8); 1.666 (1.3); 1.651 (2.3); 1.526 (1.2); 1.302 (0.5); 1.280 (0.7); 1.241 (6.3); 1.223 (12.3); 1.206 (5.9); 1.173 (3.3); 1.155 (6.6); 1.138 (3.2); 1.125 (3.8); 1.114 (2.9); 1.106 (8.7); 1.095 (5.6); 1.087 (4.0); 1.076 (2.3); 0.008 (0.7); 0.000 (22.4); −0.009 (0.7)

Compound No. 2820, solvent [DMSO], 400 MHz

12.262 (0.8); 9.816 (0.5); 9.802 (0.9); 8.113 (5.3); 3.311 (38.1); 3.266 (1.3); 3.248 (2.7); 3.234 (2.7); 3.216 (1.4); 2.589 (0.9); 2.570 (2.9); 2.551 (3.2); 2.532 (1.2); 2.524 (0.9); 2.519 (1.2); 2.510 (14.6); 2.506 (30.8); 2.501 (42.3); 2.497 (29.9); 2.492 (13.5); 2.087 (16.0); 1.521 (1.5); 1.503 (2.8); 1.485 (2.8); 1.467 (1.6); 1.131 (3.7); 1.112 (8.8); 1.093 (3.6); 0.904 (5.2); 0.886 (10.8); 0.867 (4.5); 0.008 (0.6); 0.000 (17.8); −0.009 (0.6)

Compound No. 2819, solvent [DMSO], 400 MHz

12.267 (0.7); 9.749 (0.5); 9.736 (0.9); 9.723 (0.5); 8.113 (5.3); 3.478 (3.4); 3.328 (0.7); 3.310 (2.3); 3.296 (2.6); 3.292 (2.5); 3.278 (2.3); 3.260 (0.7); 2.587 (0.9); 2.568 (3.1); 2.550 (3.2); 2.531 (1.2); 2.524 (0.7); 2.511 (11.0); 2.506 (22.0); 2.502 (29.4); 2.497 (20.5); 2.493 (9.3); 2.087 (16.0); 1.130 (3.8); 1.119 (5.4); 1.111 (8.7); 1.100 (10.7); 1.092 (3.8); 1.082 (4.8); 0.000 (8.0)

Compound No. 2815, solvent [DMSO], 400 MHz

7.229 (6.0); 3.385 (0.6); 3.367 (1.9); 3.350 (1.9); 3.332 (0.7); 3.174 (0.6); 3.169 (0.5); 3.156 (1.8); 3.139 (1.8); 3.121 (0.6); 2.511 (6.1); 2.506 (13.2); 2.501 (18.8); 2.497 (13.0); 2.492 (5.9); 2.481 (3.0); 2.463 (3.0); 2.444 (1.0); 1.999 (16.0); 1.117 (3.8); 1.104 (2.7); 1.098 (9.5); 1.087 (4.7); 1.079 (4.4); 1.069 (2.2); 1.042 (2.1); 1.024 (4.2); 1.006 (2.0); 0.000 (8.3)

Compound No. 2817, solvent [DMSO], 400 MHz

7.320 (4.7); 7.300 (3.2); 4.206 (0.7); 4.157 (6.0); 4.073 (4.7); 4.062 (0.9); 3.981 (0.8); 3.670 (16.0); 3.581 (10.7); 3.169 (0.8); 2.947 (9.8); 2.924 (1.7); 2.915 (15.8); 2.895 (1.8); 2.524 (1.6); 2.519 (1.6); 2.510 (18.4); 2.506 (40.5); 2.501 (54.4); 2.497 (38.1); 2.492 (18.1); 2.470 (2.2); 2.151 (1.4); 2.024 (13.5); 1.995 (8.6); 1.144 (0.9); 1.125 (3.5); 1.106 (7.9); 1.091 (5.2); 1.088 (3.9); 1.073 (1.9); 0.008 (0.7); 0.000 (25.9); −0.009 (0.8)

Compound No. 2863, solvent [DMSO], 400 MHz

12.255 (0.6); 9.859 (0.9); 8.118 (5.3); 3.454 (10.3); 3.445 (5.8); 3.443 (5.7); 3.439 (4.7); 3.431 (6.5); 3.429 (6.1); 3.420 (3.9); 3.411 (1.0); 3.408 (1.1); 3.401 (0.7); 3.265 (31.1); 2.591 (0.9); 2.572 (2.9); 2.554 (3.0); 2.535 (1.0); 2.524 (0.7); 2.519 (1.0); 2.511 (11.1); 2.506 (23.4); 2.502 (32.3); 2.497 (22.6); 2.492 (10.1); 2.088 (16.0); 1.131 (3.7); 1.113 (8.8); 1.094 (3.5); 0.008 (0.5); 0.000 (15.5)

Compound No. 3853, solvent [DMSO], 400 MHz

12.758 (1.0); 9.642 (1.7); 9.629 (1.0); 8.401 (15.5); 3.476 (0.6); 3.471 (0.9); 3.466 (1.1); 3.458 (3.3); 3.446 (4.4); 3.444 (4.0); 3.432 (8.5); 3.423 (5.8); 3.414 (1.1); 3.410 (1.5); 3.404 (0.8); 3.307 (29.5); 3.264 (56.4); 2.676 (3.1); 2.660 (3.1); 2.657 (3.6); 2.652 (2.9); 2.637 (3.2); 2.525 (0.8); 2.520 (1.2); 2.511 (14.0); 2.507 (29.4); 2.502 (40.5); 2.497 (28.4); 2.493 (12.9); 1.618 (1.7); 1.599 (2.9); 1.580 (2.9); 1.561 (1.7); 0.949 (7.2); 0.931 (16.0); 0.912 (6.5); 0.008 (0.7); 0.000 (21.0); −0.009 (0.6)

Compound No. 3810, solvent [DMSO], 400 MHz

12.770 (1.1); 9.586 (0.7); 9.572 (1.2); 9.558 (0.7); 8.396 (11.6); 3.307 (28.4); 3.270 (1.8); 3.252 (3.4); 3.237 (3.4); 3.220 (1.8); 2.673 (2.3); 2.658 (2.2); 2.654 (2.7); 2.650 (2.1); 2.635 (2.4); 2.525 (0.7); 2.520 (0.9); 2.511 (11.7); 2.507 (25.1); 2.502 (34.9); 2.497 (24.3); 2.493 (10.9); 1.617 (1.3); 1.598 (2.1); 1.579 (2.2); 1.560 (1.3); 1.542 (0.8); 1.524 (2.0); 1.505 (3.7); 1.488 (3.7); 1.470 (2.2); 0.949 (5.6); 0.931 (12.7); 0.912 (5.1); 0.900 (7.5); 0.882 (16.0); 0.863 (6.5); 0.008 (0.6); 0.000 (18.0); −0.009 (0.5)

Compound No. 3830, solvent [DMSO], 400 MHz

9.531 (0.7); 8.392 (6.5); 3.326 (1.0); 3.308 (19.0); 3.294 (1.4); 3.291 (1.4); 3.275 (0.8); 2.672 (1.1); 2.656 (1.1); 2.653 (1.3); 2.648 (1.0); 2.633 (1.1); 2.519 (0.6); 2.511 (8.0); 2.506 (17.4); 2.502 (24.3); 2.497 (17.0); 2.492 (7.7); 1.615 (0.6); 1.607 (0.7); 1.596 (1.1); 1.591 (1.2); 1.577 (1.2); 1.574 (1.3); 1.558 (0.9); 1.410 (0.9); 1.392 (1.8); 1.374 (1.8); 1.356 (0.7); 0.948 (2.6); 0.930 (5.8); 0.911 (2.4); 0.899 (16.0); 0.882 (15.3); 0.863 (0.6); 0.846 (0.5); 0.000 (5.0)

Compound No. 3809, solvent [DMSO], 400 MHz

12.765 (0.6); 9.524 (0.7); 9.509 (1.2); 9.496 (0.6); 8.396 (13.3); 3.332 (1.1); 3.309 (27.2); 3.300 (4.0); 3.296 (3.6); 3.282 (3.3); 3.279 (1.4); 3.264 (1.0); 2.671 (2.5); 2.652 (2.7); 2.648 (2.1); 2.633 (2.4); 2.525 (0.6); 2.520 (1.0); 2.511 (13.2); 2.507 (28.3); 2.502 (39.2); 2.498 (27.3); 2.493 (12.3); 1.616 (1.2); 1.597 (2.1); 1.578 (2.2); 1.559 (1.3); 1.118 (7.1); 1.100 (16.0); 1.082 (6.9); 0.947 (5.4); 0.929 (12.1); 0.910 (4.9); 0.000 (3.1)

Compound No. 3589, solvent [DMSO], 400 MHz

12.823 (0.7); 9.538 (0.7); 8.165 (11.5); 3.444 (2.6); 3.342 (1.0); 3.328 (1.2); 3.324 (3.1); 3.310 (3.3); 3.306 (3.3); 3.292 (3.1); 3.288 (1.2); 3.274 (1.0); 2.667 (2.4); 2.648 (2.8); 2.645 (2.2); 2.629 (2.5); 2.524 (0.7); 2.519 (1.0); 2.511 (16.3); 2.506 (35.9); 2.501 (50.4); 2.497 (35.0); 2.492 (15.7); 1.646 (1.3); 1.627 (2.3); 1.608 (2.3); 1.589 (1.4); 1.125 (7.0); 1.107 (16.0); 1.089 (6.9); 0.930 (5.6); 0.912 (12.5); 0.893 (5.0); 0.000 (13.0)

Compound No. 3585, solvent [DMSO], 400 MHz

7.458 (3.5); 3.443 (3.5); 3.392 (1.6); 3.374 (3.9); 3.357 (3.8); 3.339 (1.3); 3.169 (1.3); 3.151 (3.5); 3.133 (3.5); 3.115 (1.1); 2.591 (1.7); 2.573 (2.6); 2.553 (1.9); 2.523 (1.3); 2.518 (1.9); 2.510 (23.6); 2.505 (51.0); 2.501 (71.4); 2.496 (49.5); 2.491 (21.9); 1.635 (1.4); 1.616 (2.5); 1.597 (2.5); 1.578 (1.4); 1.108 (4.4); 1.090 (9.7); 1.073 (4.4); 1.051 (4.2); 1.034 (9.0); 1.016 (3.9); 0.930 (7.2); 0.911 (16.0); 0.893 (6.4); 0.008 (0.7); 0.002 (0.9); 0.000 (23.3); −0.009 (0.6)

Compound No. 3590, solvent [DMSO], 400 MHz

12.818 (1.0); 9.598 (1.0); 8.165 (10.3); 3.304 (53.6); 3.278 (2.0); 3.261 (3.5); 3.246 (3.5); 3.229 (2.0); 2.668 (2.7); 2.650 (3.1); 2.630 (2.6); 2.523 (1.8); 2.518 (2.6); 2.510 (37.1); 2.505 (80.3); 2.501 (112.0); 2.496 (78.0); 2.491 (35.0); 2.327 (0.6); 1.647 (1.4); 1.628 (2.4); 1.609 (2.5); 1.591 (1.4); 1.531 (2.0); 1.513 (3.7); 1.495 (3.8); 1.477 (2.1); 0.932 (6.0); 0.913 (13.5); 0.905 (7.9); 0.895 (5.8); 0.886 (16.0); 0.868 (6.6); 0.008 (0.9); 0.000 (30.1); −0.009 (0.9)

Compound No. 3610, solvent [DMSO], 400 MHz

12.807 (0.5); 9.562 (0.5); 8.161 (4.7); 3.335 (0.8); 3.317 (1.5); 3.306 (22.0); 3.285 (0.8); 2.666 (1.2); 2.648 (1.4); 2.629 (1.1); 2.523 (0.7); 2.519 (1.0); 2.510 (13.6); 2.506 (29.1); 2.501 (40.2); 2.496 (27.8); 2.492 (12.4); 1.645 (0.7); 1.627 (1.2); 1.612 (1.2); 1.607 (1.2); 1.595 (0.9); 1.589 (0.7); 1.579 (0.7); 1.417 (0.9); 1.400 (1.8); 1.381 (1.8); 1.364 (0.7); 0.931 (2.8); 0.913 (6.3); 0.902 (16.0); 0.894 (3.1); 0.886 (15.1); 0.000 (11.3)

Compound No. 3681, solvent [DMSO], 400 MHz

7.407 (3.6); 7.381 (3.3); 5.161 (1.5); 5.150 (1.4); 4.385 (0.7); 4.350 (0.7); 4.320 (1.1); 4.308 (1.0); 4.186 (1.9); 4.168 (5.8); 4.150 (6.1); 4.139 (1.9); 4.133 (2.2); 4.121 (2.2); 4.103 (2.0); 4.086 (1.5); 4.076 (0.7); 4.068 (0.5); 4.059 (0.7); 3.579 (2.1); 3.410 (0.9); 3.377 (1.1); 3.154 (0.6); 3.146 (0.6); 3.122 (1.0); 3.115 (1.0); 3.090 (0.6); 2.736 (0.7); 2.670 (0.5); 2.606 (1.8); 2.588 (3.2); 2.568 (3.0); 2.552 (1.2); 2.524 (1.5); 2.519 (2.2); 2.510 (26.4); 2.506 (56.6); 2.501 (79.0); 2.496 (55.0); 2.492 (24.3); 2.155 (0.9); 2.120 (0.9); 2.062 (0.6); 2.027 (0.7); 1.687 (2.6); 1.675 (2.1); 1.646 (3.4); 1.626 (3.1); 1.611 (3.3); 1.607 (3.3); 1.593 (2.5); 1.575 (1.6); 1.557 (0.7); 1.537 (1.2); 1.484 (0.7); 1.452 (0.6); 1.329 (0.9); 1.301 (1.0); 1.259 (1.1); 1.243 (7.6); 1.225 (16.0); 1.208 (7.4); 1.190 (4.6); 1.172 (9.2); 1.154 (4.3); 0.939 (5.5); 0.928 (4.2); 0.920 (11.9); 0.910 (7.9); 0.902 (5.5); 0.891 (3.4); 0.008 (0.7); 0.000 (26.7); −0.009 (0.8)

Compound No. 3633, solvent [DMSO], 400 MHz

12.816 (1.3); 9.670 (1.3); 8.172 (14.3); 3.486 (0.6); 3.480 (0.8); 3.475 (1.0); 3.468 (2.9); 3.456 (3.9); 3.444 (5.0); 3.440 (7.1); 3.429 (5.2); 3.422 (1.0); 3.418 (1.4); 3.411 (0.8); 3.326 (13.5); 3.306 (0.8); 3.267 (57.4); 2.671 (3.1); 2.652 (3.7); 2.633 (3.0); 2.524 (1.0); 2.519 (1.6); 2.511 (20.4); 2.506 (44.1); 2.501 (61.1); 2.497 (42.4); 2.492 (19.0); 1.648 (1.7); 1.629 (2.9); 1.610 (3.0); 1.591 (1.7); 0.932 (7.2); 0.914 (16.0); 0.895 (6.4); 0.008 (0.6); 0.000 (18.0); −0.009 (0.5)

Compound No. 3901, solvent [DMSO], 400 MHz

12.273 (1.4); 7.540 (4.0); 5.147 (2.1); 5.137 (1.9); 4.370 (0.9); 4.336 (1.0); 4.297 (1.5); 4.288 (1.4); 4.184 (2.5); 4.167 (7.4); 4.149 (7.6); 4.141 (2.3); 4.131 (2.7); 4.123 (2.0); 4.100 (1.7); 4.083 (1.6); 4.073 (0.8); 4.065 (0.6); 4.056 (0.7); 3.422 (7.0); 3.368 (1.9); 3.124 (0.8); 3.099 (1.3); 3.068 (0.7); 2.754 (0.6); 2.721 (0.9); 2.692 (0.5); 2.670 (0.6); 2.611 (2.2); 2.593 (4.5); 2.574 (4.3); 2.555 (1.9); 2.510 (31.2); 2.506 (61.5); 2.501 (82.5); 2.497 (60.3); 2.492 (29.5); 2.328 (0.5); 2.151 (1.2); 2.116 (1.2); 2.056 (0.8); 2.024 (1.0); 1.680 (3.6); 1.652 (4.0); 1.613 (2.4); 1.595 (3.5); 1.576 (4.4); 1.562 (3.5); 1.543 (2.6); 1.478 (1.0); 1.447 (0.9); 1.357 (0.6); 1.323 (1.2); 1.295 (1.4); 1.243 (8.4); 1.226 (16.0); 1.208 (8.2); 1.199 (5.5); 1.181 (9.7); 1.164 (4.6); 0.952 (6.0); 0.942 (5.4); 0.934 (12.6); 0.924 (9.1); 0.916 (6.2); 0.905 (3.8); 0.008 (0.8); 0.000 (16.6); −0.008 (0.8)

Compound No. 3805, solvent [DMSO], 400 MHz

12.210 (1.2); 7.572 (2.9); 3.380 (2.8); 3.356 (15.4); 3.328 (2.3); 3.169 (0.6); 3.159 (1.5); 3.142 (4.4); 3.124 (4.5); 3.106 (1.5); 2.670 (0.6); 2.598 (1.9); 2.579 (3.2); 2.559 (2.2); 2.523 (2.8); 2.509 (36.4); 2.505 (72.2); 2.500 (96.4); 2.496 (68.3); 2.491 (31.7); 2.327 (0.6); 1.604 (1.6); 1.585 (2.9); 1.566 (2.9); 1.547 (1.7); 1.101 (5.6); 1.083 (11.8); 1.066 (5.6); 1.046 (5.4); 1.029 (11.1); 1.011 (5.0); 0.944 (7.5); 0.926 (16.0); 0.908 (6.7); 0.008 (1.0); 0.000 (21.8); −0.009 (0.8)

Compound No. 3700, solvent [DMSO], 400 MHz

12.835 (1.1); 9.579 (1.1); 8.255 (9.4); 3.304 (81.1); 3.276 (1.9); 3.259 (3.5); 3.244 (3.3); 3.227 (1.9); 2.672 (2.6); 2.653 (2.8); 2.634 (2.3); 2.523 (2.6); 2.518 (3.6); 2.510 (42.5); 2.505 (90.5); 2.501 (125.2); 2.496 (86.1); 2.491 (38.0); 2.327 (0.7); 2.323 (0.5); 1.642 (1.3); 1.623 (2.3); 1.604 (2.3); 1.585 (1.4); 1.529 (2.0); 1.511 (3.6); 1.493 (3.6); 1.475 (2.1); 0.939 (5.9); 0.920 (13.2); 0.903 (10.8); 0.885 (16.0); 0.866 (6.5); 0.000 (14.6)

Compound No. 3743, solvent [DMSO], 400 MHz

12.828 (1.3); 9.651 (1.3); 8.261 (13.8); 3.483 (0.6); 3.477 (0.9); 3.473 (1.0); 3.466 (2.9); 3.453 (3.8); 3.437 (7.1); 3.427 (5.2); 3.415 (1.4); 3.409 (0.7); 3.304 (59.5); 3.266 (57.0); 2.674 (3.3); 2.656 (3.5); 2.636 (3.0); 2.523 (2.2); 2.518 (3.2); 2.510 (44.0); 2.505 (95.3); 2.501 (132.9); 2.496 (91.7); 2.491 (40.6); 2.332 (0.5); 2.327 (0.8); 2.323 (0.5); 1.643 (1.6); 1.624 (2.8); 1.605 (2.9); 1.586 (1.7); 0.939 (7.1); 0.921 (16.0); 0.902 (6.4); 0.000 (16.5); −0.008 (0.5)

Compound No. 3791, solvent [DMSO], 400 MHz

12.294 (0.8); 7.467 (1.8); 7.447 (1.8); 5.156 (1.5); 4.374 (0.7); 4.344 (0.8); 4.316 (1.1); 4.185 (2.3); 4.167 (7.2); 4.150 (7.3); 4.140 (2.1); 4.132 (2.5); 4.122 (2.0); 4.102 (1.6); 4.084 (1.5); 4.075 (0.7); 4.057 (0.8); 3.389 (21.6); 3.169 (0.7); 3.141 (0.7); 3.109 (1.0); 3.084 (0.5); 2.731 (0.7); 2.674 (1.0); 2.669 (1.3); 2.665 (0.9); 2.590 (3.0); 2.571 (2.8); 2.523 (4.3); 2.518 (5.8); 2.510 (77.0); 2.505 (166.2); 2.500 (231.0); 2.496 (158.8); 2.491 (69.9); 2.332 (1.0); 2.327 (1.2); 2.322 (1.0); 2.153 (0.9); 2.122 (0.9); 2.085 (1.6); 2.058 (0.7); 2.026 (0.8); 1.685 (2.6); 1.658 (3.1); 1.620 (2.8); 1.605 (3.0); 1.586 (2.2); 1.539 (1.2); 1.483 (0.7); 1.449 (0.7); 1.364 (0.5); 1.328 (0.9); 1.299 (1.1); 1.242 (7.8); 1.225 (16.0); 1.207 (7.3); 1.193 (4.7); 1.175 (9.1); 1.157 (4.2); 0.944 (5.2); 0.934 (4.2); 0.926 (11.1); 0.916 (7.6); 0.908 (5.0); 0.897 (3.2); 0.008 (1.0); 0.000 (34.4); −0.009 (0.9)

Compound No. 3721, solvent [DMSO], 400 MHz

9.541 (0.5); 8.251 (3.9); 3.333 (0.8); 3.304 (51.6); 3.283 (0.8); 2.670 (1.3); 2.651 (1.2); 2.632 (1.0); 2.523 (1.3); 2.518 (1.9); 2.510 (26.6); 2.505 (57.1); 2.500 (79.1); 2.496 (54.7); 2.491 (24.5); 1.641 (0.6); 1.621 (1.0); 1.610 (0.7); 1.602 (1.1); 1.594 (0.8); 1.584 (0.6); 1.577 (0.7); 1.415 (0.8); 1.398 (1.5); 1.379 (1.5); 1.362 (0.6); 0.938 (2.4); 0.920 (5.4); 0.901 (16.0); 0.885 (13.1); 0.000 (9.8)

Compound No. 3697, solvent [DMSO], 400 MHz

12.358 (0.7); 7.508 (1.3); 7.468 (1.2); 4.172 (4.2); 4.072 (3.0); 3.675 (16.0); 3.603 (11.3); 3.317 (261.6); 2.949 (7.1); 2.917 (10.1); 2.678 (1.1); 2.674 (2.3); 2.669 (3.3); 2.664 (2.4); 2.660 (1.1); 2.613 (0.8); 2.591 (1.6); 2.572 (1.5); 2.550 (1.7); 2.545 (1.5); 2.540 (1.2); 2.523 (9.2); 2.518 (13.5); 2.509 (184.5); 2.505 (397.6); 2.500 (556.4); 2.495 (388.7); 2.491 (173.7); 2.467 (1.7); 2.463 (1.4); 2.435 (0.6); 2.400 (0.6); 2.336 (1.2); 2.331 (2.4); 2.327 (3.4); 2.322 (2.3); 2.071 (1.4); 1.621 (1.4); 1.601 (1.7); 1.584 (1.3); 0.942 (2.8); 0.924 (6.8); 0.907 (5.2); 0.889 (1.8); 0.008 (2.0); 0.000 (65.6); −0.009 (2.0)

Compound No. 3695, solvent [DMSO], 400 MHz

7.519 (4.0); 3.628 (1.7); 3.389 (1.3); 3.371 (3.9); 3.354 (4.0); 3.336 (1.3); 3.169 (1.3); 3.150 (3.7); 3.132 (3.7); 3.114 (1.2); 2.597 (1.9); 2.578 (2.8); 2.559 (2.1); 2.523 (1.2); 2.519 (1.7); 2.510 (24.7); 2.505 (53.9); 2.501 (75.3); 2.496 (52.2); 2.491 (23.4); 1.630 (1.5); 1.611 (2.6); 1.592 (2.7); 1.574 (1.6); 1.106 (4.6); 1.089 (10.0); 1.071 (4.6); 1.051 (4.4); 1.034 (9.4); 1.016 (4.1); 0.936 (7.2); 0.918 (16.0); 0.899 (6.5); 0.000 (8.3)

Compound No. 3699, solvent [DMSO], 400 MHz

8.255 (8.8); 3.339 (3.9); 3.305 (707.1); 3.289 (4.7); 3.256 (2.4); 2.674 (5.4); 2.669 (8.9); 2.665 (5.1); 2.653 (3.0); 2.632 (2.6); 2.562 (2.5); 2.523 (20.0); 2.518 (29.2); 2.509 (406.4); 2.505 (888.3); 2.500 (1241.1); 2.495 (852.9); 2.491 (377.0); 2.450 (4.2); 2.445 (3.9); 2.331 (4.9); 2.327 (6.8); 2.322 (4.6); 2.071 (4.5); 1.622 (2.1); 1.602 (1.9); 1.122 (6.8); 1.104 (16.0); 1.086 (6.7); 0.937 (5.8); 0.918 (12.2); 0.900 (4.6); 0.008 (3.6); 0.000 (157.5); −0.009 (4.4)

Compound No. 3807, solvent [DMSO], 400 MHz

7.594 (2.8); 7.576 (3.5); 7.559 (2.5); 4.164 (5.0); 4.060 (3.7); 3.674 (16.0); 3.603 (10.8); 3.381 (0.9); 3.363 (2.6); 3.345 (2.7); 3.328 (0.9); 3.169 (0.9); 3.160 (0.8); 3.142 (2.4); 3.124 (2.4); 3.107 (0.8); 2.944 (8.8); 2.909 (13.5); 2.620 (1.0); 2.600 (3.3); 2.581 (3.7); 2.561 (2.3); 2.524 (1.1); 2.519 (1.6); 2.510 (23.5); 2.506 (51.2); 2.501 (71.6); 2.497 (49.9); 2.492 (22.3); 1.615 (0.8); 1.597 (1.7); 1.581 (2.4); 1.566 (2.3); 1.547 (1.3); 1.101 (3.0); 1.084 (6.7); 1.066 (3.0); 1.047 (2.9); 1.029 (6.4); 1.011 (2.7); 0.951 (3.1); 0.945 (5.0); 0.933 (7.8); 0.926 (11.1); 0.916 (5.7); 0.908 (4.5); 0.898 (1.9); 0.008 (0.6); 0.000 (20.7); −0.009 (0.6)

Compound No. 3170, solvent [DMSO], 400 MHz

8.251 (4.5); 3.334 (0.8); 3.310 (18.8); 3.302 (1.4); 3.299 (1.3); 3.284 (0.8); 2.704 (0.5); 2.685 (1.7); 2.666 (1.9); 2.647 (0.5); 2.520 (0.5); 2.511 (8.2); 2.506 (18.0); 2.502 (25.3); 2.497 (17.6); 2.493 (7.9); 1.610 (0.5); 1.593 (0.7); 1.577 (0.6); 1.416 (0.9); 1.398 (1.7); 1.380 (1.7); 1.362 (0.7); 1.171 (2.2); 1.152 (5.5); 1.133 (2.2); 0.901 (16.0); 0.885 (15.1); 0.000 (6.4)

Compound No. 3060, solvent [DMSO], 400 MHz

8.162 (5.1); 3.337 (0.8); 3.313 (5.2); 3.305 (1.5); 3.302 (1.5); 3.286 (0.9); 2.701 (0.6); 2.682 (2.0); 2.663 (2.1); 2.644 (0.6); 2.513 (3.8); 2.508 (8.3); 2.504 (11.5); 2.499 (8.1); 2.495 (3.8); 1.613 (0.6); 1.596 (0.8); 1.579 (0.7); 1.418 (1.0); 1.401 (1.9); 1.382 (1.9); 1.365 (0.8); 1.180 (2.5); 1.161 (6.0); 1.142 (2.5); 0.903 (16.0); 0.886 (15.2); 0.000 (3.0)

Compound No. 3303, solvent [DMSO], 400 MHz

12.758 (0.9); 9.650 (1.9); 8.398 (16.0); 3.477 (0.7); 3.471 (1.1); 3.466 (1.3); 3.459 (3.8); 3.447 (5.2); 3.432 (9.9); 3.423 (6.7); 3.411 (1.8); 3.404 (1.0); 3.313 (9.8); 3.265 (51.4); 2.710 (1.7); 2.691 (5.7); 2.672 (6.0); 2.653 (1.8); 2.512 (13.5); 2.508 (27.7); 2.503 (37.4); 2.499 (27.0); 2.494 (12.8); 1.146 (6.6); 1.127 (15.2); 1.108 (6.3); 0.000 (6.6)

Compound No. 3149, solvent [DMSO], 400 MHz

12.856 (1.0); 9.523 (1.1); 8.253 (8.3); 3.359 (0.8); 3.339 (1.3); 3.321 (4.7); 3.308 (101.1); 3.289 (3.8); 3.271 (1.3); 3.259 (0.9); 2.702 (1.2); 2.684 (4.0); 2.665 (4.6); 2.646 (1.3); 2.550 (1.0); 2.523 (2.6); 2.510 (48.7); 2.505 (103.1); 2.501 (142.0); 2.496 (99.8); 2.492 (45.9); 2.457 (0.9); 2.327 (0.8); 1.170 (5.1); 1.151 (12.1); 1.132 (5.1); 1.122 (7.5); 1.104 (16.0); 1.086 (7.2); 0.008 (1.5); 0.000 (46.5); −0.009 (1.6)

Compound No. 3145, solvent [DMSO], 400 MHz

7.519 (3.4); 3.389 (1.7); 3.372 (5.2); 3.354 (6.0); 3.336 (12.5); 3.169 (1.5); 3.151 (4.7); 3.133 (4.7); 3.115 (1.5); 2.633 (1.1); 2.614 (3.0); 2.595 (3.1); 2.576 (1.2); 2.523 (1.3); 2.519 (1.8); 2.510 (25.1); 2.506 (54.7); 2.501 (76.2); 2.496 (53.1); 2.492 (23.8); 1.172 (0.5); 1.160 (6.9); 1.141 (16.0); 1.122 (7.0); 1.107 (6.1); 1.089 (12.8); 1.071 (5.9); 1.053 (5.6); 1.035 (11.9); 1.017 (5.1); 0.008 (1.0); 0.000 (34.0); −0.009 (1.0)

Compound No. 3280, solvent [DMSO], 400 MHz

12.781 (0.6); 9.533 (0.6); 8.388 (5.0); 3.326 (1.0); 3.308 (50.8); 3.293 (1.3); 3.276 (0.8); 3.263 (0.5); 2.706 (0.5); 2.687 (1.7); 2.668 (1.9); 2.650 (0.5); 2.523 (1.3); 2.519 (1.8); 2.510 (25.7); 2.505 (56.1); 2.501 (78.1); 2.496 (54.2); 2.492 (24.3); 1.606 (0.6); 1.589 (0.7); 1.572 (0.7); 1.409 (0.9); 1.392 (1.7); 1.373 (1.8); 1.354 (1.0); 1.143 (2.1); 1.124 (5.2); 1.105 (2.1); 0.899 (16.0); 0.882 (15.4); 0.008 (0.6); 0.000 (21.4); −0.009 (0.6)

Compound No. 3039, solvent [DMSO], 400 MHz

12.840 (0.7); 9.539 (0.8); 8.164 (8.4); 3.342 (1.0); 3.327 (1.4); 3.324 (3.5); 3.308 (52.1); 3.291 (3.4); 3.288 (1.3); 3.273 (1.1); 2.699 (1.0); 2.680 (3.5); 2.670 (0.8); 2.662 (3.6); 2.643 (1.1); 2.524 (1.2); 2.519 (1.8); 2.510 (26.9); 2.506 (59.2); 2.501 (83.2); 2.496 (57.7); 2.492 (25.9); 1.179 (4.8); 1.160 (11.7); 1.141 (4.7); 1.124 (7.1); 1.106 (16.0); 1.088 (6.9); 0.008 (1.1); 0.000 (35.7); −0.009 (1.0)

Compound No. 3040, solvent [DMSO], 400 MHz

12.230 (1.7); 9.813 (1.0); 9.799 (1.8); 9.785 (0.9); 8.124 (9.7); 3.311 (75.2); 3.267 (2.1); 3.250 (4.2); 3.235 (4.2); 3.218 (2.0); 2.670 (0.7); 2.666 (0.5); 2.595 (1.4); 2.576 (4.4); 2.557 (4.6); 2.539 (2.0); 2.523 (2.8); 2.510 (37.0); 2.506 (73.5); 2.501 (97.7); 2.497 (69.9); 2.492 (32.9); 2.416 (2.9); 2.397 (3.9); 2.378 (3.0); 2.328 (0.6); 1.541 (0.5); 1.523 (2.6); 1.505 (5.8); 1.487 (7.2); 1.468 (5.0); 1.449 (2.0); 1.154 (5.1); 1.135 (11.9); 1.116 (5.2); 0.915 (6.7); 0.905 (8.4); 0.897 (14.2); 0.887 (16.0); 0.879 (6.4); 0.868 (6.8); 0.000 (1.5)

Compound No. 2929, solvent [DMSO], 400 MHz

12.236 (0.9); 9.750 (0.6); 9.736 (1.0); 9.722 (0.6); 8.153 (8.5); 3.331 (1.2); 3.315 (31.7); 3.299 (3.2); 3.295 (3.2); 3.281 (3.1); 3.277 (1.2); 3.263 (1.0); 2.599 (1.1); 2.580 (3.5); 2.561 (3.6); 2.542 (1.3); 2.524 (0.9); 2.520 (1.3); 2.511 (15.2); 2.506 (32.6); 2.502 (45.1); 2.497 (31.4); 2.493 (13.9); 2.469 (1.3); 2.451 (4.2); 2.432 (4.4); 2.413 (1.4); 1.156 (4.4); 1.137 (11.1); 1.120 (8.7); 1.109 (6.0); 1.102 (16.0); 1.090 (13.8); 1.084 (7.2); 1.071 (5.4); 0.000 (1.1)

Compound No. 3060, solvent [DMSO], 400 MHz

12.214 (0.8); 9.760 (0.8); 8.121 (4.8); 3.376 (7.0); 3.324 (0.9); 3.307 (1.5); 3.290 (1.5); 3.274 (0.8); 2.594 (0.6); 2.575 (2.0); 2.557 (2.0); 2.538 (0.8); 2.524 (0.9); 2.511 (13.8); 2.506 (28.1); 2.502 (37.9); 2.497 (26.9); 2.493 (12.4); 2.415 (1.3); 2.396 (1.8); 2.376 (1.4); 1.616 (0.6); 1.599 (0.9); 1.583 (0.7); 1.505 (0.8); 1.486 (1.3); 1.467 (1.3); 1.449 (0.8); 1.410 (1.0); 1.392 (2.1); 1.374 (2.0); 1.357 (0.8); 1.153 (2.3); 1.134 (5.4); 1.115 (2.3); 0.915 (3.3); 0.903 (16.0); 0.897 (7.5); 0.886 (15.1); 0.879 (3.1); 0.000 (1.5)

Compound No. 2930, solvent [DMSO], 400 MHz

12.232 (1.1); 9.815 (0.7); 9.801 (1.2); 9.787 (0.7); 8.153 (9.6); 3.313 (47.4); 3.270 (1.9); 3.264 (0.5); 3.252 (3.5); 3.238 (3.5); 3.220 (1.9); 2.601 (1.2); 2.582 (3.9); 2.563 (4.0); 2.544 (1.2); 2.524 (1.2); 2.519 (1.6); 2.511 (19.3); 2.506 (41.3); 2.502 (57.0); 2.497 (39.5); 2.492 (17.5); 2.471 (1.5); 2.452 (4.9); 2.433 (4.8); 2.414 (1.6); 1.523 (2.0); 1.505 (3.7); 1.488 (3.8); 1.470 (2.2); 1.157 (4.9); 1.138 (12.1); 1.119 (4.8); 1.109 (6.3); 1.091 (15.1); 1.072 (6.0); 0.905 (7.5); 0.887 (16.0); 0.868 (6.5); 0.000 (1.4)

Compound No. 3083, solvent [DMSO], 400 MHz

12.224 (1.5); 9.866 (0.8); 9.855 (1.6); 9.841 (0.8); 8.130 (12.4); 3.474 (0.5); 3.468 (0.8); 3.463 (0.9); 3.456 (3.0); 3.444 (3.8); 3.440 (3.6); 3.432 (7.4); 3.429 (7.7); 3.421 (5.4); 3.420 (5.2); 3.411 (1.0); 3.408 (1.4); 3.401 (0.8); 3.359 (17.0); 3.264 (57.1); 2.597 (1.3); 2.578 (4.4); 2.560 (4.6); 2.541 (1.5); 2.524 (1.3); 2.519 (1.9); 2.511 (23.7); 2.506 (51.1); 2.501 (71.0); 2.497 (49.7); 2.492 (22.1); 2.417 (2.9); 2.398 (3.7); 2.379 (3.1); 1.506 (1.7); 1.487 (2.8); 1.468 (2.8); 1.449 (1.7); 1.155 (5.4); 1.136 (13.5); 1.117 (5.4); 0.914 (7.2); 0.896 (16.0); 0.878 (6.3); 0.000 (1.6)

Compound No. 3021, solvent [DMSO], 400 MHz

11.706 (0.7); 7.287 (7.5); 7.284 (10.9); 5.159 (1.8); 5.147 (1.6); 4.393 (0.7); 4.358 (0.7); 4.286 (1.3); 4.275 (1.2); 4.182 (2.0); 4.164 (6.3); 4.146 (6.6); 4.128 (2.4); 4.123 (1.0); 4.114 (1.8); 4.103 (0.8); 4.096 (1.8); 4.085 (1.8); 4.078 (0.8); 4.067 (1.7); 4.058 (0.7); 4.050 (0.6); 4.040 (0.7); 3.528 (3.9); 3.420 (1.1); 3.387 (1.3); 3.126 (0.6); 3.117 (0.6); 3.094 (1.0); 3.087 (1.0); 3.065 (0.6); 3.054 (0.5); 2.755 (0.8); 2.524 (2.1); 2.519 (3.0); 2.511 (26.0); 2.506 (50.3); 2.502 (67.6); 2.497 (49.1); 2.492 (25.2); 2.481 (3.1); 2.476 (2.9); 2.462 (1.4); 2.401 (1.3); 2.383 (4.2); 2.364 (4.7); 2.356 (3.0); 2.345 (2.1); 2.337 (2.8); 2.329 (1.0); 2.324 (0.8); 2.319 (1.1); 2.150 (1.0); 2.116 (1.0); 2.053 (0.7); 2.020 (0.9); 1.724 (0.6); 1.716 (0.6); 1.686 (2.8); 1.656 (3.2); 1.629 (0.8); 1.619 (0.5); 1.614 (0.6); 1.530 (1.7); 1.508 (1.0); 1.476 (0.6); 1.326 (0.7); 1.307 (0.8); 1.282 (1.1); 1.243 (8.3); 1.225 (16.0); 1.208 (7.7); 1.173 (4.9); 1.155 (11.2); 1.137 (9.3); 1.133 (12.1); 1.120 (8.2); 1.114 (5.8); 1.101 (3.3); 1.081 (5.5); 1.062 (12.3); 1.052 (4.6); 1.043 (6.0); 1.034 (8.2); 1.015 (3.5); 0.000 (1.3)

Compound No. 2973, solvent [DMSO], 400 MHz

12.226 (1.3); 9.855 (1.5); 9.841 (0.8); 8.158 (10.7); 3.457 (7.3); 3.445 (6.4); 3.430 (8.2); 3.421 (5.8); 3.412 (1.4); 3.408 (1.7); 3.402 (1.0); 3.264 (47.5); 2.670 (0.7); 2.602 (1.3); 2.583 (4.5); 2.565 (4.7); 2.552 (0.6); 2.546 (1.4); 2.523 (1.9); 2.519 (2.8); 2.510 (35.8); 2.506 (76.3); 2.501 (105.4); 2.496 (74.4); 2.492 (33.8); 2.471 (2.0); 2.452 (5.6); 2.433 (5.4); 2.415 (1.8); 2.328 (0.7); 1.157 (5.3); 1.138 (13.0); 1.119 (5.3); 1.109 (6.9); 1.090 (16.0); 1.072 (6.5); 0.008 (1.5); 0.000 (51.1); −0.009 (1.6)

Compound No. 2925, solvent [DMSO], 400 MHz

7.254 (11.8); 3.388 (0.9); 3.370 (2.9); 3.352 (3.0); 3.335 (1.0); 3.166 (0.9); 3.149 (2.7); 3.131 (2.8); 3.113 (0.9); 2.524 (0.9); 2.519 (1.4); 2.510 (22.0); 2.506 (45.9); 2.501 (64.3); 2.496 (45.8); 2.492 (24.4); 2.472 (4.7); 2.453 (1.5); 2.381 (1.4); 2.362 (4.7); 2.343 (4.8); 2.332 (0.6); 2.324 (1.8); 1.142 (5.2); 1.123 (12.9); 1.109 (4.0); 1.104 (6.2); 1.091 (7.0); 1.070 (7.8); 1.052 (16.0); 1.040 (3.7); 1.033 (7.2); 1.023 (6.9); 1.005 (3.1); 0.008 (0.7); 0.000 (23.9); −0.009 (0.7)

Compound No. 3131, solvent [DMSO], 400 MHz

11.701 (0.7); 7.249 (12.3); 5.157 (1.5); 5.145 (1.4); 4.388 (0.7); 4.354 (0.6); 4.286 (1.2); 4.274 (1.1); 4.180 (1.7); 4.162 (5.6); 4.145 (5.9); 4.127 (2.2); 4.120 (0.7); 4.111 (0.7); 4.102 (1.9); 4.098 (0.9); 4.084 (2.0); 4.080 (2.0); 4.066 (0.9); 4.062 (1.8); 4.053 (0.6); 4.044 (0.6); 4.035 (0.6); 3.491 (3.5); 3.418 (1.1); 3.385 (1.1); 3.120 (0.5); 3.112 (0.6); 3.089 (0.9); 3.082 (0.9); 3.058 (0.5); 2.747 (0.8); 2.524 (2.1); 2.520 (2.6); 2.511 (21.1); 2.506 (45.1); 2.502 (61.8); 2.497 (42.5); 2.493 (20.2); 2.485 (4.5); 2.475 (2.1); 2.469 (2.2); 2.455 (0.6); 2.451 (0.5); 2.346 (2.1); 2.328 (3.6); 2.324 (3.7); 2.307 (3.6); 2.284 (1.5); 2.150 (0.9); 2.115 (0.8); 2.057 (0.6); 2.024 (0.8); 1.719 (0.6); 1.710 (0.6); 1.683 (2.5); 1.655 (2.8); 1.628 (0.7); 1.528 (1.5); 1.509 (0.7); 1.496 (0.9); 1.476 (1.7); 1.458 (2.8); 1.438 (2.8); 1.426 (1.8); 1.420 (1.9); 1.415 (1.6); 1.408 (1.4); 1.395 (0.9); 1.325 (0.6); 1.299 (0.7); 1.275 (0.9); 1.241 (8.2); 1.223 (16.0); 1.206 (7.5); 1.170 (5.0); 1.153 (11.2); 1.149 (5.5); 1.135 (8.9); 1.130 (10.7); 1.117 (7.6); 1.111 (5.0); 1.098 (3.0); 0.905 (5.4); 0.887 (11.5); 0.869 (7.2); 0.852 (7.9); 0.834 (3.3); 0.000 (1.6)

Compound No. 3035, solvent [DMSO], 400 MHz

7.226 (13.2); 3.386 (1.0); 3.368 (3.0); 3.351 (3.1); 3.333 (1.0); 3.167 (1.0); 3.149 (2.8); 3.132 (2.8); 3.114 (1.0); 2.525 (0.6); 2.520 (0.8); 2.511 (10.0); 2.507 (22.0); 2.502 (29.2); 2.497 (20.4); 2.493 (9.7); 2.487 (5.0); 2.468 (4.6); 2.450 (1.5); 2.331 (2.9); 2.312 (3.7); 2.293 (3.0); 1.473 (1.6); 1.454 (2.8); 1.436 (2.8); 1.417 (1.7); 1.140 (5.5); 1.121 (13.7); 1.108 (4.2); 1.102 (6.6); 1.090 (7.1); 1.073 (3.4); 1.035 (3.4); 1.017 (6.7); 0.999 (3.1); 0.892 (7.2); 0.874 (16.0); 0.856 (6.3); 0.000 (0.8)

Compound No. 2951, solvent [DMSO], 400 MHz

12.216 (0.6); 9.761 (0.7); 8.148 (4.6); 3.326 (1.4); 3.310 (95.3); 3.293 (1.5); 3.275 (0.8); 2.670 (0.6); 2.599 (0.6); 2.580 (2.0); 2.561 (2.0); 2.542 (0.8); 2.523 (2.2); 2.518 (3.2); 2.510 (35.1); 2.505 (73.6); 2.501 (100.7); 2.496 (70.4); 2.491 (31.6); 2.469 (0.8); 2.450 (2.4); 2.431 (2.3); 2.412 (0.7); 2.327 (0.6); 1.615 (0.6); 1.599 (0.8); 1.582 (0.7); 1.410 (0.9); 1.392 (2.0); 1.374 (1.9); 1.357 (0.8); 1.155 (2.3); 1.137 (5.7); 1.118 (2.4); 1.108 (3.0); 1.090 (6.8); 1.071 (2.8); 0.903 (16.0); 0.886 (15.3); 0.000 (1.3)

Compound No. 2927, solvent [DMSO], 400 MHz

14.871 (0.6); 11.755 (0.6); 8.223 (2.9); 7.339 (5.6); 7.293 (4.0); 4.161 (5.2); 4.060 (4.5); 3.673 (16.0); 3.578 (12.3); 3.311 (50.8); 2.954 (11.0); 2.915 (15.1); 2.675 (0.5); 2.667 (1.7); 2.648 (1.5); 2.537 (0.9); 2.533 (0.8); 2.523 (2.2); 2.518 (5.0); 2.510 (35.7); 2.506 (76.4); 2.501 (107.6); 2.496 (77.7); 2.492 (34.4); 2.478 (2.7); 2.458 (0.8); 2.390 (1.9); 2.371 (2.3); 2.353 (2.2); 2.334 (1.8); 2.328 (0.9); 2.323 (0.6); 2.316 (0.6); 1.189 (1.8); 1.170 (4.3); 1.151 (4.0); 1.133 (9.9); 1.114 (12.0); 1.095 (4.1); 1.086 (3.1); 1.067 (7.1); 1.048 (4.9); 1.030 (5.4); 1.011 (2.2); 0.000 (2.5)

Compound No. 3039, solvent [DMSO], 400 MHz

12.237 (0.9); 9.749 (0.7); 9.735 (1.2); 9.721 (0.7); 8.127 (9.7); 7.642 (2.1); 7.016 (5.4); 7.013 (5.4); 3.332 (1.9); 3.317 (2.6); 3.314 (4.4); 3.299 (4.0); 3.295 (3.9); 3.281 (3.4); 3.278 (1.5); 3.263 (1.1); 3.004 (0.5); 3.000 (1.4); 2.986 (1.4); 2.982 (1.5); 2.968 (1.4); 2.964 (0.5); 2.595 (1.1); 2.576 (3.7); 2.557 (3.8); 2.538 (1.2); 2.526 (0.6); 2.521 (0.8); 2.512 (9.5); 2.508 (20.3); 2.503 (28.1); 2.498 (19.5); 2.494 (8.7); 2.416 (2.3); 2.397 (2.9); 2.377 (2.5); 1.506 (1.3); 1.487 (2.2); 1.468 (2.3); 1.449 (1.4); 1.154 (4.4); 1.135 (10.8); 1.120 (7.8); 1.116 (4.8); 1.110 (0.9); 1.102 (16.0); 1.084 (6.8); 0.987 (2.9); 0.969 (6.1); 0.951 (2.8); 0.915 (5.7); 0.897 (12.5); 0.878 (5.0); 0.000 (0.6)

The present invention accordingly provides for the use of at least one compound selected from the group consisting of substituted pyridonecarboxamides of the general formula (I), and of any desired mixtures of these pyridonecarboxamides of the general formula (I) having substitution in accordance with the invention, with one or more active agrochemical ingredients in accordance with the definition below, for increasing the resistance of plants to abiotic stress not triggered by pesticides, preferably not by herbicides, especially for enhancing plant growth and/or for increasing plant yield.

The present invention further provides a spray solution for treatment of plants, comprising an amount, effective for increasing the resistance of plants to abiotic stress not triggered by pesticides, preferably not by herbicides, of at least one compound selected from the group consisting of substituted pyridonecarboxamides of the general formula (I). The abiotic stress conditions which can be relativized may include, for example, drought, cold and hot conditions, osmotic stress, waterlogging, elevated soil salinity, elevated exposure to minerals, ozone conditions, strong light conditions, limited availability of nitrogen nutrients, limited availability of phosphorus nutrients.

In one embodiment, it may be the case, for example, that the substituted pyridonecarboxamides of the general formula (I) mentioned in the invention are applied by spray application to appropriate plants or plant parts to be treated. The inventive compounds of the general formula (I) are used as envisaged in accordance with the invention preferably at a dosage between 0.0005 and 3 kg/ha, more preferably between 0.001 and 2 kg/ha, especially preferably between 0.005 and 1 kg/ha. If, in the context of the present invention, abscisic acid is used simultaneously with substituted pyridonecarboxamides of the general formula (I), for example in the context of a combined preparation or formulation, abscisic acid is preferably added in a dosage between 0.001 and 3 kg/ha, more preferably between 0.005 and 2 kg/ha, especially preferably between 0.01 and 1 kg/ha.

The term “resistance to abiotic stress” is understood in the context of the present invention to mean various kinds of benefits for plants. Such advantageous properties are manifested, for example, in the following improved plant characteristics: improved root growth with regard to surface area and depth, increased stolon or tiller formation, stronger and more productive stolons and tillers, improvement in shoot growth, increased lodging resistance, increased shoot base diameter, increased leaf area, higher yields of nutrients and constituents, for example carbohydrates, fats, oils, proteins, vitamins, minerals, essential oils, dyes, fibers, better fiber quality, earlier flowering, increased number of flowers, reduced content of toxic products such as mycotoxins, reduced content of residues or disadvantageous constituents of any kind, or better digestibility, improved storage stability of the harvested material, improved tolerance to disadvantageous temperatures, improved tolerance to drought and aridity, and also oxygen deficiency as a result of waterlogging, improved tolerance to elevated salt contents in soil and water, enhanced tolerance to ozone stress, improved compatibility with respect to herbicides and other plant treatment compositions, improved water absorption and photosynthesis performance, advantageous plant properties, for example acceleration of ripening, more homogeneous ripening, greater attractiveness to beneficial animals, improved pollination, or other advantages well known to a person skilled in the art.

More particularly, the inventive use exhibits the advantages described in spray application to plants and plant parts. Combinations of pyridonecarboxamides of the general formula (I) having substitution in accordance with the invention with substances including insecticides, attractants, acaricides, fungicides, nematicides, growth regulators, safeners, substances which influence plant maturity, and bactericides can likewise be employed in the control of plant disorders in the context of the present invention. In addition, the combined use of corresponding substituted pyridonecarboxamides of the general formula (I) with genetically modified cultivars with a view to increased tolerance to abiotic stress is likewise possible.

The further various benefits for plants mentioned above can be combined in a known manner in component form, and generally applicable terms can be used to describe them. Such terms are, for example, the following names: phytotonic effect, resistance to stress factors, less plant stress, plant health, healthy plants, plant fitness, plant wellness, plant concept, vigor effect, stress shield, protective shield, crop health, crop health properties, crop health products, crop health management, crop health therapy, plant health, plant health properties, plant health products, plant health management, plant health therapy, greening effect or regreening effect, freshness, or other terms with which a person skilled in the art is entirely familiar.

In the context of the present invention, a good effect on resistance to abiotic stress is understood to mean, without limitation,
at least an emergence improved by generally 3%, especially more than 5%, more preferably more than 10%,
at least a yield enhanced by generally 3%, especially more than 5%, more preferably more than 10%,
at least a root development improved by generally 3%, especially more than 5%, more preferably more than 10%,
at least a shoot size rising by generally 3%, especially more than 5%, more preferably more than 10%,
at least a leaf area increased by generally 3%, especially more than 5%, more preferably more than 10%,
at least a photosynthesis performance improved by generally 3%, especially more than 5%, more preferably more than 10%, and/or
at least a flower development improved by generally 3%, especially more than 5%, more preferably more than 10%,
   and the effects may occur individually or else in any combination of two or more effects.

The present invention further provides a spray solution for treatment of plants, comprising an amount, effective for increasing the resistance of plants to abiotic stress factors, of at least one compound of the general formula (I). The spray solution may comprise other customary constituents, such as solvents, formulation auxiliaries, especially water. Further constituents may include active agrochemical ingredients which are described in more detail below.

The present invention further provides for the use of corresponding spray solutions for increasing the resistance of plants to abiotic stress factors. The remarks which follow apply both to the inventive use of the compounds of the general formula (I) per se and to the corresponding spray solutions.

In accordance with the invention, it has additionally been found that the application, to plants or in their environment, of the compounds of the general formula (I) in combination with at least one fertilizer as defined below is possible.

Fertilizers which can be used in accordance with the invention together with the compounds of the general formula (I) elucidated in detail above are generally organic and inorganic nitrogen-containing compounds, for example ureas, urea/formaldehyde condensation products, amino acids, ammonium salts and ammonium nitrates, potassium salts (preferably chlorides, sulfates, nitrates), salts of phosphoric acid and/or salts of phosphorous acid (preferably potassium salts and ammonium salts). In this context, particular mention should be made of the NPK fertilizers, i.e. fertilizers which contain nitrogen, phosphorus and potassium, calcium ammonium nitrate, i.e. fertilizers which additionally contain calcium, or ammonium sulfate nitrate (general formula (NH₄)₂SO₄NH₄NO₃), ammonium phosphate and ammonium sulfate. These fertilizers are generally known to the person skilled in the art; see also, for example, Ullmann's Encyclopedia of Industrial Chemistry, 5th edition, Vol. A 10, pages 323 to 431, Verlagsgesellschaft, Weinheim, 1987.

The fertilizers may additionally comprise salts of micronutrients (preferably calcium, sulfur, boron, manganese, magnesium, iron, boron, copper, zinc, molybdenum and cobalt) and of phytohormones (for example vitamin B1 and indole-(III)-acetic acid) or mixtures of these. Fertilizers used in accordance with the invention may also contain other salts such as monoammonium phosphate (MAP), diammonium phosphate (DAP), potassium sulfate, potassium chloride, magnesium sulfate. Suitable amounts for the secondary nutrients or trace elements are amounts of 0.5% to 5% by weight, based on the overall fertilizer. Further possible constituents are crop protection agents, insecticides or fungicides, growth regulators or mixtures thereof. Further details of these are given further down.

The fertilizers can be used, for example, in the form of powders, granules, prills or compactates. However, the fertilizers can also be used in liquid form, dissolved in an aqueous medium. In this case, dilute aqueous ammonia can also be used as a nitrogen fertilizer. Further possible ingredients for fertilizers are described, for example, in Ullmann's Encyclopedia of Industrial Chemistry, 5th edition, 1987, volume A 10, pages 363 to 401, DE-A 41 28 828, DE-A 19 05 834 and DE-A 196 31 764. The general composition of the fertilizers, which, in the context of the present invention, may take the form of straight and/or compound fertilizers, for example composed of nitrogen, potassium or phosphorus, may vary within a wide range. In general, a content of 1% to 30% by weight of nitrogen (preferably 5% to 20% by weight), of 1% to 20% by weight of potassium (preferably 3% to 15% by weight) and a content of 1% to 20% by weight of phosphorus (preferably 3% to 10% by weight) is advantageous. The microelement content is usually in the ppm range, preferably in the range from 1 to 1000 ppm.

In the context of the present invention, the fertilizer and the compounds of the general formula (I) may be administered simultaneously, i.e. synchronously. However, it is also possible first to apply the fertilizer and then a compound of the formula (I), or first to apply a compound of the formula (I) and then the fertilizer. In the case of nonsynchronous application of a compound of the general formula (I) and the fertilizer, the application in the context of the present invention is, however, effected in a functional relationship, especially within a period of generally 24 hours, preferably 18 hours, more preferably 12 hours, specifically 6 hours, more specifically 4 hours, even more specifically within 2 hours. In very particular embodiments of the present invention, the inventive compound of the general formula (I) and the fertilizer are applied within a time frame of less than 1 hour, preferably less than 30 minutes, more preferably less than 15 minutes.

The active ingredients for use in accordance with the invention, optionally in combination with fertilizers, can preferably be employed in the following plants, although the enumeration which follows is not limiting.

Preferred plants are those from the group of the useful plants, ornamental plants, turfgrass types, commonly used trees which are employed as ornamentals in public and domestic areas, and forestry trees. Forestry trees include trees for the production of timber, cellulose, paper and products made from parts of the trees. The term useful plants as used here refers to crop plants which are used as plants for obtaining foods, animal feeds, fuels or for industrial purposes.

The useful plants include, for example, the following types of plants: triticale, durum (hard wheat), turf, vines, cereals, for example wheat, barley, rye, oats, hops, rice, corn and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao beans and peanuts; cucurbits, for example pumpkin/squash, cucumbers and melons; fiber plants, for example cotton, flax, hemp and jute; citrus fruit, for example, oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or also plants such as tobacco, nuts, coffee, eggplant, sugarcane, tea, pepper, grapevines, hops, bananas, latex plants and ornamentals, for example flowers, shrubs, deciduous trees and coniferous trees. This enumeration does not constitute a limitation.

The following plants are considered to be particularly suitable target crops for the application of the method of the invention: oats, rye, triticale, durum, cotton, eggplant, turf, pome fruit, stone fruit, soft fruit, corn, wheat, barley, cucumber, tobacco, vines, rice, cereals, pear, peppers, beans, soybeans, oilseed rape, tomato, bell pepper, melons, cabbage, potatoes and apples.

Examples of trees which can be improved by the method of the invention include: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved by the method of the invention include: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiate, P. ponderosa, P. contorta, P. sylvestre, P. elliottii, P. montecola, P. albicaulis, P. resinosa, P. palustris, P. taeda, P. flexilis, P. jeffregi, P. baksiana, P. strobes; from the tree species Eucalyptus: E. grandis, E. globulus, E. camadentis, E. nitens, E. obliqua, E. regnans, E. pilularus.

Particularly preferred trees which can be improved by the method of the invention are: from the tree species Pinus: P. radiate, P. ponderosa, P. contorta, P. sylvestre, P. strobes; from the tree species Eucalyptus: E. grandis, E. globulus and E. camadentis.

Particularly preferred trees which can be improved by the method of the invention are: horse chestnut, Platanaceae, linden tree and maple tree.

The present invention can also be applied to any desired turfgrasses, including cool-season turfgrasses and warm-season turfgrasses. Examples of cool-season turfgrasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festuca capillata Lam.), tall fescue (Festuca arundinacea Schreb.) and meadow fescue (Festuca elanor L.);
ryegrasses (Lolium spp.), such as annual ryegrass (Lolium multiflorum Lam.), perennial ryegrass (Lolium perenne L.) and Italian ryegrass (Lolium multiflorum Lam.);
and wheatgrasses (Agropyron spp.), such as fairway wheatgrass (Agropyron cristatum (L.) Gaertn.), crested wheatgrass (Agropyron desertorum (Fisch.) Schult.) and “western wheatgrass” (Agropyron smithii Rydb.).

Examples of further cool-season turfgrasses are beachgrass (Ammophila breviligulata Fern.), smooth bromegrass (Bromus inermis Leyss.), cattails such as Timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Examples of warm-season turfgrasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpet grass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), Blue grama (Bouteloua gracilis (H.B.K.) Lag. ex Griffiths), seashore paspalum (Paspalum vaginatum Swartz) and sideoats grama (Bouteloua curtipendula (Michx. Torr.)). Cool-season turfgrasses are generally preferred for the inventive use.

Particular preference is given to bluegrass, bentgrass and redtop, fescues and ryegrasses. Bentgrass is especially preferred.

Particular preference is given in accordance with the invention to treating plants of the respective commercially customary plant cultivars or those that are in use. Plant cultivars are understood to mean plants which have new properties (“traits”) and which have been obtained by conventional breeding, by mutagenesis or with the aid of recombinant DNA techniques. Crop plants may accordingly be plants which can be obtained by conventional breeding and optimization methods or by biotechnological and genetic engineering methods or combinations of these methods, including the transgenic plants and including the plant cultivars which are protectable or non-protectable by plant breeders' rights.

The treatment method of the invention can thus also be used for the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced into the nuclear, chloroplastic or hypochondrial genome gives the transformed plant new or improved agronomic or other properties by expressing a protein or polypeptide of interest or by downregulating or silencing (an)other gene(s) which is/are present in the plant (using for example antisense technology, cosuppression technology or RNAi technology [RNA interference]). A heterologous gene that is located in the genome is also called a transgene. A transgene that is defined by its specific presence in the plant genome is called a transformation or transgenic event.

Plants and plant cultivars which are preferably treated in accordance with the invention include all plants which have genetic material which imparts particularly advantageous, useful traits to these plants (whether obtained by breeding and/or biotechnological means).

Plants and plant cultivars which may also be treated according to the invention are those plants which are resistant to one or more abiotic stress factors. Abiotic stress conditions may include, for example, drought, cold temperature exposure, heat exposure, osmotic stress, waterlogging, increased soil salinity, increased exposure to minerals, exposure to ozone, exposure to strong light, limited availability of nitrogen nutrients, limited availability of phosphorus nutrients or lack of shade.

Plants and plant cultivars which may also be treated according to the invention are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and nonstress conditions), including, but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and resistance to lodging. Further yield traits include seed composition, such as carbohydrate content, protein content, oil content and oil composition, nutritional value, reduction in antinutritional compounds, improved processability and better storage stability.

Plants that may likewise be treated in accordance with the invention are hybrid plants that already express the characteristics of heterosis, or hybrid effect, which results in generally higher yield, enhanced vigor, better health, and better resistance against biotic and abiotic stress factors. Such plants are typically produced by crossing an inbred male-sterile parent line (the female crossbreeding parent) with another inbred male-fertile parent line (the male crossbreeding parent). Hybrid seed is typically harvested from the male-sterile plants and sold to growers. Male-sterile plants can sometimes (for example in corn) be produced by detasseling (i.e. mechanical removal of the male reproductive organs or male flowers); however, it is more typical for male sterility to be the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species (WO 1992/005251, WO 1995/009910, WO 1998/27806, WO 2005/002324, WO 2006/021972 and U.S. Pat. No. 6,229,072). However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar (e.g. WO 1991/002069).

Plants or plant cultivars (obtained by plant biotechnology methods such as genetic engineering) which may likewise be treated in accordance with the invention are herbicide-tolerant plants, i.e. plants rendered tolerant to one or more predefined herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., Science (1983), 221, 370-371), the CP4 gene of the bacterium Agrobacterium sp. (Barry et al., Curr. Topics Plant Physiol. (1992), 7, 139-145), the genes encoding a petunia EPSPS (Shah et al., Science (1986), 233, 478-481), a tomato EPSPS (Gasser et al., J. Biol. Chem. (1988), 263, 4280-4289) or an Eleusine EPSPS (WO 2001/66704). It can also be a mutated EPSPS, as described, for example, in EP-A 0837944, WO 2000/066746, WO 2000/066747 or WO 2002/026995. Glyphosate-tolerant plants can also be obtained by expressing a gene that encodes a glyphosate oxidoreductase enzyme as described in U.S. Pat. No. 5,776,760 and U.S. Pat. No. 5,463,175. Glyphosate-tolerant plants can also be obtained by expressing a gene that encodes a glyphosate acetyl transferase enzyme as described, for example, in WO 2002/036782, WO 2003/092360, WO 2005/012515 and WO 2007/024782. Glyphosate-tolerant plants can also be obtained by selecting plants containing naturally occurring mutations of the abovementioned genes, as described, for example, in WO 2001/024615 or WO 2003/013226.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One example of an such effective detoxifying enzyme is an enzyme encoding a phosphinothricin acetyltransferase (such as the bar or pat protein from Streptomyces species). Plants expressing an exogenous phosphinothricin acetyltransferase are described, for example, in U.S. Pat. No. 5,561,236; U.S. Pat. No. 5,648,477; U.S. Pat. No. 5,646,024; U.S. Pat. No. 5,273,894; U.S. Pat. No. 5,637,489; U.S. Pat. No. 5,276,268; U.S. Pat. No. 5,739,082; U.S. Pat. No. 5,908,810 and U.S. Pat. No. 7,112,665.

Further herbicide-tolerant plants are also plants that have been made tolerant to the herbicides inhibiting the enzyme hydroxyphenylpyruvate dioxygenase (HPPD).

Hydroxyphenylpyruvate dioxygenases are enzymes that catalyze the reaction in which para-hydroxyphenylpyruvate (HPP) is converted to homogentisate. Plants tolerant to HPPD inhibitors can be transformed with a gene encoding a naturally occurring resistant HPPD enzyme, or a gene encoding a mutated HPPD enzyme according to WO 1996/038567, WO 1999/024585 and WO 1999/024586. Tolerance to HPPD inhibitors can also be obtained by transforming plants with genes encoding certain enzymes enabling the formation of homogentisate despite inhibition of the native HPPD enzyme by the HPPD inhibitor. Such plants and genes are described in WO 1999/034008 and WO 2002/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928.

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyloxy(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides, as described for example in Tranel and Wright, Weed Science (2002), 50, 700-712, but also in U.S. Pat. No. 5,605,011, U.S. Pat. No. 5,378,824, U.S. Pat. No. 5,141,870 and U.S. Pat. No. 5,013,659. The production of sulfonylurea-tolerant plants and imidazolinone-tolerant plants has been described in U.S. Pat. No. 5,605,011; U.S. Pat. No. 5,013,659; U.S. Pat. No. 5,141,870; U.S. Pat. No. 5,767,361; U.S. Pat. No. 5,731,180; U.S. Pat. No. 5,304,732; U.S. Pat. No. 4,761,373; U.S. Pat. No. 5,331,107; U.S. Pat. No. 5,928,937; and U.S. Pat. No. 5,378,824; and also in the international publication WO 1996/033270. Further imidazolinone-tolerant plants have also been described, for example, in WO 2004/040012, WO 2004/106529, WO 2005/020673, WO 2005/093093, WO 2006/007373, WO 2006/015376, WO 2006/024351 and WO 2006/060634. Further sulfonylurea- and imidazolinone-tolerant plants have also been described, for example, in WO 2007/024782.

Further plants tolerant to imidazolinone and/or sulfonylurea can be obtained by induced mutagenesis, by selection in cell cultures in the presence of the herbicide or by mutation breeding, as described, for example, for soybeans in U.S. Pat. No. 5,084,082, for rice in WO 1997/41218, for sugarbeet in U.S. Pat. No. 5,773,702 and WO 1999/057965, for lettuce in U.S. Pat. No. 5,198,599 or for sunflower in WO 2001/065922.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding the following:

1) an insecticidal crystal protein from Bacillus thuringiensis or an insecticidal portion thereof, such as the insecticidal crystal proteins compiled by Crickmore et al., Microbiology and Molecular Biology Reviews (1998), 62, 807-813, updated by Crickmore et al. (2005) in the Bacillus thuringiensis toxin nomenclature (online at: http://www.lifesci.sussex.ac.uk/Home/Neil_Crickmore/Bt/), or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins (Moellenbeck et al., Nat. Biotechnol. (2001), 19, 668-72; Schnepf et al., Applied Environm. Microb. (2006), 71, 1765-1774); or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by corn event MON98034 (WO 2007/027777); or
4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bbl protein in corn events MON863 or MON88017, or the Cry3A protein in corn event MIR 604; or
5) an insecticidal secreted protein from Bacillus thuringiensis or Bacillus cereus, or an insecticidal portion thereof, such as the vegetative insecticidal proteins (VIPs) listed under the following link, for example proteins from the VIP3Aa protein class: http://www.lifesci.sussex.ac.uk/Home/Neil_Crickmore/Bt/vip.html; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 1994/21795); or
7) a hybrid insecticidal protein comprising parts from different secreted proteins from Bacillus thuringiensis or Bacillus cereus, such as a hybrid of the proteins in 1) or a hybrid of the proteins in 2) above; or
8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, the insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of the target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:

a. plants which contain a transgene capable of reducing the expression and/or the activity of the poly(ADP-ribose)polymerase (PARP) gene in the plant cells or plants, as described in WO 2000/004173 or EP 04077984.5 or EP 06009836.5;
b. plants which contain a stress tolerance-enhancing transgene capable of reducing the expression and/or the activity of the PARG-encoding genes of the plants or plant cells, as described, for example, in WO 2004/090140;
c. plants which comprise a stress-tolerance-enhancing transgene coding for a plant-functional enzyme of the nicotinamide adenine dinucleotide salvage biosynthetic pathway, including nicotinamidase, nicotinate phosphoribosyltransferase, nicotinic acid mononucleotide adenyltransferase, nicotinamide adenine dinucleotide synthetase or nicotinamide phosphoribosyltransferase as described e.g. in EP 04077624.7 or WO 2006/133827 or PCT/EP07/002433.

Plants or plant cultivars (obtained by plant biotechnology methods such as genetic engineering) which may also be treated according to the invention show altered quantity, quality and/or storage stability of the harvested product and/or altered properties of specific ingredients of the harvested product such as, for example:

1) Transgenic plants which synthesize a modified starch which, in its physicochemical characteristics, in particular the amylose content or the amylose/amylopectin ratio, the degree of branching, the average chain length, the side chain distribution, the viscosity behavior, the gelling strength, the starch granule size and/or the starch granule morphology, is changed in comparison with the synthesized starch in wild-type plant cells or plants, so that this modified starch is better suited to specific applications. These transgenic plants which synthesize a modified starch are described, for example, in EP 0571427, WO 1995/004826, EP 0719338, WO 1996/15248, WO 1996/19581, WO 1996/27674, WO 1997/11188, WO 1997/26362, WO 1997/32985, WO 1997/42328, WO 1997/44472, WO 1997/45545, WO 1998/27212, WO 1998/40503, WO 99/58688, WO 1999/58690, WO 1999/58654, WO 2000/008184, WO 2000/008185, WO 2000/28052, WO 2000/77229, WO 2001/12782, WO 2001/12826, WO 2002/101059, WO 2003/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 2000/22140, WO 2006/063862, WO 2006/072603, WO 2002/034923, EP 06090134.5, EP 06090228.5, EP 06090227.7, EP 07090007.1, EP 07090009.7, WO 2001/14569, WO 2002/79410, WO 2003/33540, WO 2004/078983, WO 2001/19975, WO 1995/26407, WO 1996/34968, WO 1998/20145, WO 1999/12950, WO 1999/66050, WO 1999/53072, U.S. Pat. No. 6,734,341, WO 2000/11192, WO 1998/22604, WO 1998/32326, WO 2001/98509, WO 2001/98509, WO 2005/002359, U.S. Pat. No. 5,824,790, U.S. Pat. No. 6,013,861, WO 1994/004693, WO 1994/009144, WO 1994/11520, WO 1995/35026 and WO 1997/20936.
2) Transgenic plants which synthesize non-starch carbohydrate polymers or which synthesize non-starch carbohydrate polymers with altered properties in comparison to wild-type plants without genetic modification. Examples are plants producing polyfructose, especially of the inulin and levan type, as described in EP 0663956, WO 1996/001904, WO 1996/021023, WO 1998/039460 and WO 1999/024593, plants producing alpha-1,4-glucans, as described in WO 1995/031553, US 2002/031826, U.S. Pat. No. 6,284,479, U.S. Pat. No. 5,712,107, WO 1997/047806, WO 1997/047807, WO 1997/047808 and WO 2000/14249, plants producing alpha-1,6-branched alpha-1,4-glucans, as described in WO 2000/73422, and plants producing alternan, as described in WO 2000/047727, EP 06077301.7, U.S. Pat. No. 5,908,975 and EP 0728213.
3) Transgenic plants which produce hyaluronan, as for example described in WO 2006/032538, WO 2007/039314, WO 2007/039315, WO 2007/039316, JP 2006/304779 and WO 2005/012529.

Plants or plant cultivars (obtained by plant biotechnology methods such as genetic engineering) which may also be treated according to the invention are plants, such as cotton plants, with altered fiber characteristics. Such plants can be obtained by genetic transformation, or by selection of plants containing a mutation imparting such altered fiber characteristics and include:

a) plants, such as cotton plants, which contain an altered form of cellulose synthase genes, as described in WO 1998/000549;
b) plants, such as cotton plants, which contain an altered form of rsw2 or rsw3 homologous nucleic acids, as described in WO 2004/053219;
c) plants, such as cotton plants, with an increased expression of sucrose phosphate synthase, as described in WO 2001/017333;
d) plants, such as cotton plants, with an increased expression of sucrose synthase as described in WO 02/45485;
e) plants, such as cotton plants, wherein the timing of the plasmodesmatal gating at the basis of the fiber cell is altered, for example through downregulation of fiber-selective 13-1,3-glucanase, as described in WO 2005/017157;
f) plants, such as cotton plants, which have fibers with altered reactivity, for example through the expression of the N-acetylglucosaminetransferase gene including nodC and chitin synthase genes, as described in WO 2006/136351.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants containing a mutation imparting such altered oil characteristics and include:

a) plants, such as oilseed rape plants, which produce oil having a high oleic acid content, as described, for example, in U.S. Pat. No. 5,969,169, U.S. Pat. No. 5,840,946 or U.S. Pat. No. 6,323,392 or U.S. Pat. No. 6,063,947;
b) plants, such as oilseed rape plants, which produce oil having a low linolenic acid content, as described in U.S. Pat. No. 6,270,828, U.S. Pat. No. 6,169,190 or U.S. Pat. No. 5,965,755;
c) plants, such as oilseed rape plants, which produce oil having a low level of saturated fatty acids, as described, for example, in U.S. Pat. No. 5,434,283.

Particularly useful transgenic plants which can be treated in accordance with the invention are plants which comprise one or more genes which code for one or more toxins are the transgenic plants which are sold under the following trade names: YIELD GARD® (for example corn, cotton, soybeans), KnockOut® (for example corn), BiteGard® (for example corn), BT-Xtra® (for example corn), StarLink® (for example corn), Bollgard® (cotton), Nucotn® (cotton), Nucotn 33B® (cotton), NatureGard® (for example corn), Protecta® and NewLeaf® (potato). Examples of herbicide-tolerant plants include are corn varieties, cotton varieties and soya bean varieties which are available under the following trade names: Roundup Ready® (tolerance to glyphosates, for example corn, cotton, soybeans), Liberty Link® (tolerance to phosphinothricin, for example oilseed rape), IMI® (tolerance to imidazolinone) and SCS® (tolerance to sulfonylurea), for example corn. Herbicide-resistant plants (plants bred in a conventional manner for herbicide tolerance) which may be mentioned include the varieties sold under the name Clearfield® (for example corn).

Particularly useful transgenic plants which may be treated according to the invention are plants containing transformation events, or a combination of transformation events, and that are listed for example in the databases for various national or regional regulatory agencies.

The compounds of the general formula (I) to be used in accordance with the invention can be converted to customary formulations, such as solutions, emulsions, wettable powders, water- and oil-based suspensions, powders, dusts, pastes, soluble powders, soluble granules, granules for broadcasting, suspoemulsion concentrates, natural compounds impregnated with active ingredient, synthetic substances impregnated with active ingredient, fertilizers, and also microencapsulations in polymeric substances. In the context of the present invention, it is especially preferred when the inventive compounds of the general formula (I) are used in the form of a spray formulation.

The present invention therefore additionally also relates to a spray formulation for enhancing the resistance of plants to abiotic stress. A spray formulation is described in detail hereinafter:

The formulations for spray application are produced in a known manner, for example by mixing the compounds of the general formula (I) for use in accordance with the invention with extenders, i.e. liquid solvents and/or solid carriers, optionally with use of surfactants, i.e. emulsifiers and/or dispersants and/or foam formers. Further customary additives, for example customary extenders and solvents or diluents, dyes, wetting agents, dispersants, emulsifiers, antifoams, preservatives, secondary thickeners, stickers, gibberellins and also water, can optionally also be used. The formulations are produced either in suitable facilities or else before or during application.

The auxiliaries used may be those substances which are suitable for imparting, to the composition itself and/or to preparations derived therefrom (for example spray liquors), particular properties such as particular technical properties and/or else special biological properties. Typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender utilized is water, it is also possible to use, for example, organic solvents as auxiliary solvents. Useful liquid solvents essentially include: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulfoxide, and also water.

It is possible to use colorants such as inorganic pigments, for example iron oxide, titanium oxide and Prussian blue, and organic colorants such as alizarin colorants, azo colorants and metal phthalocyanine colorants, and trace nutrients such as salts of iron, manganese, boron, copper, cobalt, molybdenum and zinc.

Suitable wetting agents which may be present in the formulations which can be used in accordance with the invention are all substances which promote wetting and which are conventionally used for the formulation of agrochemical active substances. Preference is given to using alkyl naphthalenesulfonates, such as diisopropyl or diisobutyl naphthalenesulfonates.

Suitable dispersants and/or emulsifiers which may be present in the formulations which can be used in accordance with the invention are all nonionic, anionic and cationic dispersants conventionally used for the formulation of active agrochemical ingredients.

Preference is given to using nonionic or anionic dispersants or mixtures of nonionic or anionic dispersants. Suitable nonionic dispersants include in particular ethylene oxide/propylene oxide block polymers, alkylphenol polyglycol ethers and tristyrylphenol polyglycol ethers, and the phosphated or sulfated derivatives thereof. Suitable anionic dispersants are especially lignosulfonates, polyacrylic acid salts and arylsulfonate-formaldehyde condensates.

Suitable antifoams which may be present in the formulations usable in accordance with the invention are all foam-inhibiting substances conventionally used for the formulation of active agrochemical ingredients. Silicone antifoams and magnesium stearate can be used with preference.

Preservatives which may be present in the formulations usable in accordance with the invention are all substances usable for such purposes in agrochemical compositions. Examples include dichlorophene and benzyl alcohol hemiformal.

Secondary thickeners which may be present in the formulations usable in accordance with the invention are all substances usable for such purposes in agrochemical compositions. Preferred examples include cellulose derivatives, acrylic acid derivatives, xanthan, modified clays and finely divided silica.

Stickers which may be present in the formulations usable in accordance with the invention include all customary binders usable in seed-dressing products. Preferred examples include polyvinylpyrrolidone, polyvinyl acetate, polyvinyl alcohol and tylose.

Suitable gibberellins which may be present in the formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz- und Schädlingsbekämpfungsmittel” [Chemistry of Crop Protection and Pest Control Compositions], vol. 2, Springer Verlag, 1970, p. 401-412).

Further additives may be fragrances, mineral or vegetable, optionally modified oils, waxes and nutrients (including trace nutrients), such as salts of iron, manganese, boron, copper, cobalt, molybdenum and zinc. Additionally present may be stabilizers, such as cold stabilizers, antioxidants, light stabilizers or other agents which improve chemical and/or physical stability.

The formulations contain generally between 0.01% and 98% by weight, preferably between 0.5% and 90%, of the compound of the general formula (I).

The inventive active ingredient, in its commercially available formulations and in the use forms prepared from these formulations, may take the form of a mixture with other active ingredients, such as insecticides, attractants, sterilants, bactericides, acaricides, nematicides, fungicides, growth regulators, herbicides, safeners, fertilizers or semiochemicals.

In addition, the described positive effect of the compounds of the general formula (I) on the plants' own defenses can be supported by an additional treatment with active insecticidal, fungicidal or bactericidal ingredients.

Preferred times for the application of compounds of the general formula (I) for enhancing resistance to abiotic stress are treatments of the soil, stems and/or leaves with the approved application rates.

The inventive compounds of the general formula (I), in their commercial formulations and in the use forms prepared from these formulations, may generally additionally be present in mixtures with other active ingredients, such as insecticides, attractants, sterilants, acaricides, nematicides, fungicides, growth regulators, plant maturity regulators or safeners.

### Biological Examples

Seeds of monocotyledonous and dicotyledonous crop plants are sown in sandy loam in plastic or wood-fiber pots, covered with soil or sand and cultivated in a greenhouse under good growth conditions. The test plants are treated at the early leaf stage (BBCH10-BBCH13). To assure uniform water supply before commencement of stress, the potted plants are supplied with water by dam irrigation prior to substance application.

The inventive compounds, formulated in the form of wettable powders (WP), are sprayed onto the green parts of the plants as an aqueous suspension at an equivalent water application rate of 600 I/ha with addition of 0.2% wetting agent (e.g. agrotin). Substance application is followed immediately by stress treatment of the plants. For this purpose, the wood-fiber pots are transferred in plastic inserts in order to prevent them from subsequently drying out too quickly.

Drought stress is induced by gradual drying out under the following conditions:

“Day”: 14 hours with illumination at 26-30° C.
“Night”: 10 hours without illumination at 18-20° C.

The duration of the respective stress phases is guided mainly by the condition of the stressed control plants. It is ended (by re-irrigating and transfer to a greenhouse with good growth conditions) as soon as irreversible damage is observed on the stressed control plants.

The end of the stress phase is followed by an approx. 4-14-day recovery phase, during which the plants are once again kept under good growth conditions in a greenhouse. The duration of the recovery phase is guided mainly by when the trial plants have attained a state which enables visual scoring of potential effects, and is therefore variable.

Once this juncture has been reached, the intensities of damage are scored visually in comparison to untreated, unstressed controls of the same age. The damage intensity is at first assessed in percent. These values are then used to calculate the efficacy of the test compounds by the following formula:

$$E = \frac{( {DI}_{s} - {DI}_{t} ) \times 100}{{DI}_{s}}$$

E: Efficacy=reduction in damage intensity as a result of treatment with test substance
DIₛ: Damage intensity of the stressed control plants
DIₜ: Damage intensity of the plants treated with test compound

The values reported in tables 1-4 below are averages from at least one trial with at least two repeats.

### Effects of Selected Compounds of the General Formula (I)

### Under Cold Stress (Table 1):

TABLE 1

| | | | | E:E |
|---|---|---|---|---|
| No. | Ex. | Dosage | Unit | (ZEAMX) |
| 1 | 2223 | 25 | g/ha | >5 |
| 2 | 265 | 10 | g/ha | >5 |
| 3 | 2128 | 250 | g/ha | >5 |

Effects of Selected Compounds of the General Formula (I) Under Drought Stress (Tables 2 to 4):

TABLE 2

| No. | Ex. | Dosage | Unit | E (ZEAMX) |
|---|---|---|---|---|
| 1 | 1212 | 25 | g/ha | >5 |
| 2 | 2223 | 250 | g/ha | >5 |
| 3 | 1578 | 250 | g/ha | >5 |
| 4 | 221 | 250 | g/ha | >5 |
| 5 | 777 | 25 | g/ha | >5 |
| 6 | 222 | 250 | g/ha | >5 |
| 7 | 223 | 25 | g/ha | >5 |
| 8 | 664 | 25 | g/ha | >5 |
| 9 | 696 | 25 | g/ha | >5 |
| 10 | 2085 | 250 | g/ha | >5 |
| 11 | 2128 | 25 | g/ha | >5 |
| 12 | 170 | 250 | g/ha | >5 |
| 13 | 3 | 250 | g/ha | >5 |
| 14 | 36 | 250 | g/ha | >5 |
| 15 | 219 | 250 | g/ha | >5 |
| 16 | 46 | 25 | g/ha | >5 |
| 17 | 266 | 25 | g/ha | >5 |
| 18 | 1159 | 250 | g/ha | >5 |
| 19 | 2270 | 25 | g/ha | >5 |
| 20 | 2817 | 25 | g/ha | >5 |
| 21 | 3920 | 25 | g/ha | >5 |
| 22 | 3940 | 25 | g/ha | >5 |
| 23 | 610 | 25 | g/ha | >5 |
| 24 | 2423 | 2.5 | g/ha | >5 |
| 25 | 59 | 25 | g/ha | >5 |
| 26 | 2290 | 250 | g/ha | >5 |

TABLE 3

| | | | | E |
|---|---|---|---|---|
| No. | Ex. | Dosage | Unit | (BRSNS) |
| 1 | 1785 | 50 | g/ha | >5 |
| 2 | 1565 | 250 | g/ha | >5 |
| 3 | 2225 | 250 | g/ha | >5 |
| 4 | 1688 | 250 | g/ha | >5 |
| 5 | 225 | 25 | g/ha | >5 |
| 6 | 233 | 25 | g/ha | >5 |
| 7 | 247 | 25 | g/ha | >5 |
| 8 | 256 | 25 | g/ha | >5 |
| 9 | 258 | 25 | g/ha | >5 |
| 10 | 665 | 250 | g/ha | >5 |
| 11 | 2016 | 25 | g/ha | >5 |
| 12 | 2020 | 25 | g/ha | >5 |
| 13 | 813 | 25 | g/ha | >5 |
| 14 | 2220 | 25 | g/ha | >5 |
| 15 | 2233 | 25 | g/ha | >5 |
| 16 | 1154 | 250 | g/ha | >5 |
| 17 | 2246 | 25 | g/ha | >5 |
| 18 | 29 | 250 | g/ha | >5 |
| 19 | 32 | 250 | g/ha | >5 |
| 20 | 13 | 250 | g/ha | >5 |
| 21 | 2251 | 250 | g/ha | >5 |
| 22 | 204 | 25 | g/ha | >5 |
| 23 | 1744 | 25 | g/ha | >5 |
| 24 | 46 | 250 | g/ha | >5 |
| 25 | 266 | 25 | g/ha | >5 |
| 26 | 933 | 25 | g/ha | >5 |

TABLE 4

| No. | Ex. | Dosage | Unit | E (TRZAS) |
|---|---|---|---|---|
| 1 | 243 | 250 | g/ha | >5 |
| 2 | 272 | 25 | g/ha | >5 |
| 3 | 274 | 250 | g/ha | >5 |
| 4 | 265 | 250 | g/ha | >5 |
| 5 | 266 | 25 | g/ha | >5 |
| 6 | 933 | 250 | g/ha | >5 |
| 7 | 4027 | 250 | g/ha | >5 |
| 8 | 1084 | 250 | g/ha | >5 |
| 9 | 770 | 25 | g/ha | >5 |
| 10 | 2840 | 250 | g/ha | >5 |
| 11 | 3639 | 25 | g/ha | >5 |
| 12 | 3920 | 25 | g/ha | >5 |
| 13 | 3940 | 25 | g/ha | >5 |
| 14 | 3699 | 25 | g/ha | >5 |
| 15 | 3807 | 25 | g/ha | >5 |
| 16 | 3917 | 25 | g/ha | >5 |
| 17 | 3170 | 25 | g/ha | >5 |
| 18 | 3149 | 25 | g/ha | >5 |
| 19 | 2581 | 250 | g/ha | >5 |

In the above tables:

Ex.=compound corresponding to Ex. from table 1
BRSNS=Brassica napus
TRZAS=Triticum aestivum
ZEAMX=Zea mays (corn)

Similar results were also achievable with further compounds of the general formula (I), even on application to different plant species.

1. A substituted pyridonecarboxamide of formula (I) and/or a salt thereof 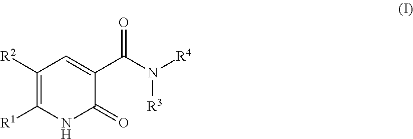
for increasing tolerance to abiotic stress in plants, where
R¹ is (C₁-C₆)-alkyl, (C₂-C₆)-alkenyl, (C₂-C₆)-alkynyl, (C₁-C₆)-haloalkyl, (C₂-C₆)-haloalkenyl, (C₂-C₆)-haloalkynyl, (C₃-C₆)-cycloalkyl, (C₃-C₆)-cycloalkyl-(C₁-C₆)-alkyl, (C₁-C₆)-alkoxy, (C₂-C₆)-alkenyloxy, (C₂-C₆)-alkynyloxy, (C₁-C₆)-haloalkoxy, (C₂-C₆)-haloalkenyloxy, (C₂-C₆)-haloalkynyloxy, cyano, halogen or optionally mono- or polysubstituted phenyl,
R² is (C₁-C₆)-alkyl, (C₂-C₆)-alkenyl, (C₂-C₆)-alkynyl, (C₁-C₆)-haloalkyl, (C₂-C₆)-haloalkenyl, (C₂-C₆)-haloalkynyl, (C₃-C₆)-cycloalkyl, (C₃-C₆)-cycloalkyl-(C₁-C₆)-alkyl, (C₁-C₆)-alkoxy, (C₂-C₆)-alkenyloxy, (C₂-C₆)-alkynyloxy, (C₁-C₆)-haloalkoxy, (C₂-C₆)-haloalkenyloxy, (C₂-C₆)-haloalkynyloxy, cyano, halogen or optionally mono- or polysubstituted phenyl,
R³ and R⁴ are each independently hydrogen, (C₁-C₆)-alkyl, (C₂-C₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
or
are (C₃-C₆)-cycloalkyl, (C₄-C₆)cycloalkenyl, (C₃-C₆)-cycloalkyl fused on one side of the ring to a 4- to 6-membered saturated or unsaturated carbocyclic ring, or (C₄-C₆)-cycloalkenyl fused on one side of the ring to a 4- to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
or
are phenyl which is unsubstituted or substituted, heteroaryl which is unsubstituted or substituted and heterocyclyl which is unsubstituted or substituted,
or
R³ is hydrogen or (C₁-C₆)-alkyl
and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
or
R³ and R⁴ together with the directly bonded nitrogen atom are an amino acid residue, specifically those which occur naturally in their racemic and respective D and L forms,
or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, hydroxycarbonyl, [(C₁-C₄)-alkoxy]-carbonyl and (C₁-C₄)-alkylthio,
or
R³ and R⁴ together with the directly bonded nitrogen atom are the —N═CR⁵—NR⁶R⁷ group where
R⁵ is hydrogen or (C₁-C₆)-alkyl and
R⁶ and R⁷ are each independently hydrogen or (C₁-C₆)-alkyl, or R⁶, R⁷ together with the directly bonded nitrogen atom form a five- to seven-membered, preferably saturated, heterocyclic ring, for example piperidinyl, pyrrolidinyl or morpholinyl.

2. The compound as claimed in claim 1, where, in formula (I),
R¹ is (C₁-C₆)-alkyl, (C₁-C₆)-haloalkyl, (C₃-C₆)-cycloalkyl, (C₁-C₆)-alkoxy or (C₁-C₆)-haloalkoxy,
R² is (C₁-C₆)-alkyl, (C₁-C₆)-haloalkyl, (C₃-C₆)-cycloalkyl, (C₁-C₆)-alkoxy, (C₁-C₆)-haloalkoxy or halogen,
R³ and R⁴ are each independently hydrogen, (C₁-C₁₆)-alkyl, (C₂-C₁₆)-alkenyl or (C₂-C₁₆)-alkynyl, where each of the 3 latter radicals unsubstituted or by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
or
are (C₃-C₆)-cycloalkyl, (C₄-C₆)cycloalkenyl, (C₃-C₆)-cycloalkyl fused on one side of the ring to a 4- to 6-membered saturated or unsaturated carbocyclic ring, or (C₄-C₆)-cycloalkenyl fused on one side of the ring to a 4- to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
or
are phenyl which is unsubstituted or substituted, heteroaryl which is unsubstituted or substituted and heterocyclyl which is unsubstituted or substituted,
or
R³ is hydrogen or (C₁-C₆)-alkyl
and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
or
R³ and R⁴ together with the directly bonded nitrogen atom are an amino acid residue, specifically those which occur naturally in their racemic and respective D and L forms,
or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, hydroxycarbonyl, [(C₁-C₄)-alkoxy]-carbonyl and (C₁-C₄)-alkylthio
or
R³ and R⁴ together with the directly bonded nitrogen atom are the —N═CR⁵—NR⁶R⁷ group and where
R⁵ is hydrogen or (C₁-C₆)-alkyl and
R⁶ and R⁷ are each independently hydrogen or (C₁-C₆)-alkyl, or
R⁶ and R⁷ together with the directly bonded nitrogen atom form a five- to seven-membered, preferably saturated, heterocyclic ring, for example piperidinyl, pyrrolidinyl or morpholinyl.

3. The compound and/or salt as claimed in claim 1, where, in formula (I),
R¹ is (C₁-C₆)-alkyl or (C₁-C₆)-haloalkyl,
R² is (C₁-C₆)-alkyl or halogen,
R³ and R⁴ are each independently hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
or
are (C₃-C₆)-cycloalkyl, (C₄-C₆)cycloalkenyl, (C₃-C₆)-cycloalkyl fused on one side of the ring to a 4- to 6-membered saturated or unsaturated carbocyclic ring, or (C₄-C₆)-cycloalkenyl fused on one side of the ring to a 4- to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]-amino, [(C₁-C₄)-alkoxy]-carbonyl, [(C₁-C₄)-haloalkoxy]-carbonyl, (C₃-C₆)-cycloalkyl which is unsubstituted or substituted, phenyl which is unsubstituted or substituted, heteroaryl which is unsubstituted or substituted and heterocyclyl which is unsubstituted or substituted,
or
are phenyl which is unsubstituted or substituted, heteroaryl which is unsubstituted or substituted and heterocyclyl which is unsubstituted or substituted,
or
R³ is hydrogen or (C₁-C₆)-alkyl
and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
or
R³ and R⁴ together with the directly bonded nitrogen atom are an amino acid residue, specifically those which occur naturally in their racemic and respective D and L forms,
or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy, hydroxycarbonyl, [(C₁-C₄)-alkoxy]-carbonyl and (C₁-C₄)-alkylthio.
or
R³ and R⁴ together with the directly bonded nitrogen atom are the —N═CR⁵—NR⁶R⁷ group and where
R⁵ is hydrogen or (C₁-C₆)-alkyl and
R⁶ and R⁷ are each independently hydrogen or (C₁-C₆)-alkyl, or R⁶, R⁷ together with the directly bonded nitrogen atom form a five- to seven-membered, preferably saturated, heterocyclic ring, for example piperidinyl, pyrrolidinyl or morpholinyl.

4. A treatment for one or more plants, comprising applying a nontoxic amount, effective for increasing the resistance of plants to abiotic stress factors, of one or more of the compounds of the formula (I) and/or a respective salt as claimed in claim 1.

5. The treatment as claimed in claim 4, wherein the abiotic stress conditions are one or more conditions selected from the group consisting of aridity, cold stress, heat stress, drought stress, osmotic stress, waterlogging, elevated soil salinity, elevated exposure to minerals, ozone conditions, strong light conditions, limited availability of nitrogen nutrients and limited availability of phosphorus nutrients.

6. The compound and/or salt as claimed in claim 1 capable of being used in spray application to one or more plants and/or plant parts in combinations with one or more active ingredients selected from the group consisting of insecticides, attractants, acaricides, fungicides, nematicides, growth regulators, safeners, plant maturity regulators and bactericides.

7. A compound of formula (I) and/or salt as claimed in claim 1, capable of being used in spray application to one or more plants and/or parts of plants in combination with one or more fertilizers.

8. A compound of formula (I) and/or salt as claimed in claim 1, capable of being used for application to one or more genetically modified cultivars, seed thereof, and/or to one or more cultivated areas on which these-cultivars grow.

9. A spray solution comprising one or more compounds of the formula (I) and/or salts as claimed in claim 1, capable of being used for enhancing resistance of one or more plants to one or more abiotic stress factors.

10. A method for increasing stress tolerance in one or more plants selected from the group of useful plants, ornamental plants, turfgrasses and trees, comprising applying a sufficient nontoxic amount of one or more compounds of the formula (I) and/or salts as claimed in claim 1 to an area where the corresponding effect is desired, and/or comprising application to the plants, seed thereof and/or to an area on which the plants grow.

11. The method as claimed in claim 10, wherein the resistance of the plants thus treated to abiotic stress is increased by at least 3% compared to untreated plants under otherwise identical physiological conditions.

12. A substituted pyridonecarboxamide of formula (I) and/or salt thereof 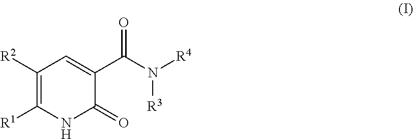
in which
R¹ is (C₁-C₆)-alkyl or (C₁-C₆)-haloalkyl,
R² is (C₁-C₆)-alkyl or halogen,
R³ is hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted,
or
is (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
or
is phenyl which is unsubstituted or substituted, heteroaryl which is unsubstituted or substituted and heterocyclyl which is unsubstituted or substituted
and
R⁴ is (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]-amino, hydroxycarbonyl, [(C₁-C₄)-alkoxy]-carbonyl, [(C₁-C₄)-haloalkoxy]-carbonyl, (C₃-C₆)-cycloalkyl, phenyl which is unsubstituted or substituted, heteroaryl which is unsubstituted or substituted and heterocyclyl which is unsubstituted or substituted,
or
is (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
or
is phenyl which is unsubstituted or substituted, heteroaryl which is unsubstituted or substituted and heterocyclyl which is unsubstituted or substituted
or
R³ is hydrogen or (C₁-C₆)-alkyl
and
R⁴ is (C₁-C₆)-alkyl, (C₁-C₆)-alkoxy, (C₂-C₆)-alkenyloxy, (C₂-C₆)-alkynyloxy or (C₂-C₆)-haloalkoxy or (C₁-C₆)-alkyl-SO₂,
or
R³ and R⁴ together with the directly bonded nitrogen atom are an amino acid residue, specifically those which occur naturally in their racemic and respective D and L forms,
or
R³ and R⁴ together with the directly bonded nitrogen atom form a four- to eight-membered heterocyclic ring which, in addition to the nitrogen atom, may also contain further ring heteroatoms, preferably up to two further ring heteroatoms from the group of N, O and S, and which unsubstituted or mean by one or more radicals from the group of halogen, cyano, nitro, (C₁-C₄)-alkyl, (C₁-C₄)-haloalkyl, (C₁-C₄)-alkoxy, (C₁-C₄)-haloalkoxy, hydroxycarbonyl, [(C₁-C₄)-alkoxy]-carbonyl and (C₁-C₄)-alkylthio,
or
R³ and R⁴ together with the directly bonded nitrogen atom are the —N═CR⁵—NR⁶R⁷ group
R⁵ is hydrogen or (C₁-C₆)-alkyl and
R⁶ and R⁷ are each independently hydrogen or (C₁-C₆)-alkyl,
or
R⁶ and R⁷ together with the directly bonded nitrogen atom form a five- to seven-membered, preferably saturated, heterocyclic ring, for example piperidinyl, pyrrolidinyl or morpholinyl,
excluding compounds of the general formula (I) in which
(a) R¹ and R² are methyl, R³ is hydrogen and R⁴ is benzyl, or
(b) when R¹ is (C₁-C₆)-haloalkyl and R² is simultaneously halogen.

13. A spray solution for treatment of one or more plants, comprising an amount, effective for increasing the resistance of plants to one or more abiotic stress factors, of one or more of the substituted pyridonecarboxamides and/or salts thereof as claimed in claim 12.
